SEQUENCE LISTING (117 sequences)

SEQ ID NO: 1 - protein, 20 aa, Veillonella parvula
APFYRIVVADARAPREGRPV

SEQ ID NO: 2 - protein, 21 aa, Veillonella parvula
FEAKVLEIDEKKNRLLVLSRR

SEQ ID NO: 3 - protein, 15 aa, Severe acute respiratory syndrome-related coronavirus
TEGALNTPKDHIGTR

SEQ ID NO: 4 - protein, 15 aa, Severe acute respiratory syndrome-related coronavirus
KEGAKTVNTSLGNRK

SEQ ID NO: 5 - protein, 15 aa, Severe acute respiratory syndrome-related coronavirus
PINTNSSPDDQIGYY

SEQ ID NO: 6 - protein, 15 aa, Severe acute respiratory syndrome-related coronavirus
PIGKGNKDEQIGYWN

SEQ ID NO: 7 - protein, 15 aa, Severe acute respiratory syndrome-related coronavirus
GLPNNTASWFTALTQ

SEQ ID NO: 8 - protein, 15 aa, Severe acute respiratory syndrome-related coronavirus
GNTIPHYSWFSGITQ

SEQ ID NO: 9 - protein, 15 aa, Severe acute respiratory syndrome-related coronavirus
QKKQQTVTLLPAADL

SEQ ID NO: 10 - protein, 15 aa, Severe acute respiratory syndrome-related coronavirus
KRGVKQSPESFDSLN

SEQ ID NO: 11 - protein, 60 aa, Severe acute respiratory syndrome-related coronavirus
LPYGANKDGIIWVATEGALNTPKDHIGTRNPANNAAIVLQLPQGTTLPKGFYAEGSRGGS

SEQ ID NO: 12 - DNA, 39 bp, synthetic construct
  misc_feature 1..39: note = Synthetic
  source 1..39: mol_type = other DNA
tatgccgcgtttgtacagacgcatagaccaacaggggag

SEQ ID NO: 13 - DNA, 40 bp, synthetic construct
  misc_feature 1..40: note = Synthetic
  source 1..40: mol_type = other DNA
tatgccgcgttttgtacagacgcatagaccaacaggggag

SEQ ID NO: 14 - DNA, 25 bp, synthetic construct
  misc_feature 1..25: note = Synthetic
  source 1..25: mol_type = other DNA
tatgccgcgtagaccaacaggggag

SEQ ID NO: 15 - protein, 52 aa, synthetic construct
  REGION 1..52: note = Synthetic
MRPEDRMFHIRAVILRALSLAFLLSLRGAGAIKADHVSTYAAFVQTHRPTGE

SEQ ID NO: 16 - protein, 47 aa, synthetic construct
  REGION 1..47: note = Synthetic
MRPEDRMFHIRAVILRALSLAFLLSLRGAGAIKADHVSTYAAFCTDA

SEQ ID NO: 17 - protein, 42 aa, synthetic construct
  REGION 1..42: note = Synthetic
MRPEDRMFHIRAVILRALSLAFLLSLRGAGAIKADHVSTYAA

SEQ ID NO: 18 - DNA, 39 bp, synthetic construct
  misc_feature 1..39: note = Synthetic
  source 1..39: mol_type = other DNA
gaccacgttgcctcttgtggtgtaaacttgtaccagttt

SEQ ID NO: 19 - DNA, 40 bp, synthetic construct
  misc_feature 1..40: note = Synthetic
  source 1..40: mol_type = other DNA
gaccacgtttgcctcttgtggtgtaaacttgtaccagttt

SEQ ID NO: 20 - protein, 40 aa, synthetic construct
  REGION 1..40: note = Synthetic
MILNKALLLGALATTVMSPCGGEDIVADHVASCGVNLYQF

SEQ ID NO: 21 - protein, 49 aa, synthetic construct
  REGION 1..49: note = Synthetic
MILNKALLLGALATTVMSPCGGEDIVADHVCLLWCKLVPVLRSLWPVHP

SEQ ID NO: 22 - protein, 10 aa, synthetic construct
  REGION 1..10: note = Synthetic
CCCDPGRCMC

SEQ ID NO: 23 - protein, 10 aa, synthetic construct
  REGION 1..10: note = Synthetic
CCLCRCWCCR

SEQ ID NO: 24 - protein, 10 aa, synthetic construct
  REGION 1..10: note = Synthetic
CCLCRCQLTC

SEQ ID NO: 25 - protein, 10 aa, synthetic construct
  REGION 1..10: note = Synthetic
CRGCRGQCCI

SEQ ID NO: 26 - protein, 10 aa, synthetic construct
  REGION 1..10: note = Synthetic
GYTCLNYCCV

SEQ ID NO: 27 - protein, 10 aa, synthetic construct
  REGION 1..10: note = Synthetic
CCMCCLCCCC

SEQ ID NO: 28 - protein, 10 aa, synthetic construct
  REGION 1..10: note = Synthetic
CCCCCCCCCC

SEQ ID NO: 29 - protein, 10 aa, synthetic construct
  REGION 1..10: note = Synthetic
CLLCKWCCFC

SEQ ID NO: 30 - protein, 10 aa, synthetic construct
  REGION 1..10: note = Synthetic
KYQQPYNNPF

SEQ ID NO: 31 - protein, 10 aa, synthetic construct
  REGION 1..10: note = Synthetic
CMYFCSGLHC

SEQ ID NO: 32 - protein, 10 aa, synthetic construct
  REGION 1..10: note = Synthetic
CCFKPRGCCC

SEQ ID NO: 33 - protein, 10 aa, synthetic construct
  REGION 1..10: note = Synthetic
CCDWGSCQCH

SEQ ID NO: 34 - protein, 10 aa, synthetic construct
  REGION 1..10: note = Synthetic
PGHYNCCCCC

SEQ ID NO: 35 - protein, 10 aa, synthetic construct
  REGION 1..10: note = Synthetic
CPCYFQCKVC

SEQ ID NO: 36 - protein, 10 aa, synthetic construct
  REGION 1..10: note = Synthetic
CCCPEGCCCC

SEQ ID NO: 37 - protein, 10 aa, synthetic construct
  REGION 1..10: note = Synthetic
CRPPSGYPCC

SEQ ID NO: 38 - protein, 10 aa, synthetic construct
  REGION 1..10: note = Synthetic
CFGCLYHCCC

SEQ ID NO: 39 - protein, 10 aa, synthetic construct
  REGION 1..10: note = Synthetic
RSRCAQCPCM

SEQ ID NO: 40 - protein, 10 aa, synthetic construct
  REGION 1..10: note = Synthetic
CCWMGYCCIC

SEQ ID NO: 41 - protein, 10 aa, synthetic construct
  REGION 1..10: note = Synthetic
CCPKGKPCCY

SEQ ID NO: 42 - protein, 10 aa, synthetic construct
  REGION 1..10: note = Synthetic
CMCEFCGCCE

SEQ ID NO: 43 - protein, 10 aa, synthetic construct
  REGION 1..10: note = Synthetic
CCQESYGTHP

SEQ ID NO: 44 - protein, 10 aa, synthetic construct
  REGION 1..10: note = Synthetic
CMMPAYQCCM

SEQ ID NO: 45 - protein, 10 aa, synthetic construct
  REGION 1..10: note = Synthetic
CMLCCMDCCI

SEQ ID NO: 46 - protein, 10 aa, synthetic construct
  REGION 1..10: note = Synthetic
CTCCTWGHCC

SEQ ID NO: 47 - protein, 10 aa, synthetic construct
  REGION 1..10: note = Synthetic
CCIVDWPMCC

SEQ ID NO: 48 - protein, 10 aa, synthetic construct
  REGION 1..10: note = Synthetic
QTTCCITISR

SEQ ID NO: 49 - protein, 10 aa, synthetic construct
  REGION 1..10: note = Synthetic
MKCCRVPCGC

SEQ ID NO: 50 - protein, 10 aa, synthetic construct
  REGION 1..10: note = Synthetic
CKTCCCDEAC

SEQ ID NO: 51 - protein, 10 aa, synthetic construct
  REGION 1..10: note = Synthetic
VENHCCCIIC

SEQ ID NO: 52 - protein, 10 aa, synthetic construct
  REGION 1..10: note = Synthetic
CVRWSCTIHW

SEQ ID NO: 53 - protein, 10 aa, synthetic construct
  REGION 1..10: note = Synthetic
SHCIPDYKGY

SEQ ID NO: 54 - protein, 10 aa, synthetic construct
  REGION 1..10: note = Synthetic
NQTSKDDWGC

SEQ ID NO: 55 - protein, 10 aa, synthetic construct
  REGION 1..10: note = Synthetic
WHGSMDPCGP

SEQ ID NO: 56 - protein, 10 aa, synthetic construct
  REGION 1..10: note = Synthetic
DTTTVRDHQN

SEQ ID NO: 57 - protein, 10 aa, synthetic construct
  REGION 1..10: note = Synthetic
MNWTTICANQ

SEQ ID NO: 58 - protein, 10 aa, synthetic construct
  REGION 1..10: note = Synthetic
NHTAQMNFDW

SEQ ID NO: 59 - protein, 10 aa, synthetic construct
  REGION 1..10: note = Synthetic
NTTHNMYEGA

SEQ ID NO: 60 - protein, 10 aa, synthetic construct
  REGION 1..10: note = Synthetic
TTDDNAHQQN

SEQ ID NO: 61 - protein, 10 aa, synthetic construct
  REGION 1..10: note = Synthetic
NDTCEDCPQW

SEQ ID NO: 62 - protein, 10 aa, synthetic construct
  REGION 1..10: note = Synthetic
TIHIENCNIM

SEQ ID NO: 63 - protein, 10 aa, synthetic construct
  REGION 1..10: note = Synthetic
DFPGQDYVGP

SEQ ID NO: 64 - protein, 10 aa, synthetic construct
  REGION 1..10: note = Synthetic
DFSCVCQVYY

SEQ ID NO: 65 - protein, 10 aa, synthetic construct
  REGION 1..10: note = Synthetic
DYPGGNFCCY

SEQ ID NO: 66 - protein, 10 aa, synthetic construct
  REGION 1..10: note = Synthetic
SHPDHQGKDW

SEQ ID NO: 67 - protein, 11 aa, synthetic construct
  REGION 1..11: note = Synthetic
TVIWDCCMHHH

SEQ ID NO: 68 - protein, 10 aa, synthetic construct
  REGION 1..10: note = Synthetic
TYDDWGGEKY

SEQ ID NO: 69 - protein, 10 aa, synthetic construct
  REGION 1..10: note = Synthetic
YLQCCVSPHK

SEQ ID NO: 70 - protein, 10 aa, synthetic construct
  REGION 1..10: note = Synthetic
TVYDCLVRCV

SEQ ID NO: 71 - protein, 10 aa, synthetic construct
  REGION 1..10: note = Synthetic
TAYDYMNKDP

SEQ ID NO: 72 - protein, 10 aa, synthetic construct
  REGION 1..10: note = Synthetic
DFMQCRVHQT

SEQ ID NO: 73 - protein, 10 aa, synthetic construct
  REGION 1..10: note = Synthetic
CMMYSQKHHD

SEQ ID NO: 74 - protein, 10 aa, synthetic construct
  REGION 1..10: note = Synthetic
CLRPMQEQLG

SEQ ID NO: 75 - protein, 10 aa, synthetic construct
  REGION 1..10: note = Synthetic
RSPRVYGHCG

SEQ ID NO: 76 - protein, 10 aa, synthetic construct
  REGION 1..10: note = Synthetic
DCCPNYDPHG

SEQ ID NO: 77 - protein, 10 aa, synthetic construct
  REGION 1..10: note = Synthetic
VGDHPNNDKG

SEQ ID NO: 78 - protein, 10 aa, synthetic construct
  REGION 1..10: note = Synthetic
DDHGFHNGGN

SEQ ID NO: 79 - protein, 10 aa, synthetic construct
  REGION 1..10: note = Synthetic
PDQPPTGNFP

SEQ ID NO: 80 - protein, 10 aa, synthetic construct
  REGION 1..10: note = Synthetic
PFPWNTTQDP

SEQ ID NO: 81 - protein, 10 aa, synthetic construct
  REGION 1..10: note = Synthetic
KYLRLGKCYR

SEQ ID NO: 82 - protein, 10 aa, synthetic construct
  REGION 1..10: note = Synthetic
KRIRTVKDHK

SEQ ID NO: 83 - protein, 10 aa, synthetic construct
  REGION 1..10: note = Synthetic
RPNQDNKDYC

SEQ ID NO: 84 - protein, 10 aa, synthetic construct
  REGION 1..10: note = Synthetic
PHNQTTTDDP

SEQ ID NO: 85 - protein, 10 aa, synthetic construct
  REGION 1..10: note = Synthetic
RDERTFCIRC

SEQ ID NO: 86 - protein, 5 aa, synthetic construct
  REGION 1..5: note = Synthetic
SGGSA

SEQ ID NO: 87 - protein, 8 aa, synthetic construct
  REGION 1..8: note = Synthetic
WSHPQFEK

SEQ ID NO: 88 - protein, 14 aa, synthetic construct
  REGION 1..14: note = Synthetic
GKPIPNPLLGLDST

SEQ ID NO: 89 - DNA, 20 bp, synthetic construct
  misc_feature 1..20: note = Synthetic
  source 1..20: mol_type = other DNA
cgttgcctcttgtggtgtaa

SEQ ID NO: 90 - DNA, 19 bp, synthetic construct
  misc_feature 1..19: note = Synthetic
  source 1..19: mol_type = other DNA
cgtttgtacagacgcatag

SEQ ID NO: 91 - protein, 15 aa, synthetic construct
  REGION 1..15: note = Synthetic
PVSKMRMATPLLMQA

SEQ ID NO: 92 - protein, 15 aa, synthetic construct
  REGION 1..15: note = Synthetic
IQHLQKDYRAYYTFL

SEQ ID NO: 93 - protein, 12 aa, synthetic construct
  REGION 1..12: note = Synthetic
GGGGTSGGSGGS

SEQ ID NO: 94 - protein, 29 aa, synthetic construct
  REGION 1..29: note = Synthetic
MVCLKFPGGSCMAALTVTLMVLSSPLALA

SEQ ID NO: 95 - protein, 20 aa, Veillonella parvula
NAAVVIKDDKTPRGTRIFGP

SEQ ID NO: 96 - protein, 18 aa, Veillonella parvula
DENAAVVIKDDKTPRGTR

SEQ ID NO: 97 - protein, 9 aa, Veillonella parvula
VIKDDKTPR

SEQ ID NO: 98 - protein, 20 aa, Veillonella parvula
APFYRIVVADARAPREGRPV

SEQ ID NO: 99 - protein, 17 aa, Veillonella parvula
YRIVVADARAPREGRPV

SEQ ID NO: 100 - protein, 9 aa, Veillonella parvula
VVADARAPR

SEQ ID NO: 101 - protein, 20 aa, Ruminococcus gnavus
FTNKKLIVDIKEVKRPDKDA

SEQ ID NO: 102 - protein, 17 aa, Ruminococcus gnavus
TNKKLIVDIKEVKRPDK

SEQ ID NO: 103 - protein, 9 aa, Ruminococcus gnavus
LIVDIKEVK

SEQ ID NO: 104 - protein, 20 aa, Bifidobacterium longum
YKPVLRLDADGKEVRTDENN

SEQ ID NO: 105 - protein, 19 aa, Bifidobacterium longum
TDENNFVVDLHLERIDHPQ

SEQ ID NO: 106 - protein, 9 aa, Bifidobacterium longum
LDADGKEVR

SEQ ID NO: 107 - protein, 20 aa, Bifidobacterium longum
FVDGRVSIEVDPRLAHDTEN

SEQ ID NO: 108 - protein, 17 aa, Bifidobacterium longum
GRVSIEVDPRLAHDTEN

SEQ ID NO: 109 - protein, 9 aa, Bifidobacterium longum
IEVDPRLAH

SEQ ID NO: 110 - protein, 20 aa, Ruminococcus gnavus
TKGEIYVDGHEVRKEPEKVR

SEQ ID NO: 111 - protein, 20 aa, Ruminococcus gnavus
RPTKGEIYVDGHEVRKEPEK

SEQ ID NO: 112 - protein, 9 aa, Ruminococcus gnavus
IYVDGHEVR

SEQ ID NO: 113 - protein, 20 aa, Veillonella parvula
FEAKVLEIDEKKNRLVLSRR

SEQ ID NO: 114 - protein, 14 aa, Veillonella parvula
EAKVLEIDEKKNRL

SEQ ID NO: 115 - protein, 9 aa, Veillonella parvula
LEIDEKKNR

SEQ ID NO: 116 - protein, 20 aa, Veillonella parvula
GDVVKAVIVRSKKGIRRQDG

SEQ ID NO: 117 - protein, 9 aa, Veillonella parvula
IVRSKKGIR

(19) United States
(12) Patent Application Publication
(10) Pub. No.: US 2025/0226057 A1
(43) Pub. Date: Jul. 10, 2025

(54) HLA-II IMMUNOPEPTIDOME METHODS AND SYSTEMS FOR ANTIGEN DISCOVERY

(71) Applicants: The Broad Institute, Inc., Cambridge, MA (US); The General Hospital Corporation, Boston, MA (US)

(72) Inventors: Ramnik Xavier, Cambridge, MA (US); Daniel Graham, Cambridge, MA (US); Martin Strazar, Cambridge, MA (US)

(21) Appl. No.: 19/016,181

(22) Filed: Jan. 10, 2025

Related U.S. Application Data
(63) Continuation of application No. PCT/US2023/070102, filed on Jul. 13, 2023.
(60) Provisional application No. 63/368,344, filed on Jul. 13, 2022.

(51) Int. Cl. G16B 20/30; G06F 30/27; G16B 40/20; G16B 40/30

(52) U.S. Cl. CPC G16B 20/30; G06F 30/27; G16B 40/20; G16B 40/30

(57) ABSTRACT

T cell responses are exquisitely antigen-specific and directed against peptide epitopes displayed by human leukocyte antigen (HLA) on the surface of presenting cells. In particular, class II HLA (HLA-II) is remarkably polymorphic, which allows for presentation of diverse peptide antigens to T cells, but also forms the basis for genetic associations with diverse immunopathologies across the spectrum of infectious disease and autoimmunity. Here, Applicants employ monoallelic immunopeptidomics to retrieve over 200,000 unique peptides presented by 41 HLA-II heterodimers covering major alleles across diverse ancestries. Applicants leveraged this expansive dataset to develop computational models that predict peptide antigens based on HLA-II binding properties and infer informative features of the protein antigens from which these peptides derive. Combining both peptide and (contextual) protein features, Applicants develop Context Aware Predictor of T cell Antigens (CAPTAn) to discover novel T cell epitopes from prokaryotes in the human microbiome and the viral pandemic pathogen SARS-COV-2.

### CROSS-REFERENCE TO RELATED APPLICATIONS

This application is a continuation application of PCT/US2023/070102, filed Jul. 13, 2023, which claims the benefit of U.S. Provisional Application No. 63/368,344, filed Jul. 13, 2022. The entire contents of the above-identified applications are hereby fully incorporated herein by reference.

### STATEMENT REGARDING FEDERALLY SPONSORED RESEARCH

This invention was made with government support under Grant No. (s) AI110495, DK043351 and DK114784 awarded by the National Institutes of Health. The government has certain rights in the invention.

### SEQUENCE LISTING

This application contains a sequence listing filed in electronic form as an xml file entitled BROD-5530US_ST26.xml, created on Jan. 10, 2025, and having a size of 134,110 bytes. The content of the sequence listing is incorporated herein in its entirety.

### TECHNICAL FIELD

The subject matter disclosed herein is generally directed to methods, systems, and products to generate one or more immunogenic peptides comprising one or more peptide binding motifs for use in immunological compositions. These compositions are derived from immunopeptidomic features of protein binding cores as well as contextual features surrounding the binding core as described herein.

### BACKGROUND

Adaptive immunity is endowed with the ability to specifically recognize and mount responses to new antigens that the host species may not have previously encountered, and then to retain memory against these antigens to protect the individual against future exposures. The cellular arm of adaptive immunity is mediated by T cells, which collectively generate a diverse clonal repertoire of T cell receptors (TCRs) that recognize peptide antigens displayed by major histocompatibility (MHC) proteins on the surface of accessory cells. The adaptive element of cell-mediated immunity is conferred by germline-encoded variants of MHC that collectively expand the spectrum of unique peptide antigens that can be displayed to T cells, and the diverse TCR repertoire generated by somatic recombination of TCR gene segments. Class I MHC complexes are expressed in most nucleated cells and generally present peptide antigen to CD8 T cells, whereas class II MHC complexes are predominantly expressed in professional antigen presenting cells (APCs) and present peptide antigen to CD4 T cells (Germain and Margulies 1993; Neefjes et al. 2011). MHCII-restricted CD4 T cells exhibit remarkable functional heterogeneity and coordinate diverse immune responses tailored towards different pathogen threats in infectious disease, but also play important roles in tolerance, cancer, autoimmunity, and allergy (Borst et al. 2018; Alfei, Ho, and Lo 2021; Jurewicz and Stern 2019; Zheng and Wakim 2021). Thus, MHCII-restricted CD4 T cell responses perform diverse functions ranging from maintaining tolerance to the microbiome to coordinating protective immunity to emerging pathogen threats.

In humans, MHC proteins are encoded by human leukocyte antigen (HLA) genes. Classical HLA-II proteins are encoded by three highly polymorphic isotypes, HLA-DP, HLA-DQ, and HLA-DR. These HLA-II isotypes are heterodimers of alpha and beta chain proteins, and each HLA-II heterodimer binds a diverse spectrum of peptides ranging in length from 12-25 amino acids. Peptides bind within a structurally defined groove in HLA-II heterodimers, and these complexes are stabilized by interactions between HLA-II and a 9-mer amino acid core sequence in the peptide designated by positions P1-9 (Stern et al. 1994). Genetic variation within the HLA locus allows each HLA heterodimer to present a diverse array of peptide antigens. At the population level, this HLA diversity facilitates community protection, and some of the most robust genetic associations in human disease map to the HLA locus (Dendrou et al. 2018). Individuals who are heterozygous at the three HLA-II loci have at least 6 unique HLA-II heterodimers with distinct peptide binding properties. The number of possible HLA-II heterodimers can increase based on individual expression of HLA-DRB paralogs or trans-allelic pairing of alpha and beta chains from HLA-DP or-DQ. To date, 5,620 classical HLA-II protein variants have been reported (Robinson et al. 2020). Multiple factors, including co-evolution with pathogens, trade-off for protection against autoimmunity, and reproductive fitness have led to selection of genetic diversity in the HLA locus allowing for presentation of a broad spectrum of antigens that the immune system forms the basis of adaptive immunity, it is important to understand the rules of antigen presentation of each HLA-II heterodimer and identify antigens that drive disease. Identifying driver antigens in disease will enable functional characterization of antigen-specific T cell responses within the context of an enormously diverse TCR repertoire and its associated functional heterogeneity.

Advances in mass spectrometry (MS)-based immunopeptidomics approaches have enabled identification of endogenously processed and presented HLA-II peptides (Garde et al. 2019; Chong et al. 2018; Klaeger et al. 2021; Vizcaíno et al. 2020; Andreatta et al. 2019; Marcu et al. 2021; van Balen et al. 2020; Abelin et al. 2019). While the peptide binding specificity of HLA-II is a key determinant of antigen presentation to T cells, additional factors impact the efficiency by which peptides are processed from their source protein antigens. The immunogenicity of a peptide antigen is related to the efficiency by which it is presented to T cells, which is in turn affected by its abundance, propensity for uptake by APCs, trafficking to lysosomes, sensitivity to proteolytic processing, and affinity and stability on HLA-II (Unanue, Turk, and Neefjes 2016; Vyas, Van der Veen, and Ploegh 2008; Hsing and Rudensky 2005). In this context, mechanisms of antigen presentation and biochemical features of antigenicity remain incompletely understood.

Here, Applicants leveraged immunopeptidomics and machine learning to define rules of antigen processing and presentation that enabled discovery of immunodominant T cell antigens and functional characterization of antigen-specific T cell responses in health and disease. Applicants demonstrated that healthy individuals possess circulating T cells that actively recognized microbiome-derived epitopes and exhibit characteristics of tissue-protective functions and IL-17 skewing. Thus, identification of rare antigen-specific T cells sheds light on the host-microbiome relationship and its role in immune homeostasis. By contrast, pathogen infection disrupts homeostasis and elicits robust immune activation. Applicants identified an immunodominant T cell epitope in SARS-COV-2 nucleoprotein associated with antiviral Th1 immunity and IFN-γ production. Taken together, defining features of antigenicity enabled discovery of bacterial and viral antigens that drive functionally heterogeneous T cell responses in humans.

Citation or identification of any document in this application is not an admission that such a document is available as prior art to the present invention.

### SUMMARY

In one aspect, described herein, a computer-implemented method to generate one or more immunogenic peptides comprising one or more peptide binding motifs for use in immunological compositions, comprising: a) receiving, by an acquisition engine communicatively coupled to a user device, one or more amino acid sequences; b) transferring, by the acquisition engine, the one or more amino acid sequences to a deployed machine learning network communicatively coupled to the acquisition engine; c) processing the one or more amino acid sequences with the deployed machine learning network, the deployed machine learning network generated and deployed from a training machine learning network; and d) generating one or more immunogenic peptides comprising one or more peptide binding motifs. In an example embodiment, the method further comprises, e) receiving, by an acquisition engine communicatively coupled to a user device, a second one or more amino acid sequences; f) receiving, by an acquisition engine communicatively coupled to a user device, a second one or more amino acid sequences; g) processing the second one or more amino acid sequences with the second deployed machine learning network, the second deployed machine learning network generated and deployed from a second training machine learning network; and h) generating one or more ligand regions of the second one or more amino acid sequences. In an example embodiment, the method further comprises, i) transferring, by the acquisition engine, the one or more immunogenic peptides comprising one or more peptide binding motifs and the one or more ligand regions to an ensemble network communicatively coupled to the acquisition engine; j) processing the one or more peptide binding motifs and the one or more ligand regions with the ensemble network; and k) generating a refined set of one or more immunogenic peptides comprising one or more peptide binding motifs. In an example embodiment, the method further comprises preparing one or more immunogenic peptides for an immunological composition.

In an example embodiment, the first and/or second deployed machine learning networks receive one or more features of the one or more amino acid sequences. In an example embodiment, the one or more features comprise binary, fractional, or both features. In an example embodiment, the fractional features comprise secreted protein features, transmembrane features, domain features, region features, relative solvent accessibility features, and disorder features. In an example embodiment, the generated one or more immunogenic peptides comprising one or more immunogenic peptide binding motifs further comprise individual probability or confidence scores. In an example embodiment, the generated one or more ligand regions further comprise individual probability or confidence scores at each position in the ligand region.

In an example embodiment, the immunogenic peptide comprising one or more peptide binding motifs is specific for one or more HLA II alleles selected from the group consisting of those in Table 1. In an example embodiment, the second one or more amino acids comprise signaling regions. In an example embodiment, the signaling regions comprise adjacent and/or distant signaling regions. In an example embodiment, the adjacent signaling regions comprise exopeptidase trimming sites and/or proline-rich cleavage motifs.

In an example embodiment, the second one or more amino acids comprises the full length of a protein. In an example embodiment, the second one or more amino acid sequences comprises one or more amino acid sequences previously described, wherein the one or more amino acid sequences previously described further comprises one or more additional sequences up to the full length of the source protein. In an example embodiment, the second one or more amino acids are expanded sequences of the first one or more amino acids. In an example embodiment, the one or more amino acid sequences are of 7 to 100, 7 to 75, 7 to 50, or 7 to 25 amino acids in length. In an example embodiment, the one or more amino acid sequences have a maximum overlap of 10, 9, 8, 7, 6, or 5 amino acids. In an example embodiment, the one or more peptide binding motifs is between 5 to 100, 5 to 75, 5 to 50, or 5 to 25 amino acids in length. In an example embodiment, the immunogenic peptide comprising one or more peptide binding motifs is 12 to 100, 12 to 75, 12 to 50, or 12 to 25 amino acids in length. In an example embodiment, the immunogenic peptide comprising one or more immunogenic peptide binding motifs is around 20 amino acids in length. In an example embodiment, the ligand region further comprises an additional 25 amino acids on either or both sides of the generated ligand region.

In an example embodiment, the first and second machine learning network independently comprises linear classifiers, logistic classifiers, Bayesian networks, random forest, neural networks, matrix factorization, hidden Markov model, support vector machine, K-means clustering, or K-nearest neighbor. In an example embodiment, the first deployed machine learning network comprises a neural network. In an example embodiment, the neural network comprises a convolutional neural network. In an example embodiment, the first deployed machine learning network comprises embedding. In an example embodiment, the second deployed machine learning network comprises a neural network. In an example embodiment, the neural network comprises a convolutional neural network. In an example embodiment, the neural network comprises a recurrent neural network. In an example embodiment, the second deployed machine learning network comprises embedding.

In an example embodiment, the first and second deployed machine learning network independently comprises unsupervised learning, supervised learning, semi-supervised learning, reinforcement learning, transfer learning, incremental learning, curriculum learning, and learning to learn. In an example embodiment, the machine learning network in (b) is trained on HLA-II allele-specific peptidomics data. In an example embodiment, the second machine learning network is trained on full length source proteins mapped back from HLA-II allele-specific peptidomics data, thereby generating the one or more regions of full length proteins affecting the one or more peptide binding motifs. In an example embodiment, the training the first deployed machine learning network comprises decoys at a 4:1, 5:1, or 6:1 ratio to immunogenic peptides.

In an example embodiment, the ensemble network comprises grid search. In an example embodiment, the second deployed machine learning network comprises bi-directional long-short term memory (LSTM). In an example embodiment, the bi-direction long-short term memory is performed more than once. In an example embodiment, the first deployed machine learning network further comprising pooling, reduction, and/or dropout steps. In an example embodiment, the parameters of the first and/or the second deployed machine learning network is tuned to minimize binary entropy loss. In an example embodiment, the immunogenic peptide comprising immunogenic peptide binding motifs are specific to HLA-II alleles specific to a subject. In an example embodiment, the one or more amino acid sequences comprise full-length protein sequences.

In an example embodiment, the one or more amino acid sequences are obtained by analyzing one or more genomic DNA sequences. In an example embodiment, the one or more genomic DNA sequences is a full genome sequence. In an example embodiment, the one or more input genome sequences is derived from a target pathogen, a commensal microorganism, or a diseased cell. In an example embodiment, the pathogen is selected from the group consisting of a bacterium, a virus, a protozoon, and an allergen. In an example embodiment, the diseased cell is a cancer cell. In an example embodiment, the one or more amino acid sequences are derived from neoantigens. In an example embodiment, the method further comprises detecting whether one or more of the antigenic epitopes is present in a sample from a subject suffering from an infection, autoimmune disease, allergy, or cancer. In an example embodiment, the immunological composition is a protective vaccine or tolerizing vaccine composition comprising one or more of the antigenic epitopes.

In one aspect, described herein, is a system to generate one or more immunogenic peptides comprising one or more peptide binding motifs for use in immunological compositions, comprising: a storage device; and a processor communicatively coupled to the storage device, wherein the processor executes application code instructions that are stored in the storage device to cause the system to: a) receive, by an acquisition engine communicatively coupled to a user device, one or more amino acid sequences; b) transfer the one or more amino acid sequences with an acquisition engine communicatively coupled to a deployed machine learning network; c) process the one or more amino acid sequences with a deployed machine learning network, the deployed machine learning network generated and deployed from a training machine learning network; and d) generate one or more immunogenic peptides comprising one or more peptide binding motifs. In an example embodiment, the system further comprises e) receive, by an acquisition engine communicatively coupled to a user device, a second one or more amino acid sequences; f) transfer the second one or more amino acid sequences with the acquisition engine communicatively coupled to a second deployed machine learning network; g) process the second one or more amino acid sequences with the second deployed machine learning network, the second deployed machine learning network generated and deployed from a second training machine learning network; and h) generate one or more ligand regions of the second one or more amino acid sequences. In an example embodiment, the system further comprises i) transfer the one or more immunogenic peptides comprising one or more peptide binding motifs and the one or more ligand regions with the acquisition engine communicatively coupled to an ensemble network; j) process the one or more peptide binding motifs and the one or more ligand regions with the ensemble network; and k) generate a refined set of the one or more immunogenic peptides comprising one or more peptide binding motifs. In an example embodiment, further comprising preparing one or more immunogenic peptides for an immunological composition.

In an example embodiment, the first and/or second deployed machine learning networks of the system receive one or more features of the one or more amino acid sequences. In an example embodiment, the one or more features of the system comprise binary, fractional, or both features. In an example embodiment, the fractional features of the system comprise secreted protein features, transmembrane features, domain features, region features, relative solvent accessibility features, and disorder features. In an example embodiment, the generated immunogenic peptide comprising one or more peptide binding motifs of the system further comprise individual probability or confidence scores. In an example embodiment, the generated one or more ligand regions further comprise individual probability or confidence scores at each position in the ligand region.

In an example embodiment, the immunogenic peptide comprising one or more peptide binding motifs of the system is specific for one or more HLA II alleles selected from the group consisting of those in Table 1. In an example embodiment, the second one or more amino acids of the system comprise signaling regions. In an example embodiment, the signaling regions of the system comprise adjacent and/or distant signaling regions. In an example embodiment, the adjacent signaling regions of the system comprise exopeptidase trimming sites and/or proline-rich cleavage motifs.

In an example embodiment, the second one or more amino acids of the system comprises the full length of a protein. In an example embodiment, the second one or more amino acid sequences of the system comprises one or more amino acid sequences previously described, wherein the one or more amino acid sequences previously described further comprises one or more additional sequences up to the full length of the source protein. In an example embodiment, the second one or more amino acids of the system are expanded sequences of the first one or more amino acids. In an example embodiment, the one or more amino acid sequences of the system are of 7 to 100, 7 to 75, 7 to 50, or 7 to 25 amino acids in length. In an example embodiment, the one or more amino acid sequences of the system have a maximum overlap of 10, 9, 8, 7, 6, or 5 amino acids. In an example embodiment, the immunogenic peptide comprising one or more peptide binding motifs of the system is between 5 to 100, 5 to 75, 5 to 50, or 5 to 25 amino acids in length. In an example embodiment, the immunogenic peptide comprising one or more peptide binding motifs of the system is 12 to 100, 12 to 75, 12 to 50, or 12 to 25 amino acids in length. In an example embodiment, the immunogenic peptides comprising one or more peptide binding motifs of the system is around 20 amino acids in length. In an example embodiment, the ligand region of the system further comprises an additional 25 amino acids on either or both sides of the generated ligand region.

In an example embodiment, the first and second machine learning network of the system independently comprises linear classifiers, logistic classifiers, Bayesian networks, random forest, neural networks, matrix factorization, hidden Markov model, support vector machine, K-means clustering, or K-nearest neighbor. In an example embodiment, the first deployed machine learning network of the system comprises a neural network. In an example embodiment, the neural network of the system comprises a convolutional neural network. In an example embodiment, the first deployed machine learning network of the system comprises embedding. In an example embodiment, the second deployed machine learning network of the system comprises a neural network. In an example embodiment, the neural network of the system comprises a convolutional neural network. In an example embodiment, the neural network of the system comprises a recurrent neural network. In an example embodiment, the second deployed machine learning network of the system comprises embedding.

In an example embodiment, the first and second deployed machine learning network of the system independently comprises unsupervised learning, supervised learning, semi-supervised learning, reinforcement learning, transfer learning, incremental learning, curriculum learning, and learning to learn. In an example embodiment, the machine learning network in (b) of the system is trained on HLA-II allele-specific peptidomics data. In an example embodiment, the second machine learning network of the system is trained on full length source proteins mapped back from HLA-II allele-specific peptidomics data, thereby generating the one or more regions of full length proteins affecting the one or more peptide binding motifs. In an example embodiment, the training the first deployed machine learning network of the system comprises decoys at a 4:1, 5:1, or 6:1 ratio to immunogenic peptides.

In an example embodiment, the ensemble network of the system comprises grid search. In an example embodiment, the second deployed machine learning network of the system comprises bi-directional long-short term memory (LSTM). In an example embodiment, the bi-direction long-short term memory of the system is performed more than once. In an example embodiment, the first deployed machine learning network of the system further comprising pooling, reduction, and/or dropout steps. In an example embodiment, the parameters of the first and/or the second deployed machine learning network of the system is tuned to minimize binary entropy loss. In an example embodiment, the immunogenic peptides comprising one or more peptide binding motifs of the system are specific to HLA-II alleles specific to a subject. In an example embodiment, the one or more amino acid sequences of the system comprise full-length protein sequences.

In an example embodiment, the one or more amino acid sequences of the system are obtained by analyzing one or more genomic DNA sequences. In an example embodiment, the one or more genomic DNA sequences of the system is a full genome sequence. In an example embodiment, the one or more input genome sequences of the system is derived from a target pathogen, a commensal microorganism, or a diseased cell. In an example embodiment, the pathogen of the system is selected from the group consisting of a bacterium, a virus, a protozoon, and an allergen. In an example embodiment, the diseased cell of the system is a cancer cell. In an example embodiment, the one or more amino acid sequences of the system are derived from neoantigens. In an example embodiment, the system further comprises detecting whether one or more of the antigenic epitopes is present in a sample from a subject suffering from an infection, autoimmune disease, allergy, or cancer. In an example embodiment, the immunological composition of the system is a protective vaccine or tolerizing vaccine composition comprising one or more of the antigenic epitopes.

In one aspect, described herein, is a computer program product, comprising: a non-transitory computer-readable storage device having computer-executable program instructions embodied thereon that when executed by a computer cause the computer to generate one or more immunogenic peptides comprising one or more peptide binding motifs for use in immunological compositions, the computer-executable program instructions comprising: a) computer-executable program instructions to receive, by an acquisition engine communicatively coupled to a user device, one or more amino acid sequences; b) computer-executable program instructions to transfer the one or more amino acid sequences with the acquisition engine communicatively coupled to a deployed machine learning network; c) computer-executable program instructions to process the one or more amino acid sequences with the deployed machine learning network, the deployed machine learning network generated and deployed from a training machine learning network and communicatively coupled to the acquisition engine; and d) computer-executable program instructions to generate a one or more immunogenic peptide binding motifs. In an example embodiment, the product further comprises e) computer-executable program instructions to receive, by an acquisition engine communicatively coupled to a user device, a second one or more amino acid sequences; f) transfer the second one or more amino acid sequences with the acquisition engine communicatively coupled to a second deployed machine learning network; g) process the second one or more amino acid sequences with the second deployed machine learning network, the second deployed machine learning network generated and deployed from a second training machine learning network; and h) generate one or more ligand regions of the second one or more amino acid sequences. In an example embodiment, the product further comprises i) transfer the one or more immunogenic peptides comprising one or more peptide binding motifs and the one or more ligand regions with the acquisition engine communicatively coupled to an ensemble network; j) process the one or more peptide binding motifs and the one or more ligand regions with the ensemble network; and k) generate a refined set of the one or more immunogenic peptides comprising one or more peptide binding motifs. In an example embodiment, further comprising preparing one or more immunogenic peptides for an immunological composition.

In an example embodiment, the first and/or second deployed machine learning networks of the product receive one or more features of the one or more amino acid sequences. In an example embodiment, the one or more features of the product comprise binary, fractional, or both features. In an example embodiment, the fractional features of the product comprise secreted protein features, transmembrane features, domain features, region features, relative solvent accessibility features, and disorder features. In an example embodiment, the generated one or more immunogenic peptide binding motifs of the product further comprise individual probability or confidence scores. In an example embodiment, the generated one or more ligand regions further comprise individual probability or confidence scores at each position in the ligand region.

In an example embodiment, the one or more immunogenic peptides comprising one or more immunogenic peptide binding motifs of the product is specific for one or more HLA II alleles selected from the group consisting of those in Table 1. In an example embodiment, the second one or more amino acids of the product comprise signaling regions. In an example embodiment, the signaling regions of the product comprise adjacent and/or distant signaling regions. In an example embodiment, the adjacent signaling regions of the product comprise exopeptidase trimming sites and/or proline-rich cleavage motifs.

In an example embodiment, the second one or more amino acids of the product comprises the full length of a protein. In an example embodiment, the second one or more amino acid sequences of the product comprises one or more amino acid sequences previously described, wherein the one or more amino acid sequences previously described further comprises one or more additional sequences up to the full length of the source protein. In an example embodiment, the second one or more amino acids of the product are expanded sequences of the first one or more amino acids. In an example embodiment, the one or more amino acid sequences of the product are of 7 to 100, 7 to 75, 7 to 50, or 7 to 25 amino acids in length. In an example embodiment, the one or more amino acid sequences of the product have a maximum overlap of 10, 9, 8, 7, 6, or 5 amino acids. In an example embodiment, the immunogenic peptide comprising one or more peptide binding motifs of the product is between 5 to 100, 5 to 75, 5 to 50, or 5 to 25 amino acids in length. In an example embodiment, the immunogenic peptides comprising one or more peptide binding motifs of the product is 12 to 100, 12 to 75, 12 to 50, or 12 to 25 amino acids in length. In an example embodiment, the immunogenic peptide comprising one or more immunogenic peptide binding motifs of the product is around 20 amino acids in length. In an example embodiment, the ligand region of the product further comprises an additional 25 amino acids on either or both sides of the generated ligand region.

In an example embodiment, the first and second machine learning network of the product independently comprises linear classifiers, logistic classifiers, Bayesian networks, random forest, neural networks, matrix factorization, hidden Markov model, support vector machine, K-means clustering, or K-nearest neighbor. In an example embodiment, the first deployed machine learning network of the product comprises a neural network. In an example embodiment, the neural network of the product comprises a convolutional neural network. In an example embodiment, the first deployed machine learning network of the product comprises embedding. In an example embodiment, the second deployed machine learning network of the product comprises a neural network. In an example embodiment, the neural network of the product comprises a convolutional neural network. In an example embodiment, the neural network of the product comprises a recurrent neural network. In an example embodiment, the second deployed machine learning network of the product comprises embedding.

In an example embodiment, the first and second deployed machine learning network of the product independently comprises unsupervised learning, supervised learning, semi-supervised learning, reinforcement learning, transfer learning, incremental learning, curriculum learning, and learning to learn. In an example embodiment, the machine learning network in (b) of the product is trained on HLA-II allele-specific peptidomics data. In an example embodiment, the second machine learning network of the product is trained on full length source proteins mapped back from HLA-II allele-specific peptidomics data, thereby generating the one or more regions of full length proteins affecting the one or more peptide binding motifs. In an example embodiment, the training the first deployed machine learning network of the product comprises decoys at a 4:1, 5:1, or 6:1 ratio to immunogenic peptides.

In an example embodiment, the ensemble network of the product comprises grid search. In an example embodiment, the second deployed machine learning network of the product comprises bi-directional long-short term memory (LSTM). In an example embodiment, the bi-direction long-short term memory of the product is performed more than once. In an example embodiment, the first deployed machine learning network of the product further comprising pooling, reduction, and/or dropout steps. In an example embodiment, the parameters of the first and/or the second deployed machine learning network of the product is tuned to minimize binary entropy loss. In an example embodiment, the immunogenic peptides comprising peptide binding motifs of the product are specific to HLA-II alleles specific to a subject. In an example embodiment, the one or more amino acid sequences of the product comprise full-length protein sequences.

In an example embodiment, the one or more amino acid sequences of the product are obtained by analyzing one or more genomic DNA sequences. In an example embodiment, the one or more genomic DNA sequences of the product is a full genome sequence. In an example embodiment, the one or more input genome sequences of the product is derived from a target pathogen, a commensal microorganism, or a diseased cell. In an example embodiment, the pathogen of the product is selected from the group consisting of a bacterium, a virus, a protozoon, and an allergen. In an example embodiment, the diseased cell of the product is a cancer cell. In an example embodiment, the one or more amino acid sequences of the product are derived from neoantigens. In an example embodiment, the system further comprises detecting whether one or more of the antigenic epitopes is present in a sample from a subject suffering from an infection, autoimmune disease, allergy, or cancer. In an example embodiment, the immunological composition of the product is a protective vaccine or tolerizing vaccine composition comprising one or more of the antigenic epitopes.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1D-Mono-allelic profiling of the HLA-II peptidome recovers thousands of peptides. 1A) Schematic of affinity tag-based immunopeptidomics workflow. Monoallelic peptidomics profiling in Expi293 cells expressing individual StrepII-tagged HLA-II heterodimers. For HLA-DQ and HLA-DP heterodimers, Expi293 with alpha chain knock-outs were leveraged to prevent heterodimer pairing with endogenous alpha chains. 1B) Quantified flow-cytometry data showing surface and total cell expression (intracellular stain) of HLA-II heterodimers used in immunopeptidomics experiments. HLA-DP was expressed in DPA1 KO cells. HLA-DQ and HLA-DR were expressed in WT cells. Percentage of relative positive cells were normalized to DPA1*02:01, DPB1*17:01, DQA1*01:03, DQB1*06:03, or DRB1*11:01, respectively. 1C) Number of unique HLA-II binding peptides and de-nested peptides per HLA-II heterodimer. 1D) Length distribution density plot of unique peptides bound to HLA-II isotypes.

FIG. 2A-2H—Clustering of HLA-II allele-specific peptide ligands reveals binding register motifs. 2A-C) We assembled a catalogue of peptide motif preferences for approximately 87 of the major HLA-II alleles. GibbsCluster 2.0 was used to identify unique HLA-II ligand regions from three datasets (this work, Abelin et al., 20219 and IEDB). Ligands from the three datasets were merged, except where indicated. Euclidean distance-based clustering of primary motifs represented as amino acid probabilities at P1-P9 derived from unique binding registers. Peptide motifs for each HLA heterodimer are clustered by gene: HLA-DP,-DQ,-DR. 2D) Multidimensional scaling (MDS) of primary and secondary motif amino acid probability matrices based on Euclidean distance. Primary motifs are diverse and segregate by HLA-II gene (HLA-DP, -DQ,-DR), suggesting diverse sequence-specific preferences in peptide binding. In contrast, secondary motifs share common features such as Pro- and Gly-rich sequences, suggesting features that may facilitate promiscuous binding across HLA alleles. 2E) Example primary and secondary motifs for DQA1*01:03, DQB1*03:06. 2F) Number of sequences grouped to primary and secondary motifs for each of the 87 HLA heterodimers. Lines represent slopes (ratios between sequences in primary versus secondary motifs) with the average slope equal to 1.80, demonstrating that HLA-II binding peptides conforming to primary motifs thus outnumber secondary motifs by approximately 2-fold. 2G) An example of HLA-II (gray cartoon) and peptide (ribbon) complex (PDB 3LQZ). Peptide is grayed as a spectrum from the N terminus (P1) to the C (P9) terminus, and side chains are shown as sticks. HLA-II-bound peptides adopt an extended helical conformation resembling a polyproline helix type II (PPII), where the side chain of every third residue aligns in the same direction. Position of TCR bound to peptide-HLA-II is shown as a dashed line. 2H) Schematic of peptide-HLA-II-TCR interaction. Hydrogen bonds between peptide and HLA-II are shown as dotted lines, and position TCR is shown as a dashed line. The backbone of the stretched PPII peptide is conducive to forming hydrogen bonds with the absolutely conserved asparagine residues (Asn62a, Asn69a, and Asn82B) of HLA-II. Because these hydrogen bonds do not involve side chains of the peptide, any peptides rich in Pro and Gly have a propensity to adopt a PPII conformation and bind different HLA-II heterodimers promiscuously, as observed for peptides classified as secondary motifs (FIG. 2D). This contrasts with peptides characterized by primary motifs that utilize amino acid side chains to interact specifically with HLA-II heterodimers.

FIG. 3A-3F—HLA-II isotypes exhibit unique structural features that dictate peptide-binding specificity. 3A) Uncertainty quantified as entropy at P1-P9 for representative motifs for each HLA-II heterodimer. A single motif was obtained per heterodimer in up to three separate datasets (see Methods). Lower entropy at a given amino acid position in the peptide indicates preferential binding of specific amino acids. HLA-DR heterodimers exhibit amino acid binding preferences at P1, P4, P6, and P9 anchor residues, while HLA-DP heterodimers exhibit preferences at P1, P6, and P9 and HLA-DQ heterodimers at P3 and P4. 3B-C) Contribution of HLA-II alpha chains to peptide-binding preferences. Variance of a single motif per heterodimer (see Methods) at P1-P9 by alpha and beta chains for HLA-DP and-DQ using a generalized linear model with Dirichlet distribution over amino acid probabilities. HLA-DP peptide binding specificity can be largely explained by features of the beta chain. This trend was also observed for HLA-DQ with the notable exception of P3, where the alpha chain contributes most significantly to peptide binding specificity. 3D) Surface representation of HLA-DP5 (PDB 3WEX), HLA-DQ (PDB 6PY2), and HLA-DR (PDB 3TOE) bound to a peptide (ribbon and stick). HLA-II is grayed by sequence conservation among HLA-II heterodimers within genes reported in IMGT with highly conserved residues in maroon and variable residues in turquoise. Compared to HLA-DP, site 1 and site 2 of HLA-DQ are more variable. The variation in site 1 of HLA-DQ occurs in a region that interacts with HLA-DM and site 2 interacts with P3 of the peptide, suggesting that HLA-DQ heterodimers preferentially select ligands through interactions with the middle region of the peptide at P3 and P4, as illustrated. 3E) HLA-DQ (top; PDB 4MAY) and HLA-DP (bottom) heterodimers exhibit variability within the floor of P4 and P6 pockets, respectively. Properties of HLA-DQ beta chain residues β26 and β28 determine P4 residue preferences. Properties of HLA-DP alpha chain residues α11 and β11 determine the width and specificity of the P6 pocket. Observed anchor pocket variability is correlated with the entropy shown in FIG. 3A, where HLA-DQ has lower entropy at P4 than HLA-DP and vice versa for P6. 3F) Comparison of the position of HLA-II-bound peptides in the peptide binding groove. Alpha chains of each HLA-II heterodimers were aligned, and one representative HLA-II (PDB 3LQZ) is shown in cartoon. HLA-II-bound peptides are shown in ribbon, and side chains of P4 are shown in line. The P4 binding pocket for HLA-DQ is deeper compared to HLA-DP, which has conserved Phe24B in the floor of the P4 binding pocket as shown in FIG. 3E. The deeper insertion of P4 side chain towards the groove results in a close positioning of P3 side chains with the HLA-DQ alpha chain. HLA-DQ peptides are from PDB entries 6PY2, 6MFF, 4GG6, 4MAY, 5KSA, 6U3N, and 1JK8. HLA-DP peptides are from PDB entries 3LQZ, 3WEX, 4P4K, 4P5K, and 4P5M.

FIG. 4A-4G—Design and performance of machine learning models to predict HLA-II peptide ligands. 4A) Overview of the end-to-end modeling pipeline and schematic of the machine learning models that detect binding cores (Core Models). Layers in the Core Model neural network are associated with an activation or aggregation function and output tensor dimensions. Asterisks (*) represent tensor components of arbitrary dimensions. The key functions to capture the epitope diversity are convolutional layers (able to model multiple, different amino acid patterns), the pooling layer (selecting patterns with strongest activations), reduce (selecting strongest activation anywhere in the sequence). The sigmoid units return probabilities in range 0-100% which are calibrated using the binary entropy loss. 4B-C) Area under receiver-operating characteristic (ROC) and precision-recall curve in five-fold cross-validation experiments. The dataset of origin is shown on the right. The Core Models achieve highest classification performance in HLA-II heterodimers profiled in this work and re-processed data that recovers ligands associated with both primary and secondary motifs. The proposed model architecture has the capacity to capture heterogeneous groups of motifs that appear anywhere in the sequence up to length 50 amino acids. Comparison between the best two models yields 81 wins for Core Model (AVP, P<0.05, paired T-test over five data set splits, AVP, Table 1) and 84 (AUC, P<0.05, paired T-test, Table 1), with the remaining comparisons non-significant (P>0.05). 4D) Definition of merged ligand regions for the three isotypes. 4E) Architecture of the recurrent neural network implementation of the Context models. The input is a sequence of amino acids (represented as 20 dimensional binary encoding) of arbitrary length (*). The parameter settings are the number of convolutional filters C=128 and window size m=20, LSTM embedding dimension H=32, initial dropout probability p=0.1 and initial decision functions dimension F=48. Plate notations represent modules of bidirectional LSTM layers (2 repeats) and decision functions (4 repeats), where the output of module k is the input to module k+1. 4F) Schematic of Ensemble model formulation. The predictions of Core and Context models are aggregated at each position in the protein using a weighted sum and aggregate score of the Context model in the interval [a, b] around a target position by either maximum or average, optimized by grid search (Methods). 4G) Classification performance on top 30, 100, 300, 1000 predicted epitopes for each allele. The non-overlapping ligand regions of length 20 amino acids prioritized by each method are sorted by predicted confidence and compared with observed data. The plus sign represents median accuracy at each threshold.

FIG. 5A-5E—Context models are associated with structural and phyisico-chemical properties of antigen source protein amino acid sequence. 5A) Design of experiments with predicted structural features. The same peptide ligand datasets used as for training the Core Models are used. Established methods are used to predict various structural features associated with each amino acid: TMHMM (membrane topology), HMMER (existence of PFAM domains), Interproscan (signal peptides) and Netsurfp (relative solvent accessibility, disorder). Hydrophobicity is computed from single amino acids and assigned nominal values hydrophobic=0, neutral-0.5, hydrophilic=1. 5B) Enrichment analysis of each structural feature in HLA-II ligands versus decoys. Bars show the percentage difference in probability in ligands versus decoys, pdiff-pligand-pdecoy, where pligand and pdecoy are the fraction of amino acids pertaining to the ligands and decoys, respectively. 5C) Enrichment of structural features in amino acids prioritized by NetMHCIIpan and the Context Model computed as a difference of percentage of a feature within predicted amino acids versus background. Full lines show mean probabilities of a feature, binned by predicted confidence percentiles by each method. Dashed lines represent minimum and maximum value across five replicates corresponding to data splits (Methods). Grayed inset values are Spearman correlations coefficients between predicted values and presence of structural features. These analyses suggest that features associated with antigenicity include secreted proteins, outer membrane regions of transmembrane proteins, and structured PFAM domains. Conversely, disordered regions and helices were less likely to contain HLA-II binding ligands. 5D-E) Comparison in achievable validation performance for context models with or without memory (CNN or LSTM) and structural features in terms of area under ROC curve (D) or area under precision-recall curve (E). While the models based only on short, 10-15 mer, sequences benefit from structural features (CNN), the difference in performance vanishes for memory-based (recurrent) models (LSTM). These comparisons show that factors affecting ligand recognition beyond binding core motifs can be inferred from whole protein sequence given sufficiently large datasets, while not providing explicit computations of those features and requiring the protein sequence alone as the sole input requirement.

FIG. 6A-6F—The ensemble CAPTAn models accurately predict microbial epitopes presented by human DCs identified by immunopeptidomics. 6A) A consortium of commensal bacteria from the microbiome (n=6 species) was cultured with monocyte-derived dendritic cells (DCs), and HLA-II immunopeptidomics was performed. Recovered peptides are shown per bacteria species. Peptides are reported with either a 1% FDR or 1% FDR and additional quality filtering using spectral quality (Supplemental Figure). 6B) Peptides were deconvoluted based on HLA-type of DC donor. Motif of filtered bacterial peptides using Gibbs Cluster 2.0 and most similar primary motifs for HLA-II-alleles. A majority of peptides conformed to the motif for HLA-DRB1*03:01. 6C) CAPTAn CoreContext20 model accurately predicts HLA-II peptide ligands derived from commensal microbes. Displayed is the number of times each method ranked first (wins) in prioritization of peptides for different cutoffs: top 1, 3, 10, 30 and 100 peptides with highest confidence. The peptides predicted by the CAPTAn model correspond to observed peptide ligands for most prediction cutoffs and HLA-II alleles. 6D) Healthy human subjects generate T cell responses towards microbiome epitopes. Cytokine responses were measured in PBMCs after stimulation in vitro with synthetic peptides. Eight peptides (DC1-8) were selected from CAPTAn-predictions and/or peptidomics experiments. Negative controls: DMSO, CLIP, IGRP. Positive controls: Infl_NP, C_tet. 6E) (SEQ ID NO: 1-2) Source of DC2 and DC7 peptides, including number of genes encoding them in the gut microbiomes and the prevalence of their expression based on metatranscriptomic profiling in HMP2 (Lloyd-Price et al. 2019). 6F) Peptide DC7 from V. parvula WP_156697519.1 was used to generate HLA-DRB1*03:01 tetramers. Tetramer staining and enrichment was performed on PBMCs from a healthy donor prior to analysis by FACS and gating on CD45+CD3+ T cells (see Supplemental Figure for gating strategy).

FIG. 7A-7H—Ensemble CAPTAn models recapitulate published SARS-COV-2 epitopes and uncover novel DQ6 epitopes in the viral nucleoprotein antigen. 7A) CAPTAn predictions for SARS-COV-2. Number of predicted unique 15-mer peptides across HLA-DR,-DP and-DQ alleles. Black line represents the expected number of predictions and is fitted using a linear model log 10 (regions)=log 10 (protein length)+isotype. 7B) Comparison of CAPTAn predictions of SARS-COV-2 nucleoprotein epitopes versus observed CD4 T cell responses from 25 human studies (Grifoni et al. 2021). Isotype-specific context model predictions are associated with response frequency of viral nucleoprotein ligand regions. The black line shows the lower bound CD4 T cell response frequency (%) of epitopes containing each amino acid. The gray lines are confidence scores of the context models for HLA-DP,-DQ and-DR. Gray rectangle corresponds to predicted DQA1*03:01, DQB1: 06:03 epitope at N135-150. 7C) Correlation between context model predictions (for DP, DQ and DR) and lower bound CD4 T cell response frequency (%) for all ORFs in SARS-Cov-2 proteome. Nucleoprotein correlations are shown in large dots aligned with B), while other ORFs are shown in black. 7D) (SEQ ID NO: 3-10) CAPTAn predictions for SARS-COV-2 nucleoprotein (N) restricted to DQA1*03:01, DQB1: 06:03. The dark gray line shows the predicted confidence of epitopes mapped to the amino acid sequence of N. Gray lines show predictions for six other human coronavirus strains (CVHN1, CVHN2, CVHN5, CVHOC, CVH22, CVHNL). Five epitopes with highest scores are printed in dark gray (SARS2) and the corresponding region in another coronavirus strain is shown below. The strongest epitope candidate at residue N135 (TEGALNTPKDHIGTR (SEQ ID NO: 3)) is printed on the top. The arrow highlights an aspartate residue (D) present in SARS-COV-2 nucleoprotein that is absent everywhere that determines P6 of DQA1*03:01, DQB1: 06:03 binding motifs. The two panels on the bottom show multiple sequence alignment and homology (number of strains agreeing in an amino acid position). 7E) (SEQ ID NO: 11) Multiple sequence alignment between N protein regions containing N135-150 of SARS-Cov-2 variants of concern. 7F) Number of mutations relative to the reference SARS-COV-2 strain as cataloged by covid-19.uniprot.org/on Jan. 31, 2022. 7G) Validation of T cell reactivity to N135-DQA1*01:03-DQB1*06:03. Nine TCRs were selected from a convalescent COVID-19 patient based on their provenance from T cells with high cytokine secretion upon restimulation in vitro with pooled N peptides (manuscript submitted). TCRs were screened against N135 and N73 presented by DQA1*01:03-DQB1*06:03. In this system, BW5147.3 cells expressing HLA-DQ fused to peptide N135 at the N-terminus and CD3zeta on the C-terminus were co-cultured overnight with Expi293F cells expressing TCR and CD3. Surface HLA-DQ and 4-1BB expression on BW5147.3 cells were analyzed by flow-cytometry. 4-1BB expression is a surrogate activation marker indicating that TCR clone 21 reacts specifically with nucleoprotein peptide N135 presented by HLA-DQA1*01:03-DQB1*06:03. 7H) N135 peptide-presenting HLA-DQ tetramer staining of TCR clones expressed on Expi293F cells.

FIG. 8A-8B—Workflow of mono-allelic HLA-II immunopeptidomics. 8A) (SEQ ID NO: 12-21) Generation and validation of CRISPR-Cas9 knockout cell lines. Chromatogram of target sequences. Gray line indicates guide RNA binding sequences. Dark gray arrow indicates the cleavage site of the signal peptide. Amino acid sequences modified by CRISPR-Cas9 were colored in light gray. *, stop codon. 8B) Detailed experimental schematic. Cells from two independent transfections were used for immunoprecipitation and peptide elution. Variables between replicates are shown in light gray text. Unique peptides were filtered and used to calculate length distribution and for clustering.

FIG. 9A-9F—Logos of primary and secondary motifs for each HLA allele in the study. Motifs are represented as information of amino acids at positions P1-P9 (bits) derived from unique binding registers by running Gibbs Cluster 2.0 with 2 motifs. Motifs are hierarchically clustered by Euclidean distance and Ward's linkage (represented as dendrograms in the main FIG. 2) and split into five clusters for visualization. When peptides from multiple data sources are combined to form a motif, a single letter abbreviation is used: this work (T), Abelin et al., 2019 (A) and IEDB (I). Peptide motifs for each HLA heterodimer are clustered by gene: 9A) HLA-DP primary motifs, 9B) HLA-DP secondary motifs, 9C) HLA-DQ primary motifs, 9D) HLA-DQ secondary motifs, 9E) HLA-DR primary motifs, 9F) HLA-DR secondary motifs.

FIG. 10A-10D—Selection of consensus motifs and variance of alpha and beta chains. 10A-B) Consensus motifs derived from three datasets (this work, Abelin et. al 2019, IEDB). A single motif is selected for each allele based on maximum Kullback-Leibler divergence computed in GibbsCluster 2.0. The line thickness represents the percentile of Euclidean distance between a pair of motifs among all possible pairs. HLA-DP beta chains (A) and HLA-DQ alpha chains (B) with most represented alleles are colored according to legend. 10C-D) Detailed view of sequence variation near P1 and P3. Cartoon representation of HLA-DP (C left, PDB 3WEX), HLA-DQ (C and D right, PDB 4MAY; D left, PDB 6PY2). Sequence variance near P1 (C) and P3 (D) is colored from conserved to variable. Bound peptides are shown in ribbon.

FIG. 11A-11E—Assembly of the training dataset and inspection of CAPTAn-core models. 11A) Numbers of unique peptide ligands obtained from HLA-II mono-allelic datasets provided by this work, IEDB, and Abelin et. al (2019). Training sets for each allele-specific Core Model were obtained by merging the datasets to obtain the HLA-II binding ligands. The non-HLA-II binding length matched decoys were added at a ratio of 1:5 (epitope: decoy). 11B) Mean confidence scores assigned by the Core Model to ligands (triangles) and decoys (circles) for 84 alleles with mono-allelic peptidomics data, depending on the number of available unique ligands —positive training examples. 11C) Calibration of predicted probabilities. Two of the heterodimers for each HLA-DP and -DQ profiled in this study with most ligands were selected, as well as DRB1*11:01 with most ligands in DR isotypes and DRB1*03:02 with a small number of publicly available epitopes. Peptides associated with different values of predicted binding confidence (0-100%) were grouped into bins of 5% increments (x-axis). The fraction of correct predictions in each bin is shown as accuracy (%, y-axis). The output predictive probability can thus be interpreted as a likelihood of binding in comparison to randomly selected peptides, which is accurately calibrated for the Core Model. While the best other method was NetMHCIIpan, which also showed significant correspondence between predictions and observations, the raw scores showed over- or under-confidence in multiple cases likely due to different training sets. 11D) (SEQ ID NO: 22-85) Visualization of parameters in the first layer (convolution) of binding core neural network models for DQA1*01:01, DQB1*06:03. Each allele-specific model includes 64 position weight matrices of length 9 or 15 aa (motifs). The weights are limited to non-negative values and information content in bits is computed against the background amino acid distribution in the human proteome. Motifs are hierarchically clustered based on Euclidean distance and representative examples are visualized below the dendrograms. In the case of DQA1*01:01, DQB1*06:03, they reveal a background distribution which recapitulates expected depletion in cysteine, methionine, characteristic for MS-based assays (Sachs et al. 2020; Abelin et al. 2019). In addition, motifs reveal characteristic P1 amino acids (tyrosine, phenylalanine, branched-chain amino acids), proline importance and DQB1*06:03-specific aspartate and threonine, demonstrating sequence properties beyond amino acid frequencies at binding positions, some of which may be shared between the alleles and are addressed using Context models below. 11E) Comparison of binding core motifs for observed and predicted ligands. Two GibbsCluster motifs are shown for observed peptides (left), and top predicted peptides in held-out data for three representative alleles, where models are available. Comparison of most confident predicted sequences reveals binding core motifs comparable to FIG. 2 (main text) for all predictors, but a more pronounced presence of secondary, P-rich motifs for the Core models. The latter can be present on the boundary between the binding core sequence and peptide flanking region (DQA1*01:01, DQB1*06:03).

FIG. 12A-12E—Details on training the CAPTAn-context and CAPTAn models. 12A) Area under the receiver-operating characteristic curve (AUC) for validation data split during context model training. 12B) Area under the precision-recall curve (average precision, AVP) for validation data split during context model training. 12C) Calibration of context models. Ligand regions associated with different values of predicted binding confidence (0-100%) were grouped into bins of 5% increments (x-axis). The fraction of correct prediction in each bin is shown as accuracy (%). 12D) Inference of weights yielding the ensemble model. Results of weighting between core and context models in a grid search. Using 30% of the training data, the weighting (w) between core and context models is selected to maximize the accuracy in the top 100 predicted regions. 12E) Comparison of context model predictions for 3,523 secreted human proteins. Secreted proteins are defined by the presence of a signal peptide (sp) of median length 23 amino acids on the N-terminus. The comparison shows maximum confidence of the HLA-DP,-DQ, and -DR context models if the signal peptide is removed (-sp, removal of all sp amino acids except starting M) or randomly mutated (mt sp, respecting the amino acid prior distribution). The original proteins are marked as WT. The confidence values are compared between protein variants using the Wilcoxon paired test.

FIG. 13A-13K—Immunopeptidomics, peptide-allele deconvolution and validation of predictions. 13A) Theoretical vs. measured retention time of human and unfiltered microbial peptides. Theoretical retention time was generated using DeepLC (v0.2.2), which bases predictions on the hydrophobicities of amino acids in individual peptide sequences. 13B) Theoretical vs. measured retention time of human and quality filtered microbial peptides. Quality filtering metrics included: SpectrumMill score >7, backbone cleavage score >8, scored peak intensity >60%, and a ppm <|3|. 13C) Log 10 total peptide intensities of HLA-II peptides of human and microbial peptides 13D) Log 10 protein iBAQ values of human and microbial proteins detected during proteomic analysis. 13E) DC-microbe coculture and immunopeptidomics. Maximum confidence score of the Core model from 9 alleles of host HLA-II type for each peptide. 13F) Deconvolution of peptides mapping to alleles with confidence score >50%. 13G) Gibbs Cluster motifs obtained from all microbial peptides (right). 13H) Microbial proteins statistics and predictions. Enrichment of proteins with at least one PFAM domain, secreted proteins and transmembrane proteins. The properties of 505 recovered proteins are compared to 18,999 proteins constituting the proteomes of A. muciniphila, B. thetaiotaomicron, R. gnavus, V. parvula, C. clostridioforme and B. longum. Probability of recovering a protein given its annotation with a PFAM domain, transmembrane or signal peptide (secreted protein). 13I) Cytokine responses in PBMCs from three healthy DRB1*03:01 positive donors to predicted peptide epitopes (DC1-8), negative controls (DMSO, CLIP, IGRP) and positive control Infl_NP, C_tet. 13J) Gating strategy for identification of DC7-specific CD4 T cells after negative magnetic enrichment of T cells and positive magnetic enrichment of tetramer-bound cells. PBMCs were stained with DC7 loaded DRB1*03:01 tetramers, and identified as single, live, CD45+CD3+CD4+ T cells. A dump channel with CD19 and CD14 was used to identify and exclude non-T cells. 13K) Staining of peripheral CD4+ and CD8+ T cells from three DRB1*03:01 positive healthy donors with DRB1*03:01 tetramers loaded with DC7 peptide from V. parvula WP_156697519.1. See F for gating strategy.

FIG. 14—A block diagram depicting a portion of a communications and processing architecture of a typical system to acquire one or more amino acids from a database or user and perform machine learning methods resulting in one or more immunogenic peptides comprising one or more peptide binding motifs for use in immunological compositions, in accordance with certain examples of the technology disclosed herein.

FIG. 15A-15C—A block flow diagram depicting methods to generate one or more immunogenic peptides comprising one or more peptide binding motifs for use in immunological compositions with optional embodiments further described herein, in accordance with certain examples of the technology disclosed herein.

FIG. 16—A block diagram depicting a computing machine and modules, in accordance with certain examples of the technology disclosed herein. The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

The embodiments disclosed herein can utilize machine learning to generate one or more immunogenic peptides comprising one or more peptide binding motifs for use in immunological compositions, as further defined below, which in turn allows for activating immune responses to infection, cancer, allergy, autoimmune diseases, and also in direct killing of cancer cells. In one aspect, technologies herein provide methods generate one or more immunogenic peptides comprising one or more peptide binding motifs for use in immunological compositions. These methods use various machine learning methods to generate one or more immunogenic peptides comprising one or more peptide binding motifs from one or more amino acid sequences. These methods optionally comprise various second machine learning methods to generate one or more ligand regions from a second one or more amino acid sequences. These methods further optionally comprise an ensemble network that generates a refined set of the one or more immunogenic peptides comprising one or more peptide binding motifs from the combination of the one or more immunogenic peptides comprising one or more peptide binding motifs with the one or more ligand regions. In another aspect, the technology includes applications and systems to generate one or more immunogenic peptides comprising one or more peptide binding motifs for use in immunological compositions. For example, applications may be provided to individual users capable of communicating through wireless means. In one aspect, technology includes products to generate one or more immunogenic peptides comprising one or more peptide binding motifs for use in immunological compositions to operate on user computing devices. The application may be a downloadable application or application programming interface for use on a computing device that generates one or more immunogenic peptides comprising one or more peptide binding motifs. The data may include amino acid sequence data. The amino acid sequence data may comprise of various lengths or the entire protein from which the amino acid is derived.

Standard techniques related to making and using aspects of the invention may or may not be described in detail herein. Various aspects of computing systems and specific computer programs to implement the various technical features described herein are well known.

### Example System Architectures

Turning now to the drawings, in which like numerals represent like (but not necessarily identical) elements throughout the figures, example embodiments are described in detail.

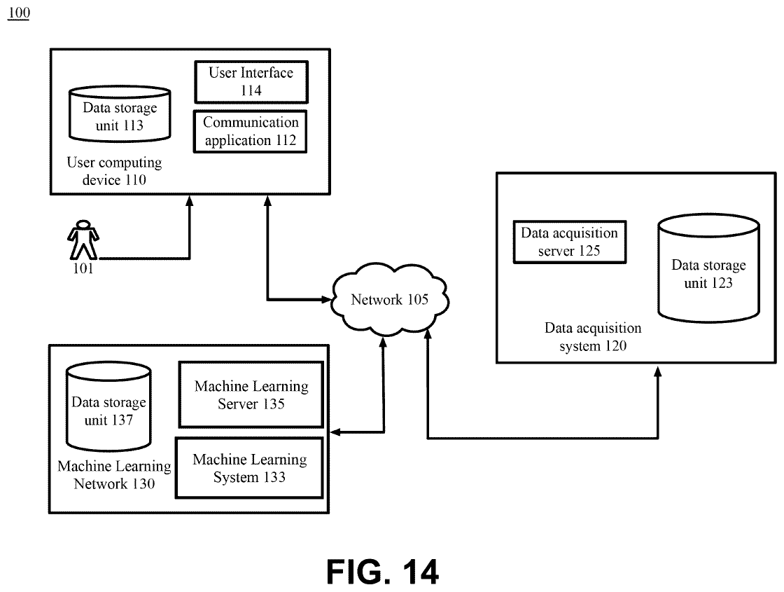

FIG. 14 is a block diagram depicting a system 100 to generate one or more immunogenic peptides comprising one or more peptide binding motifs for use in immunological compositions and perform machine learning on one or more amino acid sequences. In one example embodiment, a user 101 associated with a user computing device 110 must install an application, and or make a feature selection to obtain the benefits of the techniques described herein.

As depicted in FIG. 14, the system 100 includes network computing devices/systems 110, 120, and 130 that are configured to communicate with one another via one or more networks 105 or via any suitable communication technology.

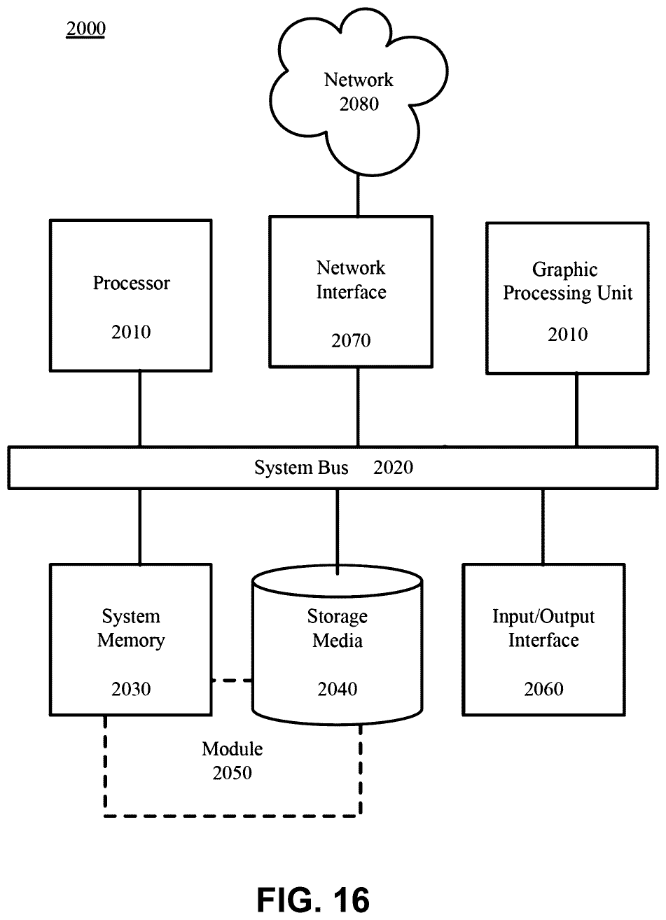

Each network 105 includes a wired or wireless telecommunication means by which network devices/systems (including devices 110, 120, and 130) can exchange data. For example, each network 105 can include any of those described herein such as the network 2080 described in FIG. 16 or any combination thereof or any other appropriate architecture or system that facilitates the communication of signals and data. Throughout the discussion of example embodiments, it should be understood that the terms “data” and “information” are used interchangeably herein to refer to text, images, audio, video, or any other form of information that can exist in a computer-based environment. The communication technology utilized by the devices/systems 110, 120, and 130 may be similar networks to network 105 or an alternative communication technology.

Each network computing device/system 110, 120, and 130 includes a computing device having a communication module capable of transmitting and receiving data over the network 105 or a similar network. For example, each network device/system 110, 120, and 130 can include any computing machine 2000 described herein and found in FIG. 16 or any other wired or wireless, processor-driven device. In the example embodiment depicted in FIG. 14, the network devices/systems 110, 120, and 130 are operated by user 101, data acquisition system operators, and machine learning network operators, respectively.

The user computing device 110 includes a user interface 114. The user interface 114 may be used to display a graphical user interface and other information to the user 101 to allow the user 101 to interact with the data acquisition system 120, the machine learning network 130, and others. The user interface 114 receives user input for data acquisition and/or machine learning and displays results to user 101. In another example embodiment, the user interface 114 may be provided with a graphical user interface by the data acquisition system 120 and or the machine learning network 130. The user interface 114 may be accessed by the processor of the user computing device 110. The user interface may display 114 may display a webpage associate with the data acquisition system 120 and/or the machine learning network 130. The user interface 114 may be used to provide input, configuration data, and other display direction by the webpage of the data acquisition system 120 and/or the machine learning network 130. In another example embodiment, the user interface 114 may be managed by the data acquisition system 120, the machine learning network 130, or others. In another example embodiment, the user interface 114 may be managed by the user computing device 110 and be prepared and displayed to the user 101 based on the operations of the user computing device 110.

The user 101 can use the communication application 112 on the user computing device 110, which may be, for example, a web browser application or a stand-alone application, to view, download, upload, or otherwise access documents or web pages through the user interface 114 via the network 105. The user computing device 110 can interact with the web servers or other computing devices connected to the network, including the data acquisition server 125 of the data acquisition system 120 and the machine learning server 135 of the machine learning network 130. In another example embodiment, the user computing device 110 communicates with devices in the data acquisition system 120 and/or the machine learning network 130 via any other suitable technology, including the example computing system described below.

The user computing device 110 also includes a data storage unit 113 accessible by the user interface 114, the communication application 112, or other applications. The example data storage unit 113 can include one or more tangible computer-readable storage devices. The data storage unit 113 can be stored on the user computing device 110 or can be logically coupled to the user computing device 110. For example, the data storage unit 113 can include on-board flash memory and/or one or more removable memory accounts or removable flash memory. In another example embodiments, the data storage unit 113 may reside in a cloud-based computing system.

An example data acquisition system 120 comprises a data storage unit 123 and an acquisition server 125. The data storage unit 123 can include any local or remote data storage structure accessible to the data acquisition system 120 suitable for storing information. The data storage unit 123 can include one or more tangible computer-readable storage devices, or the data storage unit 123 may be a separate system, such as a different physical or virtual machine or a cloud-based storage service.

In one aspect, the data acquisition server 125 communicates with the user computing device 110 and/or the machine learning network 130 to transmit requested data. The data may include one or more amino acids and/or second one or more amino acid sequences.

An example machine learning network 130 comprises a machine learning system 133, a machine learning server 135, and a data storage unit 137. The machine learning server 135 communicates with the user computing device 110 and/or the data acquisition system 120 to request and receive data. The data may comprise the data types previously described in reference to the data acquisition server 125.

The machine learning system 133 receives an input of data from the machine learning server 135. The machine learning system 133 can comprise one or more functions to implement any of the mentioned training methods to learn a one or more immunogenic peptides comprising one or more peptide binding motifs of one or more amino acid sequences or one or more ligand regions. In a preferred embodiment, the machine learning program may comprise a neural network. In one example embodiment, the neural network may comprise a convolutional neural network. In another example embodiment, the neural network may comprise a recurrent neural network. In another example embodiment, the neural network may comprise bi-directional long-short term memory (LSTM). Any suitable architecture may be applied to learn one or more immunogenic peptides comprising one or more peptide binding motifs of one or more amino acid sequences or one or more ligand regions of a second one or more amino acid sequences.

The data storage unit 137 can include any local or remote data storage structure accessible to the machine learning network 130 suitable for storing information. The data storage unit 137 can include one or more tangible computer-readable storage devices, or the data storage unit 137 may be a separate system, such as a different physical or virtual machine or a cloud-based storage service.

In an alternate embodiment, the functions of either or both data acquisition system 120 and the machine learning network 130 may be performed by the user computing device 110.

It will be appreciated that the network connections shown are examples, and other means of establishing a communications link between the computers and devices can be used. Moreover, those having ordinary skill in the art having the benefit of the present disclosure will appreciate that the user computing device 110, data acquisition system 120, and the machine learning network 130 illustrated in FIG. 14 can have any of several other suitable computer system configurations. For example, a user computing device 110 embodied as a mobile phone or handheld computer may not include all the components described above.

In example embodiments, the network computing devices and any other computing machines associated with the technology presented herein may be any type of computing machine such as, but not limited to, those discussed in more detail with respect to FIG. 16. Furthermore, any modules associated with any of these computing machines, such as modules described herein, or any other modules (scripts, web content, software, firmware, or hardware) associated with the technology presented herein may by any of the modules discussed in more detail with respect to FIG. 16. The computing machines discussed herein may communicate with one another as well as other computer machines or communication systems over one or more networks, such as network 105. The network 105 may include any type of data or communications network, including any of the network technology discussed with respect to FIG. 16.

### Example Processes

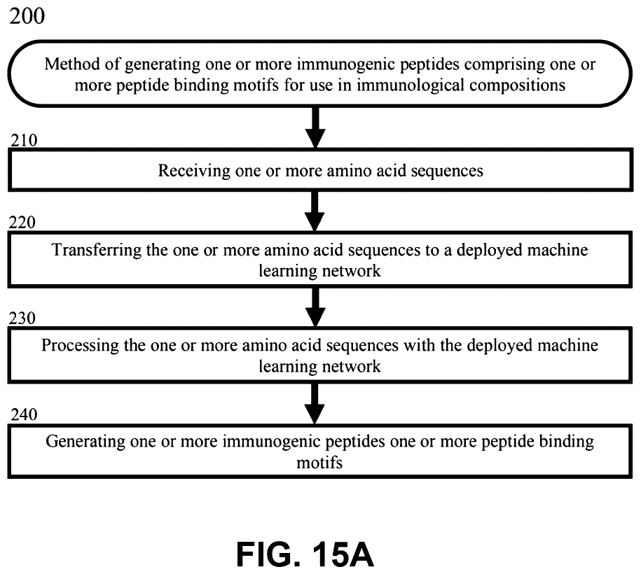

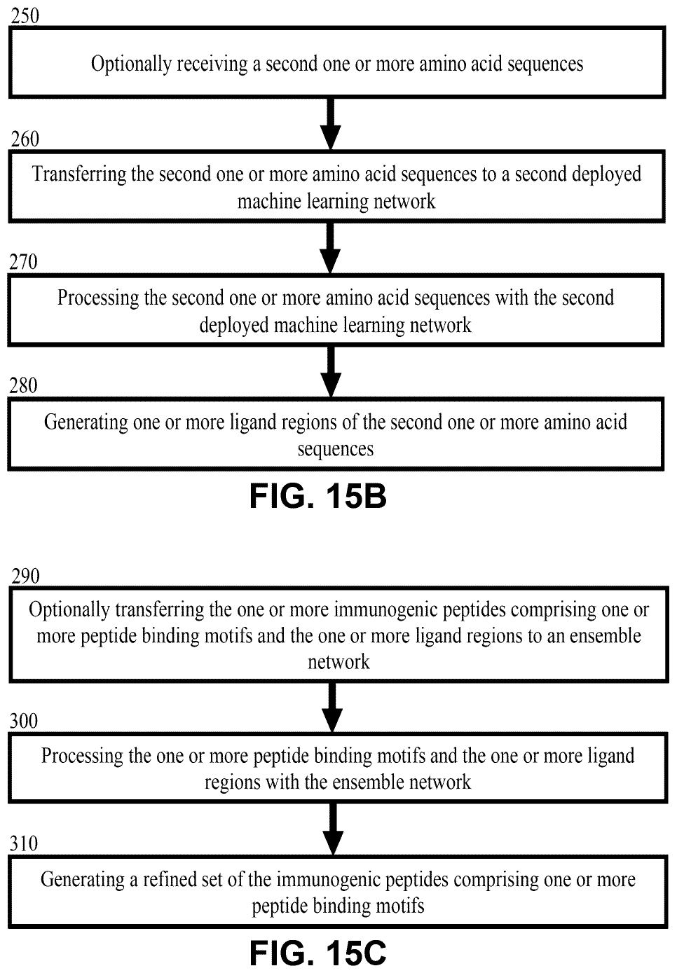

The example methods illustrated in FIG. 15 is described hereinafter with respect to the components of the example architecture 100. The example methods also can be performed with other systems and in other architectures including similar elements.

Referring to FIG. 15, and continuing to refer to FIG. 14 for context, a block flow diagram illustrates methods 200 to generate one or more immunogenic peptides comprising one or more peptide binding motifs for use in immunological compositions, in accordance with certain examples of the technology disclosed herein.

### Immune System and Adaptive Immunity

The innate and adaptive immune system comprise the functional subsystems of the immune system. Both subsystems comprise of humoral immunity components and cell-mediated components. The innate immune system provides a broad approach to subdue and eliminate pathogens and is the first line of defense against infection. On the other hand, the adaptive immune system creates immunological memory to specifically target pathogens the body has previously combatted. This memory is formed when the adaptive immune system interacts with the molecular structure of invasive organisms, also known as antigens.

Humoral immune response comprises of B cells producing antibodies unique to specific antigens. If a B cell comes into contact with an antigen, then the antigen is taken inside the B cell and stimulates the division of antigen bound B cell. This results in increased defensive response and improved memory of the pathogen. Cell-mediated immunity comprises, for example, macrophages, natural killer cells, and T-cells. Macrophages directly target pathogens and eliminate them via phagocytosis, while natural killer cells secrete and recognize cytotoxic granules to annihilate pathogens. T-cells target compromised body cells displaying epitopes of foreign antigens on their surface and induces apoptosis.

The molecules associated with presenting cell surface peptides are known as molecules of the major histocompatibility complex (MHC). The MHC comprises of proteins that allow the immune system (e.g., T cells) to bind and recognize itself. There are two subgroups of MHC proteins involved in antigen presentation, MHC class I (MHC I) and MHC class II (MHC II). MHC I are expressed in most cells and present epitopes to T-cells. T-cells, also known as cytotoxic T lymphocytes (CTLs), comprise of CD8 receptors and T-cell receptors (TCRs). If a T-cell binds to a cell (via the CD8 receptor) and recognizes an epitope (via the TCR), then the T-cell triggers the cell to undergo apoptosis. MHC II are normally expressed in in macrophages, B cells, dendritic cells, and other antigen presenting cells. In general, MHC II presents epitopes to recruit helper T-cells. Similar to T-cells, helper T-cells also comprise TCRs but also comprise CD4 receptors. The CD4 receptors of the helper T-cell binds to a MHC II molecule and searches for epitopes via the TCR. If the TCE recognizes an epitope, the T-cell releases cytokines thereby polarizing and recruiting additional immune responses.

Structurally, MHC I and MHC II proteins are similar in that they are both heterodimers comprising both α and β subunits. However, MHC I comprises of three polymorphic α-subunits with an invariant β-subunit, while MHC II comprises of two polymorphic α- and β-subunits. Consequently, the function of the subunits varies by class. For example, the α₁ and α₂ domains of MHC I form the peptide binding groove while α₁ and β₁ form the binding groove of MHC II. Additionally, the immunoglobin-like domain in MHC I that interacts with the CD8 receptor is the α₃-subunit, while the immunoglobin domains in MHC II that interact with CD4 are the α₂- and β₂-subunits. MHC I also comprises of a B2 microglobulin subunit but it participates in the stabilization and recognition of the peptide-MHC complex by a CD8 co-receptor. See Wieczorek, M.; et al. Major Histocompatibility Complex (MHC) Class I and MHC Class II Proteins: Conformational Plasticity in Antigen Presentation. Frontiers in Immunology, 2017, 8. In an example embodiment, peptide binding motifs demonstrate binding and/or enhancing binding to MHC molecules.

### HLA System

The MHC in humans is known as the human leukocyte antigen (HLA) system. Similar to other MHCs, the HLA system comprises of major and minor classes. The major classes of the HLA system are the most polymorphic. Both the HLA MHC-I (HLA-I) and the HLA MHC-II (HLA-II) comprise of 3 major classes: HLA-A; HLA-B; HLA-C, corresponding to the α-subunits of HLA-I and HLA-DP; HLA-DQ; HLA-DR each of which corresponding to α- and β-subunits of HLA-II. Since the major classes correspond to the peptide binding groove and have a high allelic variation, the range of peptides capable of binding to HLA-I and HLA-II is numerous. The peptide binding groove typically comprise of eight anti-parallel β-sheets and two anti-parallel α-helices. The minor class of HLA-I and HLA-II comprise: HLA-E; HLA-H; HLA-G corresponding to the β-subunit of HLA-I and HLA-DM; HLA-DO corresponding to domains whose function is processing and loading of peptides onto the HLA-II protein.

The terms “molecules of the major histocompatibility complex (MHC)”, “MHC molecule”, “MHC protein” or “HLA protein” as used herein refer to proteins capable of binding peptides resulting from the proteolytic cleavage of proteins as well as representing potential epitopes, transporting them to and presenting on the cell surface to T cells such as CTLs or T-helper cells. In an example embodiment, peptide binding motifs demonstrate binding and/or enhancing binding to HLA molecules.

### Peptide Antigen

In block 210, the machine learning network 130 receives an input of one or more amino acid sequences. The machine learning network 130 may receive the one or more amino acid sequences from the user computing device 110, the data acquisition system 120, or any other suitable source of amino acid sequence data via the network 105 to the machine learning network 130, discussed in more detail in other sections herein. The acquisition engine comprises any software or hardware individually or in combination described herein and/or known in the art that is capable of or allows for fetching or receiving the one or more amino acids thereby allowing access to the one or more amino acids by the machine learning network 130 or the data acquisition system 120.

Peptide-MHC complex formation is unique to each MHC class. MHC 1-peptide complexes begin with protein degradation via the proteasome in a cell. As a result, both endogenous peptides and invasive peptides may bind with MHC I. Example endogenous peptides may be those derived from protein turnover, defective ribosomal products, or peptides resulting from cancerous transformation. Example invasive peptides may be those derived from pathogens. These peptides enter the endoplasmic reticulum (ER) and form a complex with MHC I through non-covalent binding between the amino acid sequence of the peptide and the amino acid sequence of the MHC I protein.

MHC-II peptide complexes begin with endocytosis of exogenous proteins into the endosome. The exogenous proteins undergo proteolysis. Meanwhile, MHC-II proteins fold in the ER with an invariant chain (IC) protein. The proteolytically derived peptides and MCH-II meet within the endosome. Before complexation between the peptides and MHC-II proteins, the IC is cleaved by a cathepsin protease leaving behind a class II-associated invariant chain peptide (CHIP) bound to the MHC-II protein. The CHIP peptide is exchanged for higher affinity peptides. Consequently, in both MHC-I and MHC-II, only peptides with strong affinity for a particular MHC protein will form an MHC-peptide complex.

Peptide-MHC affinity is influenced by a peptide's structure. For example, MHC-I is capable of binding peptides of around 8-11 amino acids in length, while MHC-II is capable of binding peptides of around 11-30 amino acids in length. Shorter peptides that bind to MHC-I comprise of residues whose side chains anchor the peptide to the HLA protein. Example anchor residues may be P2 or P5/6 and PQ. These residues are typically oriented toward the interior of the peptide binding groove and away from the TCR. The other residues typically point toward the TCR thereby mediating epitope specificity. Longer peptides also comprise of anchor residues, but peptides comprise of more variation in their sequence. Example anchor residues comprise P1, P4, P6, and P9. The backbone of MHC-II peptides typically binds to the peptide binding groove. In both the MHC-I and the MHC-II, affinity is conditional on the interactions between the peptide sidechains with the geometry, charge distribution, and hydrophobicity of the binding groove.

After a peptide forms a complex with a MHC protein, the complex is transported to and presented on the cell surface. Developing methods and systems to generate the sequence of one or more peptide antigens can allow for the manipulation of the MHC system, thereby allowing for the development of, for example, peptide vaccines. See, Ryan J. Malonis, Jonathan R. Lai, and Olivia Vergnolle Chemical Reviews 2020 120 (6), 3210-3229.

In an example embodiment, the one or more amino acids sequences are of 7 to 100, to 75, to 50, or to 25 amino acids in length. In an example embodiment, the one or more amino acid sequences have a maximum overlap of 10, 9, 8, 7, 6, or 5 amino acids.

In an example embodiment, the one or more amino acids comprise amino acids of one or more pathogens or disease. In an example embodiment, the one or more pathogens comprise bacteria, viruses, fungi, and/or protists. In an example embodiment, the one or more diseases comprise of cancer. In an example embodiment, the cancer comprises of carcinoma, sarcoma, leukemia, lymphoma, multiple myeloma, melanoma, brain and spinal cord tumors, germ cell tumors, neuroendocrine tumors, and/or carcinoid tumors.

In block 220, the one or more amino acid sequences is transferred over a network via the transfer engine from the user associated device 100 or the data acquisition system 120 to the machine learning network 130. The transfer engine comprises any software or hardware individually or in combination described herein that is capable of moving or transferring the one or more amino acid sequences thereby allowing access within the machine learning network 130. The machine learning network 130 receives input of the one or more amino acids and passes the one or more amino acids to the data storage unit 137 for temporary or permanent storage or directly to the machine learning server 135.

In block 230, the machine learning system 133 processes the data of the one or more amino acids using a machine learning method described further herein. In an example embodiment, the machine learning system 133 receives, through methods previously described, receives one or more features of the one or more amino acid sequences. In an example embodiment, the one or more features comprise binary, fractional, or both features. In an example embodiment, the binary features are represented as a vector. The vector can be of any length. The vector length may be determined, in part, by the features one seeks to describe with the machine learning model. For example, binary features may comprise of the order of residues, the type of residues, or function of the residues. In general, a binary feature is any feature that can be described as absent (i.e., 0) or present (i.e., 1). For example, a residue comprising a charge may be represented as a 1 whereas a residue without a charge may be represented as a 0. In an example embodiment, the fractional features comprise secreted protein features, transmembrane features, domain features, region features, relative solvent accessibility features, and disorder features. Features may comprise of characteristics corresponding to the aspect they describe. For example, a domain feature may comprise of information regarding protein structure, folding, function, evolution, or design.

In block 240, the machine system 133 generates output data comprising information containing one or more immunogenic peptides comprising one or more peptide binding motifs. In an example embodiment, the machine learning system of 133 further generates one or more immunogenic peptides comprising one or more peptide binding motifs further comprise individual probability or confidence scores. In an example embodiment, one or more immunogenic peptides comprising one or more peptide binding motifs is around 20 amino acids in length. In an example embodiment, the one or more immunogenic peptides comprising peptide binding motifs are specific to HLA-II alleles specific to a subject. In an example embodiment, the immunological composition is a protective vaccine or tolerizing vaccine composition comprising one or more of the antigenic epitopes.

### HLA Alleles

The one or more immunogenic peptides comprising one or more peptide binding motifs may bind, or may be capable of binding, to proteins encoded by certain HLA alleles. HLA genes may be polymorphic and have many different alleles, allowing them to fine-tune the immune system. The nomenclature of HLA genes is well known in the art, e.g., as described in Marsh SGE et al., Nomenclature for factors of the HLA system, 2010, Tissue Antigens. 2010 April; 75 (4): 291-455, which is incorporated by reference in its entirety.

The HLA alleles may encode HLA protein capable of epitope binding. In some embodiments, the proteins encoded by HLA alleles include HLA proteins encoded by encoded by HLA-A*02:01, HLA-A*25:01, HLA-A*30:01, HLA-B*18:01, HLA-B*44:03, HLA-C*12:03, HLA-B*16:01, HLA-A*02:01, HLA-B*07:02, HLA-C*07:02; HLA-A*01:01; HLA-A*02:06; HLA-A*26:01; HLA-A*02:07; HLA-A*29:02; HLA-A*02:03; HLA-A*30:02; HLA-A*32:01; HLA-A*68:02; HLA-A*02:05; HLA-A*02:02; HLA-A*36:01; HLA-A*02:11; HLA-A*02:04; HLA-B*35:01; HLA-B*51:01; HLA-B*40:01; HLA-B*40:02; HLA-B*07:02; HLA-B*07:04; HLA-B*08:01; HLA-B*13:01; HLA-B*46:01; HLA-B*52:01; HLA-B*44:02; HLA-B*40:06; HLA-B*13:02; HLA-B*56:01; HLA-B*54:01; HLA-B*15:02; HLA-B*35:07; HLA-B*27:05; HLA-B*15:03; HLA-B*42:01; HLA-B*55:02; HLA-B*45:01; HLA-B*50:01; HLA-B*35:03; HLA-B*49:01; HLA-B*58:02; HLA-B*15:17; HLA-C*57:02; HLA-C*04:01; HLA-C*03:04; HLA-C*01:02; HLA-C*07:01; HLA-C*06:02; HLA-C*03:03; HLA-C*08:01; HLA-C*15:02; HLA-C*12:02; HLA-C*02:02; HLA-C*05:01; HLA-C*03:02; HLA-C*16:01; HLA-C*08:02; HLA-C*04:03; HLA-C*17:01; or HLA-C*17:04. In some embodiments, the HLA-1 is encoded by HLA-A*02:01, HLA-A*25:01, HLA-A*30:01, HLA-B*18:01, HLA-B*44:03, HLA-C*12:03, HLA-B*16:01, HLA-A*02:01, HLA-B*07:02, or HLA-C*07:02. In some embodiments, the proteins encoded by HLA alleles include HLA proteins encoded by HLA-A*02:01, HLA-A*25:01, HLA-A*30:01, HLA-B*18:01, HLA-B*44:03, HLA-C*12:03, HLA-B*16:01, HLA-A*02:01, HLA-B*07:02, HLA-C*07:02, or a combination thereof.

In one example, the one or more immunogenic peptides comprising one or more peptide binding motifs binds, or is capable of binding, to an HLA protein encoded by HLA-A*02:01. In another example, the one or more immunogenic peptides comprising one or more peptide binding motifs binds, or is capable of binding, to an HLA protein encoded by HLA-A*25:01. In another example, the one or more immunogenic peptides comprising one or more peptide binding motifs binds, or is capable of binding, to an HLA protein encoded by HLA-A*30:01. In another example, the one or more immunogenic peptides comprising one or more peptide binding motifs binds, or is capable of binding, to an HLA protein encoded by HLA-B*18:01. In another example, the one or more immunogenic peptides comprising one or more peptide binding motifs binds, or is capable of binding, to an HLA protein encoded by HLA-B*44:03. In another example, the one or more immunogenic peptides comprising one or more peptide binding motifs binds, or is capable of binding, to an HLA protein encoded by HLA-C*12:03. In another example, the one or more immunogenic peptides comprising one or more peptide binding motifs binds, or is capable of binding, to an HLA protein encoded by HLA-B*16:01. In another example, the one or more immunogenic peptides comprising one or more peptide binding motifs binds, or is capable of binding, to an HLA protein encoded by HLA-A*02:01. In another example, the one or more immunogenic peptides comprising one or more peptide binding motifs binds, or is capable of binding, to an HLA protein encoded by HLA-B*07:02. In another example, the one or more immunogenic peptides comprising one or more peptide binding motifs binds, or is capable of binding, to an HLA protein encoded by HLA-C*07:02. In another example, the one or more immunogenic peptides comprising one or more peptide binding motifs binds, or is capable of binding, to an HLA protein encoded by HLA-A*01:01. In another example, the one or more immunogenic peptides comprising one or more peptide binding motifs binds, or is capable of binding, to an HLA protein encoded by HLA-A*02:06. In another example, the one or more immunogenic peptides comprising one or more peptide binding motifs binds, or is capable of binding, to an HLA protein encoded by HLA-A*26:01. In another example, the one or more immunogenic peptides comprising one or more peptide binding motifs binds, or is capable of binding, to an HLA protein encoded by HLA-A*02:07. In another example, the one or more immunogenic peptides comprising one or more peptide binding motifs binds, or is capable of binding, to an HLA protein encoded by HLA-A*29:02. In another example, the one or more immunogenic peptides comprising one or more peptide binding motifs binds, or is capable of binding, to an HLA protein encoded by HLA-A*02:03. In another example, the one or more immunogenic peptides comprising one or more peptide binding motifs binds, or is capable of binding, to an HLA protein encoded by HLA-A*30:02. In another example, the one or more immunogenic peptides comprising one or more peptide binding motifs binds, or is capable of binding, to an HLA protein encoded by HLA-A*32:01. In another example, the one or more immunogenic peptides comprising one or more peptide binding motifs binds, or is capable of binding, to an HLA protein encoded by HLA-A*68:02. In another example, the one or more immunogenic peptides comprising one or more peptide binding motifs binds, or is capable of binding, to an HLA protein encoded by HLA-A*02:05. In another example, the one or more immunogenic peptides comprising one or more peptide binding motifs binds, or is capable of binding, to an HLA protein encoded by HLA-A*02:02. In another example, the one or more immunogenic peptides comprising one or more peptide binding motifs binds, or is capable of binding, to an HLA protein encoded by HLA-A*36:01. In another example, the one or more immunogenic peptides comprising one or more peptide binding motifs binds, or is capable of binding, to an HLA protein encoded by HLA-A*02:11. In another example, the one or more immunogenic peptides comprising one or more peptide binding motifs binds, or is capable of binding, to an HLA protein encoded by HLA-A*02:04. In another example, the one or more immunogenic peptides comprising one or more peptide binding motifs binds, or is capable of binding, to an HLA protein encoded by HLA-B*35:01. In another example, the one or more immunogenic peptides comprising one or more peptide binding motifs binds, or is capable of binding, to an HLA protein encoded by HLA-B*51:01. In another example, the one or more immunogenic peptides comprising one or more peptide binding motifs binds, or is capable of binding, to an HLA protein encoded by HLA-B*40:01. In another example, one or more immunogenic peptides comprising the one or more peptide binding motifs binds, or is capable of binding, to an HLA protein encoded by HLA-B*40:02. In another example, the one or more immunogenic peptides comprising one or more peptide binding motifs binds, or is capable of binding, to an HLA protein encoded by HLA-B*07:02. In another example, the one or more immunogenic peptides comprising one or more peptide binding motifs binds, or is capable of binding, to an HLA protein encoded by HLA-B*07:04. In another example, the one or more immunogenic peptides comprising one or more peptide binding motifs binds, or is capable of binding, to an HLA protein encoded by HLA-B*08:01. In another example, one or more immunogenic peptides comprising the one or more peptide binding motifs binds, or is capable of binding, to an HLA protein encoded by HLA-B*13:01. In another example, the one or more immunogenic peptides comprising one or more peptide binding motifs binds, or is capable of binding, to an HLA protein encoded by HLA-B*46:01. In another example, the one or more immunogenic peptides comprising one or more peptide binding motifs binds, or is capable of binding, to an HLA protein encoded by HLA-B*52:01. In another example, one or more immunogenic peptides comprising the one or more peptide binding motifs binds, or is capable of binding, to an HLA protein encoded by HLA-B*44:02. In another example, the one or more immunogenic peptides comprising one or more peptide binding motifs binds, or is capable of binding, to an HLA protein encoded by HLA-B*40:06. In another example, the one or more immunogenic peptides comprising one or more peptide binding motifs binds, or is capable of binding, to an HLA protein encoded by HLA-B*13:02. In another example, the one or more immunogenic peptides comprising one or more peptide binding motifs binds, or is capable of binding, to an HLA protein encoded by HLA-B*56:01. In another example, the one or more immunogenic peptides comprising one or more peptide binding motifs binds, or is capable of binding, to an HLA protein encoded by HLA-B*54:01. In another example, the one or more immunogenic peptides comprising one or more peptide binding motifs binds, or is capable of binding, to an HLA protein encoded by HLA-B*15:02. In another example, the one or more immunogenic peptides comprising one or more peptide binding motifs binds, or is capable of binding, to an HLA protein encoded by HLA-B*35:07. In another example, the one or more immunogenic peptides comprising one or more peptide binding motifs binds, or is capable of binding, to an HLA protein encoded by HLA-B*27:05. In another example, the one or more immunogenic peptides comprising one or more peptide binding motifs binds, or is capable of binding, to an HLA protein encoded by HLA-B*15:03. In another example, the one or more immunogenic peptides comprising one or more peptide binding motifs binds, or is capable of binding, to an HLA protein encoded by HLA-B*42:01. In another example, the one or more immunogenic peptides comprising one or more peptide binding motifs binds, or is capable of binding, to an HLA protein encoded by HLA-B*55:02. In another example, the one or more immunogenic peptides comprising one or more peptide binding motifs binds, or is capable of binding, to an HLA protein encoded by HLA-B*45:01. In another example, the one or more immunogenic peptides comprising one or more peptide binding motifs binds, or is capable of binding, to an HLA protein encoded by HLA-B*50:01. In another example, the one or more immunogenic peptides comprising one or more peptide binding motifs binds, or is capable of binding, to an HLA protein encoded by HLA-B*35:03. In another example, the one or more immunogenic peptides comprising one or more peptide binding motifs binds, or is capable of binding, to an HLA protein encoded by HLA-B*49:01. In another example, the one or more immunogenic peptides comprising one or more peptide binding motifs binds, or is capable of binding, to an HLA protein encoded by HLA-B*58:02. In another example, the one or more immunogenic peptides comprising one or more peptide binding motifs binds, or is capable of binding, to an HLA protein encoded by HLA-B*15:17. In another example, the one or more immunogenic peptides comprising one or more peptide binding motifs binds, or is capable of binding, to an HLA protein encoded by HLA-C*57:02. In another example, the one or more immunogenic peptides comprising one or more peptide binding motifs binds, or is capable of binding, to an HLA protein encoded by HLA-C*04:01. In another example, the one or more immunogenic peptides comprising one or more peptide binding motifs binds, or is capable of binding, to an HLA protein encoded by HLA-C*03:04. In another example, the one or more immunogenic peptides comprising one or more peptide binding motifs binds, or is capable of binding, to an HLA protein encoded by HLA-C*01:02. In another example, the one or more immunogenic peptides comprising one or more peptide binding motifs binds, or is capable of binding, to an HLA protein encoded by HLA-C*07:01. In another example, the one or more immunogenic peptides comprising one or more peptide binding motifs binds, or is capable of binding, to an HLA protein encoded by HLA-C*06:02. In another example, the one or more immunogenic peptides comprising one or more peptide binding motifs binds, or is capable of binding, to an HLA protein encoded by HLA-C*03:03. In another example, the one or more immunogenic peptides comprising one or more peptide binding motifs binds, or is capable of binding, to an HLA protein encoded by HLA-C*08:01. In another example, the one or more immunogenic peptides comprising one or more peptide binding motifs binds, or is capable of binding, to an HLA protein encoded by HLA-C*15:02. In another example, the one or more immunogenic peptides comprising one or more peptide binding motifs binds, or is capable of binding, to an HLA protein encoded by HLA-C*12:02. In another example, the one or more immunogenic peptides comprising one or more peptide binding motifs binds, or is capable of binding, to an HLA protein encoded by HLA-C*02:02. In another example, the one or more immunogenic peptides comprising one or more peptide binding motifs binds, or is capable of binding, to an HLA protein encoded by HLA-C*05:01. In another example, the one or more immunogenic peptides comprising one or more peptide binding motifs binds, or is capable of binding, to an HLA protein encoded by HLA-C*03:02. In another example, the one or more immunogenic peptides comprising one or more peptide binding motifs binds, or is capable of binding, to an HLA protein encoded by HLA-C*16:01. In another example, the one or more immunogenic peptides comprising one or more peptide binding motifs binds, or is capable of binding, to an HLA protein encoded by HLA-C*08:02. In another example, the one or more immunogenic peptides comprising one or more peptide binding motifs binds, or is capable of binding, to an HLA protein encoded by HLA-C*04:03. In another example, the one or more immunogenic peptides comprising one or more peptide binding motifs binds, or is capable of binding, to an HLA protein encoded by HLA-C*17:01. In another example, the one or more immunogenic peptides comprising one or more peptide binding motifs binds, or is capable of binding, to an HLA protein encoded by HLA-C*17:04. Additional examples of HLA alleles include those described in e.g., Table 1.

### Ligand Regions

Optionally, in block 250, the machine learning network 130 receives an input of a second one or more amino acid sequences. The machine learning network 130 may comprise of one or more machine learning networks (e.g., a first and second machine learning network). The machine learning network 130 comprising of more than one machine learning network necessarily comprises of more than one machine learning model (either the same or different type of model) and the necessary hardware as described herein. The machine learning network 130 may receive the second one or more amino acid sequences from the user computing device 110, the data acquisition system 120, or any other suitable source of amino acid sequence data via the network 105 to the machine learning network 130, discussed in more detail in other sections herein.

In an example embodiment, the second one or more amino acids comprise signaling regions. In an example embodiment, the signaling regions comprise adjacent and/or distant signaling regions. In an example embodiment, the adjacent signaling regions comprise exopeptidase trimming sites and/or proline-rich cleavage motifs. In an example embodiment, the second one or more amino acids comprises the full length of a protein. In an example embodiment, the second set of one or more amino acids are expanded sequences of the one or more amino acids of block 210. In an example embodiment, the second one or more amino acid sequences comprise the one or more amino acid sequences of block 210, wherein the one or more amino acid sequences of block 210 includes one or more additional sequences up to the full length of the source protein. In an example embodiment, the one or more amino acid sequences comprise full-length protein sequences. In an example embodiment, the one or more amino acid sequences are obtained by analyzing one or more genomic DNA sequences. In an example embodiment, the one or more genomic DNA sequences is a full genome sequence. In an example embodiment, the one or more input genome sequences is derived from a target pathogen, a commensal microorganism, or a diseased cell. In an example embodiment, the pathogen is selected from the group consisting of a bacterium, a virus, a protozoon, and an allergen. In an example embodiment, the diseased cell is a cancer cell. In an example embodiment, the one or more amino acid sequences are derived from neoantigens.

### Pathogens

In example embodiments, the one or more amino acids are derived from a pathogen and the one or more immunogenic peptides comprising one or more peptide binding motifs bind to HLA alleles are identified or predicted. In example embodiments, the infectious agent mutates to express peptides capable of binding different HLA-alleles. Thus, peptides for generating an immunological composition or vaccine require prediction of novel peptide binding to subject specific HLA alleles.

Examples of pathogenic bacteria include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia 43eoantig), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Examples of fungi include without limitation any one or more of (or any combination of), Aspergillus, Blastomyces, Candidiasis, Coccidiodomycosis, Cryptococcus neoformans, Cryptococcus gatti, Histoplasma, Mucroymcosis, Pneumocystis, Sporothrix, fungal eye infections ringwork, Exserohilum, and Cladosporium. In example embodiments, the fungus is a yeast. Examples of yeast include without limitation one or more of (or any combination of), Aspergillus species, a Geotrichum species, a Saccharomyces species, a Hansenula species, a Candida species, a Kluyveromyces species, a Debaryomyces species, a Pichia species, or combination thereof. In example embodiments, the fungus is a mold. Example molds include, but are not limited to, a Penicillium species, a Cladosporium species, a Byssochlamys species, or a combination thereof.

In example embodiments, the pathogen may be a protozoon. Examples of protozoa include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

In example embodiments, the pathogen may be a virus. The virus may be a DNA virus, a RNA virus, or a retrovirus. Example of RNA viruses include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In example embodiments, the virus may be a retrovirus. Example retroviruses include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In example embodiments, the virus is a DNA virus. Example DNA viruses that include one or more of (or any combination of) viruses from the Family Myoviridae, Podoviridae, Siphoviridae, Alloherpesviridae, Herpesviridae (including human herpes virus, and Varicella Zoster virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others.

The coronavirus may be a positive-sense single stranded RNA family of viruses, infecting a variety of animals and humans. In one example, the coronavirus is SARS-COV-2. SARS-COV is one type of coronavirus infection, as well as MERS-COV. Example sequences of the SARS-COV-2 are available at GISAID accession no. EPI ISL 402124 and EPI ISL 402127-402130, and described in DOI: 10.1101/2020.01.22.914952. Further deposits of the example SARS-COV2 are deposited in the GISAID platform include EP_ISL_402119-402121 and EP_ISL 402123-402124; see also GenBank Accession No. MN908947.3.

Additional example viruses further comprise Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\0.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O′nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno 49eoantig virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

### Neoantigens

In an example embodiment, the one or more amino acids are derived from tumor antigens and the one or more immunogenic peptides comprising one or more peptide binding motifs bind to HLA alleles. In example embodiments, the tumor antigens are neoantigens. In a further aspect, the invention provides methods for identifying tumor neoantigen-comprising peptides, wherein the methods comprise identifying for a given HLA allele, the peptides binding said HLA allele in a tumor cell from a tumor of a patient.

As described herein, there is a large body of evidence in both animals and humans that mutated epitopes are effective in inducing an immune response and that cases of spontaneous tumor regression or long-term survival correlate with CD8+ T-cell responses to mutated epitopes (Buckwalter and Srivastava PK. “It is the antigen(s), stupid” and other lessons from over a decade of vaccitherapy of human cancer. Seminars in immunology 20:296-300 (2008); Karanikas et al, High frequency of cytolytic T lymphocytes directed against a tumor-specific mutated antigen detectable with HLA tetramers in the blood of a lung carcinoma patient with long survival. Cancer Res. 61:3718-3724 (2001); Lennerz et al, The response of autologous T cells to a human melanoma is dominated by mutated neoantigens. Proc Natl Acad Sci USA.102:16013 (2005)) and that “immunoediting” can be tracked to alterations in expression of dominant mutated antigens in mice and man (Matsushita et al, Cancer exome analysis reveals a T-cell-dependent mechanism of cancer immunoediting Nature 482:400 (2012); DuPage et al, Expression of tumor-specific antigens underlies cancer immunoediting Nature 482:405 (2012); and Sampson et al, Immunologic escape after prolonged progression-free survival with epidermal growth factor receptor variant III peptide vaccination in patients with newly diagnosed glioblastoma J Clin Oncol. 28:4722-4729 (2010)).

Sequencing technology has revealed that each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). These mutated proteins are valuable targets for the host's immune response to the tumor as, unlike native proteins, they are not subject to the immune-dampening effects of self-tolerance. Therefore, mutated proteins are more likely to be immunogenic and are also more specific for the tumor cells compared to normal cells of the patient. The mutated proteins can be referred to as neoantigens. The term “neoantigen” or “neoantigenic” means a class of tumor antigens that arises from a tumor-specific mutation(s) which alters the amino acid sequence of genome encoded proteins.

Methods provided herein can be used to generate subject specific peptides that are presented by a tumor for any neoplasia. By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

### Autoimmune Diseases

In an example embodiment, the one or more amino acids are derived from self-antigen peptides and the one or more immunogenic peptides comprising one or more peptide binding motifs bind to HLA alleles. In example embodiments, the self-antigens are aberrantly targeted by the host immune system. As used throughout the present specification, the terms “autoimmune disease” or “autoimmune disorder” are used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behçet's disease; coeliac disease; dermatomyositis; diabetes mellitus type I; Goodpasture's syndrome; Graves' disease; Guillain-Barré syndrome (GBS); Hashimoto's disease; idiopathic thrombocytopeniaurpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

In block 260, the second one or more amino acid sequences is transferred over a network via the transfer engine from the user associated device 100 or the data acquisition system 120 to the machine learning network 130. The machine learning network 130 receives input of the one or more amino acids and passes the one or more amino acids to the data storage unit 137 for temporary or permanent storage or directly to the machine learning server 135.

In block 270, the machine learning system 133 processes the data of the second one or more amino acids using additional methods described further herein. In an example embodiment, the machine learning system 133 receives, through methods previously described, receives one or more features of the one or more amino acid sequences. In an example embodiment, the one or more features comprise binary, fractional, or both features. In an example embodiment, the fractional features comprise secreted protein features, transmembrane features, domain features, region features, relative solvent accessibility features, and disorder features.

In block 280, the machine system 133 generates output data comprising information containing one or more ligand regions. In an example embodiment, the machine learning system of 133 further generates one or more ligand regions further comprise individual probability or confidence scores at each position in the ligand region. In an example embodiment, the ligand region further comprises an additional 25 amino acids on either or both sides of the generated ligand region.

Optionally, in block 290, the one or more immunogenic peptides comprising one or more peptide binding motifs and the one or more ligand regions is transferred over a network via the transfer engine from the user associated device 100 or the data acquisition system 120 to the machine learning network 130 or is already located in the machine learning network 130. The machine learning network 130 receives input of the one or more immunogenic peptides comprising one or more peptide binding motifs and the one or more ligand regions and passes the one or more immunogenic peptides comprising one or more peptide binding motifs and the one or more ligand regions to the data storage unit 137 for temporary or permanent storage or directly to the machine learning server 135.

In block 300, an ensemble network, operably contained in the machine learning system 133, processes the data of the one or more immunogenic peptides comprising one or more peptide binding motifs and the one or more ligand regions into output data comprising information of a refined set of the one or more immunogenic peptides comprising one or more peptide binding motifs. In an example embodiment, the ensemble network is a combinatorial network comprising of the first and second machine learning network. In an example embodiment, the ensemble network comprising the first and second machine learning network take the output from the two machine learning network and combines them to generate a set of the immunogenic peptides comprising one or more peptide binding motifs. In an example embodiment, the ensemble network comprises of a linear regression model that takes as input the output from the two machine learning models. In an example embodiment, the ensemble network comprises grid searching.

In block 310, the machine system 133 generates output data comprising information containing a refined set of the one or more immunogenic peptides comprising one or more peptide binding motifs. In example embodiments, a refined set comprises of a reduced set of immunogenic peptides. In example embodiments, the refined set reorders or revaluates the immunogenic peptides.

At the conclusion of block 240, 280, and/or 310 the one or more immunogenic peptides comprising one or more peptide binding motifs, the one or more ligand regions, and/or the refined set of the one or more immunogenic peptides comprising one or more peptide binding motifs is transmitted back to the user via the network 105. In example embodiments, the resulting user information is stored on the data storage unit 137. In example embodiments, the resulting user information is immediately transmitted to the user's device. In example embodiments, the resulting user information is transmitted across the network 105 to the data acquisition system for subsequent access by the user associated device 100 or machine learning system 130.

In one aspect, Referring to FIG. 15, and continuing to refer to FIG. 14 for context, a block flow diagram illustrates methods 200 to generate one or more immunogenic peptides comprising one or more peptide binding motifs for use in immunological compositions, in accordance with certain examples of the technology disclosed herein. In block 210, the machine learning network 130 receives an input of one or more amino acid sequences. The machine learning network 130 may receive the one or more amino acid sequences from the user computing device 110, the data acquisition system 120, or any other suitable source of amino acid sequence data via the network 105 to the machine learning network 130, discussed in more detail in other sections herein. The acquisition engine comprises any software or hardware individually or in combination described herein and/or known in the art that is capable of or allows for fetching or receiving the one or more amino acids thereby allowing access to the one or more amino acids by the machine learning network 130 or the data acquisition system 120.

In block 220, the one or more amino acid sequences is transferred over a network via the transfer engine from the user associated device 100 or the data acquisition system 120 to the machine learning network 130. The transfer engine comprises any software or hardware individually or in combination described herein that is capable of moving or transferring the one or more amino acid sequences thereby allowing access within the machine learning network 130. The machine learning network 130 receives input of the one or more amino acids and passes the one or more amino acids to the data storage unit 137 for temporary or permanent storage or directly to the machine learning server 135.

In block 230, the machine learning system 133 processes the data of the one or more amino acids using a convolutional neural network as described further herein. In an example embodiment, the machine learning system 133 receives, through methods previously described, receives one or more features of the one or more amino acid sequences. In an example embodiment, the one or more features comprise binary, fractional, or both features. In an example embodiment, the fractional features comprise secreted protein features, transmembrane features, domain features, region features, relative solvent accessibility features, and disorder features.

In block 240, the machine system 133 generates output data comprising information containing one or more immunogenic peptides comprising one or more peptide binding motifs. In an example embodiment, the machine learning system of 133 further generates one or more immunogenic peptides comprising one or more peptide binding motifs further comprise individual probability or confidence scores. In an example embodiment, one or more immunogenic peptides comprising one or more peptide binding motifs is around 20 amino acids in length. In an example embodiment, the one or more immunogenic peptides comprising peptide binding motifs are specific to HLA-II alleles specific to a subject. In an example embodiment, the immunological composition is a protective vaccine or tolerizing vaccine composition comprising one or more of the antigenic epitopes.

In block 250, the machine learning network 130 receives an input of a second one or more amino acid sequences. The machine learning network 130 may receive the second one or more amino acid sequences from the user computing device 110, the data acquisition system 120, or any other suitable source of amino acid sequence data via the network 105 to the machine learning network 130, discussed in more detail in other sections herein.

In block 260, the second one or more amino acid sequences is transferred over a network via the transfer engine from the user associated device 100 or the data acquisition system 120 to the machine learning network 130. The machine learning network 130 receives input of the one or more amino acids and passes the one or more amino acids to the data storage unit 137 for temporary or permanent storage or directly to the machine learning server 135.

In block 270, the machine learning system 133 processes the data of the second one or more amino acids using a recurrent neural network as described further herein. In an example embodiment, the machine learning system 133 receives, through methods previously described, receives one or more features of the one or more amino acid sequences. In an example embodiment, the one or more features comprise binary, fractional, or both features. In an example embodiment, the fractional features comprise secreted protein features, transmembrane features, domain features, region features, relative solvent accessibility features, and disorder features.

In block 280, the machine system 133 generates output data comprising information containing one or more ligand regions. In an example embodiment, the machine learning system of 133 further generates one or more ligand regions further comprise individual probability or confidence scores at each position in the ligand region. In an example embodiment, the ligand region further comprises an additional 25 amino acids on either or both sides of the generated ligand region.

In block 290, the one or more immunogenic peptides comprising one or more peptide binding motifs and the one or more ligand regions is transferred over a network via the transfer engine from the user associated device 100 or the data acquisition system 120 to the machine learning network 130 or is already located in the machine learning network 130. The machine learning network 130 receives input of the one or more immunogenic peptides comprising one or more peptide binding motifs and the one or more ligand regions and passes the one or more immunogenic peptides comprising one or more peptide binding motifs and the one or more ligand regions to the data storage unit 137 for temporary or permanent storage or directly to the machine learning server 135.

In block 300, an ensemble network, operably contained in the machine learning system 133, processes the data of the one or more immunogenic peptides comprising one or more peptide binding motifs and the one or more ligand regions into output data comprising information of a refined set of the one or more immunogenic peptides comprising one or more peptide binding motifs.

In block 310, the machine system 133 generates output data comprising information containing a refined set of the one or more immunogenic peptides comprising one or more peptide binding motifs. The one or more immunogenic peptides comprising one or more peptide binding motifs, the one or more ligand regions, and the refined set of the one or more immunogenic peptides comprising one or more peptide binding motifs is transmitted back to the user via the network 105. In example embodiments, the resulting user information is stored on the data storage unit 137. In example embodiments, the resulting user information is immediately transmitted to the user's device. In example embodiments, the resulting user information is transmitted across the network 105 to the data acquisition system for subsequent access by the user associated device 100 or machine learning system 130.

The ladder diagrams, scenarios, flowcharts and block diagrams in the figures and discussed herein illustrate architecture, functionality, and operation of example embodiments and various aspects of systems, methods, and computer program products of the present invention. Each block in the flowchart or block diagrams can represent the processing of information and/or transmission of information corresponding to circuitry that can be configured to execute the logical functions of the present techniques. Each block in the flowchart or block diagrams can represent a module, segment, or portion of one or more executable instructions for implementing the specified operation or step. In example embodiments, the functions/acts in a block can occur out of the order shown in the figures and nothing requires that the operations be performed in the order illustrated. For example, two blocks shown in succession can executed concurrently or essentially concurrently. In another example, blocks can be executed in the reverse order. Furthermore, variations, modifications, substitutions, additions, or reduction in blocks and/or functions may be used with any of the ladder diagrams, scenarios, flow charts and block diagrams discussed herein, all of which are explicitly contemplated herein.

The ladder diagrams, scenarios, flow charts and block diagrams may be combined with one another, in part or in whole. Coordination will depend upon the required functionality. Each block of the block diagrams and/or flowchart illustration as well as combinations of blocks in the block diagrams and/or flowchart illustrations can be implemented by special purpose hardware-based systems that perform the aforementioned functions/acts or carry out combinations of special purpose hardware and computer instructions. Moreover, a block may represent one or more information transmissions and may correspond to information transmissions among software and/or hardware modules in the same physical device and/or hardware modules in different physical devices.

The present techniques can be implemented as a system, a method, a computer program product, digital electronic circuitry, and/or in computer hardware, firmware, software, or in combinations of them. The system may comprise distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as those described herein.

The computer program product can include a program tangibly embodied in an information carrier (e.g., computer readable storage medium or media) having computer readable program instructions thereon for execution by, or to control the operation of, data processing apparatus (e.g., a processor) to carry out aspects of one or more embodiments of the present invention. It will be understood that each block of the flowchart illustrations and/or block diagrams, and combinations of blocks in the flowchart illustrations and/or block diagrams, can be implemented by computer readable program instructions.

The computer readable program instructions can be performed on general purpose computing device, special purpose computing device, or other programmable data processing apparatus to produce a machine, such that the instructions, which execute via, at least partially, by one or more processors that are temporarily configured (e.g., by software) or permanently configured to perform the functions/acts specified in the flowchart and/or block diagram block or blocks. The processors, either: temporarily or permanently; or partially configured, may comprise processor-implemented modules. The present techniques referred to herein may, in example embodiments, comprise processor-implemented modules. Functions/acts of the processor-implemented modules may be distributed among the one or more processors. Moreover, the functions/acts of the processor-implements modules may be deployed across a number of machines, where the machines may be located in a single geographical location or distributed across a number of geographical locations.

The computer readable program instructions can also be stored in a computer readable storage medium that can direct one or more computer devices, programmable data processing apparatuses, and/or other devices to carry out the function/acts of the processor-implemented modules. The computer readable storage medium containing all or partial processor-implemented modules stored therein, comprises an article of manufacture including instructions which implement aspects, operations, or steps to be performed of the function/act specified in the flowchart and/or block diagram block or blocks.

Computer readable program instructions described herein can be downloaded to a computer readable storage medium within a respective computing/processing devices from a computer readable storage medium. Optionally, the computer readable program instructions can be downloaded to an external computer device or external storage device via a network. A network adapter card or network interface in each computing/processing device can receive computer readable program instructions from the network and forward the computer readable program instructions for permanent or temporary storage in a computer readable storage medium within the respective computing/processing device.

Computer readable program instructions described herein can be assembler instructions, instruction-set-architecture (ISA) instructions, machine instructions, machine dependent instructions, microcode, firmware instructions, state-setting data, or either source code or object code. The computer readable program instructions can be written in any programming language such as compiled or interpreted languages. In addition, the programming language can be object-oriented programming language (e.g., “C++”) or conventional procedural programming languages (e.g., “C”) or any combination thereof may be used to as computer readable program instructions. The computer readable program instructions can be distributed in any form, for example as a stand-alone program, module, subroutine, or other unit suitable for use in a computing environment. The computer readable program instructions can execute entirely on one computer or on multiple computers at one site or across multiple sites connected by a communication network, for example on user's computer, partly on the user's computer, as a stand-alone software package, partly on the user's computer and partly on a remote computer or entirely on a remote computer or server. If the computer readable program instructions are executed entirely remote, then the remote computer can be connected to the user's computer through any type of network or the connection can be made to an external computer. In examples embodiments, electronic circuitry including, but not limited to, programmable logic circuitry, field-programmable gate arrays (FPGA), or programmable logic arrays (PLA) can execute the computer readable program instructions. Electronic circuitry can utilize state information of the computer readable program instructions to personalize the electronic circuitry, to execute functions/acts of one or more embodiments of the present invention.

Example embodiments described herein include logic or a number of components, modules, or mechanisms. Modules may comprise either software modules or hardware-implemented modules. A software module may be code embodied on a non-transitory machine-readable medium or in a transmission signal. A hardware-implemented module is a tangible unit capable of performing certain operations and may be configured or arranged in a certain manner. In example embodiments, one or more computer systems (e.g., a standalone, client or server computer system) or one or more processors may be configured by software (e.g., an application or application portion) as a hardware-implemented module that operates to perform certain operations as described herein.

In example embodiments, a hardware-implemented module may be implemented mechanically or electronically. In example embodiments, hardware-implemented modules may comprise permanently configured dedicated circuitry or logic to execute certain functions/acts such as a special-purpose processor or logic circuitry (e.g., a field programmable gate array (FPGA) or an application-specific integrated circuit (ASIC)). In example embodiments, hardware-implemented modules may comprise temporary programmable logic or circuitry to perform certain functions/acts. For example, a general-purpose processor or other programmable processor.

The term “hardware-implemented module” encompasses a tangible entity. A tangible entity may be physically constructed, permanently configured, or temporarily or transitorily configured to operate in a certain manner and/or to perform certain functions/acts described herein. Hardware-implemented modules that are temporarily configured need not be configured or instantiated at any one time. For example, if the hardware-implemented modules comprise a general-purpose processor configured using software, then the general-purpose processor may be configured as different hardware-implemented modules at different times.

Hardware-implemented modules can provide, receive, and/or exchange information from/with other hardware-implemented modules. The hardware-implemented modules herein may be communicatively coupled. Multiple hardware-implemented modules operating concurrently, may communicate through signal transmission, for instance appropriate circuits and buses that connect the hardware-implemented modules. Multiple hardware-implemented modules configured or instantiated at different times may communicate through temporarily or permanently archived information, for instance the storage and retrieval of information in memory structures to which the multiple hardware-implemented modules have access. For example, one hardware-implemented module may perform an operation, and store the output of that operation in a memory device to which it is communicatively coupled. Consequently, another hardware-implemented module may, at some time later, access the memory device to retrieve and process the stored information. Hardware-implemented modules may also initiate communications with input or output devices and can operate on information from the input or output devices.

In example embodiments, the present techniques can be at least partially implemented in a cloud or virtual machine environment.

### Immunological Compositions and Vaccines

In example embodiments, the immunological composition is a protective vaccine or tolerizing vaccine composition comprising one or more of the antigenic epitopes to treat any disease or condition described herein (e.g., tumor or infection). The term “vaccine” or “immunological composition” are used interchangeably and are meant to refer in the present context to a pooled sample of one or more antigenic peptides, for example at least one, at least two, at least three, at least four, at least five, or more antigenic peptides. A “vaccine” is to be understood as including a protective vaccine, which is a composition for generating immunity for the prophylaxis and/or treatment of diseases (e.g., neoplasia/tumor). A “vaccine” is also to be understood as including a tolerizing vaccine, which is a composition for reducing immunity for the prophylaxis and/or treatment of diseases (e.g., autoimmune disease). A protective vaccine may be formulated with antigenic epitopes specific for a pathogen or for a cancer cell. Accordingly, vaccines are medicaments which comprise antigens and are intended to be used in humans or animals for generating specific defense and protective substance by vaccination. A “vaccine composition” can include a pharmaceutically acceptable excipient, carrier or diluent.

The vaccine may include one or more peptides identified according to the present invention. For example, 1 to 10 peptides. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

The vaccine of the present invention may ameliorate a disease as described herein. By “ameliorate” is meant decrease, suppress, attenuate, diminish, arrest, or stabilize the development or progression of a disease (e.g., a neoplasia, tumor, infection, etc.).

The terms “treat,” “treated,” “treating,” “treatment,” and the like are meant to refer to reducing or ameliorating a disorder and/or symptoms associated therewith (e.g., a neoplasia or tumor). “Treating” may refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

In example embodiments, a protective vaccine is used to treat cancer, in particular, a cancer caused by a virus expressing a large T antigen (LT). Additional examples of cancers and cancer conditions that can be treated with the therapy of this document include, but are not limited to, a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. Thus, the population of subjects described herein may be suffering from one of the above cancer types or any other cancer type described herein. In other embodiments, the population of subjects may be all subjects suffering from solid tumors, or all subjects suffering from liquid tumors.

Cancers that can be treated using the therapy described herein may include, among others, cases which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

The therapy described herein is also applicable where the subject has no detectable neoplasia but is at high risk for disease recurrence.

Also of special interest is the treatment of patients in need thereof who have undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the claimed neoplastic vaccine or immunogenic composition compositions to delay disease relapse.

The present invention is based, at least in part, on the ability to present the immune system of the patient with one or more HLA allele specific peptides.

### Producing Antigenic Peptides

One of skill in the art from this disclosure and the knowledge in the art will appreciate that there are a variety of ways in which to produce, or generate, antigens. In general, such antigens may be produced either in vitro or in vivo. Tumor specific antigens or antigens may be produced in vitro as peptides or polypeptides, which may then be formulated into a neoplasia vaccine or immunogenic composition and administered to a subject. As described in further detail herein, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific antigens or antigens may be produced in vivo by introducing molecules (e.g., DNA, RNA, viral expression systems, and the like) that encode tumor specific antigens or antigens into a subject, whereupon the encoded tumor specific antigens or antigens are expressed. The methods of in vitro and in vivo production of antigens or antigens is also further described herein as it relates to pharmaceutical compositions and methods of delivery of the therapy. By an isolated “polypeptide” or “peptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

In example embodiments, the present invention includes modified antigenic or antigenic peptides. As used herein in reference to peptides, the terms “modified”, “modification” and the like refer to one or more changes that enhance a desired property of the antigenic peptide, where the change does not alter the primary amino acid sequence of the antigenic peptide. “Modification” includes a covalent chemical modification that does not alter the primary amino acid sequence of the antigenic peptide itself. Such desired properties include, for example, prolonging the in vivo half-life, increasing the stability, reducing the clearance, altering the immunogenicity or allergenicity, enabling the raising of particular antibodies, cellular targeting, antigen uptake, antigen processing, MHC affinity, MHC stability, or antigen presentation. Changes to an antigenic peptide that may be carried out include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids. Modified peptides also include analogs. By “analog” is meant a molecule that is not identical, but has analogous functional or structural features. For example, a tumor specific neo-antigen polypeptide analog retains the biological activity of a corresponding naturally-occurring tumor specific neo-antigen polypeptide, while having certain biochemical modifications that enhance the analog's function relative to a naturally-occurring polypeptide. Such biochemical modifications could increase the analog's protease resistance, membrane permeability, or half-life, without altering, for example, ligand binding. An analog may include an unnatural amino acid.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Modified peptides may include a spacer or a linker. The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins or RNA sequences. However, in example embodiments, the constituent amino acids of a spacer may be selected to influence some property of the molecule such as the folding, net charge, or hydrophobicity of the molecule.

Suitable linkers for use in an embodiment of the present invention are well known to those of skill in the art and include, but are not limited to, straight or branched-chain carbon linkers, heterocyclic carbon linkers, or peptide linkers. The linker is used to separate two antigenic peptides by a distance sufficient to ensure that, in a preferred embodiment, each antigenic peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180.

The clinical effectiveness of protein therapeutics is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide sequence are generally soluble in water at room temperature, and have the general formula R (0-CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, but certain embodiments have a molecular weight between 500 and 20,000 while other embodiments have a molecular weight between 4,000 and 10,000. The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to a polypeptide of the present disclosure via a terminal reactive group (a “spacer”). The spacer is, for example, a terminal reactive group which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxy succinylimide. Another activated polyethylene glycol which may be bound to a free amino group is 2,4-bis(0-methoxypolyethyleneglycol)-6-chloro-s-triazine which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C.

The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, CA). This obviates the need for an additional conjugation step during the manufacturing process. Moreover, established molecular biology techniques enable control of the side chain composition of the polypeptide chains, allowing optimization of immunogenicity and manufacturing properties.

For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties of proteins and can also be important in protein stability, secretion, and subcellular localization. Proper glycosylation can be essential for biological activity. In fact, some genes from eucaryotic organisms, when expressed in bacteria (e.g., E. coli) which lack cellular processes for glycosylating proteins, yield proteins that are recovered with little or no activity by virtue of their lack of glycosylation.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the DNA level, particularly by mutating the DNA encoding the polypeptide at preselected bases such that codons are generated that will translate into the desired amino acids. Another means of increasing the number of carbohydrate moieties on the polypeptide is by chemical or enzymatic coupling of glycosides to the polypeptide.

Removal of carbohydrates may be accomplished chemically or enzymatically, or by substitution of codons encoding amino acid residues that are glycosylated. Chemical deglycosylation techniques are known, and enzymatic cleavage of carbohydrate moieties on peptides can be achieved by the use of a variety of endo- and exo-glycosidases.

Dihydrofolate reductase (DHFR)-deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300 (1-2): 125-30). As with modifications with other conjugates (e.g., PEG), various techniques for site-specific polysialylation are available (see, e.g., T. Lindhout et al., PNAS 10⁸ (18) 7397-7402 (2011)).

Additional suitable components and molecules for conjugation include, for example, thyroglobulin; albumins such as human serum albumin (HAS); tetanus toxoid; Diphtheria toxoid; polyamino acids such as poly (D-lysine: D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more immunogenic peptides comprising one or more peptide binding motifs of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake, incorporation and expression of exogenous genetic material (exogenous DNA) from its surroundings and taken up through the cell membrane(s). Transformation occurs naturally in some species of bacteria, but it can also be effected by artificial means in other cells.

Furthermore, albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin. (See WO2011/051489).

Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule. A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein (e.g., diabetes).

A polypeptide may also be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Purification methods such as cation exchange chromatography may be used to separate conjugates by charge difference, which effectively separates conjugates into their various molecular weights. For example, the cation exchange column can be loaded and then washed with −20 mM sodium acetate, pH −4, and then eluted with a linear (0 M to 0.5 M) NaCl gradient buffered at a pH from about 3 to 5.5, e.g., at pH −4.5. The content of the fractions obtained by cation exchange chromatography may be identified by molecular weight using conventional methods, for example, mass spectroscopy, SDS-PAGE, or other known methods for separating molecular entities by molecular weight.

In example embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure contemplates the use of other modifications, currently known or developed in the future, of the one or more immunogenic peptides comprising peptide binding motifs to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Application Nos. 2007/0134197 and 2006/0258607.

### In Vitro Peptide/Polypeptide Synthesis

Proteins, peptides, or peptide binding motifs may be made by any technique known to those of skill in the art, including the expression of proteins, polypeptides, peptides, or peptide binding motifs through standard molecular biological techniques, the isolation of proteins, peptides, peptide binding motifs from natural sources, in vitro translation, or the chemical synthesis of proteins, peptides, or peptide binding motifs. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides, peptides, and peptide binding motifs are known to those of skill in the art.

Immunogenic peptides comprising peptide binding motifs, also referred to herein as peptides, can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963). In example embodiments, antigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding an antigenic peptide of the invention may be used to produce the antigenic peptide in vitro. The polynucleotide may be, e.g., DNA, cDNA, PNA, CNA, RNA, either single- and/or double-stranded, or native or stabilized forms of polynucleotides, such as e.g., polynucleotides with a phosphorothiate backbone, or combinations thereof and it may or may not contain introns so long as it codes for the peptide. In an example embodiment, in vitro translation is used to produce the peptide. Many exemplary systems exist that one skilled in the art could utilize (e.g., Retic Lysate IVT Kit, Life Technologies, Waltham, MA).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The antigenic peptides may be provided in the form of RNA or cDNA molecules encoding the desired antigenic peptides. One or more antigenic peptides of the invention may be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the antigenic peptide fused in the same reading frame to a marker sequence that allows, for example, for purification of the encoded polypeptide, which may then be incorporated into the personalized neoplasia vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more antigenic peptides fused in the same reading frame to create a single concatemerized antigenic peptide construct capable of producing multiple antigenic peptides.

In example embodiments, isolated nucleic acid molecules having a nucleotide sequence at least 60% identical, at least 65% identical, at least 70% identical, at least 75% identical, at least 80%) identical, at least 85% identical, at least 90% identical, at least 95% identical, or at least 96%), 97%, 98% or 99% identical to a polynucleotide encoding an antigenic peptide of the present invention, can be provided. By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5%> of the total number of nucleotides in the reference sequence are allowed.

The isolated antigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the antigenic peptides. Recombinant expression vectors are replicable DNA constructs which have synthetic or cDNA-derived DNA fragments encoding an antigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as MI 3 and filamentous single-stranded DNA phages.

Suitable host cells for expression of a polypeptide include prokaryotes, yeast, insect or higher eukaryotic cells under the control of appropriate promoters. Prokaryotes include gram negative-or gram-positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-FPLC) steps employing hydrophobic RP-FTPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein. Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### In Vivo Peptide/Polypeptide Synthesis

The present invention also contemplates the use of nucleic acid molecules as vehicles for delivering antigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment, antigenic peptides may be administered to a patient in need thereof by use of an mRNA vaccine (see, e.g., Sahin, U, Kariko, K and Tureci, O (2014). mRNA-based therapeutics-developing a new class of drugs. Nat Rev Drug Discov 13:759-780; Weissman D, Karikó K. mRNA: Fulfilling the Promise of Gene Therapy. Mol Ther. 2015; 23 (9): 1416-1417. doi: 10.1038/mt.2015.138; Kowalski P S, Rudra A, Miao L, Anderson D G. Delivering the Messenger: Advances in Technologies for Therapeutic mRNA Delivery. Mol Ther. 2019; 27 (4): 710-728. doi: 10.1016/j.ymthe.2019.02.012; Magadum A, Kaur K, Zangi L. mRNA-Based Protein Replacement Therapy for the Heart. Mol Ther. 2019; 27 (4): 785-793. doi: 10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7 (5): 319-334. doi: 10.4155/tde-2016-0006; and Khalil A S, Yu X, Umhoefer J M, et al. Single-dose mRNA therapy via biomaterial-mediated sequestration of overexpressed proteins. Sci Adv. 2020; 6 (27): eaba2422). In an exemplary embodiment, mRNA encoding for an antigenic peptide is delivered using lipid nanoparticles (see, e.g., Reichmuth, et al., 2016) and administered directly to tumor tissue. In an exemplary embodiment, mRNA encoding for an antigenic peptide is delivered using biomaterial-mediated sequestration (see, e.g., Khalil, et al., 2020) and administered directly to tumor tissue.

In one embodiment, antigens may be administered to a patient in need thereof by use of a plasmid. These are plasmids which usually consist of a strong viral promoter to drive the in vivo transcription and translation of the gene (or complementary DNA) of interest (Mor, et al., (1995), The Journal of Immunology 155 (4): 2039-2046). Intron A may sometimes be included to improve mRNA stability and hence increase protein expression (Leitner et al. (1997), The Journal of Immunology 159 strong (12): 6112-6119). Plasmids also include a polyadenylation/transcriptional termination signal, such as bovine growth hormone or rabbit beta-globulin polyadenylation sequences (Alarcon et al., (1999), Adv. Parasitol. Advances in Parasitology 42:343-410; Robinson et al., (2000). Adv. Virus Res. Advances in Virus Research 55:1-74; Bohmet al., (1996). Journal of Immunological Methods 193 (1): 29-40.). Multi cistronic vectors are sometimes constructed to express more than one immunogen, or to express an immunogen and an immunostimulatory protein (Lewis et al., (1999). Advances in Virus Research (Academic Press) 54:129-88).

Because the plasmid is the “vehicle” from which the immunogen is expressed, optimizing vector design for maximal protein expression is essential (Lewis et al., (1999). Advances in Virus Research (Academic Press) 54:129-88). One way of enhancing protein expression is by optimizing the codon usage of pathogenic mRNAs for eukaryotic cells. Another consideration is the choice of promoter. Such promoters may be the SV40 promoter or Rous Sarcoma Virus (RSV). Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (EVI) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42:343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42:343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42:343-410; Lewis et al., (1999). Advances in Virus Research (Academic Press) 54:129-88).

Alternative delivery methods may include aerosol instillation of naked DNA on mucosal surfaces, such as the nasal and lung mucosa, (Lewis et al., (1999). Advances in Virus Research (Academic Press) 54:129-88) and topical administration of pDNA to the eye and vaginal mucosa (Lewis et al., (1999) Advances in Virus Research (Academic Press) 54:129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex Vivo Cytosolic Delivery of Functional Macromolecules to Immune Cells, Sharei et al, PLOS ONE| DOI: 10.1371/journal.pone. Ol 18803 Apr. 13, 2015).

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshiaet al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition may include separate DNA plasmids encoding, for example, one or more antigenic peptides/polypeptides as identified in according to the invention. As discussed herein, the exact choice of expression vectors can depend upon the peptide/polypeptides to be expressed and is well within the skill of the ordinary artisan. The expected persistence of the DNA constructs (e.g., in an episomal, non-replicating, non-integrated form in the muscle cells) is expected to provide an increased duration of protection.

One or more antigenic peptides of the invention may be encoded and expressed in vivo using a viral based system (e.g., an adenovirus system, an adeno associated virus (AAV) vector, a poxvirus, or a lentivirus). In one embodiment, the neoplasia vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207 (2): 240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. patent application No. 20080254008, hereby incorporated by reference). The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8:275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571, US20040013648, US20070025970, US20090111106 and U.S. Pat. No. 7,259,015. In another embodiment lentiviral vectors are used to deliver vectors to the brain of those being treated for a disease.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

In an embodiment herein the delivery is via a lentivirus. Zou et al. administered about 10 ut of a recombinant lentivirus having a titer of 1×10⁹ transducing units (TU)/ml by an intrathecal catheter. These sorts of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹ TU/ml, more preferably at least 10⁹ TU/ml. Other methods of concentration such as ultrafiltration or binding to and elution from a matrix may be used.

In other embodiments the amount of lentivirus administered may be 1.×. 10⁵ or about 1.×. 10⁵ plaque forming units (PFU), 5.×.10⁵ or about 5.×. 10⁵ PFU, 1.×.10⁶ or about 1.×.10⁶ PFU, 5.×.10⁶ or about 5.×. 10⁶ PFU, 1.×.10⁷ or about 1.×. 10⁷PFU, 5.×.10⁷ or about 5.×. 10⁷ PFU, 1.×. 10⁸ or about 1.×. 10⁸ PFU, 5.×. 10⁸ or about 5.×. 10⁸ PFU, 1.×. 10⁹ or about 1.×. 10⁹ PFU, 5.×. 10⁹ or about 5.×. 10⁹ PFU, 1.×. 10¹⁰ or about 1.×. 10¹⁰ PFU or 5.×. 10¹⁰ or about 5.×. 10¹⁰ PFU as total single dosage for an average human of 75 kg or adjusted for the weight and size and species of the subject. One of skill in the art can determine suitable dosage. Suitable dosages for a virus can be determined empirically. Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Adl 1, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Adl 1 vectors are described in U.S. Pat. No. 6,913,922. C6 adenovirus vectors are described in U.S. Pat. Nos. 6,780,407; 6,537,594; 6,309,647; 6,265, 189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both Ela and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention and can even be used for co-delivery of a large number of heterologous inserts/genes.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10 pu, about 4×10 pu, about 1×10 pu, about 2×10 pu, about 4×10 pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include Pol III promoters such as U6 or HI. The use of a Pol II promoter and intronic cassettes can be used to express guide RNA (gRNA).

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage may be adjusted to balance the therapeutic benefit against any side effects. In an embodiment herein, the AAV dose is generally in the range of concentrations from about 1×10 to 1×10 genomes AAV, from about 1×10 to 1×10 genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment effectively activating a cellular immune response for a neoplasia vaccine or immunogenic composition can be achieved by expressing the relevant antigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety). In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardiet al., Hum Vaccin Immunother. 2012 July; 8 (7): 961-70; and Moss, Vaccine. 2013; 31 (39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature, such as: U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756, 101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and Panicali, D. Proc. Natl. Acad. Sci. 1982; 79; 4927-493, Panicali D. Proc. Natl. Acad. Sci. 1983; 80 (17): 5364-8, Mackett, M. Proc. Natl. Acad. Sci. 1982; 79:7415-7419, Smith G L. Proc. Natl. Acad. Sci. 1983; 80 (23): 7155-9, Smith G L. Nature 1983; 302:490-5, Sullivan V J. Gen. Vir. 1987; 68:2587-98, Perkus M Journal of Leukocyte Biology 1995; 58:1-13, Yilma T D. Vaccine 1989; 7:484-485, Brochier B. Nature 1991; 354:520-22, Wiktor, TJ. Proc. Natl Acd. Sci. 1984; 81:7194-8, Rupprecht, CE. Proc. Natl Acd. Sci. 1986; 83:7947-50, Poulet, H Vaccine 2007; 25 (July): 5606-12, Weyer J. Vaccine 2009; 27 (November): 7198-201, Buller, R M Nature 1985; 317 (6040): 813-5, Buller R M. J. Virol. 1988; 62 (3): 866-74, Flexner, C. Nature 1987; 330 (6145): 259-62, Shida, H. J. Virol. 1988; 62 (12): 4474-80, Kotwal, GJ. J. Virol. 1989; 63 (2): 600-6, Child, SJ. Virology 1990; 174 (2): 625-9, Mayr A. Zentralbl Bakteriol 1978; 167 (5,6): 375-9, Antoine G. Virology. 1998; 244 (2): 365-96, Wyatt, LS. Virology 1998; 251 (2): 334-42, Sancho, MC. J. Virol. 2002; 76 (16); 8313-34, Gallego-Gomez, JC. J. Virol. 2003; 77 (19); 10606-22), Goebel S J. Virology 1990; (a,b) 179:247-66, Tartaglia, J. Virol. 1992; 188 (1): 217-32, Najera J L. J. Virol. 2006; 80 (12): 6033-47, Najera, J L. J. Virol. 2006; 80:6033-6047, Gomez, CE. J. Gen. Virol. 2007; 88:2473-78, Mooij, P. Jour. Of Virol. 2008; 82:2975-2988, Gomez, CE. Curr. Gene Ther. 2011; 11:189-217, Cox, W. Virology 1993; 195:845-50, Perkus, M. Jour. Of Leukocyte Biology 1995; 58:1-13, Blanchard T J. J Gen Virology 1998; 79 (5): 1159-67, Amara R. Science 2001; 292:69-74, Hel, Z., J. Immunol. 2001; 167:7180-9, Gherardi M M. J. Virol. 2003; 77:7048-57, Didierlaurent, A. Vaccine 2004; 22:3395-3403, Bissht H. Proc. Nat. Aca. Sci. 2004; 101:6641-46, McCurdy L H. Clin. Inf. Dis 2004; 38:1749-53, Earl P L. Nature 2004; 428:182-85, Chen Z. J. Virol. 2005; 79:2678-2688, Najera J L. J. Virol. 2006; 80 (12): 6033-47, Nam J H. Acta. Virol. 2007; 51:125-30, Antonis A F. Vaccine 2007; 25:4818-4827, B Weyer J. Vaccine 2007; 25:4213-22, Ferrier-Rembert A. Vaccine 2008; 26 (14): 1794-804, Corbett M. Proc. Natl. Acad. Sci. 2008; 105 (6): 2046-51, Kaufman H L., J. Clin. Oncol. 2004; 22:2122-32, Amato, RJ. Clin. Cancer Res. 2008; 14 (22): 7504-10, Dreicer R. Invest New Drugs 2009; 27 (4): 379-86, Kantoff P W. J. Clin. Oncol. 2010, 28, 1099-1 105, Amato R J. J. Clin. Can. Res. 2010; 16 (22): 5539-47, Kim, DW. Hum. Vaccine. 2010; 6:784-791, Oudard, S. Cancer Immunol. Immunother. 2011; 60:261-71, Wyatt, LS. Aids Res. Hum. Retroviruses. 2004; 20:645-53, Gomez, CE. Virus Research 2004; 105:11-22, Webster, DP. Proc. Natl. Acad. Sci. 2005; 102:4836-4, Huang, X. Vaccine 2007; 25:8874-84, Gomez, CE. Vaccine 2007a; 25:2863-85, Esteban M. Hum. Vaccine 2009; 5:867-871, Gomez, CE. Curr. Gene therapy 2008; 8 (2): 97-120, Whelan, KT. Plos one 2009; 4 (6): 5934, Scriba, TJ. Eur. Jour. Immuno. 2010; 40 (1): 279-90, Corbett, M. Proc. Natl. Acad. Sci. 2008; 105:2046-2051, Midgley, CM. J. Gen. Virol. 2008; 89:2992-97, Von Krempelhuber, A. Vaccine 2010; 28:1209-16, Perreau, M. J. Of Virol. 2011; October: 9854-62, Pantaleo, G. Curr Opin HIV-AIDS. 2010; 5:391-396, each of which is incorporated herein by reference.

In another embodiment the vaccinia virus is used in the neoplasia vaccine or immunogenic composition to express an antigen. (Rolph et al., Recombinant viruses as vaccines and immunological tools. Curr Opin Immunol 9:517-524, 1997). The recombinant vaccinia virus is able to replicate within the cytoplasm of the infected host cell and the peptide of interest can therefore induce an immune response. Moreover, Poxviruses have been widely used as vaccine or immunogenic composition vectors because of their ability to target encoded antigens for processing by the MHC pathway by directly infecting immune cells, in particular antigen-presenting cells, but also due to their ability to self-adjuvant.

In another embodiment ALVAC is used as a vector in a neoplasia vaccine or immunogenic composition. ALVAC is a canarypox virus that can be modified to express foreign transgenes and has been used as a method for vaccination against both prokaryotic and eukaryotic antigens (Horig H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6:2219-28; Musey L, Ding Y, Elizaga M, et al. HIV-1 vaccination administered intramuscularly can induce both systemic and mucosal T cell immunity in HIV-1-uninfected individuals. J Immunol 2003; 171:1094-101; Paoletti E. Applications of pox virus vectors to vaccination: an update. Proc Natl Acad Sci USA 1996; 93:11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J Clin Oncol 1999; 17:332-7).

In another embodiment a Modified Vaccinia Ankara (MVA) virus may be used as a viral vector for an antigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. January 2012; 61 (1): 19-29). Methods to make and use recombinant MVA has been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles of the vaccine or immunogenic composition are administered to patients in need thereof. Dosages of expressed antigen can range from a few to a few hundred micrograms, e.g., 5 to 500 μg. The vaccine or immunogenic composition can be administered in any suitable amount to achieve expression at these dosage levels. The viral particles can be administered to a patient in need thereof or transfected into cells in an amount of about at least 10^{3.5} pfu; thus, the viral particles are preferably administered to a patient in need thereof or infected or transfected into cells in at least about 104 pfu to about 10⁶ pfu; however, a patient in need thereof can be administered at least about 10⁸ pfu such that a more preferred amount for administration can be at least about 10⁷ pfu to about 10⁹ pfu. Doses as to NYVAC are applicable as to ALVAC, MVA, MVA-BN, and avipoxes, such as canarypox and fowlpox.

### Machine Learning Embodiments

Machine learning is a field of study within artificial intelligence that allows computers to learn functional relationships between inputs and outputs without being explicitly programmed. Machine learning involves a module comprising algorithms that may learn from existing data by analyzing, categorizing, or identifying the data. Such machine-learning algorithms operate by first constructing a model from training data to make predictions or decisions expressed as outputs. In example embodiments, the training data includes data for one or more identified features and one or more outcomes, for example one or more amino acids and one or more immunogenic peptides comprising one or more peptide binding motifs or one or more ligand regions. Although example embodiments are presented with respect to a few machine-learning algorithms, the principles presented herein may be applied to other machine-learning algorithms.

Data supplied to a machine learning algorithm can be considered a feature, which can be described as an individual measurable property of a phenomenon being observed. The concept of feature is related to that of an independent variable used in statistical techniques such as those used in linear regression. The performance of a machine learning algorithm in pattern recognition, classification and regression is highly dependent on choosing informative, discriminating, and independent features. Features may comprise numerical data, categorical data, time-series data, strings, graphs, or images. Features of the invention may further comprise one or more amino acids. These one or more amino acids may include additional features. In one example embodiment, the one or more features comprise binary, fractional, or both features. In one example embodiment, the fractional features comprise secreted protein features, transmembrane features, domain features, region features, relative solvent accessibility features, and disorder features.

In general, there are two categories of machine learning problems: classification problems and regression problems. Classification problems, also referred to as categorization problems, aim at classifying items into discrete category values. Training data teaches the classifying algorithm how to classify. In example embodiments, features to be categorized may include one or more amino acid sequences, which can be provided to the classifying machine learning algorithm and then placed into categories of, for example, one or more immunogenic peptides comprising one or more peptide binding motifs or one or more ligand regions. Regression algorithms aim at quantifying and correlating one or more features. Training data teaches the regression algorithm how to correlate the one or more features into a quantifiable value. In example embodiments, features such as one or more amino acid sequences can be provided to the regression machine learning algorithm resulting in one or more continuous values, for example one or more immunogenic peptides comprising one or more peptide binding motifs or one or more ligand regions. As used herein, a machine learning network may be used to describe the entirety of the system to carry out a machine learning process and comprise of a machine learning model. Furthermore, machine learning model and machine learning module are used herein interchangeably.

### Embedding

In one example, the machine learning module may use embedding to provide a lower dimensional representation, such as a vector, of features to organize them based off respective similarities. In some situations, these vectors can become massive. In the case of massive vectors, particular values may become very sparse among a large number of values (e.g., a single instance of a value among 50,000 values). Because such vectors are difficult to work with, reducing the size of the vectors, in some instances, is necessary. A machine learning module can learn the embeddings along with the model parameters. In example embodiments, features such as one or more amino acids, the one or more features comprise binary, fractional, secreted protein features, transmembrane features, domain features, region features, relative solvent accessibility features, disorder features, or any combination thereof can be mapped to vectors implemented in embedding methods. In example embodiments, embedded semantic meanings are utilized. Embedded semantic meanings are values of respective similarity. For example, the distance between two vectors, in vector space, may imply two values located elsewhere with the same distance are categorically similar. Embedded semantic meanings can be used with similarity analysis to rapidly return similar values. In an example embodiment, an amino acid is mapped to a vector such that vector value of the amino acids is influenced by the relative position of all other amino acids. In an example embodiment, the one or more amino acids are mapped with corresponding features such as binary, fractional, secreted protein features, transmembrane features, domain features, region features, relative solvent accessibility features, disorder features, or any combination thereof to a vector. In example embodiments, the methods herein are developed to identify meaningful portions of the vector and extract semantic meanings between that space.

### Training Methods

In example embodiments, the machine learning module can be trained using techniques such as unsupervised, supervised, semi-supervised, reinforcement learning, transfer learning, incremental learning, curriculum learning techniques, and/or learning to learn. Training typically occurs after selection and development of a machine learning module and before the machine learning module is operably in use. In one aspect, the training data used to teach the machine learning module can comprise input data such as one or more amino acid sequences and the respective target output data such as one or more immunogenic peptides comprising one or more peptide binding motifs or one or more ligand regions. In an example embodiment, a machine learning network is trained on HLA-II allele-specific peptidomic data. In an example embodiment, the HLA-II allele-specific peptidomics data is specific for one or more HLA II alleles selected from the group consisting of those in Table 1. In an example embodiment, a machine learning network is trained on full length source proteins mapped back from HLA-II allele-specific peptidomics data. In an example data, a machine learning network is trained with decoys at a 5:1, 4:1, or 6:1 ratio to immunogenic peptides. A decoy may comprise a random length-matched peptide with no reported binding to any allele, or an immunogenic peptide with 1, 2, 3, 4, 4, 5, 6, 8, 9, or 10 mutations.

In an example embodiment, unsupervised learning is implemented. Unsupervised learning can involve providing all or a portion of unlabeled training data to a machine learning module. The machine learning module can then determine one or more outputs implicitly based on the provided unlabeled training data. In an example embodiment, supervised learning is implemented. Supervised learning can involve providing all or a portion of labeled training data to a machine learning module, with the machine learning module determining one or more outputs based on the provided labeled training data, and the outputs are either accepted or corrected depending on the agreement to the actual outcome of the training data. In some examples, supervised learning of machine learning system(s) can be governed by a set of rules and/or a set of labels for the training input, and the set of rules and/or set of labels may be used to correct inferences of a machine learning module.

In an example embodiment, semi-supervised learning is implemented. Semi-supervised learning can involve providing all or a portion of training data that is partially labeled to a machine learning module. During semi-supervised learning, supervised learning is used for a portion of labeled training data, and unsupervised learning is used for a portion of unlabeled training data. In one example embodiment, reinforcement learning is implemented. Reinforcement learning can involve first providing all or a portion of the training data to a machine learning module and as the machine learning module produces an output, the machine learning module receives a “reward” signal in response to a correct output. Typically, the reward signal is a numerical value and the machine learning module is developed to maximize the numerical value of the reward signal. In addition, reinforcement learning can adopt a value function that provides a numerical value representing an expected total of the numerical values provided by the reward signal over time.

In an example embodiment, transfer learning is implemented. Transfer learning techniques can involve providing all or a portion of a first training data to a machine learning module, then, after training on the first training data, providing all or a portion of a second training data. In example embodiments, a first machine learning module can be pre-trained on data from one or more computing devices. The first trained machine learning module is then provided to a computing device, where the computing device is intended to execute the first trained machine learning model to produce an output. Then, during the second training phase, the first trained machine learning model can be additionally trained using additional training data, where the training data can be derived from kernel and non-kernel data of one or more computing devices. This second training of the machine learning module and/or the first trained machine learning model using the training data can be performed using either supervised, unsupervised, or semi-supervised learning. In addition, it is understood transfer learning techniques can involve one, two, three, or more training attempts. Once the machine learning module has been trained on at least the training data, the training phase can be completed. The resulting trained machine learning model can be utilized as at least one of trained machine learning module.

In one example embodiment, incremental learning is implemented. Incremental learning techniques can involve providing a trained machine learning module with input data that is used to continuously extend the knowledge of the trained machine learning module. Another machine learning training technique is curriculum learning, which can involve training the machine learning module with training data arranged in a particular order, such as providing relatively easy training examples first, then proceeding with progressively more difficult training examples. As the name suggests, difficulty of training data is analogous to a curriculum or course of study at a school.

In one example embodiment, learning to learn is implemented. Learning to learn, or meta-learning, comprises, in general, two levels of learning: quick learning of a single task and slower learning across many tasks. For example, a machine learning module is first trained and comprises of a first set of parameters or weights. During or after operation of the first trained machine learning module, the parameters or weights are adjusted by the machine learning module. This process occurs iteratively on the success of the machine learning module. In another example, an optimizer, or another machine learning module, is used wherein the output of a first trained machine learning module is fed to an optimizer that constantly learns and returns the final results. Other techniques for training the machine learning module and/or trained machine learning module are possible as well.

In some examples, after the training phase has been completed but before producing predictions expressed as outputs, a trained machine learning module can be provided to a computing device where a trained machine learning module is not already resident, in other words, after training phase has been completed, the trained machine learning module can be downloaded to a computing device. For example, a first computing device storing a trained machine learning module can provide the trained machine learning module to a second computing device. Providing a trained machine learning module to the second computing device may comprise one or more of communicating a copy of trained machine learning module to the second computing device, making a copy of trained machine learning module for the second computing device, providing access to trained machine learning module to the second computing device, and/or otherwise providing the trained machine learning system to the second computing device. In example embodiments, a trained machine learning module can be used by the second computing device immediately after being provided by the first computing device. In some examples, after a trained machine learning module is provided to the second computing device, the trained machine learning module can be installed and/or otherwise prepared for use before the trained machine learning module can be used by the second computing device.

After a machine learning model has been trained it can be used to output, estimate, infer, predict, generate, or determine, for simplicity these terms will collectively be referred to as results. A trained machine learning module can receive input data and operably generate results. As such, the input data can be used as an input to the trained machine learning module for providing corresponding results to kernel components and non-kernel components. For example, a trained machine learning module can generate results in response to requests. In example embodiments, a trained machine learning module can be executed by a portion of other software. For example, a trained machine learning module can be executed by a result daemon to be readily available to provide results upon request.

In example embodiments, a machine learning module and/or trained machine learning module can be executed and/or accelerated using one or more computer processors and/or on-device co-processors. Such on-device co-processors can speed up training of a machine learning module and/or generation of results. In some examples, trained machine learning module can be trained, reside, and execute to provide results on a particular computing device, and/or otherwise can make results for the particular computing device.

Input data can include data from a computing device executing a trained machine learning module and/or input data from one or more computing devices. In example embodiments, a trained machine learning module can use results as input feedback. A trained machine learning module can also rely on past results as inputs for generating new results. In example embodiments, input data can comprise one or more amino acid sequences and, when provided to a trained machine learning module, results in output data such as one or more immunogenic peptides comprising one or more peptide binding motifs or one or more ligand regions. The one or more immunogenic peptides comprising one or more peptide binding motifs and one or more ligand regions can then be provided to an ensemble network. The ensemble network can use the one or more immunogenic peptides comprising one or more peptide binding motifs and one or more ligand regions to generate a refined set of the one or more peptide binding motifs, thereby generating a refined one or more immunogenic peptides comprising one or more peptide binding motifs for use in immunological compositions. As such, the immunological composition-related technical problem of generating one or more immunogenic peptides comprising one or more peptides can be solved using the herein-described techniques that utilize machine learning to produce one or more immunogenic peptides comprising one or more peptide binding motifs and one or more ligand regions used in the ensemble network.

### Algorithms

Different machine-learning algorithms have been contemplated to carry out the embodiments discussed herein. For example, linear regression (LiR), logistic regression (LoR), Bayesian networks (for example, naive-bayes), random forest (RF) (including decision trees), neural networks (NN) (also known as artificial neural networks), matrix factorization, a hidden Markov model (HMM), support vector machines (SVM), K-means clustering (KMC), K-nearest neighbor (KNN), a suitable statistical machine learning algorithm, and/or a heuristic machine learning system for classifying or evaluating one or more immunogenic peptides comprising one or more peptide binding motifs for use in immunological compositions.

### Linear Regression (LiR)

In one example embodiment, linear regression machine learning is implemented. LiR is typically used in machine learning to predict a result through the mathematical relationship between an independent and dependent variable, such as one or more amino acid sequences and one or more immunogenic peptides comprising one or more peptide binding motifs or one or more ligand regions, respectively. A simple linear regression model would have one independent variable (x) and one dependent variable (y). A representation of an example mathematical relationship of a simple linear regression model would be y=mx+b. In this example, the machine learning algorithm tries variations of the tuning variables m and b to optimize a line that includes all the given training data.

The tuning variables can be optimized, for example, with a cost function. A cost function takes advantage of the minimization problem to identify the optimal tuning variables. The minimization problem preposes the optimal tuning variable will minimize the error between the predicted outcome and the actual outcome. An example cost function may comprise summing all the square differences between the predicted and actual output values and dividing them by the total number of input values and results in the average square error.

To select new tuning variables to reduce the cost function, the machine learning module may use, for example, gradient descent methods. An example gradient descent method comprises evaluating the partial derivative of the cost function with respect to the tuning variables. The sign and magnitude of the partial derivatives indicate whether the choice of a new tuning variable value will reduce the cost function, thereby optimizing the linear regression algorithm. A new tuning variable value is selected depending on a set threshold. Depending on the machine learning module, a steep or gradual negative slope is selected. Both the cost function and gradient descent can be used with other algorithms and modules mentioned throughout. For the sake of brevity, both the cost function and gradient descent are well known in the art and are applicable to other machine learning algorithms and may not be mentioned with the same detail.

LiR models may have many levels of complexity comprising one or more independent variables. Furthermore, in an LiR function with more than one independent variable, each independent variable may have the same one or more tuning variables or each, separately, may have their own one or more tuning variables. The number of independent variables and tuning variables will be understood to one skilled in the art for the problem being solved. In example embodiments, one or more amino acid sequences are used as the independent variables to train a LiR machine learning module, which, after training, is used to estimate, for example, one or more immunogenic peptides comprising one or more peptide binding motifs or one or more ligand regions.

### Logistic Regression (LoR)

In one example embodiment, logistic regression machine learning is implemented. Logistic Regression, often considered a LiR type model, is typically used in machine learning to classify information, such as one or more amino acid sequences into categories such as one or more immunogenic peptides comprising one or more peptide binding motifs or one or more ligand regions. LoR takes advantage of probability to predict an outcome from input data. However, what makes LoR different from a LiR is that LoR uses a more complex logistic function, for example a sigmoid function. In addition, the cost function can be a sigmoid function limited to a result between 0 and 1. For example, the sigmoid function can be of the form ƒ(x)=1/(1+e^{-x}), where x represents some linear representation of input features and tuning variables. Similar to LiR, the tuning variable(s) of the cost function are optimized (typically by taking the log of some variation of the cost function) such that the result of the cost function, given variable representations of the input features, is a number between 0 and 1, preferably falling on either side of 0.5. As described in LiR, gradient descent may also be used in LoR cost function optimization and is an example of the process. In example embodiments, one or more amino acid sequences are used as the independent variables to train a LoR machine learning module, which, after training, is used to estimate, for example, one or more immunogenic peptides comprising one or more peptide binding motifs or one or more ligand regions.

### Bayesian Network

In one example embodiment, a Bayesian Network is implemented. BNs are used in machine learning to make predictions through Bayesian inference from probabilistic graphical models. In BNs, input features are mapped onto a directed acyclic graph forming the nodes of the graph. The edges connecting the nodes contain the conditional dependencies between nodes to form a predicative model. For each connected node the probability of the input features resulting in the connected node is learned and forms the predictive mechanism. The nodes may comprise the same, similar or different probability functions to determine movement from one node to another. The nodes of a Bayesian network are conditionally independent of its non-descendants given its parents thus satisfying a local Markov property. This property affords reduced computations in larger networks by simplifying the joint distribution.

There are multiple methods to evaluate the inference, or predictability, in a BN but only two are mentioned for demonstrative purposes. The first method involves computing the joint probability of a particular assignment of values for each variable. The joint probability can be considered the product of each conditional probability and, in some instances, comprises the logarithm of that product. The second method is Markov chain Monte Carlo (MCMC), which can be implemented when the sample size is large. MCMC is a well-known class of sample distribution algorithms and will not be discussed in detail herein.

The assumption of conditional independence of variables forms the basis for Naïve Bayes classifiers. This assumption implies there is no correlation between different input features. As a result, the number of computed probabilities is significantly reduced as well as the computation of the probability normalization. While independence between features is rarely true, this assumption exchanges reduced computations for less accurate predictions, however the predictions are reasonably accurate. In example embodiments, one or more amino acid sequences are mapped to the BN graph to train the BN machine learning module, which, after training, is used to estimate one or more immunogenic peptides comprising one or more peptide binding motifs or one or more ligand regions.

### Random Forest

In one example embodiment, random forest is implemented. RF consists of an ensemble of decision trees producing individual class predictions. The prevailing prediction from the ensemble of decision trees becomes the RF prediction. Decision trees are branching flowchart-like graphs comprising of the root, nodes, edges/branches, and leaves. The root is the first decision node from which feature information is assessed and from it extends the first set of edges/branches. The edges/branches contain the information of the outcome of a node and pass the information to the next node. The leaf nodes are the terminal nodes that output the prediction. Decision trees can be used for both classification as well as regression and is typically trained using supervised learning methods. Training of a decision tree is sensitive to the training data set. An individual decision tree may become over or under-fit to the training data and result in a poor predictive model. Random forest compensates by using multiple decision trees trained on different data sets. In example embodiments, one or more amino acid sequences are used to train the nodes of the decision trees of a RF machine learning module, which, after training, is used to estimate one or more immunogenic peptides comprising one or more peptide binding motifs or one or more ligand regions.

### Neural Networks

In one example embodiment, Neural Networks are implemented. NNs are a family of statistical learning models influenced by biological neural networks of the brain. NNs can be trained on a relatively large dataset (e.g., 50,000 or more) and used to estimate, approximate, or predict an output that depends on a large number of inputs/features. NNs can be envisioned as so-called “neuromorphic” systems of interconnected processor elements, or “neurons”, and exchange electronic signals, or “messages”. Similar to the so-called “plasticity” of synaptic neurotransmitter connections that carry messages between biological neurons, the connections in NNs that carry electronic “messages” between “neurons” are provided with numeric weights that correspond to the strength or weakness of a given connection. The weights can be tuned based on experience, making NNs adaptive to inputs and capable of learning. For example, an NN for generating one or more immunogenic peptides comprising one or more peptide binding motifs for use in immunological compositions is defined by a set of input neurons that can be given input data such as one or more amino acid sequences. The input neuron weighs and transforms the input data and passes the result to other neurons, often referred to as “hidden” neurons. This is repeated until an output neuron is activated. The activated output neuron produces a result. In example embodiments, one or more amino acid sequences are used to train the neurons in a NN machine learning module, which, after training, is used to generate one or more immunogenic peptides comprising one or more peptide binding motifs.

### Recurrent Neural Network (RNN)

In an example embodiment, a recurrent neural network is implemented. RNNs are class of NNs further attempting to replicate the biological neural networks of the brain. RNNs comprise of delay differential equations on sequential data or time series data to replicate the processes and interactions of the human brain. RNNs have “memory” wherein the RNN can take information from prior inputs to influence the current output. RNNs can process variable length sequences of inputs by using their “memory” or internal state information. Where NNs may assume inputs are independent from the outputs, the outputs of RNNs may be dependent on prior elements with the input sequence. See Sherstinsky, Alex. “Fundamentals of recurrent neural network (RNN) and long short-term memory (LSTM) network.” Physica D: Nonlinear Phenomena 404 (2020): 132306.

### Long Short-Term Memory (LSTM)

In an example embodiment, a Long Short-term Memory is implemented. LSTM are a class of RNNs designed to overcome vanishing and exploding gradients. In RNNs, long term dependencies become more difficult to capture because the parameters or weights either do not change with training or fluctuate rapidly. This occurs when the RNN gradient exponentially decreases to zero, resulting in no change to the weights or parameters, or exponentially increases to infinity, resulting in large changes in the weights or parameters. This exponential effect is dependent on the number of layers and multiplicative gradient. LSTM overcomes the vanishing/exploding gradients by implementing “cells” within the hidden layers of the NN. The “cells” comprise three gates: an input gate, an output gate, and a forget gate. The input gate reduces error by controlling relevant inputs to update the current cell state. The output gate reduces error by controlling relevant memory content in the present hidden state. The forget gate reduces error by controlling whether prior cell states are put in “memory” or forgotten. The gates use activation functions to determine whether the data can pass through the gates. While one skilled in the art would recognize the use of any relevant activation function, example activation functions are sigmoid, tanh, and RELU. See Zhu, Xiaodan, et al. “Long short-term memory over recursive structures.” International Conference on Machine Learning. PMLR, 2015.

### Convolutional Neural Network (CNN)

In an example embodiment, a convolutional neural network is implemented. CNNs is a class of NNs further attempting to replicate the biological neural networks, but of the animal visual cortex. CNNS process data with a grid pattern to learn spatial hierarchies of features. A typical CNN comprises of three layers: convolution, pooling, and fully connected. The convolution and pooling layers extract features, such as those described herein. The convolutional layer comprises of multiple mathematical operations such as of linear operations, a specialized type being a convolution. The fully connected layer combines the extracted features into an output. The input data, such as one or more amino acids and optionally corresponding features, may be represented in a grid, i.e., an array of numbers. A grid of parameters, called a kernel, operates as an optimizable feature extractor and is applied to each position in the grid. Extracted features may become hierarchically more complex as one layer feeds its output into the next layer.

See Yamashita, R., et al Convolutional neural networks: an overview and application in radiology. Insights Imaging 9, 611-629 (2018).

### Deep Learning

In example embodiments, deep learning is implemented. Deep learning expands the neural network by including more layers of neurons. A deep learning module is characterized as having three “macro” layers: (1) an input layer which takes in the input features, and fetches embeddings for the input, (2) one or more intermediate (or hidden) layers which introduces nonlinear neural net transformations to the inputs, and (3) a response layer which transforms the final results of the intermediate layers to the prediction. In example embodiments, one or more amino acid sequences are used to train the neurons of a deep learning module, which, after training, is used to estimate one or more immunogenic peptides comprising one or more peptide binding motifs.

### Matrix Factorization

In example embodiments, Matrix Factorization is implemented. Matrix factorization machine learning exploits inherent relationships between two entities drawn out when multiplied together. Generally, the input features are mapped to a matrix F which is multiplied with a matrix R containing the relationship between the features and a predicted outcome. The resulting dot product provides the prediction. The matrix R is constructed by assigning random values throughout the matrix. In this example, two training matrices are assembled. The first matrix X contains training input features, and the second matrix Z contains the known output of the training input features. First the dot product of R and X are computed and the square mean error, as one example method, of the result is estimated. The values in R are modulated and the process is repeated in a gradient descent style approach until the error is appropriately minimized. The trained matrix Ris then used in the machine learning model. In example embodiments, one or more amino acid sequences are used to train the relationship matrix R in a matrix factorization machine learning module. After training, the relationship matrix R and input matrix F, which comprises vector representations of one or more amino acid sequences, results in the prediction matrix P comprising one or more immunogenic peptides comprising one or more peptide binding motifs.

### Hidden Markov Model

In example embodiments, a hidden Markov model is implemented. A HMM takes advantage of the statistical Markov model to predict an outcome. A Markov model assumes a Markov process, wherein the probability of an outcome is solely dependent on the previous event. In the case of HMM, it is assumed an unknown or “hidden” state is dependent on some observable event. A HMM comprises a network of connected nodes. Traversing the network is dependent on three model parameters: start probability; state transition probabilities; and observation probability. The start probability is a variable that governs, from the input node, the most plausible consecutive state. From there each node i has a state transition probability to node j. Typically the state transition probabilities are stored in a matrix Mᵢⱼ wherein the sum of the rows, representing the probability of state i transitioning to state j, equals 1. The observation probability is a variable containing the probability of output o occurring. These too are typically stored in a matrix Nₒⱼ wherein the probability of output o is dependent on state j. To build the model parameters and train the HMM, the state and output probabilities are computed. This can be accomplished with, for example, an inductive algorithm. Next, the state sequences are ranked on probability, which can be accomplished, for example, with the Viterbi algorithm. Finally, the model parameters are modulated to maximize the probability of a certain sequence of observations. This is typically accomplished with an iterative process wherein the neighborhood of states is explored, the probabilities of the state sequences are measured, and model parameters updated to increase the probabilities of the state sequences. In example embodiments, one or more amino acid sequences are used to train the nodes/states of the HMM machine learning module, which, after training, is used to estimate one or more immunogenic peptides comprising one or more peptide binding motifs.

### Support Vector Machine

In example embodiments, support vector machines are implemented. SVMs separate data into classes defined by n-dimensional hyperplanes (n-hyperplane) and are used in both regression and classification problems. Hyperplanes are decision boundaries developed during the training process of a SVM. The dimensionality of a hyperplane depends on the number of input features. For example, a SVM with two input features will have a linear (1-dimensional) hyperplane while a SVM with three input features will have a planer (2-dimensional) hyperplane. A hyperplane is optimized to have the largest margin or spatial distance from the nearest data point for each data type. In the case of simple linear regression and classification a linear equation is used to develop the hyperplane. However, when the features are more complex a kernel is used to describe the hyperplane. A kernel is a function that transforms the input features into higher dimensional space. Kernel functions can be linear, polynomial, a radial distribution function (or gaussian radial distribution function), or sigmoidal. In example embodiments, one or more amino acid sequences are used to train the linear equation or kernel function of the SVM machine learning module, which, after training, is used to estimate one or more immunogenic peptides comprising one or more peptide binding motifs.

### K-Means Clustering

In one example embodiment, K-means clustering is implemented. KMC assumes data points have implicit shared characteristics and “clusters” data within a centroid or “mean” of the clustered data points. During training, KMC adds a number of k centroids and optimizes its position around clusters. This process is iterative, where each centroid, initially positioned at random, is re-positioned towards the average point of a cluster. This process concludes when the centroids have reached an optimal position within a cluster. Training of a KMC module is typically unsupervised. In example embodiments, one or more amino acid sequences are used to train the centroids of a KMC machine learning module, which, after training, is used to estimate one or more immunogenic peptides comprising one or more peptide binding motifs.

### K-Nearest Neighbor

In one example embodiment, K-nearest neighbor is implemented. On a general level, KNN shares similar characteristics to KMC. For example, KNN assumes data points near each other share similar characteristics and computes the distance between data points to identify those similar characteristics but instead of k centroids, KNN uses k number of neighbors. The k in KNN represents how many neighbors will assign a data point to a class, for classification, or object property value, for regression. Selection of an appropriate number of k is integral to the accuracy of KNN. For example, a large k may reduce random error associated with variance in the data but increase error by ignoring small but significant differences in the data. Therefore, a careful choice of k is selected to balance overfitting and underfitting. Concluding whether some data point belongs to some class or property value k, the distance between neighbors is computed. Common methods to compute this distance are Euclidean, Manhattan or Hamming to name a few. In some embodiments, neighbors are given weights depending on the neighbor distance to scale the similarity between neighbors to reduce the error of edge neighbors of one class “out-voting” near neighbors of another class. In one example embodiment, k is 1 and a Markov model approach is utilized. In example embodiments, one or more amino acids are used to train a KNN machine learning module, which, after training, is used to estimate one or more immunogenic peptides comprising one or more peptide binding motifs.

To perform one or more of its functionalities, the machine learning module may communicate with one or more other systems. For example, an integration system may integrate the machine learning module with one or more email servers, web servers, one or more databases, or other servers, systems, or repositories. In addition, one or more functionalities may require communication between a user and the machine learning module.

Any one or more of the modules described herein may be implemented using hardware (e.g., one or more processors of a computer/machine) or a combination of hardware and software. For example, any module described herein may configure a hardware processor (e.g., among one or more hardware processors of a machine) to perform the operations described herein for that module. In some example embodiments, any one or more of the modules described herein may comprise one or more hardware processors and may be configured to perform the operations described herein. In example embodiments, one or more hardware processors are configured to include any one or more of the modules described herein.

Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices. The multiple machines, databases, or devices are communicatively coupled to enable communications between the multiple machines, databases, or devices. The modules themselves are communicatively coupled (e.g., via appropriate interfaces) to each other and to various data sources, to allow information to be passed between the applications so as to allow the applications to share and access common data.

### Example Computing Device

FIG. 16 depicts a block diagram of a computing machine 2000 and a module 2050 in accordance with certain examples. The computing machine 2000 may comprise, but are not limited to, remote devices, work stations, servers, computers, general purpose computers, Internet/web appliances, hand-held devices, wireless devices, portable devices, wearable computers, cellular or mobile phones, personal digital assistants (PDAs), smart phones, smart watches, tablets, ultrabooks, netbooks, laptops, desktops, multi-processor systems, microprocessor-based or programmable consumer electronics, game consoles, set-top boxes, network PCs, mini-computers, and any machine capable of executing the instructions. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The one or more processor 2010 may be configured to execute code or instructions to perform the operations and functionality described herein, manage request flow and address mappings, and to perform calculations and generate commands. Such code or instructions could include, but is not limited to, firmware, resident software, microcode, and the like. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), tensor processing units (TPUs), a graphics processing unit (“GPU”), a field programmable gate array (“FPGA”), a programmable logic device (“PLD”), a radio-frequency integrated circuit (RFIC), a controller, a state machine, gated logic, discrete hardware components, any other processing unit, or any combination or multiplicity thereof. In example embodiments, each processor 2010 can include a reduced instruction set computer (RISC) microprocessor. The processor 2010 may be a single processing unit, multiple processing units, a single processing core, multiple processing cores, special purpose processing cores, co-processors, or any combination thereof. According to certain examples, the processor 2010 along with other components of the computing machine 2000 may be a virtualized computing machine executing within one or more other computing machines. Processors 2010 are coupled to system memory and various other components via a system bus 2020.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random-access memory (“RAM”), static random-access memory (“SRAM”), dynamic random-access memory (“DRAM”), and synchronous dynamic random-access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 is coupled to system bus 2020 and can include a basic input/output system (BIOS), which controls certain basic functions of the processor 2010 and/or operate in conjunction with, a non-volatile storage device such as the storage media 2040.

In example embodiments, the computing device 2000 includes a graphics processing unit (GPU) 2090. Graphics processing unit 2090 is a specialized electronic circuit designed to manipulate and alter memory to accelerate the creation of images in a frame buffer intended for output to a display. In general, a graphics processing unit 2090 is efficient at manipulating computer graphics and image processing and has a highly parallel structure that makes it more effective than general-purpose CPUs for algorithms where processing of large blocks of data is done in parallel.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any electromagnetic storage device, any semiconductor storage device, any physical-based storage device, any removable and non-removable media, any other data storage device, or any combination or multiplicity thereof. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The module 2050 may comprise one or more hardware or software elements, as well as an operating system, configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. Each of the operating system, one or more application programs, other program modules, and program data or some combination thereof, may include an implementation of a networking environment. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for coupling in operation the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including cursor control devices, touch-screens, scanners, electronic digitizers, sensors, receivers, touchpads, trackballs, cameras, microphones, alphanumeric input devices, any other pointing devices, or any combinations thereof. The I/O interface 2060 may couple the computing machine 2000 to various output devices including video displays (The computing device 2000 may further include a graphics display, for example, a plasma display panel (PDP), a light emitting diode (LED) display, a liquid crystal display (LCD), a projector, a cathode ray tube (CRT), or any other display capable of displaying graphics or video), audio generation device, printers, projectors, tactile feedback devices, automation control, robotic components, actuators, motors, fans, solenoids, valves, pumps, transmitters, signal emitters, lights, and so forth. The I/O interface 2060 may couple the computing device 2000 to various devices capable of input and out, such as a storage unit. The devices can be interconnected to the system bus 2020 via a user interface adapter, which can include, for example, a Super I/O chip integrating multiple device adapters into a single integrated circuit.

The computing machine 2000 may operate in a networked environment using logical connections through the network interface 2070 to one or more other systems or computing machines across the network 2080. The network 2080 may include a local area network (“LAN”), a wide area network (“WAN”), an intranet, an Internet, a mobile telephone network, storage area network (“SAN”), personal area network (“PAN”), a metropolitan area network (“MAN”), a wireless network (“WiFi;”), wireless access networks, a wireless local area network (“WLAN”), a virtual private network (“VPN”), a cellular or other mobile communication network, Bluetooth, near field communication (“NFC”), ultra-wideband, wired networks, telephone networks, optical networks, copper transmission cables, or combinations thereof or any other appropriate architecture or system that facilitates the communication of signals and data. The network 2080 may be packet switched, circuit switched, of any topology, and may use any communication protocol. The network 2080 may comprise routers, firewalls, switches, gateway computers and/or edge servers. Communication links within the network 2080 may involve various digital or analog communication media such as fiber optic cables, free-space optics, waveguides, electrical conductors, wireless links, antennas, radio-frequency communications, and so forth.

Information for facilitating reliable communications can be provided, for example, as packet/message sequencing information, encapsulation headers and/or footers, size/time information, and transmission verification information such as cyclic redundancy check (CRC) and/or parity check values. Communications can be made encoded/encrypted, or otherwise made secure, and/or decrypted/decoded using one or more cryptographic protocols and/or algorithms, such as, but not limited to, Data Encryption Standard (DES), Advanced Encryption Standard (AES), a Rivest-Shamir-Adelman (RSA) algorithm, a Diffie-Hellman algorithm, a secure sockets protocol such as Secure Sockets Layer (SSL) or Transport Layer Security (TLS), and/or Digital Signature Algorithm (DSA). Other cryptographic protocols and/or algorithms can be used as well or in addition to those listed herein to secure and then decrypt/decode communications.

The processor 2010 may be connected to the other elements of the computing machine 2000 or the various peripherals discussed herein through the system bus 2020. The system bus 2020 represents one or more of any of several types of bus structures, including a memory bus or memory controller, a peripheral bus, an accelerated graphics port, and a processor or local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. It should be appreciated that the system bus 2020 may be within the processor 2010, outside the processor 2010, or both. According to certain examples, any of the processor 2010, the other elements of the computing machine 2000, or the various peripherals discussed herein may be integrated into a single device such as a system on chip (“SOC”), system on package (“SOP”), or ASIC device.

Examples may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing examples in computer programming, and the examples should not be construed as limited to any one set of computer program instructions. Further, a skilled programmer would be able to write such a computer program to implement an example of the disclosed examples based on the appended flow charts and associated description in the application text. Therefore, disclosure of a particular set of program code instructions is not considered necessary for an adequate understanding of how to make and use examples. Further, those ordinarily skilled in the art will appreciate that one or more aspects of examples described herein may be performed by hardware, software, or a combination thereof, as may be embodied in one or more computing systems. Moreover, any reference to an act being performed by a computer should not be construed as being performed by a single computer as more than one computer may perform the act.

The examples described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

A “server” may comprise a physical data processing system (for example, the computing device 2000 as shown in FIG. 16) running a server program. A physical server may or may not include a display and keyboard. A physical server may be connected, for example by a network, to other computing devices. Servers connected via a network may operate in the capacity of a server machine or a client machine in a server-client network environment, or as a peer machine in a distributed (e.g., peer-to-peer) network environment. The computing device 2000 can include clients' servers. For example, a client and server can be remote from each other and interact through a network. The relationship of client and server arises by virtue of computer programs in communication with each other, running on the respective computers.

The example systems, methods, and acts described in the examples and described in the figures presented previously are illustrative, not intended to be exhaustive, and not meant to be limiting. In alternative examples, certain acts can be performed in a different order, in parallel with one another, omitted entirely, and/or combined between different examples, and/or certain additional acts can be performed, without departing from the scope and spirit of various examples. Plural instances may implement components, operations, or structures described as a single instance. Structures and functionality that may appear as separate in example embodiments may be implemented as a combined structure or component. Similarly, structures and functionality that may appear as a single component may be implemented as separate components. Accordingly, such alternative examples are included in the scope of the following claims, which are to be accorded the broadest interpretation to encompass such alternate examples. The terminology used herein was chosen to best explain the principles of the embodiments, the practical application or technical improvement over technologies found in the marketplace, or to enable others of ordinary skill in the art to understand the embodiments disclosed herein.

Further embodiments are illustrated in the following Examples which are given for illustrative purposes only and are not intended to limit the scope of the invention.

### EXAMPLES

### Example 1—HLA-II Immunopeptidome Profiling and Deep Learning Reveal Features of Antigenicity to Inform Antigen Discovery

### INTRODUCTION

Adaptive immunity is endowed with the ability to specifically recognize and mount responses to new antigens that the host species may not have previously encountered, and then to retain memory against these antigens to protect the individual against future exposures. The cellular arm of adaptive immunity is mediated by T cells, which collectively generate a diverse clonal repertoire of T cell receptors (TCRs) that recognize peptide antigens displayed by major histocompatibility (MHC) proteins on the surface of accessory cells. The adaptive element of cell-mediated immunity is conferred by germline-encoded variants of MHC that collectively expand the spectrum of unique peptide antigens that can be displayed to T cells, and the diverse TCR repertoire generated by somatic recombination of TCR gene segments. Class I MHC complexes are expressed in most nucleated cells and generally present peptide antigen to CD8 T cells, whereas class II MHC complexes are predominantly expressed in professional antigen presenting cells (APCs) and present peptide antigen to CD4 T cells (Germain and Margulies 1993; Neefjes et al. 2011). MHCII-restricted CD4 T cells exhibit remarkable functional heterogeneity and coordinate diverse immune responses tailored towards different pathogen threats in infectious disease, but also play important roles in tolerance, cancer, autoimmunity, and allergy (Borst et al. 2018; Alfei, Ho, and Lo 2021; Jurewicz and Stern 2019; Zheng and Wakim 2021). Thus, MHCII-restricted CD4 T cell responses perform diverse functions ranging from maintaining tolerance to the microbiome to coordinating protective immunity to emerging pathogen threats.

In humans, MHC proteins are encoded by human leukocyte antigen (HLA) genes. Classical HLA-II proteins are encoded by three highly polymorphic isotypes, HLA-DP, HLA-DQ, and HLA-DR. These HLA-II isotypes are heterodimers of alpha and beta chain proteins, and each HLA-II heterodimer binds a diverse spectrum of peptides ranging in length from 12-25 amino acids. Peptides bind within a structurally defined groove in HLA-II heterodimers, and these complexes are stabilized by interactions between HLA-II and a 9-mer amino acid core sequence in the peptide designated by positions P1-9 (Stern et al. 1994). Genetic variation within the HLA locus allows each HLA heterodimer to present a diverse array of peptide antigens. At the population level, this HLA diversity facilitates community protection, and some of the most robust genetic associations in human disease map to the HLA locus (Dendrou et al. 2018). Individuals who are heterozygous at the three HLA-II loci have at least 6 unique HLA-II heterodimers with distinct peptide binding properties. The number of possible HLA-II heterodimers can increase based on individual expression of HLA-DRB paralogs or trans-allelic pairing of alpha and beta chains from HLA-DP or-DQ. To date, 5,620 classical HLA-II protein variants have been reported (Robinson et al. 2020). Multiple factors, including co-evolution with pathogens, trade-off for protection against autoimmunity, and reproductive fitness have led to selection of genetic diversity in the HLA locus allowing for presentation of a broad spectrum of antigens that the immune system forms the basis of adaptive immunity, it is important to understand the rules of antigen presentation of each HLA-II heterodimer and identify antigens that drive disease. Identifying driver antigens in disease will enable functional characterization of antigen-specific T cell responses within the context of an enormously diverse TCR repertoire and its associated functional heterogeneity.

Advances in mass spectrometry (MS)-based immunopeptidomics approaches have enabled identification of endogenously processed and presented HLA-II peptides (Garde et al. 2019; Chong et al. 2018; Klaeger et al. 2021; Vizcaíno et al. 2020; Andreatta et al. 2019; Marcu et al. 2021; van Balen et al. 2020; Abelin et al. 2019). While the peptide binding specificity of HLA-II is a key determinant of antigen presentation to T cells, additional factors impact the efficiency by which peptides are processed from their source protein antigens. The immunogenicity of a peptide antigen is related to the efficiency by which it is presented to T cells, which is in turn affected by its abundance, propensity for uptake by APCs, trafficking to lysosomes, sensitivity to proteolytic processing, and affinity and stability on HLA-II (Unanue, Turk, and Neefjes 2016; Vyas, Van der Veen, and Ploegh 2008; Hsing and Rudensky 2005). In this context, mechanisms of antigen presentation and biochemical features of antigenicity remain incompletely understood.

Here, Applicants leveraged immunopeptidomics and machine learning to define rules of antigen processing and presentation that enabled discovery of immunodominant T cell antigens and functional characterization of antigen-specific T cell responses in health and disease. Applicants demonstrate that healthy individuals possess circulating T cells that actively recognize microbiome-derived epitopes and exhibit characteristics of tissue-protective functions and IL-17 skewing. Thus, identification of rare antigen-specific T cells sheds light on the host-microbiome relationship and its role in immune homeostasis. By contrast, pathogen infection disrupts homeostasis and elicits robust immune activation. Applicants identified an immunodominant T cell epitope in SARS-COV-2 nucleoprotein associated with antiviral Th1 immunity and IFN-γ production. Taken together, defining features of antigenicity enabled discovery of bacterial and viral antigens that drive functionally heterogeneous T cell responses in humans.

### Results

### Deep Monoallelic HLA-II Immunopeptidomic Profiling by Mass Spectrometry.

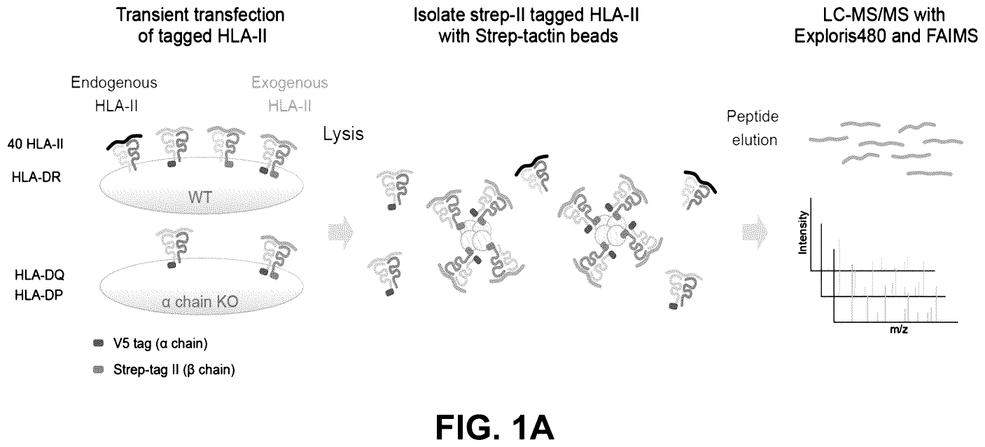

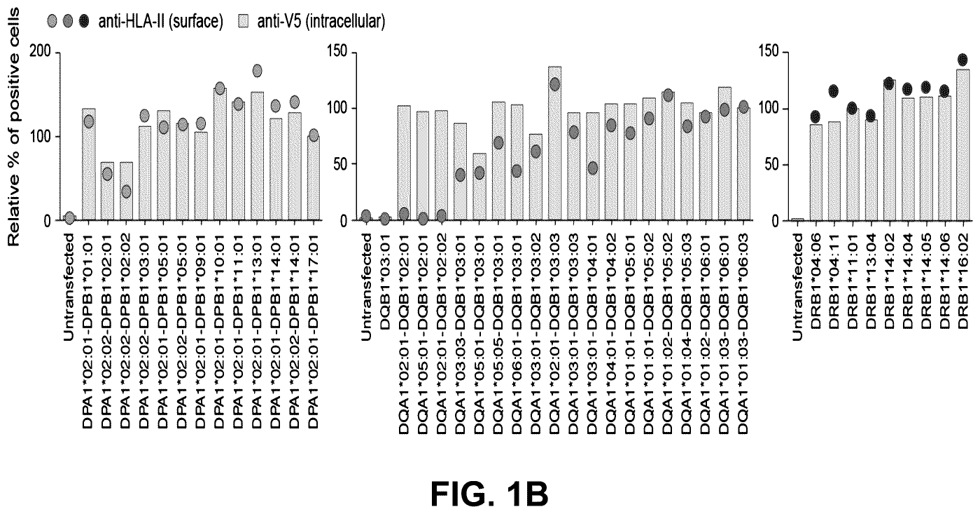

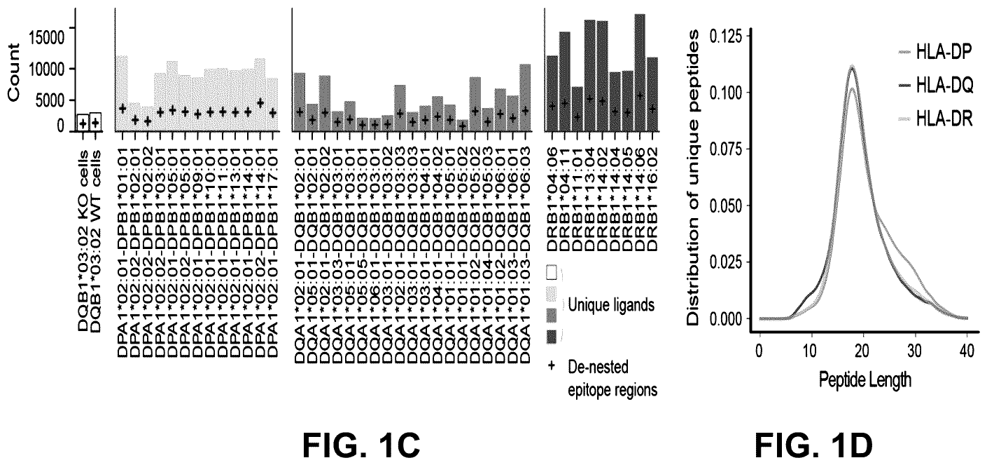

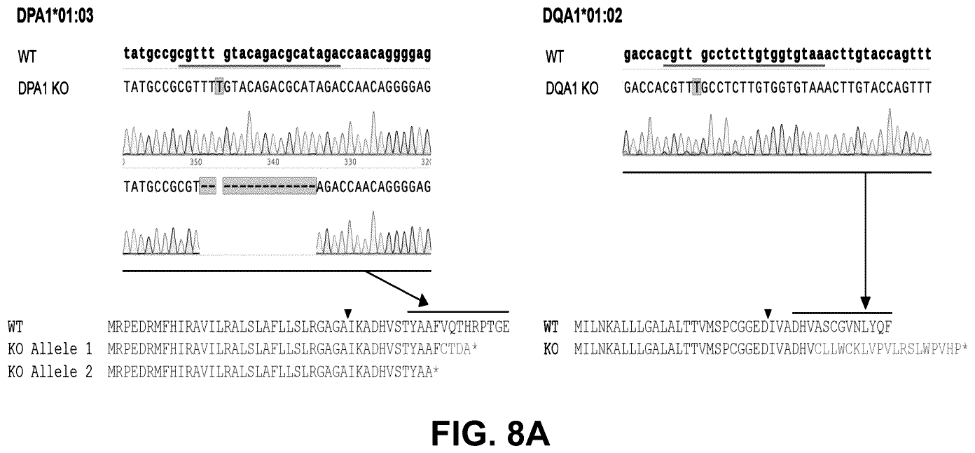

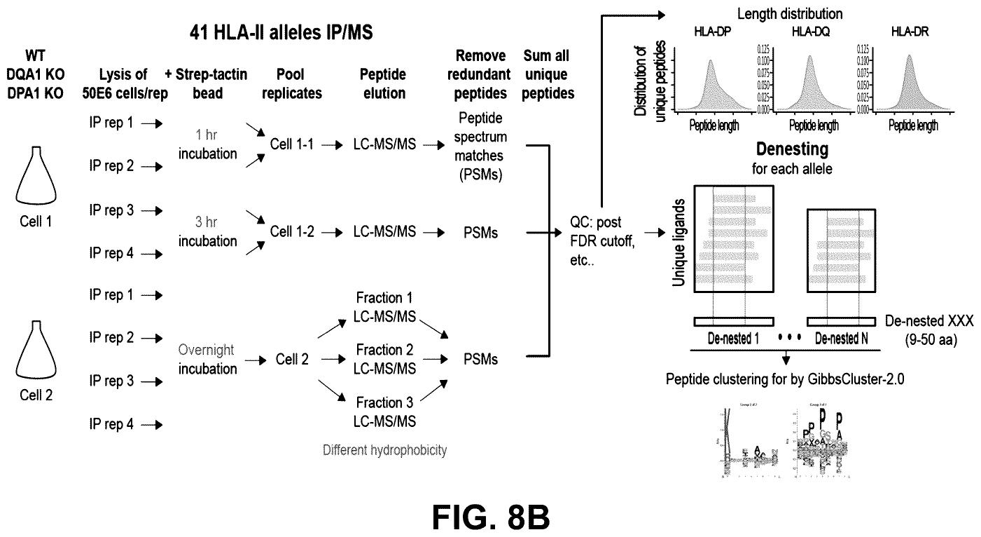

Despite the recognized importance of HLA diversity for expanding the scope of peptides presented to T cells, the general and allele-specific rules of antigen presentation remain incompletely understood. To address this shortcoming, Applicants set out to generate allele-specific HLA-II peptidome data for a broad range of HLA-II alleles across multiple ancestries and HLA-II isotypes (FIG. 1A and Table 1). First, Applicants generated expression constructs for HLA alpha and beta chains to facilitate expression in Expi293F cells. A Strep-tag II was appended to HLA-II beta chains for affinity purification, which was critical for certain alleles, because commercially available pan-HLA antibodies have different detection sensitivities across HLA-II heterodimers, especially for HLA-DQ (FIG. 1B). Second, Applicants generated HLA-alpha chain knockout (KO) cell lines to prevent heterodimer pairing with endogenous alpha chains in Expi293F cells (FIG. 8A). Third, HLA-DM was co-expressed with all HLA-II heterodimers to facilitate peptide exchange for high-affinity ligands. Lastly, after purification of Strep-tagged HLA-II heterodimers, Applicants eluted peptide ligands using offline fractionation in combination with FAIMS (Klaeger et al. 2021) in the mass spectrometry workflow and implemented improvements to peptide identification through optimized database search strategies (see methods).

Altogether, Applicants performed monoallelic immunopeptidomic profiling on 41 unique HLA-II heterodimers, including 19 from HLA-DQ, 13 from HLA-DP, and 9 from HLA-DR (FIG. 1C). Applicants identified 203,022 unique peptide ligands in total. For each HLA-II heterodimer, Applicants detected between 2,500 to 12,500 unique ligands (FIG. 1C) with length distributions in the expected range of 12-25mers (Abelin et al. 2019) (FIG. 1D). Applicants noted HLA-DQ bound a small population of shorter peptides while HLA-DP bound peptides of longer length, suggesting that the three HLA isotypes may provide further diversity on antigen presentation through their different length preferences. Taken together, this deep immunopeptidome dataset has substantial potential for uncovering the properties of HLA-II peptide preferences and features of antigenicity associated with antigen processing.

### Immunopeptidomics Reveals HLA-II Allele-Specific Ligand Preferences.

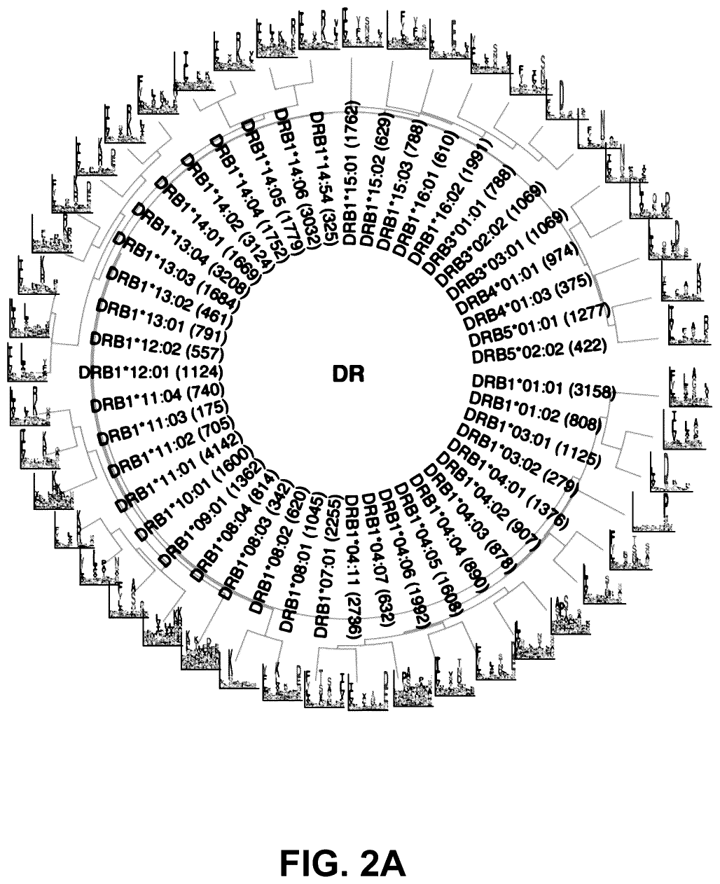

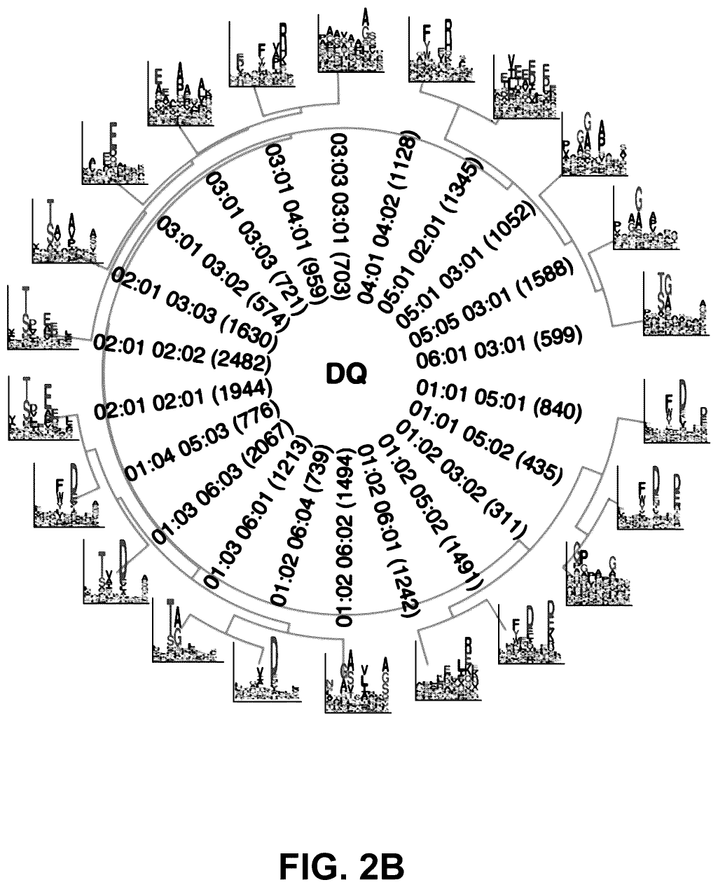

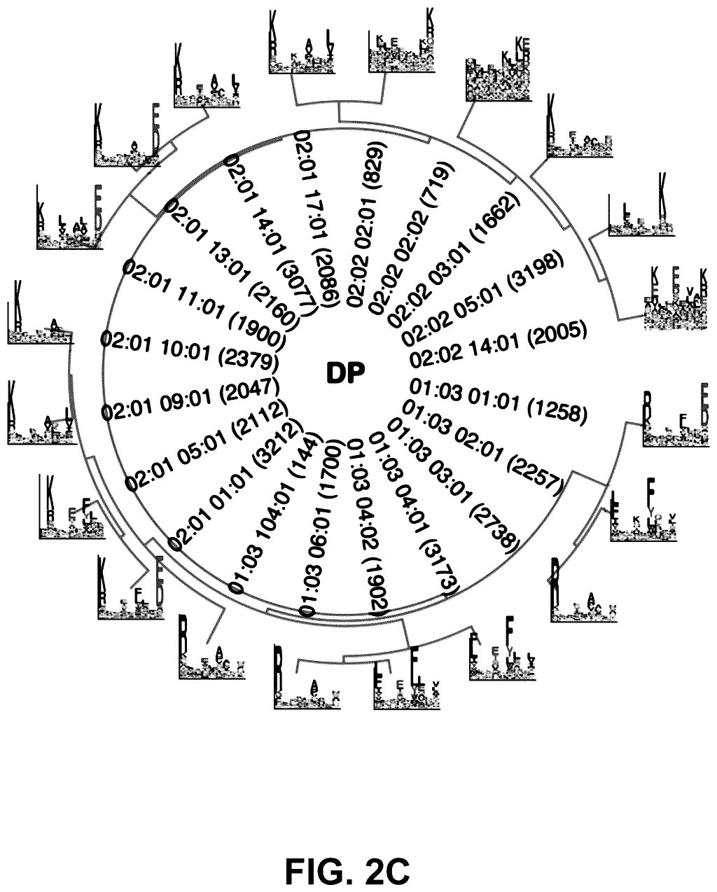

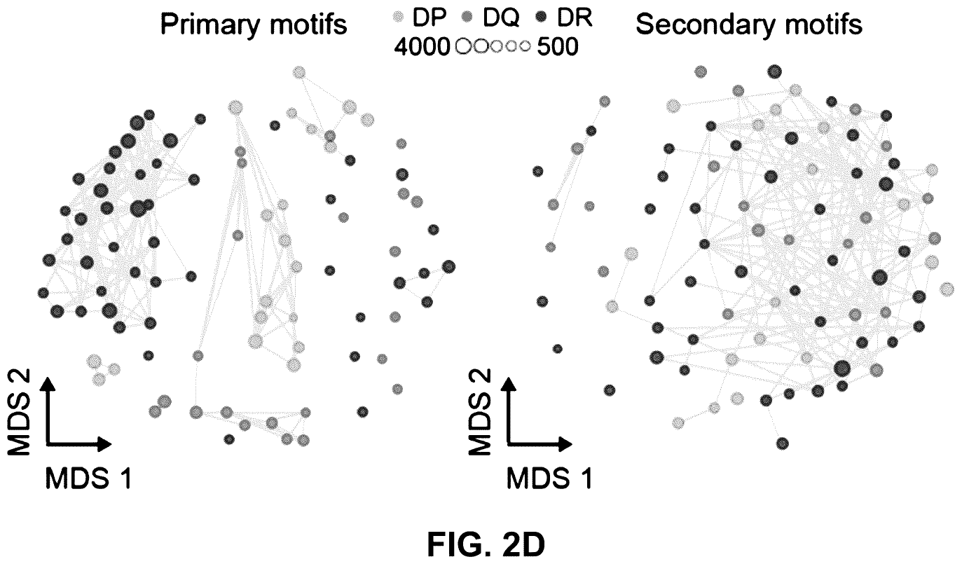

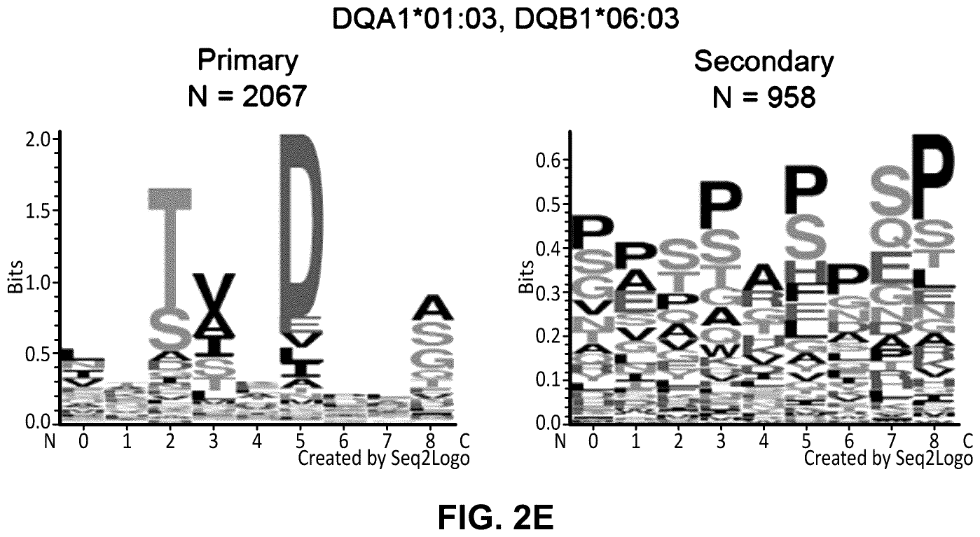

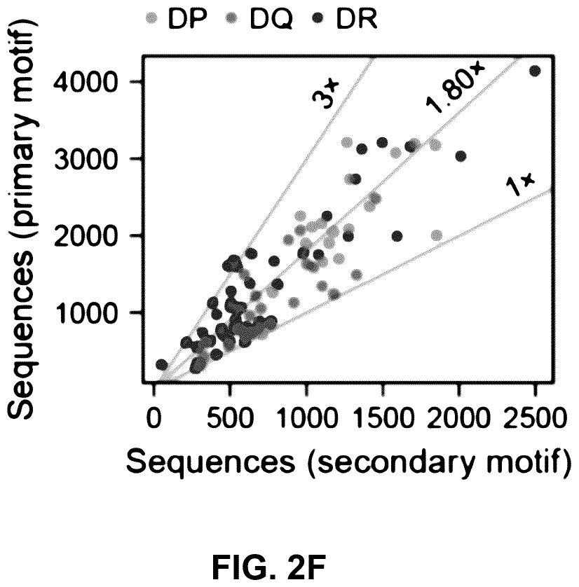

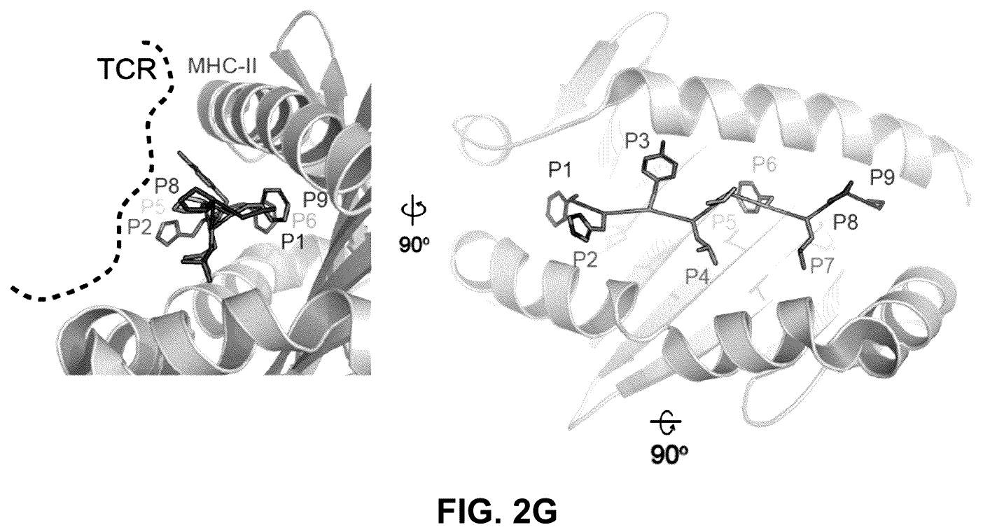

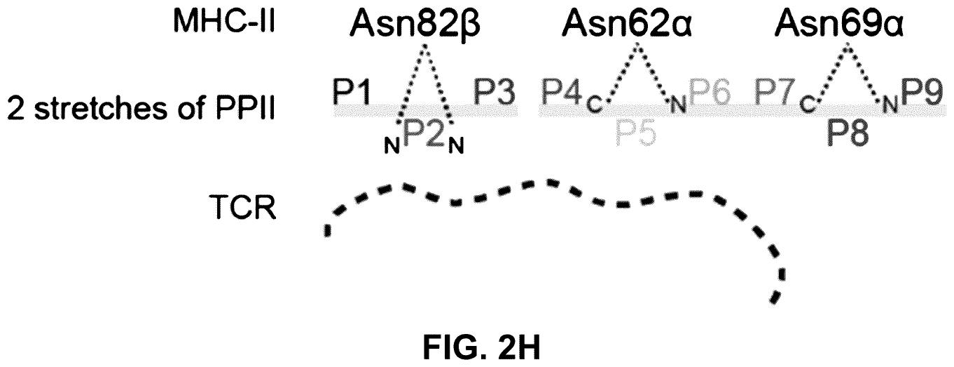

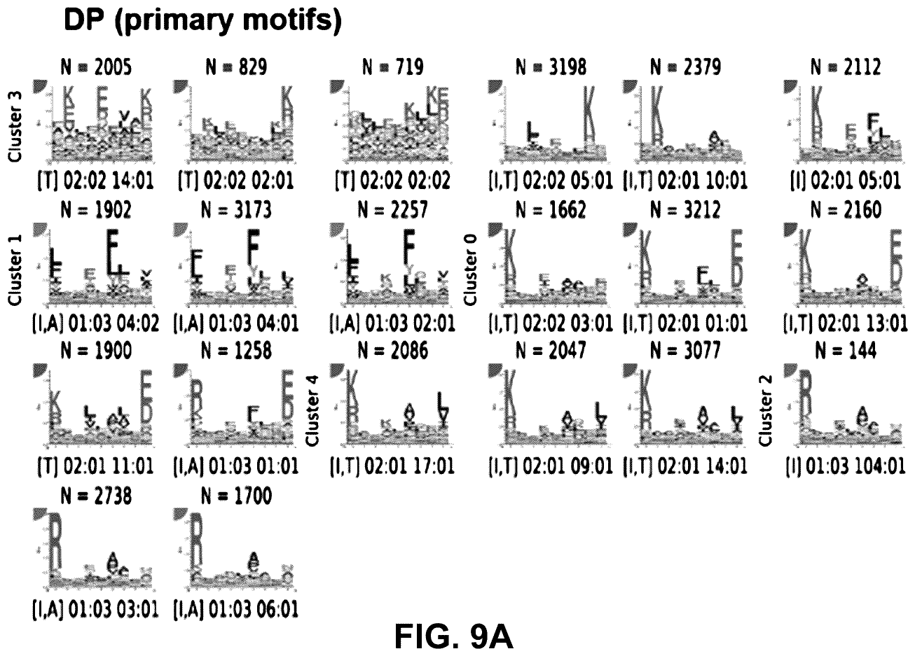

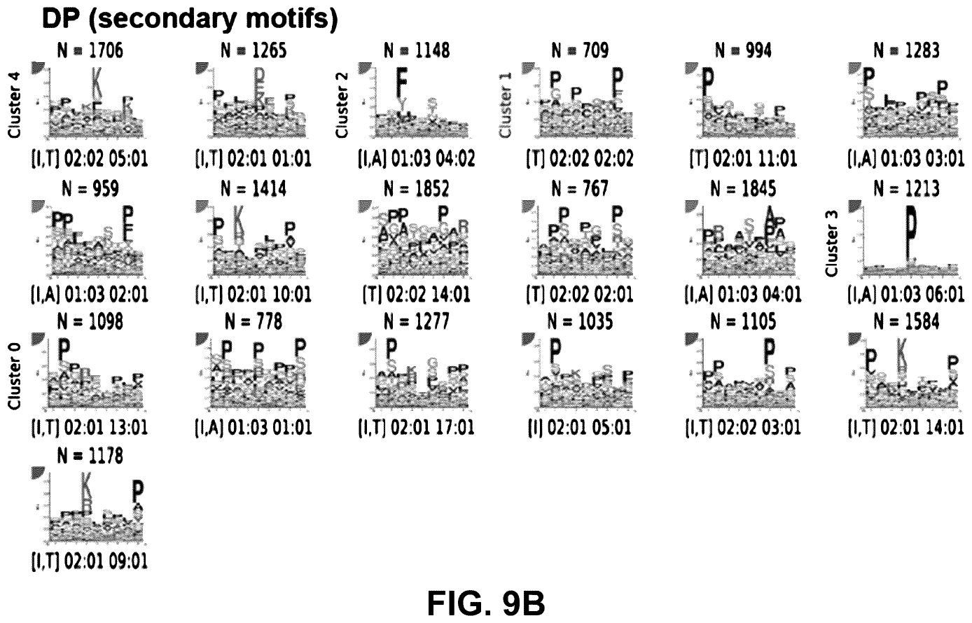

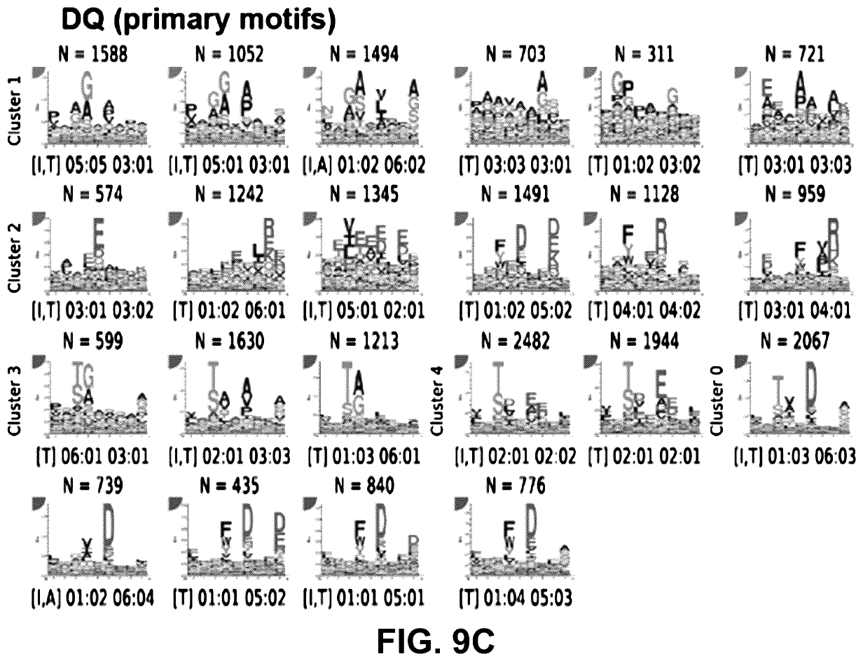

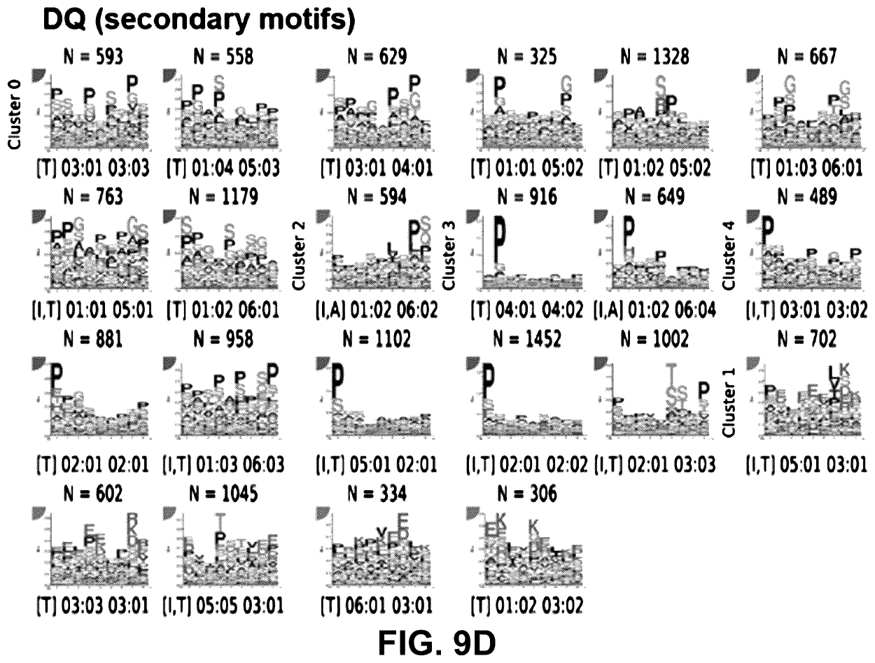

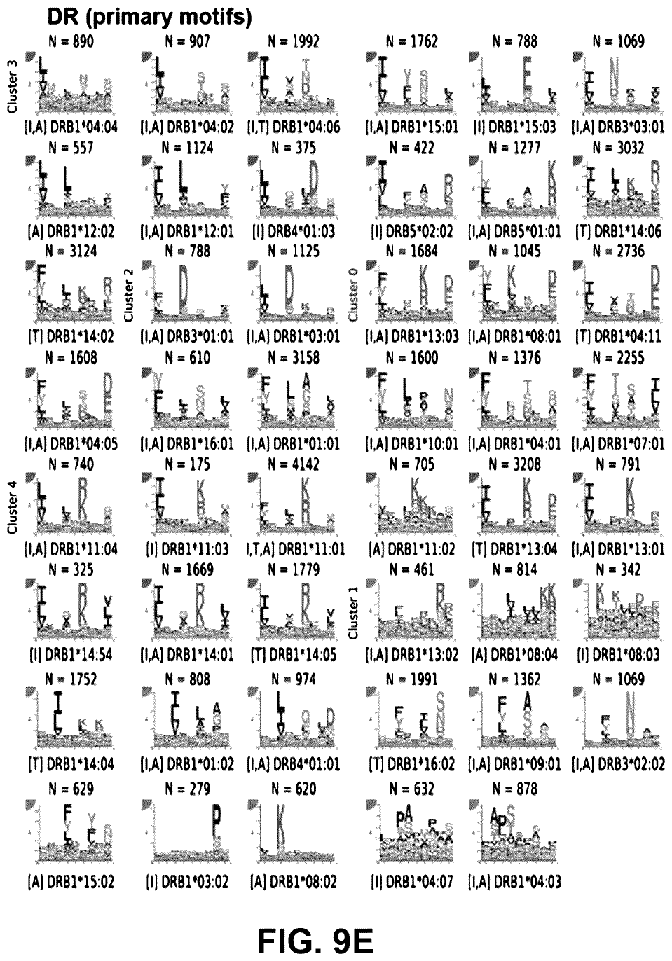

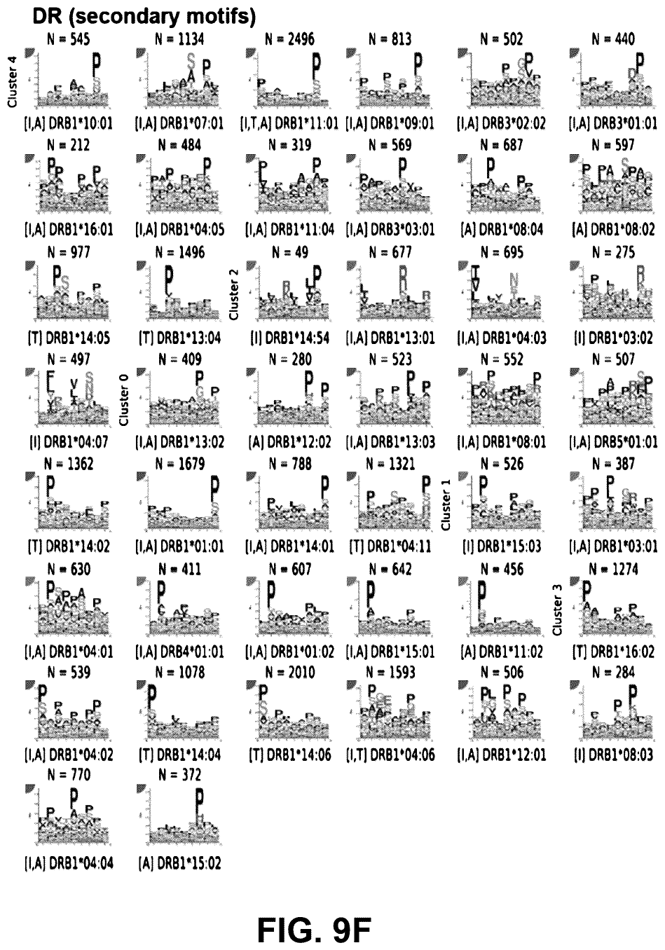

To identify unique peptide motifs associated with specific binding to HLA-II heterodimers, Applicants assembled a comprehensive catalog of ligands from monoallelic datasets. Altogether, this dataset included 87 HLA-II alleles and 665,239 peptides (this work, IEDB and (Abelin et al. 2019)). Peptides were de-nested into non-overlapping epitope regions in human proteins (FIG. 8B). GibbsCluster 2.0 (Andreatta, Alvarez, and Nielsen 2017) was used to infer up to two motifs per allele (Methods, FIG. 9). Clustering of unique HLA-II peptide regions revealed binding core motifs, with strongest amino acid enrichment at positions P1, P4, P6 and P9 (FIG. 2A-C and FIG. 9). This is consistent with the known configuration in which peptides interact with the HLA-II binding groove. HLA-II-bound peptides adopt an extended helical conformation resembling two stretches of a polyproline helix type II (PPII) where the side chains of every third residue align in the same direction (FIG. 2G-H). In this conformation, the side chains of residues at P1 and P9 point towards the peptide binding groove of HLA-II, and the side chains at P2, P5, and P8 align with one another, pointing outwards from the HLA-II groove to form contacts with cognate TCR.

Most of the motif features Applicants identified captured HLA-II peptide binding preferences driven by interactions with the peptide side chain. However, a closer inspection of the data revealed a secondary group of motifs associated with a smaller number of peptides (FIG. 9). While joint clustering of primary motifs shows clear isotype-specific grouping, a similar analysis in the secondary motif space revealed a mixing of secondary motifs, hinting at the existence of promiscuous HLA-II binding patterns (FIG. 2D). To illustrate this observation, Applicants provide a representative example showing a novel HLA-DQ primary motif (P4T/S and P6D) and proline rich secondary motif (FIG. 2E). Secondary motifs represented on average 35.7% of unique epitope regions related to each allele, therefore primary motifs cover 1.80× more sequences (FIG. 2F). The appearance of these secondary motifs suggests promiscuous binding across different HLA-II alleles, and their patterns and emergence were consistent in all data sources used in this study (FIG. 9). In peptides with secondary motifs, the backbone of the stretched PPII helix can efficiently form hydrogen bonds with neighboring residues of HLA-II. Hydrogen bonds formed with the absolutely conserved asparagine residues (Asn62α, Asn69α, and Asn82β) of HLA-II are known to be sufficient to hold the peptide in place (Stern et al. 1994). Because these hydrogen bonds do not involve side chains of the peptide, any peptides rich in Pro or Gly may form a PPII conformation and bind HLA-II promiscuously (FIG. 2H). Notably, collagen is rich in Pro and Gly, and there are validated collagen epitopes associated with rheumatoid arthritis (Bowes and Kenten 1948; Chemin et al. 2016; Dessen et al. 1997; Di Sante et al. 2015; Kjellén et al. 1998; Jones et al. 2006). This structural analysis reveals that peptides conforming to secondary motifs can be plausible, but less specific HLA-II binders, which emerge upon deep monoallelic peptidome profiling.

### HLA-II Isotypes Exhibit Unique Peptide Binding Properties.

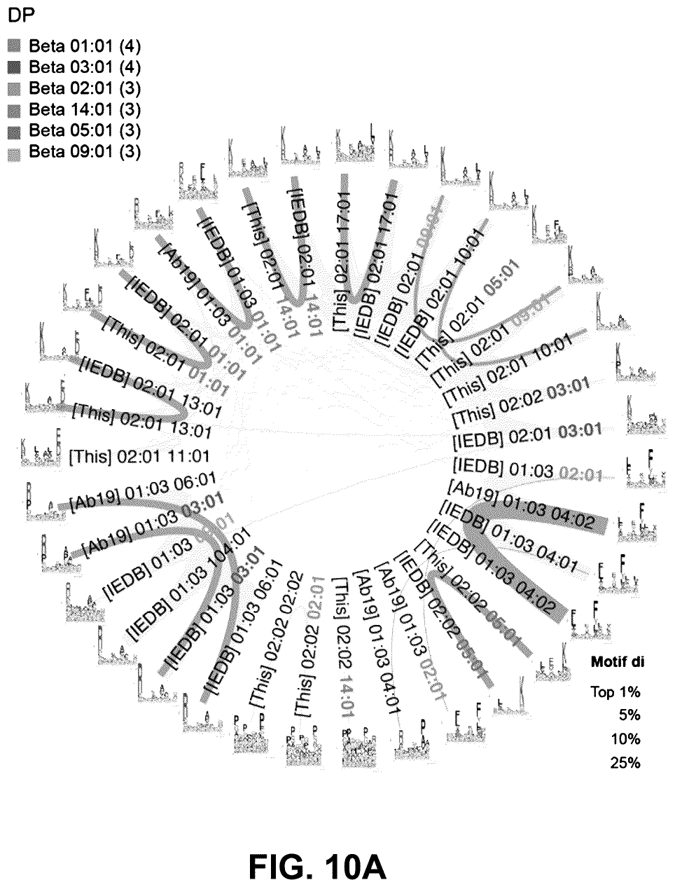

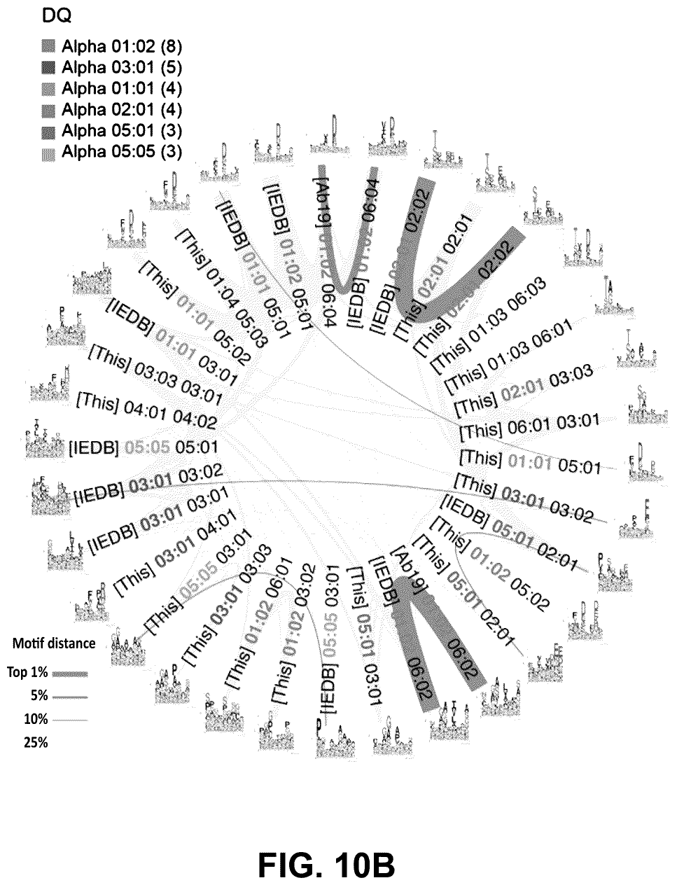

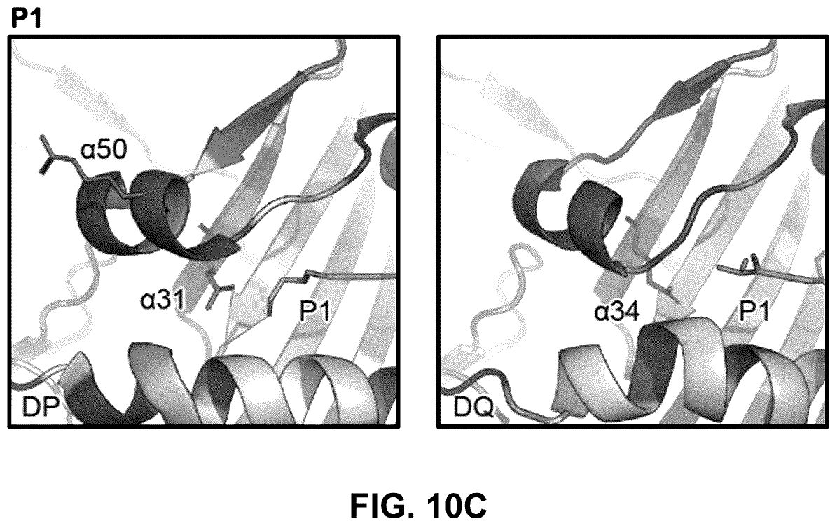

Having assembled a comprehensive dataset from monoallelic profiling of HLA-DR,-DP, and -DQ, Applicants sought to define isotype-specific peptide binding rules. First, Applicants selected a single, consensus motif for each allele based on GibbsCluster divergence score and aligned the motifs for different alleles based on P1 (Methods). This enabled a comparison of the amino acid distributions at positions P1-P9 across heterodimers. Motifs corresponding to the same heterodimers derived from different data sets were largely consistent and clustered together (FIG. 10A-B).

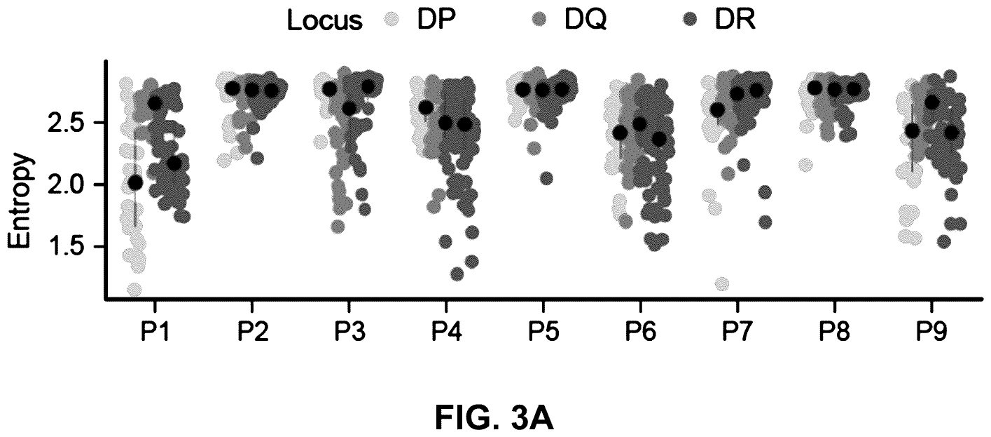

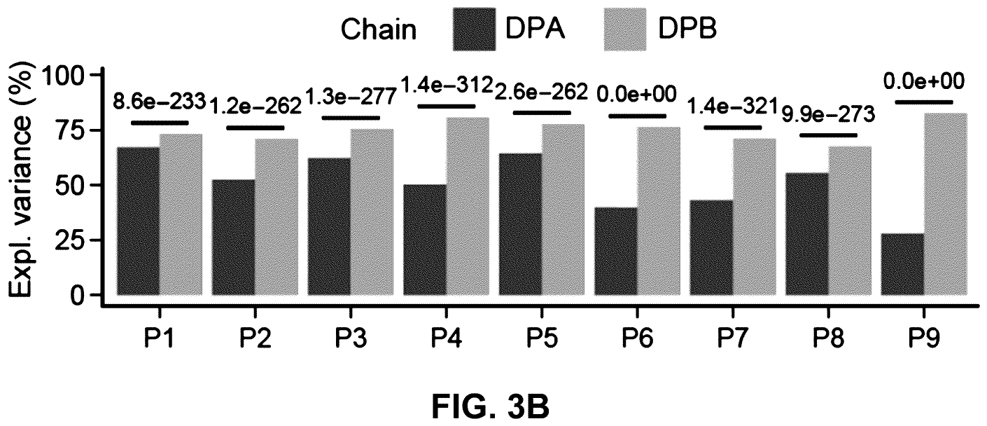

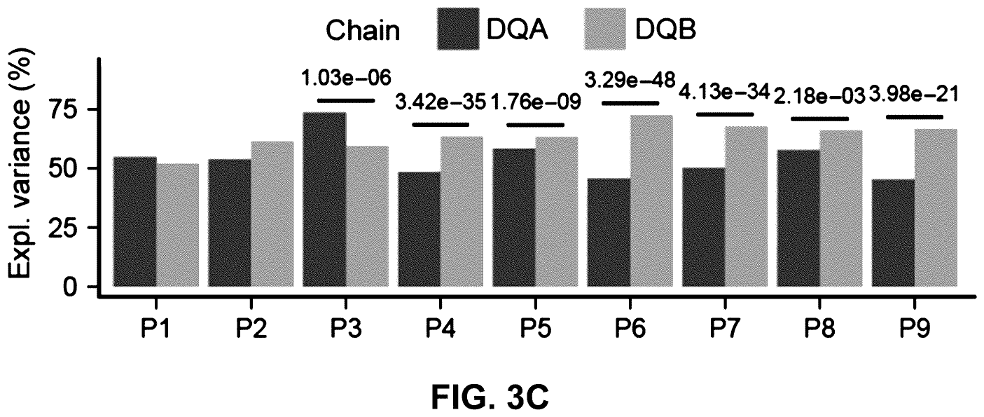

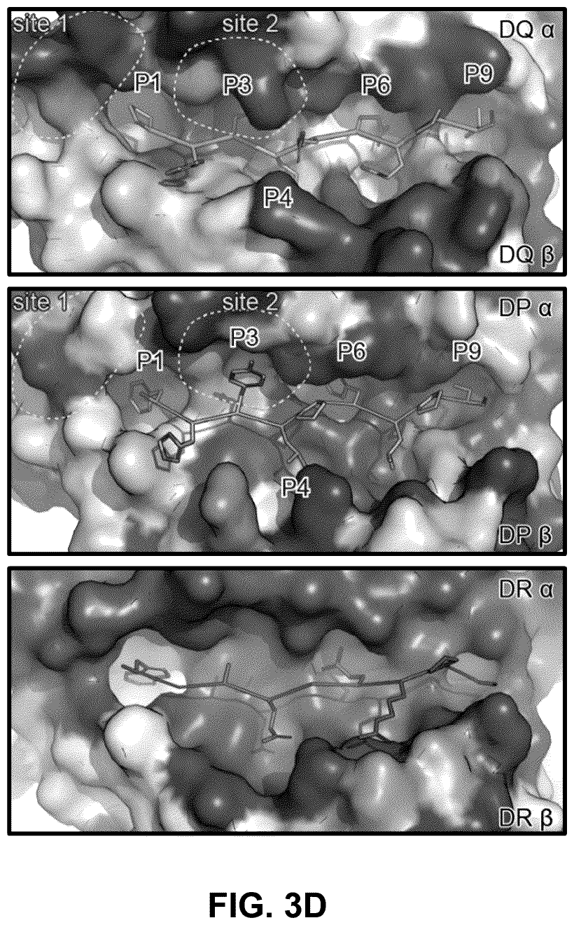

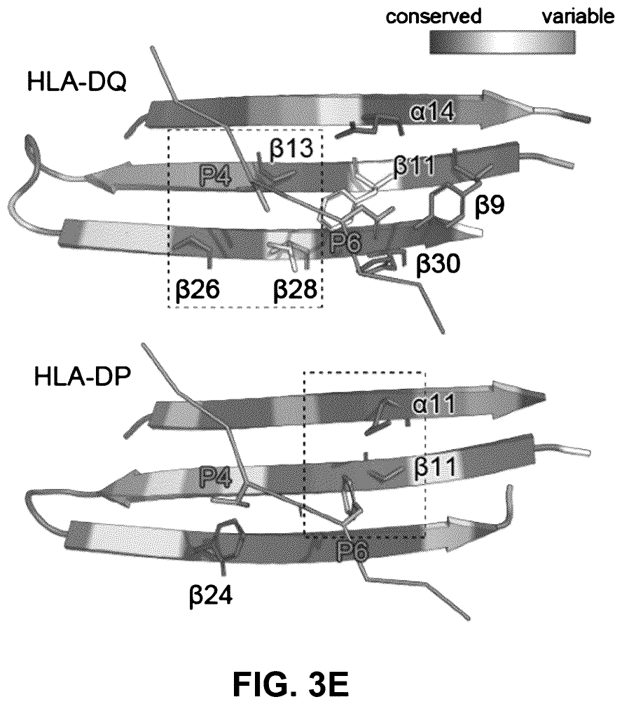

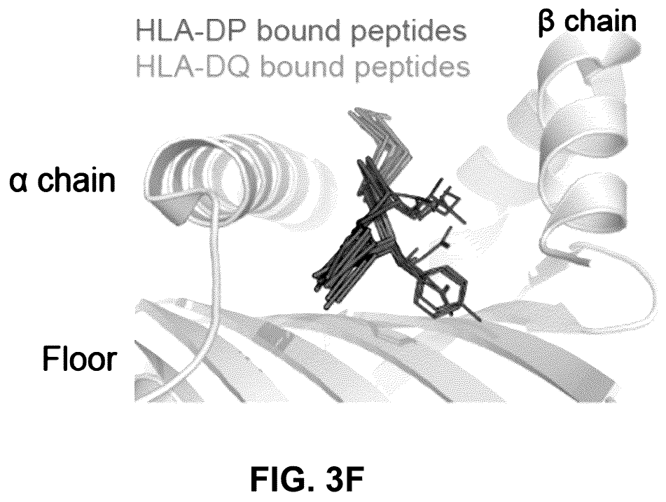

The importance of each position was quantified by Shannon entropy—the amount of information (in bits) required to specify an amino acid distribution at a position—which is low for strongly skewed distributions (Methods). This revealed that HLA-DR heterodimers exhibit amino acid binding preferences at P1, P4, P6, and P9 anchor residues. HLA-DP heterodimers exhibit preferences at P1, P6, and P9; and HLA-DQ heterodimers are unique in showing preferences at P3 and P4 (FIG. 3A). Given that HLA-II isotypes exhibit different anchor preferences, Applicants sought to further define differences in peptide binding properties. Based on the fact that HLA-DR alpha is monomorphic, genetic variants in the beta chain dominate in determining peptide binding properties. This is different for HLA-DP and HLA-DQ, where alpha and beta chains exhibit genetic diversity at the population level. To assess potential contribution of the alpha chains, Applicants modeled the motif probability matrices using Dirichlet regression, conditioned on the alpha/beta chain variant number (Methods). Similar to HLA-DR, the beta chain is responsible for explaining a larger proportion of variance in motif probability matrices in the case of HLA-DP (FIG. 3B). Importantly, for HLA-DQ this holds only for P4-P9, while P3 was significantly more affected by the alpha chain (P=1.03e-6, likelihood ratio test, see also FIG. 10A-B). This observation suggests that unique structural features of the peptide-binding groove specify different binding preferences of the HLA isotypes.

To explain the peptide motif binding preferences at the HLA-II isotype level, Applicants calculated the sequence variation at each amino acid position of HLA-II amongst the 3 isotypes and analyzed the sequence variation in three-dimensional structure (FIG. 3D-E). Most of the peptide-binding groove of the HLA-DR beta chain is highly variable, allowing for presentation of diverse peptides even if the HLA-DR alpha chain is invariant. For alpha chains of HLA-DP and-DQ, two sites show clear differences in sequence variation that likely explain peptide binding preferences (FIG. 3D). Site 1 mediates peptide binding at P1, and site 2 mediates binding at P3. Site 1 is critical for interaction with HLA-DM to facilitate peptide exchange, and amino acid residues in site 1 are also involved in peptide binding at P1 through interactions outside the binding core (Stern et al. 1994; Ghosh et al. 1995; Pos et al. 2012; Dessen et al. 1997; Reyes-Vargas et al. 2020; Schulze et al. 2013; Yin et al. 2014). Site 1 of HLA-DQ is highly variable suggesting HLA-DQ heterodimers might have different sensitivity to HLA-DM (FIG. 3D and FIG. 10C). Also, site 2 is variable for HLA-DQ (but not for HLA-DP), indicating that HLA-DQ exhibits variation in the alpha chain near the P3 pocket to specify the peptide P3 anchor position (FIG. 10D). This observation is indeed consistent with the statistical analysis of the peptidomics data where P3 has lower entropy for HLA-DQ bound peptides, and amino acid preferences at P3 are determined by the alpha chain rather than beta chain.

In addition to the P1 and P3 pockets, HLA-DQ and HLA-DP heterodimers exhibit variability within P4 and P6 pockets, respectively. This is evident in the peptide entropy calculations (FIG. 3A), where HLA-DQ has lower entropy at P4, and HLA-DP has lower entropy at P6. For HLA-DQ, the peptide binding preferences of the P4 pocket are determined by residues β26 and β28 of the beta chain (FIG. 3E). In contrast, these residues are fairly conserved in HLA-DP. Instead, the width and peptide binding specificity of the P6 pocket in HLA-DP is determined by residues α11 and β11 of the alpha and beta chains, respectively (FIG. 3E). Together, these features impact the peptide binding mode for the HLA-II isotypes. For HLA-DQ-bound peptides, the middle of the peptide backbone and the P4 side chain are positioned deeper inside the peptide binding groove, compared to peptides associated with HLA-DP and-DR (FIGS. 3D and 3F). By stabilizing the peptide binding through the middle of the peptide, HLA-DQ may compensate for the weak binding at P1 due to the variation in site 1. All these observations are consistent with the statistical analyses of the motifs identified by immunopeptidomics (FIG. 3A-C). In summary, systematic analysis of peptide binding preferences, HLA-II sequence variation, and structural analyses provide insight into the mechanisms of peptide binding to HLA-II and help explain how the three isotypes and dozens of HLA-II alleles diversify the immunopeptidome.

### Mono-Allelic HLA-II Immunopeptidome Data Enables Training of Improved Binding Core Classifiers.

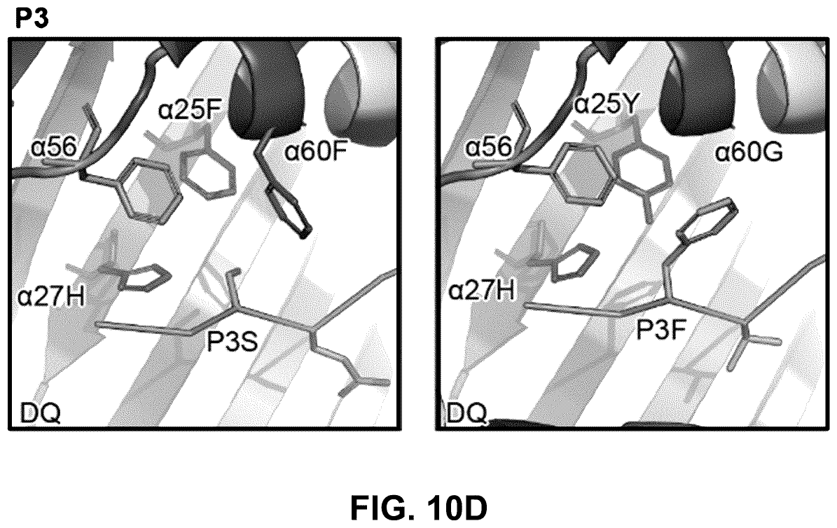

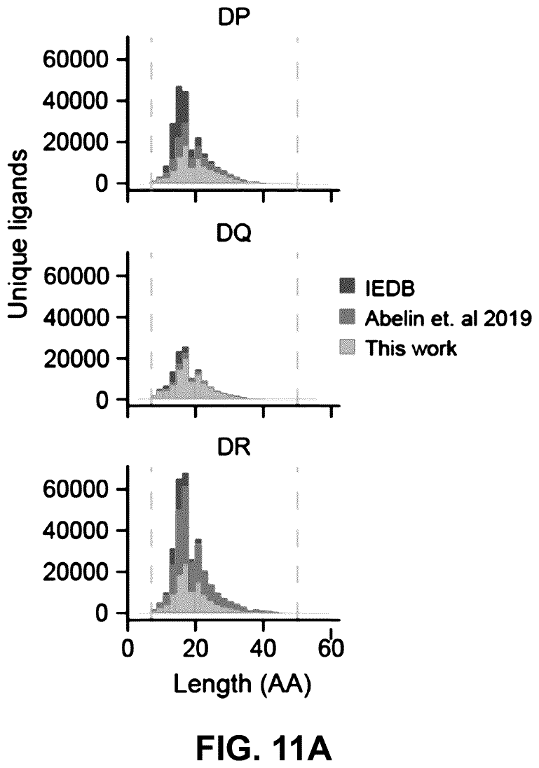

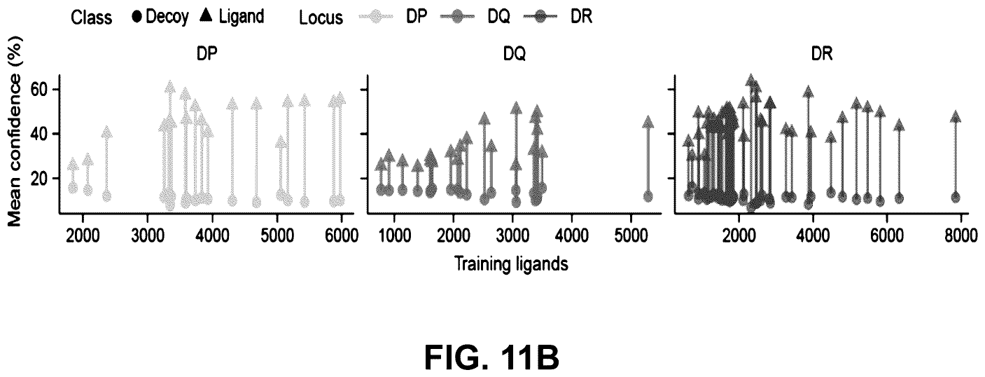

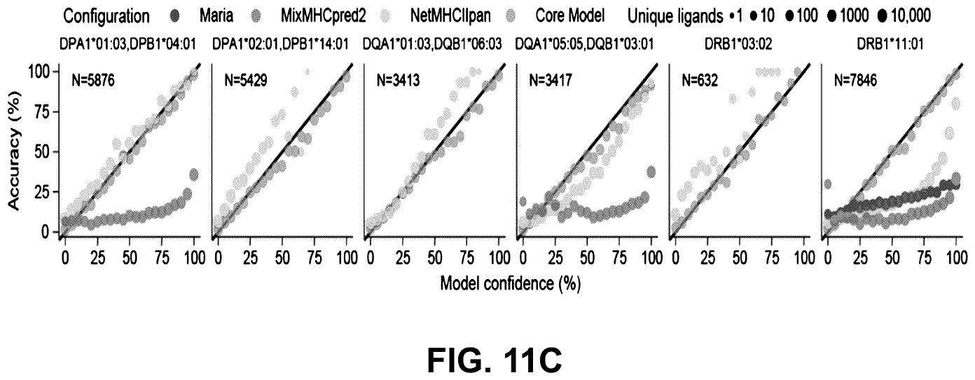

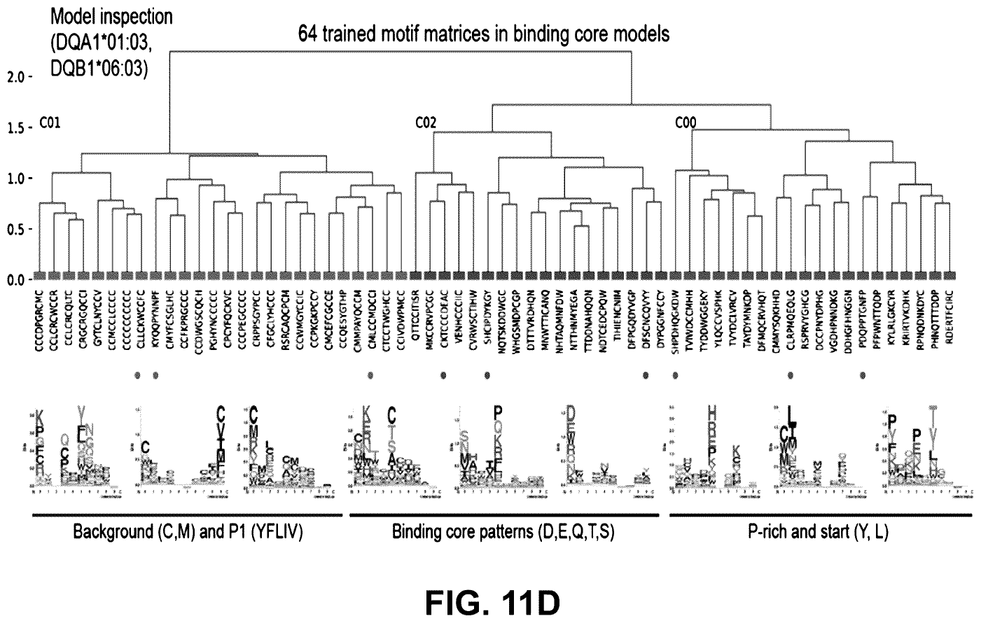

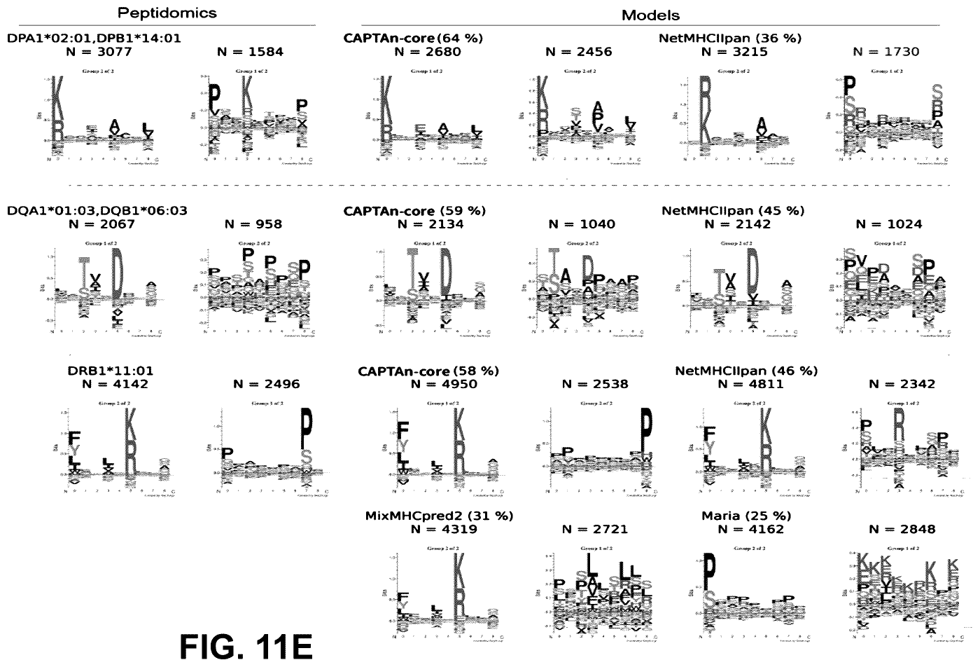

Deep immunopeptidome profiling revealed novel allele-specific peptide-binding preferences that can be explained by structural features of HLA-II proteins. These efforts notably increased allele coverage in mono-allelic immunopeptidome profiling, as Applicants recovered more than a thousand ligands for 29 alleles which had less than 100 previously reported. Compared to existing mono-allelic data (Abelin et al. 2019; Vita et al. 2019), the immunopeptidomics revealed 308,182 additional allele-specific ligands, for a total of 665,239 ligands (FIG. 11A). This dataset represents a powerful training set for a machine-learning approach to better predict HLA-II allele-specific T cell antigens. Here, allele-specific ligands are referred to as positives, which are defined as non-overlapping peptide ligands. Each positive was coupled with five decoys, random length-matched peptides with no reported binding to any allele. Peptides were allocated into five data splits, such that peptides from proteins in the same UniRef50 families could not be present in two different splits, to minimize redundancy (Methods). In total, 1,427,430 peptides containing 237,905 positives were coupled with 87 alleles, each having at least 50 unique ligands in each data split.

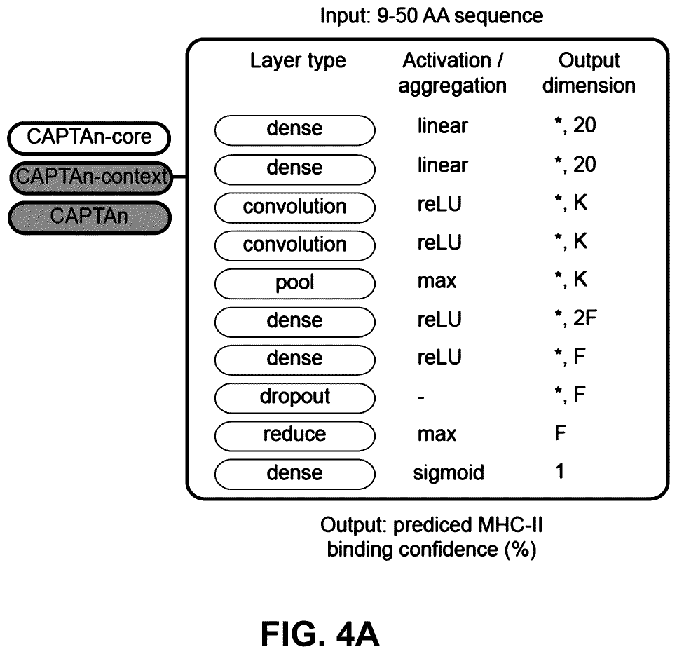

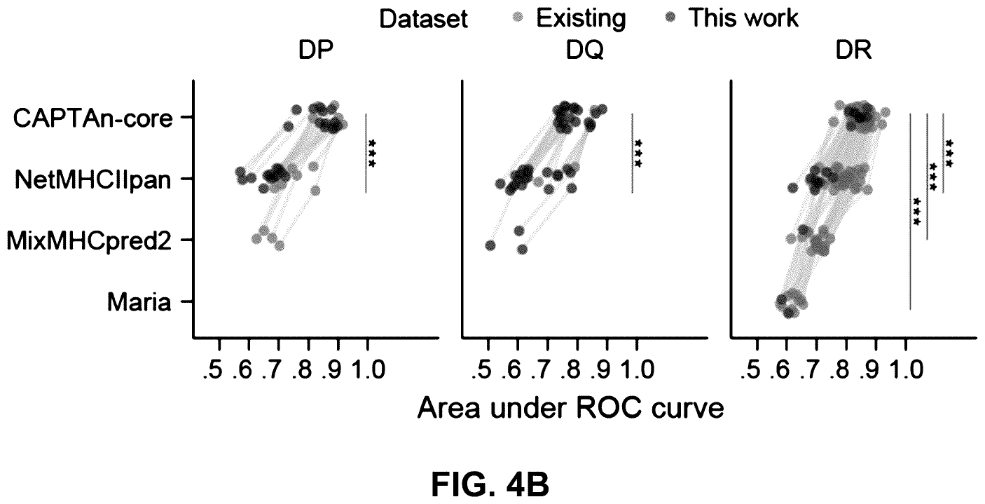

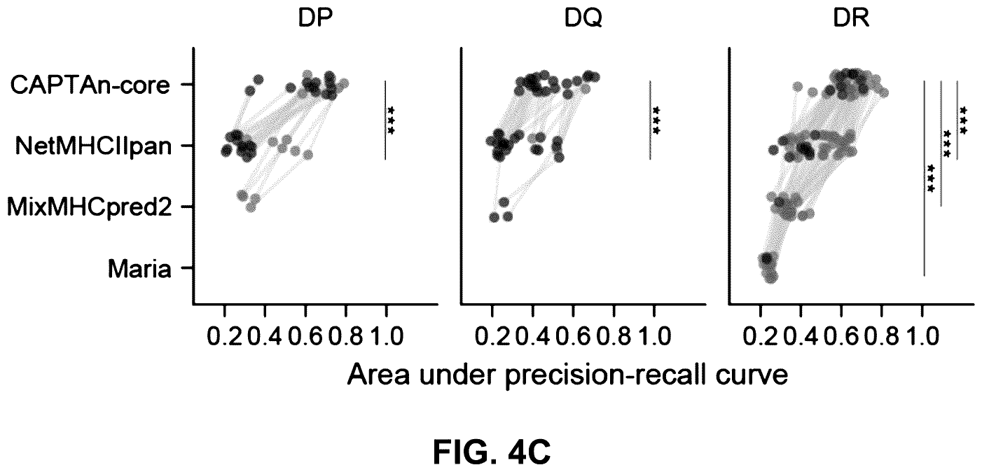

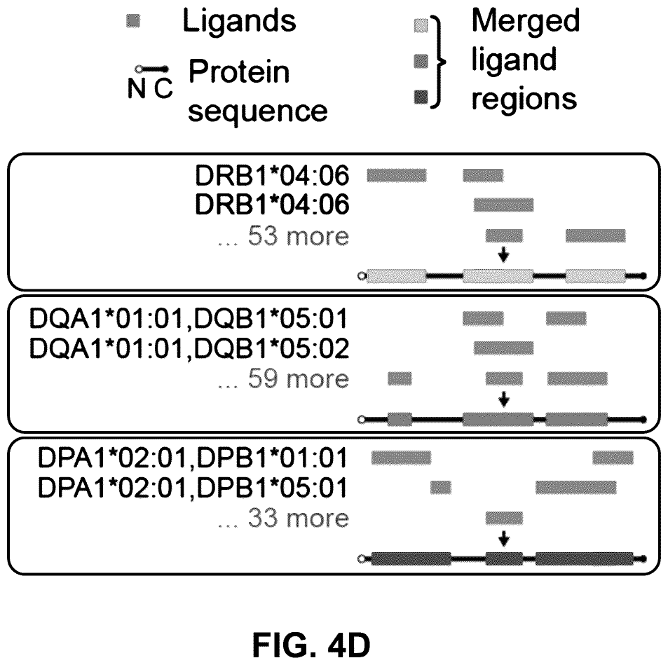

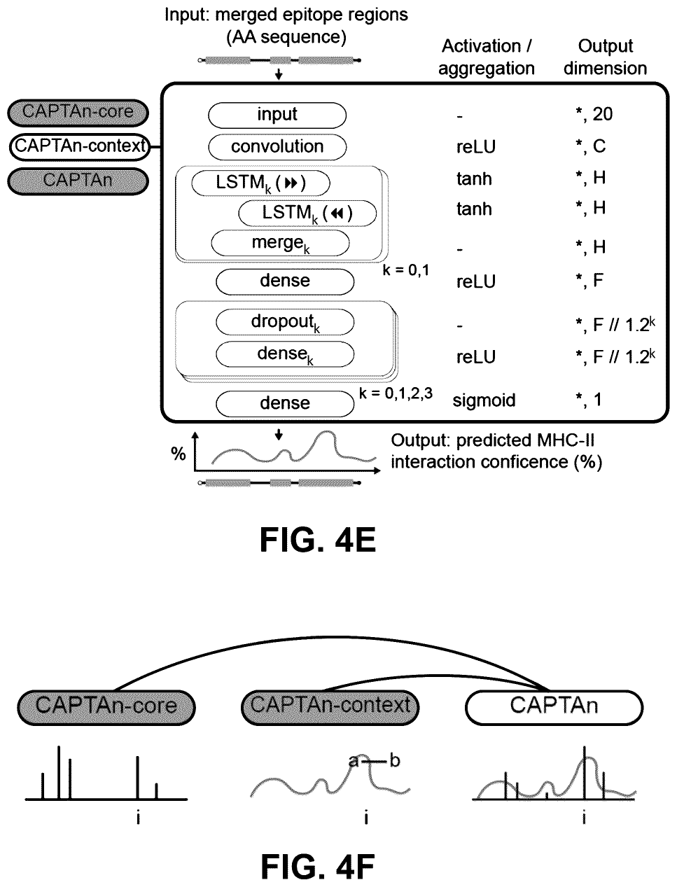

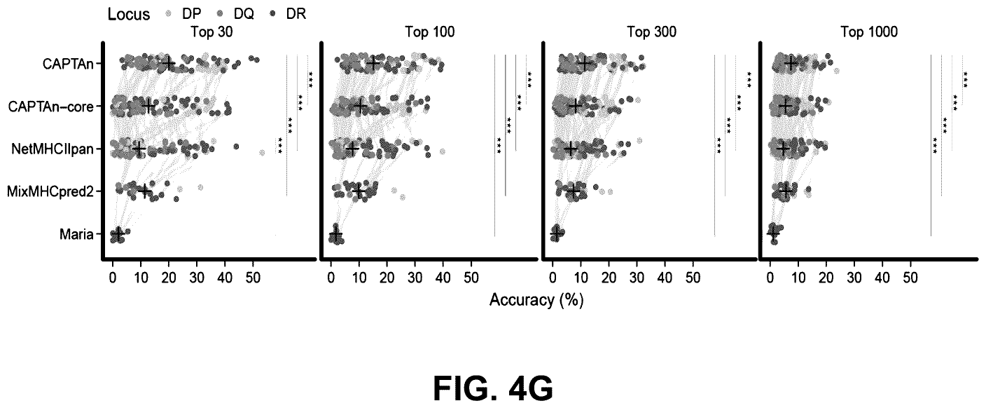

Applicants utilized this immunopeptidomics dataset to develop Context Aware Predictor of T cell Antigens (CAPTAn). The first component of the modeling framework is CAPTAn-core, which systematically infers HLA-II allele-specific peptide binding preferences (FIG. 4A). It seeks short peptide motifs (which may represent HLA-II binding cores and other patterns) in sequences up to 50 amino acids long constituting potential HLA-II ligands. Its key component is a convolutional neural network that infers motif detectors, which may be activated anywhere along a peptide. The strongest among these activations in a variable-length sequence are reduced to a finite number of numerical votes. A final combination of votes gives the confidence score—a probability that a given peptide sequence will bind to a specified HLA-II allele (see Methods and Supplementary Notes).

To benchmark the performance of the CAPTAn-core, Applicants compared it to state-of-the-art MHC-II binding and presentation predictors. NetMHCIIpan supports all 87 alleles in the training dataset, whereas Maria and MixMHCIIpred support 14 and 29 alleles, respectively (Racle et al. 2019; Chen et al. 2019; Reynisson, Barra, et al. 2020). NetMHCIIpan supports all the alleles due to extrapolation from the binding groove sequence, even if empirical immunopeptidomics or binding data for an allele are not available for training, while the other models rely on the inclusion of alleles in their training data. CAPTAn-core yielded calibrated estimates of allele-specific binding probabilities owing to the optimization of binary entropy loss (Methods). Whereas other neural network-based models also showed significant predictive performance, their probability estimates showed over- or under-confidence, likely due to usage of different training sets (FIG. 11B). The confidence score also reflects the underlying training set size, as binders for alleles with more training data get assigned higher mean scores and show increased margins when compared to decoys (FIG. 11C). Inspection of the trained motif detectors revealed prominent binding patterns and other sequence characteristics consistent with those identified in the peptidomics datasets (FIG. 11D). These observations emphasize the key role of expansive, high quality data sets in practical application and interpretation of model predictions.

All predictors were able to predict observed peptide ligands in held-out data splits as measured by area under receiver-operator characteristic, AUC, which quantifies the ability to separate positive examples (true HLA-II binders) in an unbalanced data set (FIG. 4B). Similarly, all predictors were capable of identifying observed peptide ligands in held-out data splits as measured by area under precision-recall curve, which estimates the expected precision achieved by a classifier (FIG. 4C) and comparison of motifs from prioritized sequences (FIG. 11E). Overall, these data affirm that empirical data from monoallelic immunopeptidomics conveys valuable information that is otherwise difficult to infer using MHC-II binding groove sequence alone. Taken together, the curated data sets of thousands of monoallelic HLA-II ligands coupled with model training result in accurate classification of peptide ligands for all profiled HLA-II alleles.

Modeling of HLA-II Peptide Ligand Regions within Whole Proteins Enables Discovery of Context-Dependent Features of Peptide Selection.

While the physico-chemical properties of the peptide 9-mer binding core sequence are necessary for HLA-II binding, contextual factors can impact the likelihood that a given peptide is presented. The efficiency of peptide selection, processing, and loading onto HLA-II is dictated by contextual features of the source protein from which a peptide is derived. These include subcellular localization or topology of the source protein, accessibility of the peptide within that protein, proximity to protease cleavage sites, and many other latent features. While multiple tools and databases exist to expose such annotations, Applicants hypothesized that signals relevant to HLA-II-peptide binding are discoverable through machine learning and large datasets. Applicants use terms “contextual features” and “context models” to leverage information in proteins harboring the retrieved peptides, without pre-imposing specific latent feature groups.

Applicants reasoned that combining multiallelic and monoallelic immunopeptidomes may allow learning contextual factors shared between HLA-II variants while also compensating for limited coverage of a single monoallelic experiment. Thus, Applicants set out to design context models, which predict presentable ligand regions based on the whole amino acid sequence of the proteins from which they originate. In this context, a ligand region is defined as a contiguous segment on a protein encompassing one or more HLA-II peptide ligands detected by immunopeptidomics (FIG. 4D). These ligand regions were combined and merged for each HLA-II isotype (HLA-DP,-DQ, and-DR) allowing Applicants to combine monoallelic profiling data with deconvoluted multiallelic datasets (Methods, (Marcu et al. 2021; Abelin et al. 2019; Racle et al. 2019)). In total, this yielded 65,648 ligand regions in 15,692 proteins, representing a 9.8% increase over using monoallelic data alone.

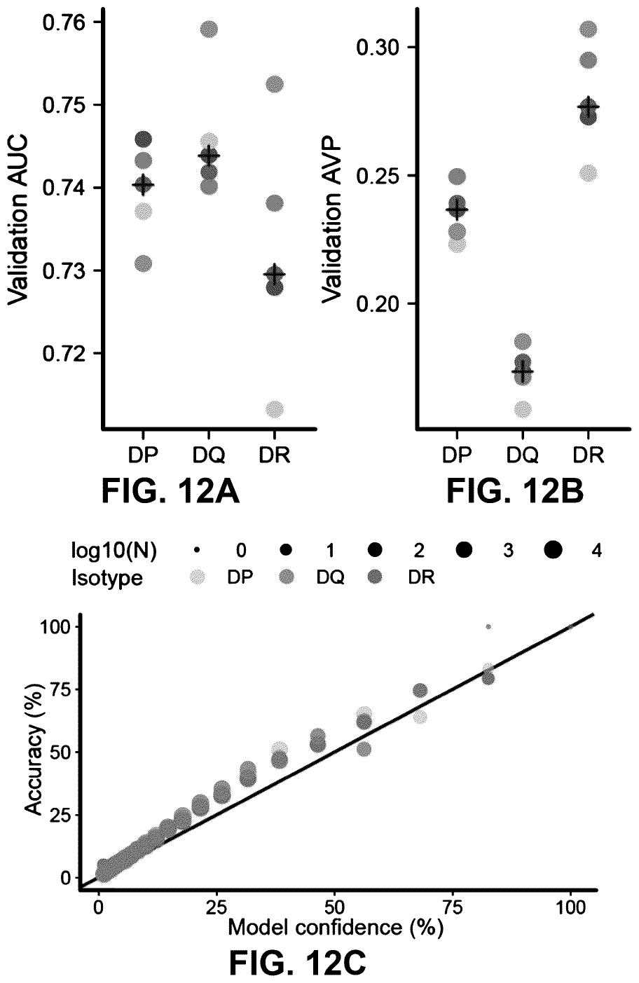

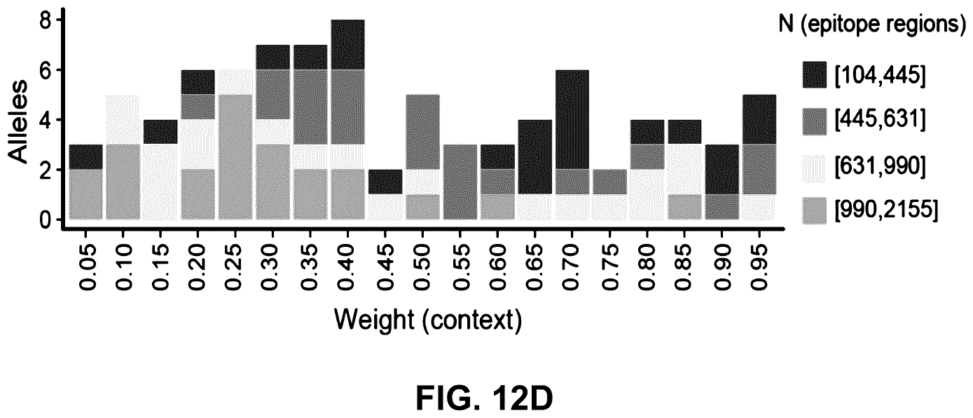

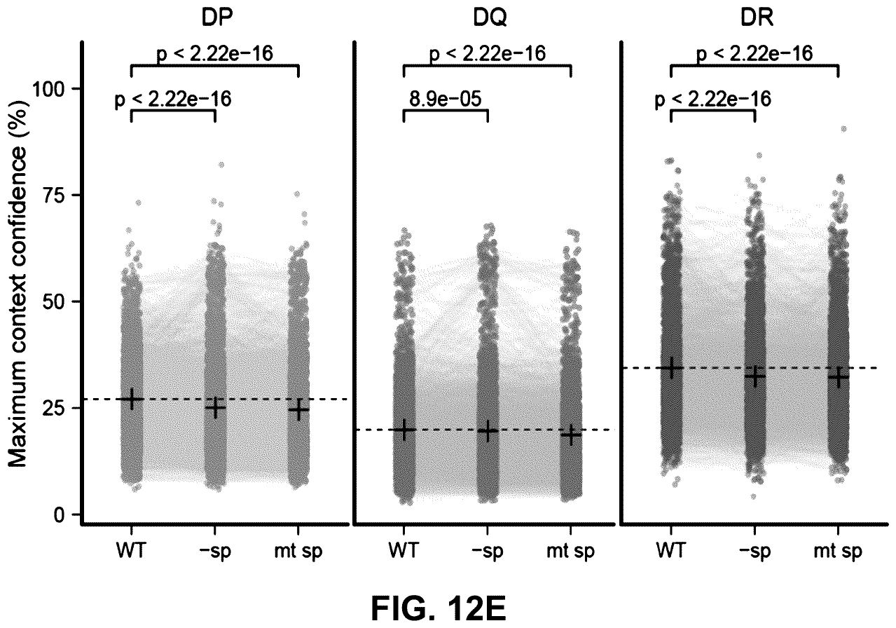

Applicants leveraged the immunopeptidomics dataset to train the context model, which is a neural network based on convolutional layers and multiple layers of bi-directional long-short term memory (LSTM), scanning a protein from both termini and memorizing potential informative parts (FIG. 4E). For each amino acid, it assigns a probability of it belonging to an HLA-II ligand region, which is inferred based on the whole protein sequence. A tailored routine samples protein groups of similar length to maximize efficiency and diversity during training (Methods). One model for each isotype and five data splits result in 15 model instances able to confidently predict ligand regions FIG. 12B-C). The calibrated predictions show 64% accuracy for HLA-DP, 63% for -DQ and 73% for-DR (FIG. 12E), a 6.1-10.7-fold increase over prior ligand region coverage.

Applicants hypothesized that the utility of the context models might improve allele-specific ligand predictions in two ways; first by conveying information from the whole protein sequence, and second by learning shared properties of peptide ligands across multiple, similar alleles. To this end, Applicants developed an ensemble model for CAPTAn. This approach works by optimizing an allele-specific weighted sum between core and the context model of the corresponding HLA-II alleles and isotypes (FIG. 4F; Methods; Suppl. Notes). For alleles with the highest numbers of ligands (the top quartile) the average weight of the context model was 29.1%, and it was 59.1% for those with the lowest numbers, showing that a small dataset can be compensated by the context model (FIG. 12E). Next, Applicants benchmarked the performance of CAPTAn. To simulate a practical application scenario, Applicants prioritized allele-specific ligands within 15,174 human proteins. The CAPTAn-core and CAPTAn both show improved performance over NetMHCIIpan, MARIA and MixMHCIIpred. This was the case for 74 out of 87 HLA-II alleles (at top 30 predicted epitopes), and 280 out of 348 from all combinations of alleles and thresholds (top 30, 100, 300, 1000 predictions) (FIG. 4G). For DRB1*11:01 with 7,192 independent ligands in the training data, CAPTAn achieved a mean accuracy of 49.3% in top 30 predictions, a 28-fold improvement over prior probability of 1.7%. For DRB1*14:54 with only 376 ligands in the training data, the accuracy of 16.0% represents a 242-fold improvement over a prior probability of 0.066%. These observations confirm that context models benefit from information in protein regions beyond binding cores and can improve allele-specific ligand prioritization within large proteomes.

Context Models Reveal Features of Antigenicity Associated with Peptides Selected for Presentation by HLA-II.

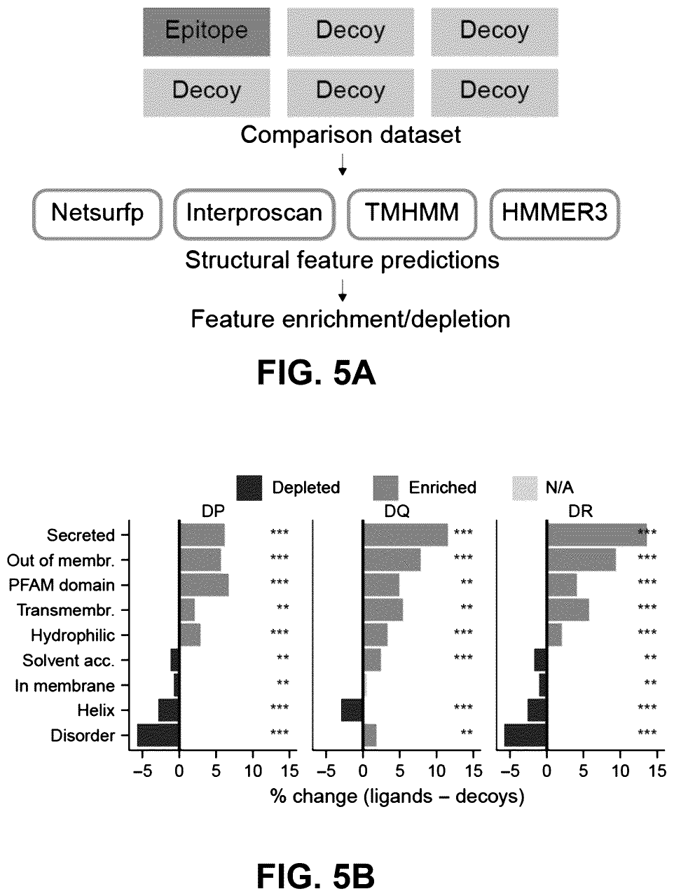

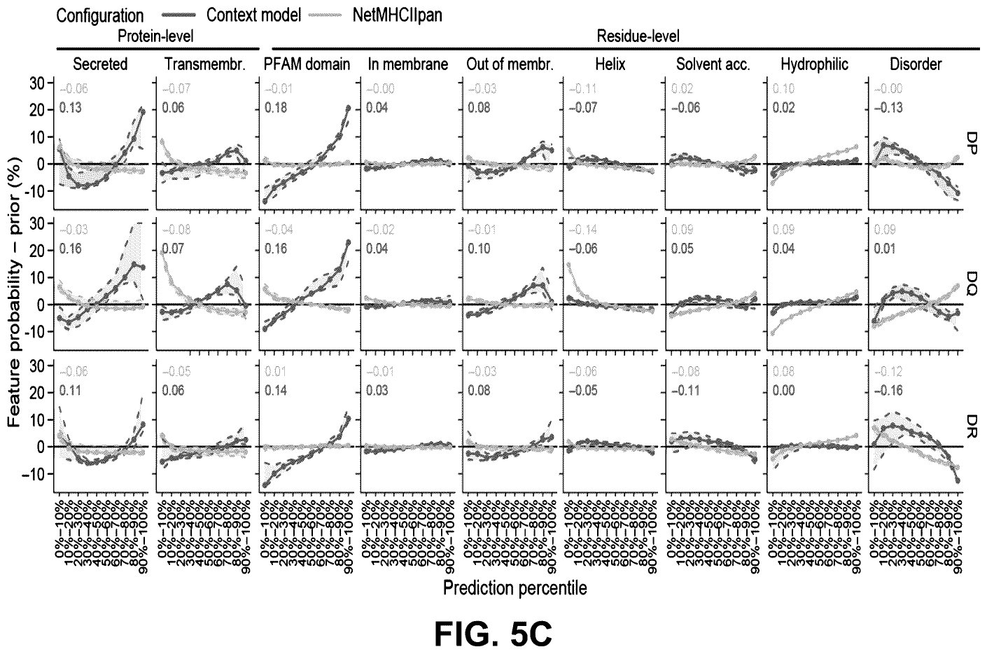

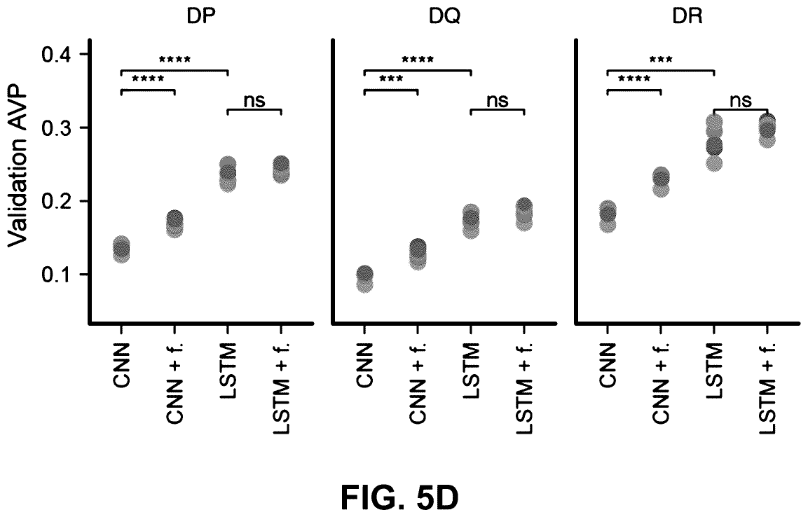

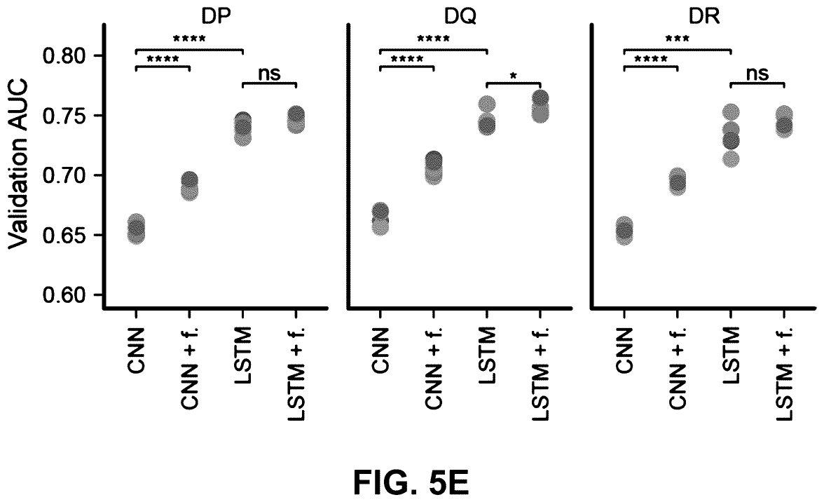

Having demonstrated improved performance with the CAPTAn-context model, Applicants set out to determine which contextual features influence the predictions. Presentation of HLA-II ligands depends on factors associated with antigen trafficking, processing, and loading (J. S. Blum, Wearsch, and Cresswell 2013) variables which have been successfully used in supervised approaches to augment peptide and protein antigen prioritization (Graham et al. 2018; Ricci et al. 2021). Applicants reasoned that the CAPTAn models are sufficiently well trained to perform this antigen prioritization task in an objective manner. Nevertheless, relevant protein structural annotations can provide further context to the ligand dataset and explain contributions of distant sequence regions conveyed by the model. To perform a comparison, Applicants used tools to predict structural annotations commonly applied to antigen prediction. These include solvent accessibility and disorder (Netsurfp, (Klausen et al. 2019)), PFAM protein families and domains (HMMER3, (Johnson, Eddy, and Portugaly 2010; Mistry et al. 2021)), transmembrane proteins and regions (TMHMM, (Krogh et al. 2001)) and secreted proteins (Interproscan/Signalp, (Nielsen 2017)), as outlined in FIG. 5A. Notably, HLA-II peptide ligands were more likely to be present in secreted proteins and extracellular/lumenal-facing regions of transmembrane proteins (FIG. 5B). The observed enrichment of epitopes in PFAM domains may reflect greater conservation or more structured regions. This is corroborated by decreased probability of peptide ligands for HLA-DP and HLA-DR deriving from disordered regions of protein antigens. Importantly, Applicants also observed reduced probability of HLA-II peptide ligands deriving from alpha helices, which is consistent with the fact that HLA-II preferentially binds peptides in a PPII type helix conformation (FIG. 2G) that is less tightly wound compared to a typical alpha-helix. Together, these identified binding correlates may improve predictions and suggest that HLA-II ligands often derive from structurally and functionally conserved regions.

While structural context features can be used for model training and predictions directly, Applicants assessed whether they may be implicitly accounted for in the CAPTAn context models. To address this question, Applicants sorted amino acids from all human proteins by increasing confidence of belonging to a ligand region as predicted by CAPTAn. As the model confidence increases, Applicants observed clear enrichment for features associated with secreted and transmembrane proteins, PFAM domains, and hydrophilicity (FIG. 5C). In contrast, Applicants observed depletion of features associated with protein disorder and alpha-helices (FIGS. 5B and C). To further demonstrate the ability of CAPTAn to utilize contextual features of antigenicity, Applicants focused on signal peptides, well-defined regions at the N-terminus of secreted and transmembrane proteins. Here, a relevant intervention can be made removing or mutating signal peptides by in silico mutagenesis. Doing so resulted in a systematic decrease of predicted context scores, showing how a known biological sorting signal can influence the context model (FIG. 12E). Collectively, structural features associated with HLA-II peptide ligands were effectively identified by the CAPTAn-context model, which outperformed other methods trained on short peptide sequences alone.

Having demonstrated that CAPTAn is capable of identifying relevant contextual features of HLA-II ligands, Applicants sought to determine the extent to which this explains its improved performance relative to other methods. The association of CAPTAn's predictions with structural features might be explained both by the context models learning from the whole protein sequence, or simply by virtue of it yielding many correct predictions. To test whether enrichment of these structural features follows from the neural network's ability to memorize potentially distant contextual signals, Applicants compared models based on whole protein sequences (LSTM) and models based on 20-mer peptides alone (CNN). The latter uses the same architecture excluding only the memory layers (Methods). Each model type was trained twice, with and without direct inclusion of all annotated structural features on top of amino acid sequences. In CNN models, the structural features significantly increased the classification performance (FIG. 5D-E, AUC T-test P-value=2.2e-6, 5.7e-6, 1.3e-7 for HLA-DP, -DQ, -DR, respectively). On the other hand, while LSTM outperformed both CNN versions (AUC T-test P-value=1.1e-8, 1.1-7, 0.00013), it did not significantly benefit from inclusion of annotated structural features in most cases (AUC T-test P-value=0.073, 0.028, 0.14). Since the structural features are predicted by related probabilistic sequence models, it is within the capacity of the LSTM-based context model to infer some of these factors given enough training data. Taken together, factors affecting epitope recognition beyond binding core motifs can be inferred without explicit provision of annotations, while retaining the protein sequence as a sole input requirement.

CAPTAn Enables Discovery of Microbiome-Derived Antigens Associated with Immune Homeostasis.

After developing and benchmarking CAPTAn performance, Applicants set out to test its utility in immunological contexts. While the models were primarily trained on eukaryotic proteins and peptides presented on HLA-II expressed in cell lines, many relevant T cell antigens are derived from microbes and processed by specialized antigen presenting cells (APCs). In this context, Applicants hypothesized that CAPTAn learned fundamental features of antigenicity that are conserved across antigen sources and that are preferentially selected by primary APCs such as dendritic cells (DCs). Thus, Applicants evaluated the performance of CAPTAn in predicting microbial antigens that are; (i) presented by primary human DCs on HLA-II and (ii) elicit a T cell response in healthy subjects.

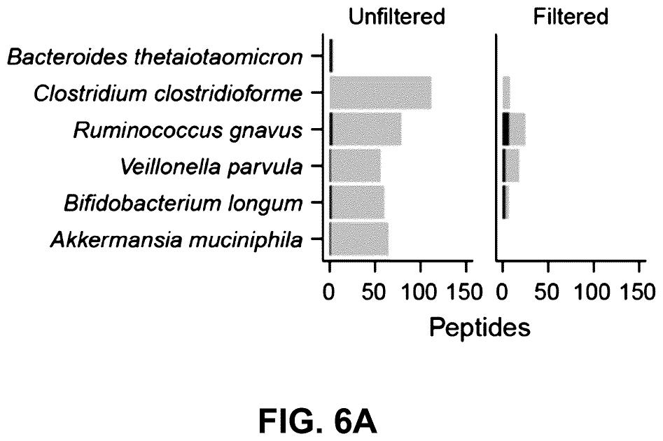

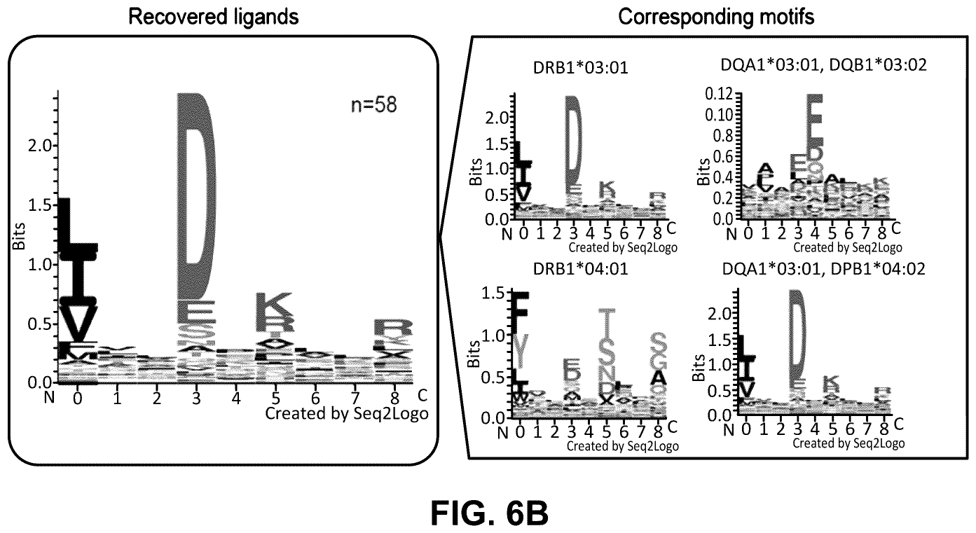

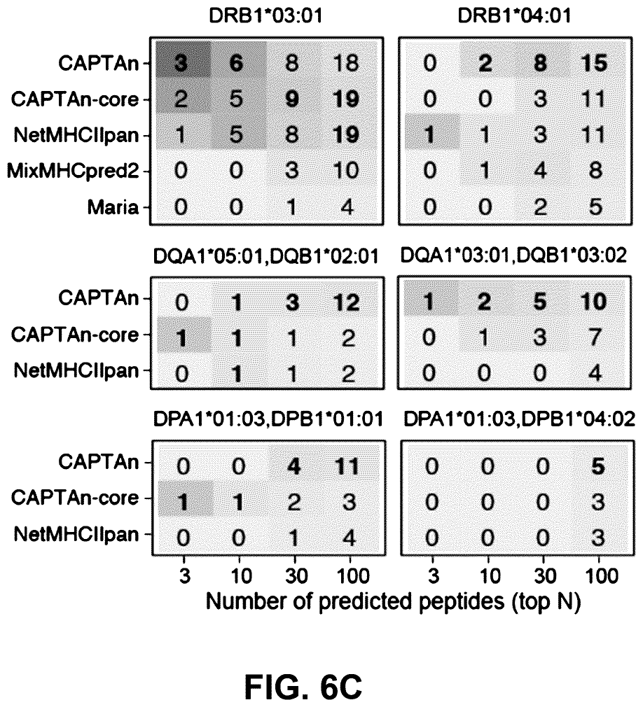

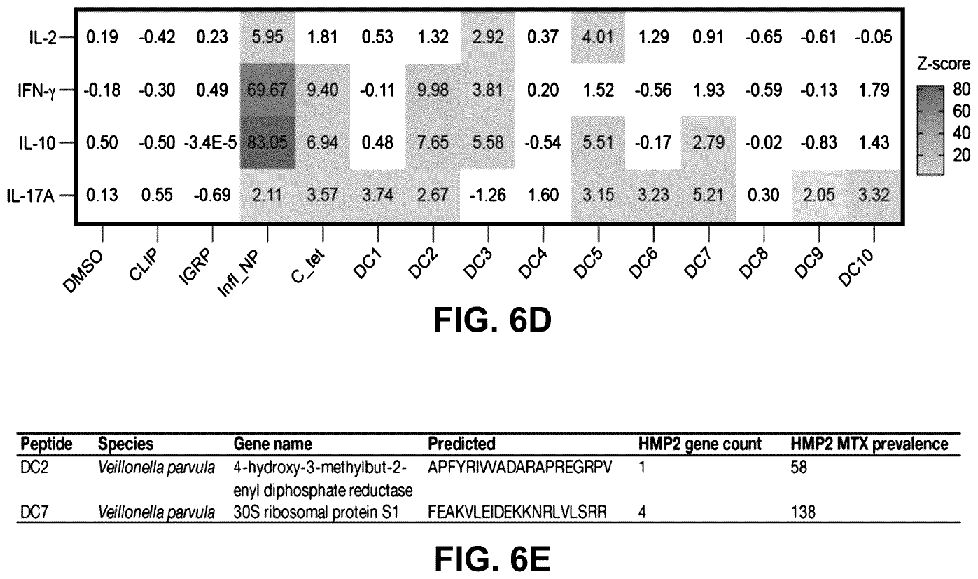

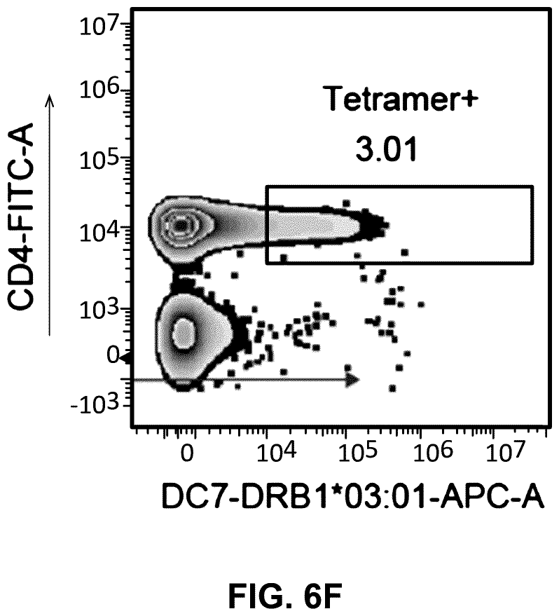

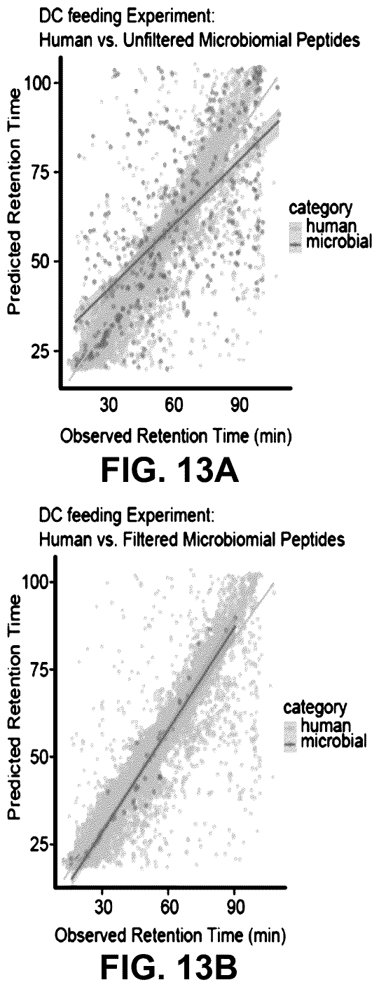

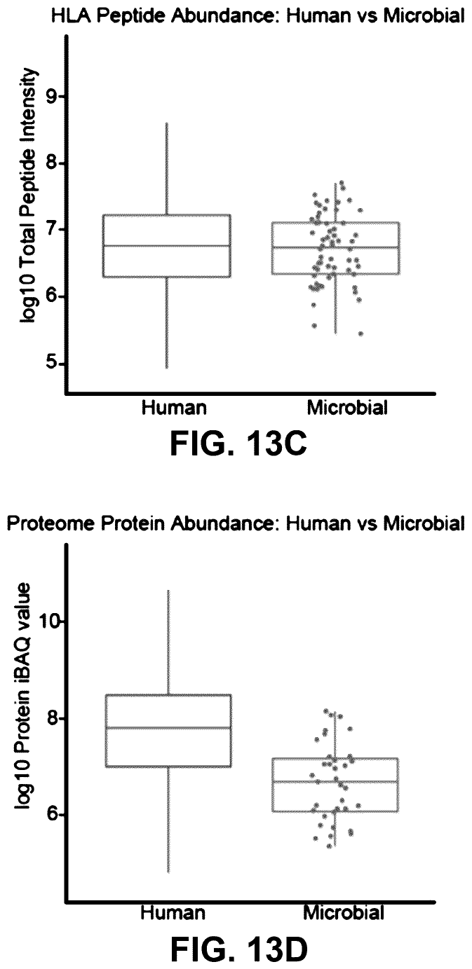

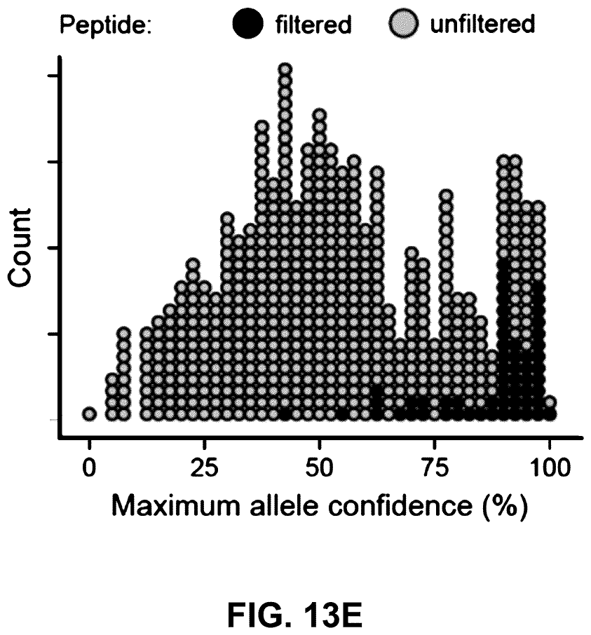

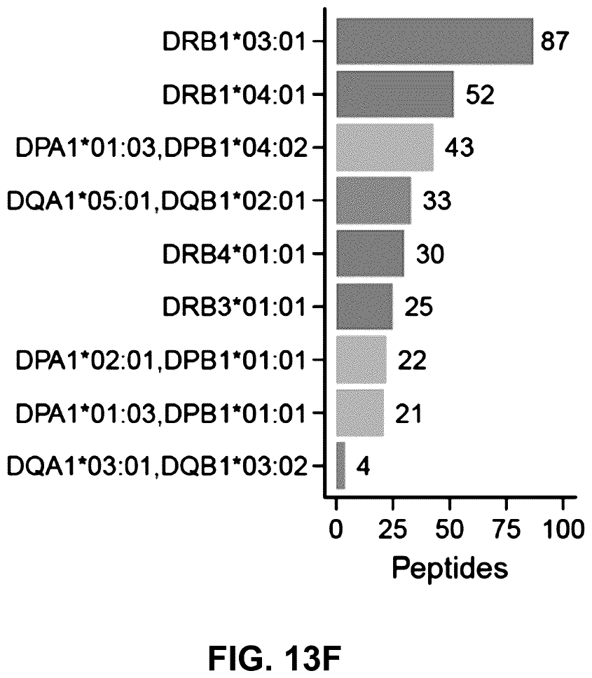

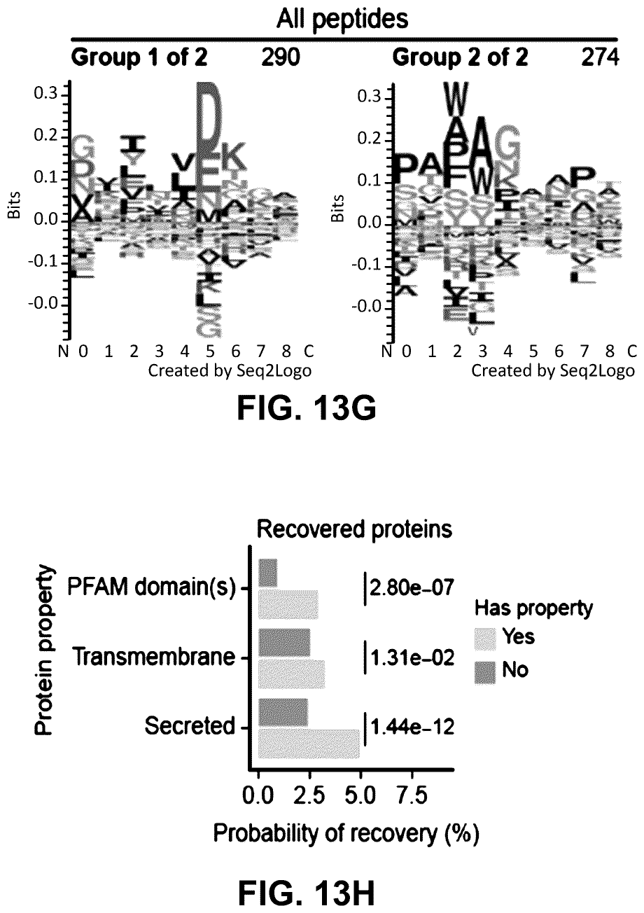

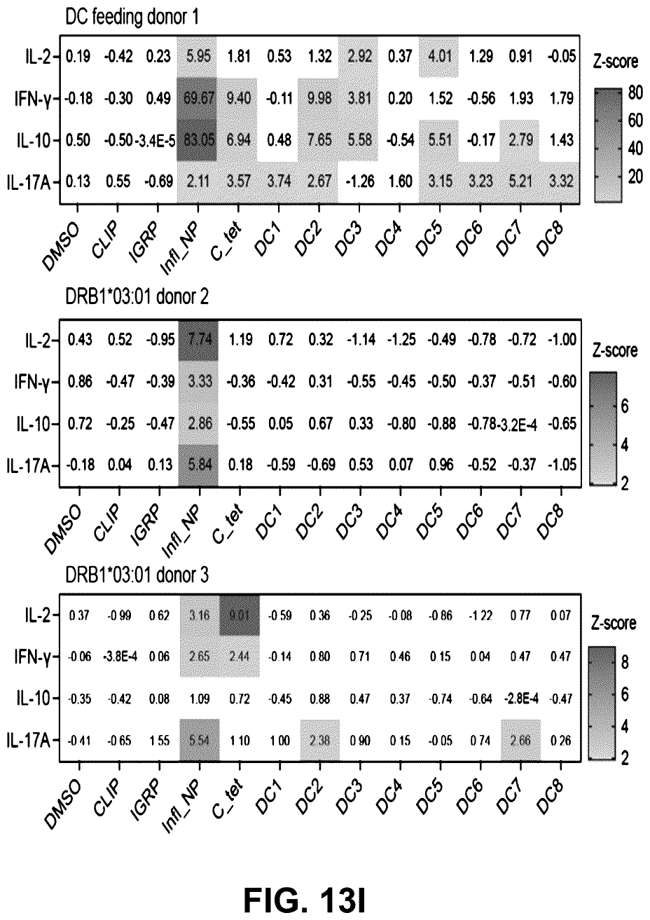

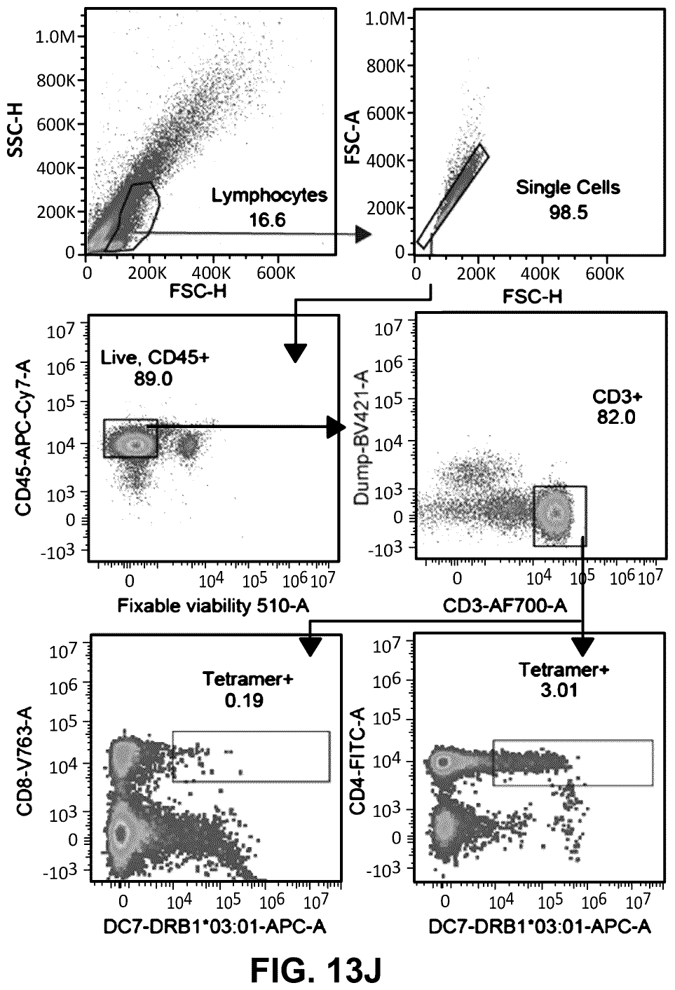

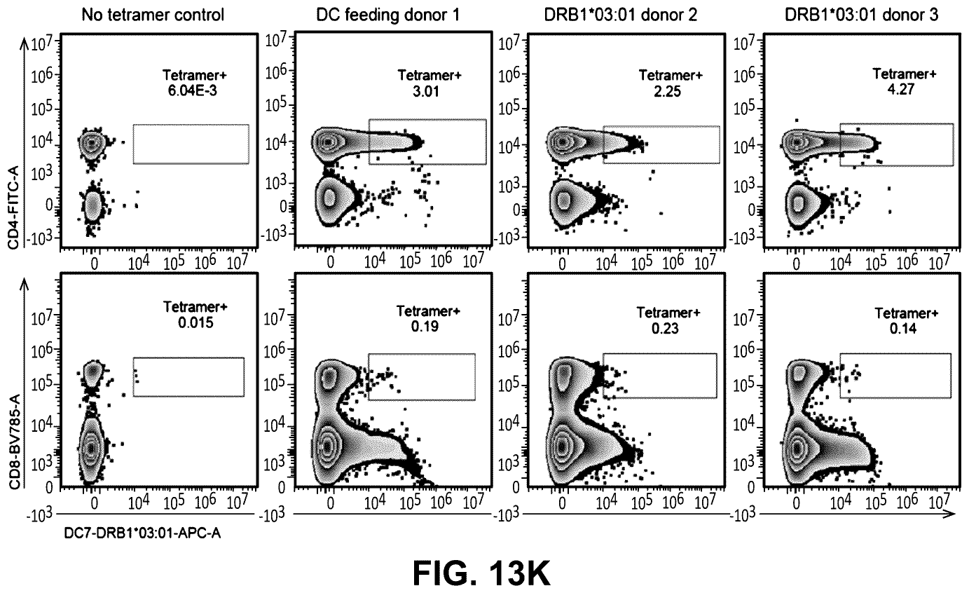

First, Applicants assembled a mock community of 6 prevalent and abundant bacterial strains from the human gut microbiome that represent species associated with health or immune-mediated disorders. This included strains associated with inflammatory bowel disease, systemic sclerosis and IgG4 related diseases (Veillonella parvula, Clostridium clostridioforme, and Ruminococcus gnavus (Lloyd-Price et al. 2019; Franzosa et al. 2019; Plichta et al. 2021), and strains typically considered as health-associated commensals (Akkermansia muciniphila, Bacteroides thetaiotaomicron and Bifidobacterium longum (Brown et al. 2019; Depommier et al. 2019; Henrick et al. 2021)). Microbes were co-cultured with monocyte-derived dendritic cells (MDDCs) from an HLA-typed donor. Subsequently, MDDCs were harvested after zero or six hour incubations and subjected to HLA-II immunoprecipitation and immunopeptidomics (FIG. 6A). Applicants recovered 592 HLA-II peptide ligands mapped to 516 microbial proteins, using a non-species specific estimated 1% FDR cutoff when comparing true peptides contained in the database from all species to their corresponding reverse sequence decoys (Methods). Unique sequences passing the non-species specific FDR cutoff were termed “unfiltered” (534), while those passing an additional mass spectra quality filter to achieve a bacteria specific FDR of 0% (see Methods) were termed “filtered” (58). Most peptides were contributed by B. thetaiotaomicron (162, 30%, unfiltered), R. gnavus (25, 45%, filtered) and V. parvula (18, 31%, filtered). Corroborating the filtering strategy, peptides showed improved correspondence between predicted and measured retention times (FIG. 13A-B). Compared to unfiltered, the filtered peptides from immunopeptidomics were more likely to derive from bacterial proteins detected in the proteomes of the DCs post bacterial feeding (confirmed) obtained from the HLA depleted cell lysates (FIG. 6B, 13 out of 58 vs. 10 out of 534, P=2.286e-13, Chi-square test). While bacterial peptides had comparable intensity to human peptides in the immunopeptidome, bacterial proteins detected with the proteomic workflow had lower intensities than human proteins (FIG. 13C-D). Both sets were used for downstream analysis, acknowledging that the smaller “filtered” set does not contain false positives.

The majority of bacterial peptides detected were confidently associated with the donor HLA-II alleles. CAPTAn-core was used to assign a reference allele for the supported donor HLA alleles: 2/2 DRB1 alleles, 3/4 HLA-DPA/DPB pairings, 2/4 HLA-DQA/DQB pairings and DRB3*01:01 and DRB4*01:01. In total, 57 filtered (98.2%) and 260 unfiltered peptides (48.6%) were associated with an HLA-II allele with confidence >50% (FIG. 13E). The assignments were predominantly to DRB1*03:01, DRB1*04:01 and DPA*01:03, DPB1*04:02, suggesting these alleles contributed to most of the recovered HLA-II ligands (FIG. 13F). The filtered peptides yielded a motif with prominent features associated with DRB1*03:01, including aspartate at P4 (FIG. 6C), while all peptides yielded two motifs, including a prominent proline and glycine enrichment (FIG. S6G). Proteins related to recovered peptides showed enrichment of structural features. The combined proteome of the six strains consists of 18,999 proteins, where secreted proteins were more than two times as likely to contain a recovered peptide (119 proteins, P=1.44e-12, Chi-square test) and the recovery of proteins with PFAM domains (499, P=2.80e-07) and transmembrane proteins (151, P=1.313e-2) was also significant (FIG. 13H). In agreement with microbial protein antigen prediction methods (Ricci et al. 2021), sequence conservation and structural signatures contribute to antigen presentation.

Having a validation dataset of microbial ligands presented by HLA-II in human MDDCs, Applicants set out to validate CAPTAn predictions relative to other prediction methods and discover new microbial antigens. To this end, Applicants focused on the set of 592 recovered peptides derived from the 516 proteins. Using each model and its supported alleles, Applicants predicted non-overlapping 20 amino acid regions and ranked them on predicted confidence. The CAPTAn model achieved most correct predictions across different thresholds and alleles (FIG. 6C). The improvement of CAPTAn was most notable for DRB1*03:01 with likely the largest number of related ligands, and DQA1*03:01, DQB1*03:02, in part because immunopeptidome data for the latter allele was provided exclusively in this study.

Applicants next determined whether T cell responses to microbiome-derived epitopes could be detected in healthy human subjects. Applicants focused on DRB1*03:01-associated microbial peptides and selected eight for validation based on prediction scores derived from CAPTAn and detection in previously published stool metatranscriptomics data from HMP2 (Table 2) (Lloyd-Price et al. 2019). Seven of these peptides were both predicted and observed peptides from immunopeptidomics (DC1-DC7). Applicants also sought to determine if the CAPTAn model could predict peptides invoking T cell responses that were not observed in peptidomics datasets due to the limit of detection of MS approaches. Thus, Applicants also selected one peptide, DC8, predicted with high confidence, but not observed in peptidomics.

Peptide restimulation of PBMCs from the same donor in the MDDC peptidomics experiment revealed cytokine responses to seven out of eight predicted peptides (FIG. 6D). Six of these induced IL-17A, a cytokine associated with mucosal immune responses, which is consistent with the notion that T cell responses towards microbiome peptides are tailored towards mucosal tissues where commensals encounter T cells (FIG. 6D). Applicants performed restimulations of PBMCs from two additional donors HLA-typed for DRB1*03:01 and confirmed IL-17A reactivity to V. parvula peptides DC2 and DC7 in two of three total donors (FIG. 13I). DC7 is a peptide derived from the bifunctional 4-hydroxy-3-methylbut-2-enyl diphosphate reductase/30S ribosomal protein S1, whereas DC2 is derived from the 30S ribosomal protein S16 and shows sequence conservation with other microbiome strains in the Firmicutes phylum. Both peptides were encoded by genes expressed in the human gut microbiome, with DC7 being detected in 20% of metatranscriptomic samples in HMP2 (FIG. 6E). To confirm that the cytokine response to DC7 is driven by cognate TCR recognition, Applicants produced fluorescently tagged tetramer reagents comprised of DC7 peptide loaded on HLA-DRB1*03:01. Applicants observed robust tetramer staining in CD4 T cells (and not CD8 T cells) from PBMCs of HLA-matched donors, revealing the frequency of DC7-specific T cells and their HLA restriction, which complements functional cytokine data (FIG. 6F, FIG. 13J-K). These findings demonstrate that the adaptive immune system actively recognizes microbiome-derived antigens, suggesting that T cells specific to these antigens are specialized for mucosal protection and circulate systemically. Given the complexity of the potential antigen landscape in the microbiome, CAPTAn can facilitate identification of conserved T cell epitopes.

### CAPTAn Enables Discovery and Validation of Novel Conserved Epitopes in the SARS-CoV-2 Nucleoprotein.

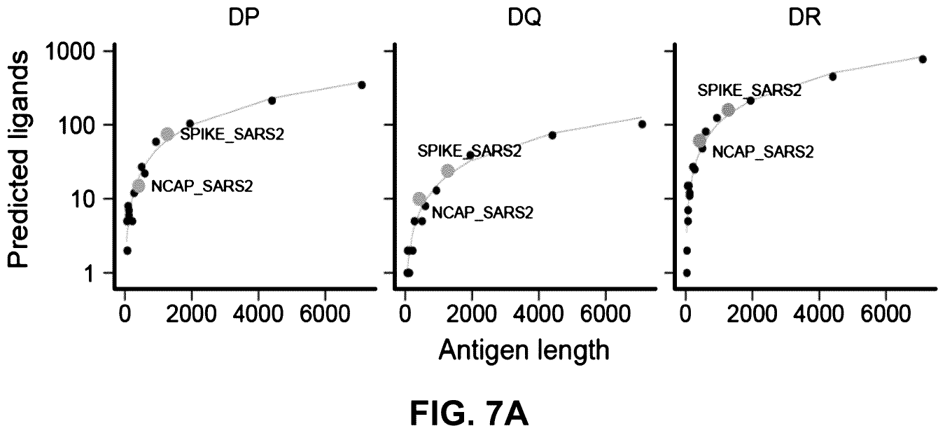

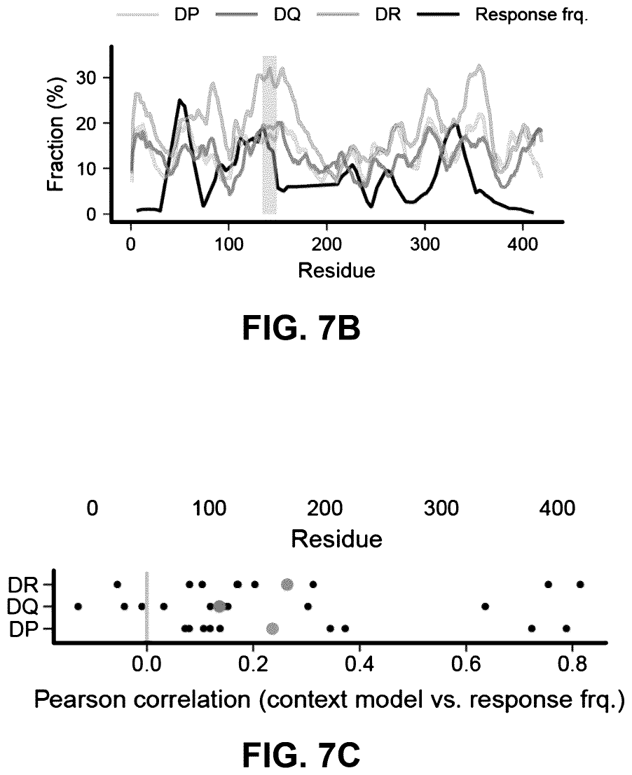

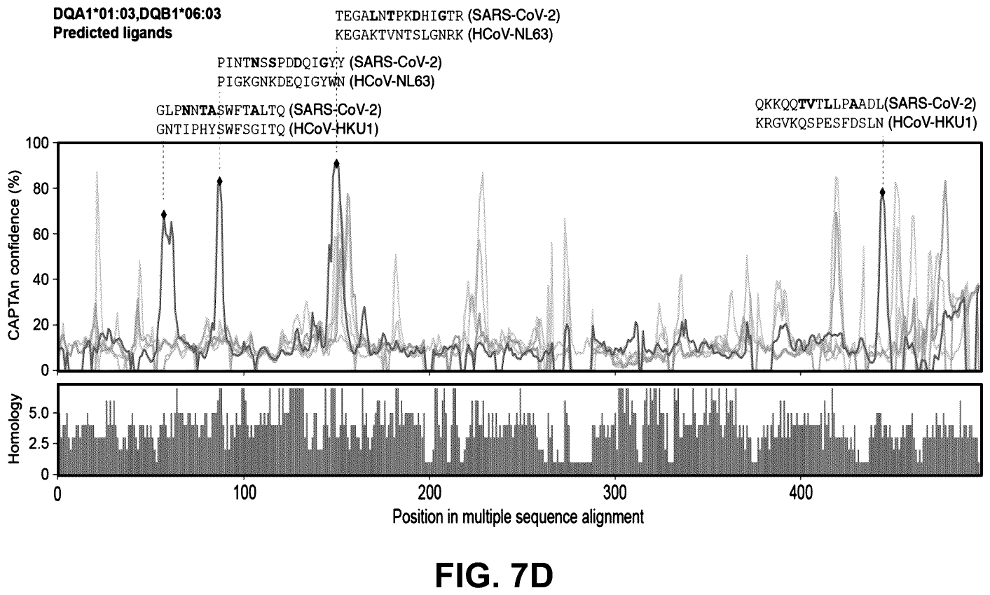

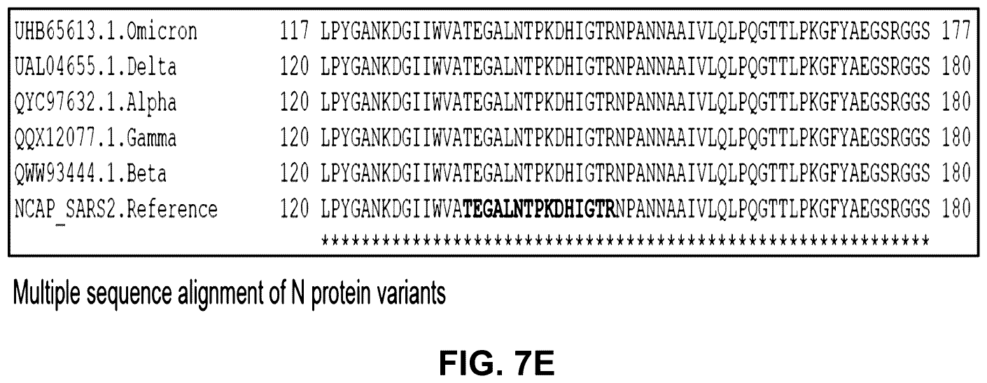

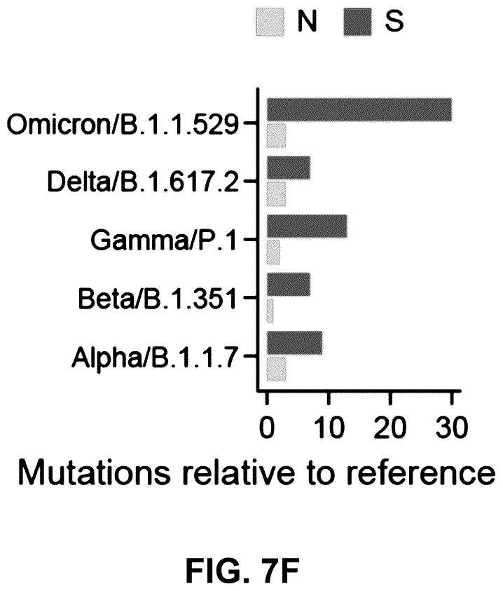

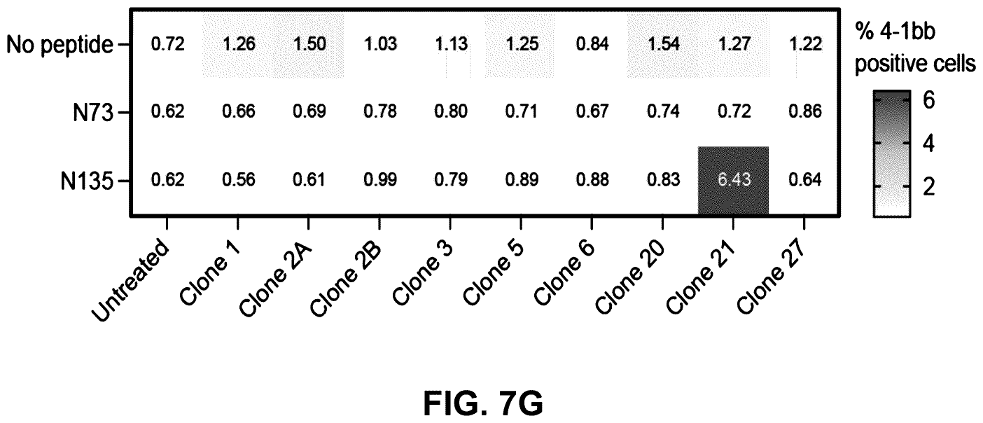

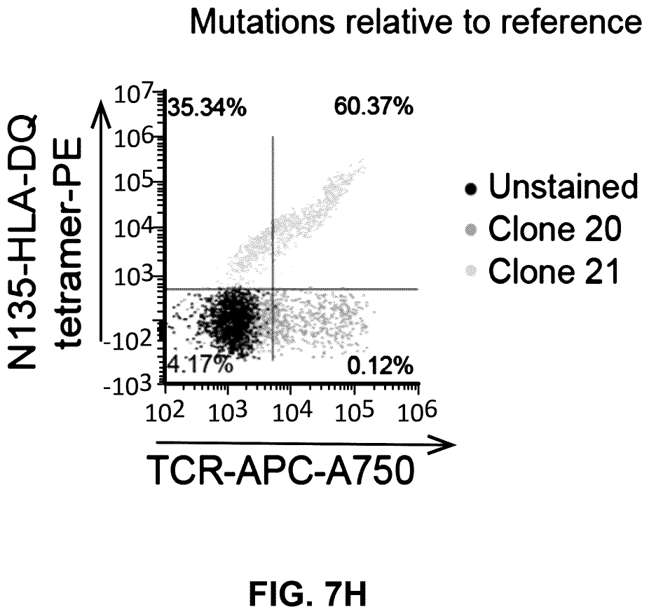

The adaptive immune system is particularly adept at responding to emerging infectious diseases and conferring durable, protective memory. In the context of SARS-COV-2, measuring neutralizing antibody responses has been a reliable metric of protective immunity; however, accumulating evidence highlights the critical role of T-cell responses, particularly with the emergence of new variants and waning antibody titers over time (May et al. 2021). Whereas measuring antibody responses to pathogens is a powerful approach for quantifying adaptive immunity, measuring antigen-specific T cell responses is confounded by the diversity of HLA haplotypes and highly personalized nature of HLA-II restricted T cell responses. Thus, Applicants applied CAPTAn to generate allele-specific predictions for the SARS-COV-2 proteome, yielding 1,615 unique ligand regions for 59 HLA-II alleles and a total of 2,920 allele-specific ligands. The spike(S) protein and nucleoprotein (N) had more predicted HLA-II ligand regions than expected by chance, adjusting for their lengths. Using a log-linear model, Applicants observed 22.05, 12.21 and 3.15 excess ligand regions for DR, DP and DQ, respectively, while N had 18.9 and 3.6 excess regions for DR and DQ, respectively (FIG. 7A). This suggests that S and N might have more potential to induce an immune response, which likely requires T cell help (Shrock et al. 2020). The predictions aligned with publicly available data for CD4+ T cell responses to SARS-COV-2 from 25 studies with HLA-II allele-specific and non-specific resolution (Grifoni et al. 2021). Here, the context model predictions correlated positively with lower-bound population response frequencies (FIG. 7B-C). This alignment was significant for 8 out of 10 assayed virus ORFs, showing the context model, through integrating information from several HLA-II alleles, associates with population-level immunogenicity potential.

It is notable that the majority of globally deployed SARS-COV-2 vaccine strategies focus on eliciting adaptive immune responses to the S protein, with a particular emphasis on neutralizing antibody responses. Given the predicted enrichment of T cell epitopes in the N protein, Applicants hypothesized that N protein may encode an immunodominant antigen. To test this hypothesis, Applicants identified a convalescent subject who had recovered from SARS-COV-2 infection and generated a robust B-cell response to S and T-cell responses to S and N (manuscript submitted). Applicants utilized CAPTAn to generate HLA-II personalized T cell epitope predictions for this subject and identified a particularly striking ligand region at N135-150 for DQA1*01:03, DQB1*06:03, an HLA-DQ variant present in 5.7% individuals of European-descent and more rare in other ancestries (FIG. 7D; Table 1). This epitope was predicted with CAPTAn confidence of 90.8% and was not predicted by either NetMHCIIpan or observed in the aforementioned public database (Grifoni et al. 2021). From all predicted epitopes with >70% confidence, only N135 associated with highly conserved regions among distantly related endemic human coronaviruses (FIG. 7D). Relative to these viral strains, the exceptional DQA1*01:03, DQB1*06:03 binding confidence to N135 stems from emergence of amino acids L, T, D, G at positions 1, 3, 6, 9 relative to the binding core. The wider region containing N135 is not only conserved among endemic coronaviruses, but also perfectly conserved in recent SARS-COV-2 variants-of-concern: alpha, beta, gamma, delta and omicron (FIG. 7D). Given the lower rate of mutations in N relative to S (FIG. 7F), vaccine strategies incorporating N protein may expand the landscape of T cell epitopes to promote a robust, pan-variant response.

Next, Applicants sought to determine if the convalescent individual mounted a T cell response to this predicted N epitope. Applicants previously characterized T cell responses in this individual by stimulating PBMCs in vitro with a tiled N peptide pool (manuscript submitted). Subsequently, activated T cells were sorted and subjected to single cell transcriptomics and TCR repertoire profiling. Applicants focused on clonally expanded TCRs from CD4+ T cells exhibiting an antiviral Th1 phenotype associated with IFN-γ production. Applicants screened 9 TCRs for reactivity against 2 N epitopes predicted by CAPTAn. The patient-specific TCR clone 21 reacted specifically with nucleoprotein peptide N135 presented by DQA1*01:03, DQB1*06:03, validating the immunogenicity and allele-specificity of N135-150. (FIGS. 7G and 7H). Taken together, the analyses suggest that N protein is enriched for conserved, putative T cell epitopes, which can be recovered via personalized CAPTAn HLA-II binding and context predictions, providing value for future widespread use in antigen discovery across infectious and autoimmune diseases.

### DISCUSSION

A growing body of evidence has shown the role of CD4+ T cells in shaping immune responses to infection, cancer, allergy, autoimmune diseases, and also in direct killing of cancer cells (Oh and Fong 2021; Gao, Hsu, and Li 2021; Reynolds and Finlay 2017; Renz and Skevaki 2021; Lipsitch et al. 2020; Sette and Crotty 2021). Peptide presentation by HLA-II is a requisite step that dictates CD4+ T cell responses, therefore defining the peptide binding rules by individual HLA-II heterodimers is crucial for understanding the functional basis of adaptive immunity in health and disease. The diversity of peptide binding and highly polymorphic nature of HLA-II make it difficult to predict what can be presented by individuals. Therefore, Applicants extensively characterized monoallelic HLA-II immunopeptidomes, including many alleles that had not been previously examined. The antigen prediction model trained with newly generated deep monoallelic peptidomics data outperformed existing antigen prediction methods and also revealed biological principles of antigen processing, such as the contribution of distant amino acids and three-dimensional structural features.

Structural analyses of sequence variation within HLA-II isotypes further provides insight into how humans cover diverse sequences of antigens through the three HLA-II isotypes (FIG. 3 and FIG. 9). HLA-DR presents peptides having cores starting with hydrophobic residues and has strong preferences at anchor positions on both ends. HLA-DP enables presentation of peptides with bulky basic residues at P1 (van Balen et al. 2020; Racle et al. 2019; Abelin et al. 2019). The analyses showed that HLA-DQ is notable for having strong binding preferences in the middle positions of peptide ligands. Unlike the other HLA-II isotypes, HLA-DQ does not exhibit strong amino acid preferences for P1 positions in peptide ligands, instead it has a stronger preference for P3. The unique binding properties of HLA-DQ are likely correlated with the evolution of this protein in the human population, where significant genetic variation is found in the alpha chain near P1 and P3 pockets. This attribute accommodates binding to diverse peptide sequences without limitations on the end sequences, especially at P1. Understanding the molecular mechanisms of peptide binding to diverse HLA-II heterodimers provides key insights into how the adaptive immune system is capable of responding to emerging pathogens, autoantigens, and tumor antigens. These principles have additional value in informing new approaches to antigen discovery and epitope prediction.

Prediction of HLA-II ligands has benefited immensely from profiling of peptide ligands presented by HLA-II. Mass spectrometry-based immunopeptidomics (Peters, Nielsen, and Sette 2020) and yeast display have been used for peptide ligand screening (Rappazzo, Huisman, and Birnbaum 2020), and these datasets have proven valuable for training epitope prediction tools. As a flagship of the latest modeling approaches, NetMHCIIpan and MixMHCIIpred are able to increase the amount of training data by deconvolution of multiallelic immunopeptidomics datasets and inclusion of HLA-II allele-specific binding groove sequence variants (Alvarez et al. 2019; Racle et al. 2019). Here, Applicants demonstrate added value in empirically generating HLA-II monoallelic immunopeptidomic data coupled with structural analyses of HLA-II heterodimers. These efforts reveal concerted patterns of peptide amino acid motifs associated with the three HLA-II isotypes and also motif hierarchies associated with structurally related families of HLA-II variants (FIG. 2, (Jensen et al. 2018)). Sharing peptide motif information between the single allele ligandomes also compensates for potentially missed ligands inherent in mass spectrometry-based methods. Here, Applicants combined the precision of allele-specific binding cores with shared features of antigenicity via the context model. Applicants incorporated protein context awareness into the design of HLA-II peptide ligand predictions that are affected by information from the whole protein sequence. Training of the underlying recurrent neural network required more sequences than were available for any single allele, and was made possible only when defining merged ligand regions from HLA-II alleles within each isotype, simultaneously sharing information between the alleles and seeking signals from all protein regions. The latter include adjacent signals, such as exopeptidase trimming sites and proline-rich cleavage motifs (Barra et al. 2018) as well as distant signal peptides, whose removal notably altered the predictions. Future models trained on diverse measurements, including transcriptional profiling of single cells or primary APCs could elucidate additional features important in HLA-II presentation.

Applicants have shown how simultaneous modeling of multiple monoallelic ligandomes and whole protein recovers contextual cues and benefits predictions without explicit provision of structural annotations as employed previously (Graham et al. 2018; Chen et al. 2019). Microbial antigens can be predicted based on transmembrane domains, secretory pathway signals, intrinsically structured and exposed regions, tandem repeats (Ricci et al. 2021), synonymous codon usage, or disorder (Carmona et al. 2012). In an unbiased manner, peptides prioritized by CAPTAn were associated with structural features reflecting antigen secretion, membrane localization, domain homology and residue accessibility. These findings suggest that considering antigen context beyond peptide binding cores will continue to improve binding predictions. It is also intriguing to consider whether recent machine learning protein folding and contact map prediction models (Tunyasuvunakool et al. 2021; Baek et al. 2021; Bepler and Berger 2021), in combination with high-quality immunopeptidomics datasets, could capture additional nuanced signatures of antigen processing (internalization, trafficking, proteolysis) or peptide features associated with HLA-II binding pocket interactions. In addition, inclusion of structural information for peptide-MHC-II complexes and cognate TCR sequences may enable computational approaches for predicting TCR specificity and immunodominant epitopes together (Glanville et al. 2017).

Models encompassing a wide range of population HLA-II diversity, peptide ligands and their antigen contextual features will improve identification of immunodominant T cell antigens and ultimately expand the understanding of regulatory mechanisms governing adaptive immunity. In this context, Applicants deployed CAPTAn to enable functional characterization of microbiome-specific T cells. Commensal microbes at mucosal sites are increasingly recognized for their roles in immune conditioning, whereby they promote education and maturation of the immune system. The microbiome also encodes a vast array of potential antigens that can promote diversification of the antigen receptor repertoire, elicit positive selection of lymphocytes in the periphery, and enforce peripheral tolerance directly or in a bystander-dependent manner (Honda and Littman 2016; Plichta et al. 2019). CAPTAn identified a series of commensal T cell antigens that are associated with diverse cytokine responses. In particular, Applicants identified a DRB1*03:01-restricted peptide derived from V. parvula 4-hydroxy-3-methylbut-2-enyl diphosphate reductase. Peripheral T cells responded to restimulation with this epitope by producing IL-17A, suggesting that healthy subjects actively recognize microbiome antigens and are imprinted by the mucosal microenvironment to produce IL-17. Microbiome antigens can also function by molecular mimicry to modulate cross-reactive T cells. A recent study identified dual-reactive T cells specific for microbiome epitopes and SARS-COV-2, suggesting that pre-existing immunity to the microbiome can poise the adaptive immune system to respond by mimicry (Bartolo et al. 2021). Thus, it is valuable to be able to identify the antigen specificity of T cells so as to determine their fate and contribution to immunity. The computational experimental workflow also identified a novel immunogenic HLA-DQ restricted SARS-COV-2 epitope derived from the nucleoprotein, which is strictly conserved amongst emerging viral variants. These efforts show potential to assist in the design of HLA-optimized vaccines for SARS-COV-2, which is important for peptide-based vaccines that induce lasting CD4+ T cell responses (Heitmann et al. 2022) and are crucial for preventing severe disease and prophylaxis towards new variants of concern (Keeton et al. 2022; Naranbhai et al. 2022). Thus, T cell antigen prediction methods that faithfully consider HLA diversity and contextual features of antigenicity have great value in surveillance of epitopes in the constantly evolving human virome, the microbiome, and in host-derived antigens such as autoantigens or neoantigens (Ahmed et al. 2019).

### Experimental Model and Subject Details

### Human Samples

Blood samples (PBMCs and leukapheresis samples) from healthy donors were acquired from STEMCELL Technologies after obtaining a signed consent form. IRB approval was obtained from WCG IRB (Puyallup, WA, USA).

### Bacterial Strains

Bacterial strains were all isolated from healthy human stool samples. The microbial isolates used in the experiment consistent of commercially available strains:

Veillonella parvula SKV38 (NCBI GCF_902810435), Akkermansia muciniphila (strain ATCC BAA-835/Muc, Uniprot ID UP000001031), Bacteroides thetaiotaomicron (strain ATCC 29148, Uniprot ID UP000001414) and laboratory-cultivated isolates from human stool samples (to be made public upon publication): Ruminococcus gnavus (RJX1147), Clostridium clostridioforme (RJX1175), Bifidobacterium longum (RJX1250). The related assembled proteomes were used in the downstream analyses. All bacterial strains were grown from previously frozen cultures and plated on Brain Heart Infusion (BHI) agar supplemented with trace minerals (ATCC), trace vitamins (ATCC), 0.5% D-fructose (Sigma), 0.5% D-maltose (Sigma), 0.5% L-cysteine (Sigma), and 1% vitamin K-hemin (BD). Plates were grown for 2 days and a single colony was inoculated into the identical BHI supplemented media in broth form. Broth cultures were grown for 48 hours in 50 mL of supplemented BHI for all strains except A. muciniphila which was grown for 72 hrs. Cultures were next centrifuged at 4000×g, and the pellet was washed twice with reduced PBS (0.05% cysteine). Bacterial communities were then created by combining individual strains at equal CFUs.

### Cell Culture and Transfection

Expi293F™ cells (ThermoFisher Scientific) were cultured with Expi293™ expression medium (ThermoFisher Scientific) in a 37° C. incubator with 8% CO2. For 1 ml transfections, 500 ng of HLA-II plasmid DNA and 500 ng of HLA-DM were prepared in 50 μl of Opti-MEM™ (ThermoFisher Scientific), and 2.7 μl of ExpiFectamine™ 293 reagent (ThermoFisher Scientific) were separately prepared in 50 μl of Opti-MEM. DNA and ExpiFectamine™ were mixed after 5 min at room temperature, and the mixture was further incubated for 30 min at room temperature. pLX307 Luciferase (Addgene plasmid #117734; n2t.net/addgene: 117734; RRID: Addgene_117734) was used for mock transfection (Hong et al. 2019). The mixture was then added to 1 ml of 3×10⁶ cells, and the cells were cultured for 48 hours. Cell density and viability were calculated, and the cells were harvested by centrifugation for 5 min at 500×g at 4° C. Cells were washed once with cold PBS, and cell pellets were flash frozen in liquid nitrogen and stored at −80° C. until further analysis. Small aliquots of cells were taken before freezing and tested for HLA-II expression by flow-cytometry.

### DC-Microbe Coculture

To generate monocyte-derived dendritic cells (MDDCs), leukapheresis samples (Stemcell) were obtained from a human donor, monocytes were purified by immunomagnetic negative selection, and cells were differentiated in vitro. Leukapheresis samples were treated with RBC lysis buffer (Bioleged) and washed twice in selection buffer (PBS, 2% FBS, 1 mM EDTA). Immunomagnetic positive selection of monocytes was performed following manufacturer recommended protocol (EasySep, Stemcell), and isolated monocytes were resuspended at 1×10\hat{ }6 cells/mL in differentiation media (RPMI 1640 supplemented with 10% FBS, 1% P/S, 1% L-glutamine, 400 UI/mL human IL-4 and 1000 UI/mL human GM-CSF), plated in 15 cm tissue culture treated dishes at a volume of 30 mL and incubated at 37° C., 5% CO2. On days 3 and 5 of culture, cytokines were added by removing 1/2 of the media from differentiating cells, centrifuging media to recover cells at 350×g for 5 min at RT, and then resuspending recovered cells in differentiation media containing 2-fold concentrations of IL-4 and GM-CSF before adding back to original dishes. On day 7, differentiated MDDCs were co-cultured for 6 h at 37° C., 5% CO2 with bacterial communities at a ratio of 1:20 MDDC: CFU bacteria. A 0 h control was performed by adding bacteria as above then immediately washing cells. For both time points, MDDCs were collected with a cell scraper, and washed twice in PBS by centrifugation at 350×g for 5 min at RT, and then stored as cell pellets at −80° C.

### Generation of BW_MHCIIz Stable Cell Lines

BW5147.3 cells (ATCC) were cultured with DMEM supplemented with 10% FBS in a 37° C. incubator with 5% CO2. Stable cells for HLA-II were generated by serial lentivirus transduction of HLA-II alpha and beta chains. Lentiviruses were used after collection or thawed at 4° C. Viral supernatant was added to BW5147.3 cells with 8 μg/ml of Polybrene (sigma), and the mixture was centrifuged at 800 g for up to 2 hours at 32° C. Supernatant was removed, and the cells were resuspended in fresh media. Cells were cultured for one or two days, and transduced cells were selected by 2.5 mg/ml of puromycin for 2 days. HLA-II expression was confirmed by flow-cytometry using fluorescence-conjugated HLA-DQ specific antibody (HLA-DQ, Biolegend clone HLADQ1) as described above.

### Method Details

### Mono-Allelic HLA-II DNA Construct Design

Full-length gene sequences were obtained from the IPD-IMGT/HLA webpage (www.ebi.ac.uk/ipd/imgt/hla/allele.html) (Robinson et al. 2020). Flexible linker (SGGSA (SEQ ID NO: 86)) and Strep-tag II (WSHPQFEK (SEQ ID NO: 87)) were fused to the C-terminus of beta chain and a small linker (GS) and V5 tag (GKPIPNPLLGLDST (SEQ ID NO: 88)) were fused to the C-terminus of alpha chain. All the classical HLA-II constructs contain the kozak sequence followed by epitope-tagged beta chain, P2A ribosomal skip sequence, the epitope-tagged alpha chain, and two stop codons. For HLA-DQB1*03:02, two stop codons were added 3 prime to the Strep-tag II. HLA-DM (HLA-DMB*01:01 and HLA-DMA*01:01) was constructed similarly without any epitope tags. All the recombinant constructs were synthesized as gblocks from IDT and cloned in house, or synthesized from and cloned by GenScript. All the constructs were inserted into pLX307 using Nhel and Spel restriction enzyme sites.

### Generation of HLA-DQAI and HLA-DPA1 Knockout Cell Lines by CRISPR-Cas9 Genome Editing

An Alt-R CRISPR-Cas9 crRNA (CGTTGCCTCTTGTGGTGTAA (SEQ ID NO: 89)) targeting the DQA1*01:02 locus and an Alt-R CRISPR-Cas9 crRNA (CGTTTGTACAGACGCATAG (SEQ ID NO: 90)) targeting the DPA1*01:03 locus were synthesized from IDT. 100 mM guide RNA (gRNA) duplex was prepared from crRNA and Alt-R CRISPR-Cas9 tracrRNA (IDT) in nuclease-free IDTE buffer (IDT) by incubating at 95° C. for 5 min and cooling down to ambient RT on the bench. Ribonucleoprotein (RNP) complex was prepared by incubating recombinant Cas9 protein with NLS (PNA Bio) and gRNA in PBS for 20 min at RT at 20 mM and 40 mM final concentration, respectively. RNP complex was delivered to low-passage Expi293F™ cells using SF Cell Line 4D-Nucleofector™ X Kit S (Lonza) according to the manufacturer's protocol. After 3 days of recovery, cells were diluted in a fresh medium supplemented with 6 mM Glutamax and plated in non-treated 96-well plates to a final density of 0.5 cells/well. Cells were cultured without shaking until colonies were visible. Wells containing only one foci were selected and expanded. Genome editing was confirmed by Sanger sequencing. Genomic DNA was extracted using the QiAamp DNA mini kit (Qiagen). DNA fragments containing the gRNA target region were PCR amplified, and the amplified PCR fragments were sequenced. For clones with heterozygous sequencing results, the PCR amplified fragments were further cloned into pGEX6P-1, and individual colonies were cultured and sequenced. Clones with desired knockout events were selected based on the sequencing result and used for further study.

### HLA-II Expression Check by Flow-Cytometry

Up to a million cells were used for sample preparation and flow-cytometry. Cells were washed once with cold PBS containing 0.2% BSA (PBS-BSA) by centrifugation for 5 min at 500×g at 4° C. 0.3 μl HLA-II specific antibodies were incubated with cells in 40 μl PBS-BSA for 20 min on ice. Cells were washed twice with 200 μl of PBS-BSA and resuspended in 100 μl of PBS-BSA. For whole-cell protein staining and for detection of cytosolic epitope tags, BD Cytofix/Cytoperm™ Fixation/Permeabilization kit (BD Biosciences) was used. Cells were washed once with PBS-BSA, and the cell pellet was incubated in 100 μl of Fixation/Permeabilization solution on ice for 20 min in the dark. Cells were washed twice with 200 μl of 1 BD Perm/Wash™ buffer (wash buffer) and resuspended in 40 μl of wash buffer. 0.3 μl of fluorescence-conjugated HLA-II specific antibodies (HLA-DP, BD Bioscience clone B7/21; HLA-DQ, Biolegend clone HLADQ1; HLA-DR, Biolegend clone L243; HLA-DM, Santa Cruz clone MaP.DM1), 0.5 μl of anti-V5 (R&D systems clone 1036H), or 1 μl of anti-Strep-tag II (iba) was added to the cells and incubated for 30 min on ice in the dark. Cells were washed twice with the 200 μl of wash buffer and resuspended in 100 μl of wash buffer. Strep-tag II antibody was detected by fluorescence-conjugated goat anti-Mouse IgG (Thermo) for an additional 20 min after wash. Cells were washed again and prepared as described above. Surface and whole cell expression were analyzed by CytoFLEX S Flow Cytometer (Beckman Coulter Life Sciences), and the data were analyzed using FCS Express 7 (De Novo Software).

### HLA-II Bound Peptide Strep Tactin Immunoprecipitation

Affinity-tagged HLA-peptide complexes were isolated from cells expressing Strep-tag-II HLA alleles and negative control cell lines that expressed only endogenous HLA-II. Strep-tactin sepharose resin (Cytiva) was prepared by washing three times with 1 mL cold PBS. Following bead preparation, frozen pellets containing between 150×10\hat{ }6 and 240×10\hat{ }6 cells expressing Strep-tag-II HLA-II were thawed on ice for 20 min and gently lysed in 1.2 mL cold lysis buffer [20 mM Tris-Cl pH 8, 100 mM NaCl, 6 mM MgCl2, 1.5% (v/v) Triton X-100, 60 mM octyl glucoside, 0.2 mM of 2-Iodoacetamide, 1 mM EDTA pH 8, 1 mM PMSF, 1× complete EDTA-free protease inhibitor cocktail (Roche, Basel, Switzerland)] per 50×10\hat{ }6 cells. Lysates were then split into approximately 50×10\hat{ }6 cell aliquots, incubated on ice for 30 min with ≥250 units of benzonase nuclease (Sigma-Aldrich, St. Louis, MO) and inverted once after 15 minutes to degrade DNA/RNA. Cells were centrifuged at 15,000×g at 4° C. for 20 min to remove cellular debris and insoluble materials, and cleared supernatants were transferred to tubes with a volume corresponding to 150 μL of Strep-tactin resin slurry and incubated end/over/end at 4° C. for one of three time points: 1 hour, 3 hours, or overnight to allow binding of Strep-tag-II tagged HLA molecules.

Next, a 10 uM PE fritted plate (Agilent, S7898A) used for washing beads was sawed in half to allow bead washes from different length incubations (1 hr and 3 hr) to be washed simultaneously. The plate was activated with 1 mL of acetonitrile and washed with 3 mL of room temperature PBS, and the bead-lysate mixtures were spun at 1500×g at 4° C. for 1 min. HLA-II depleted cell lysates were frozen at −80° C. for downstream proteome analysis. HLA-II bound Strep-tactin resin was resuspended in 1 mL cold PBS and transferred to the pre-washed 10 uM PE fritted plate, followed by a 500 uL tube rinse. The plate was then placed on a Waters Positive Pressure Manifold and washed to remove non-specific binders using two washes with 2 mL of cold complete wash buffer (20 mM Tris, pH 8.0, 100 mM NaCl, 1 mM EDTA, 6 mM Octyl β-d-glucopyranoside, 0.2 mM Iodoacetamide) and two washes with 2 mL of 10 mM Tris pH 8.0 buffer (PSI<5). The 10 uM PE fritted plate with dry Strep-tactin beads from the one hour incubation was wrapped with parafilm and stored at 4° C. until the 3 hour incubation was completed and beads were washed on the other half of the plate. The two half plates were then placed back together on top of the desalting plate, enabling a single desalt to be performed prior to LC-MS/MS analysis.

### Pan HLA-II Antibody Immunoprecipitation for DC Feeding Samples

Pan-HLA-II IP samples were lysed with the same cold lysis buffer used for the Strep-tactin IP (1.2 ml lysate per ˜50×10\hat{ }6 cells). Each lysate was incubated on ice for 30 min with 2 uL of Benzonase and inverted after 15 min. The lysates were then centrifuged at 15,000 ref for 20 min at 4° C. and the supernatants were transferred to another set of eppendorf tubes containing ˜37.5 uL pre-washed Gammabind Plus Sepharose beads (Millipore Sigma, GE17-0886-01) and 15 μl of HLA-II antibody mix (9 uL TAL-1B5 (abcam, ab20181), 3 uL EPR11226 (abcam, ab157210), 3 uL B-K27 (abcam, ab47342)). The HLA complexes were captured on the beads by incubating on a rotor at 4° C. for 3 hr then washed and prepared for desalting in the same way as the Strep tactin IP samples.

HLA-II Bound Elution and Desalting with a Positive-Pressure Manifold and 96 Well Plate

HLA peptides were eluted and desalted from beads as follows: 1 well per 50×10\hat{ }6 replicate of the tC18 40 mg Sep-Pak desalting plate (Waters, Milford, MA) were activated two times with 1 mL of methanol (MeOH), 500 uL of 99.9% acetonitrile (ACN)/0.1% formic acid (FA), then washed four times with 1 mL of 1% FA. The two halves of the 10 uM PE fritted filter plate containing the Strep tactin beads were put together and placed on top of the Sep-Pak plate and 200 uL of 3% ACN/5% FA was added to the beads. Then 1 uL of 100 fmol Biognosys internal retention time (iRT) standards (SKU: Ki-3002-2) was spiked into each sample. One wash of 400 uL of 1% FA was pushed through the 10 uM filter plate. The beads were then incubated with 500 uL of 10% acetic acid (AcOH) three times for 5 min to dissociate bound peptides from the HLA molecules. The beads were rinsed once with 1 mL 1% FA and the filter plate was removed. The Sep-Pak desalt plate was rinsed with 1 mL 1% FA an additional three times. The peptides were eluted from the Sep-Pak desalt plate using 250 uL of 15% ACN/1% FA and 2×250 uL of 50% ACN/1% FA. Eluted HLA-peptides were frozen and dried via vacuum concentration. Dried peptides were stored at −80° C. until LC-MS/MS analysis.

### Immunopeptidome Fractionation

Briefly, peptides were loaded on Stage-tips with 2 punches of SDB-XC material (Empore 3M). HLA-I and HLA-II peptides were eluted in three fractions with increasing concentrations of ACN (HLA-I: 5%, 10% and 30% in 0.1% NH4OH, pH 10, HLA-II: 5%, 15%, and 40% in 0.1% NH4OH, pH 10) (Klaeger et al., 2021). Fractions were then frozen and dried via vacuum concentration prior to LC-MS/MS analysis.

### Immunopeptidome Sequencing by LC-MS/MS

Samples were reconstituted in 3% ACN/5% FA prior to separation using a Proxeon Easy NanoLC 1200 (Thermo Scientific, San Jose, CA) fitted with a PicoFrit (New Objective, Woburn, MA) 75 μm inner diameter capillary with a 10 μm emitter packed at 1000 psi of pressure with He to ˜25-40 cm with 1.9 μm particle size/200 Å pore size of C18 Reprosil beads (Dr. Maisch GmbH, Ammerbuch, Germany) and heated at 50° C. during separation. The column was equilibrated with 10× bed volume of buffer A [0.1% (v/v) FA and 3% (v/v) ACN], samples were loaded in 4 uL 3% (v/v) ACN/5% (v/v) FA, and peptides were eluted with a linear gradient from 2%-30% of Buffer B [0.1% (v/v) FA and 80% (v/v) ACN] over 84 min, 30%-90% Buffer B over 6 min, then held at 90% Buffer B for 15 min to wash the column. Linear gradients for sample elution were run at a rate of 200 nL/min and yielded ˜9-12 s median peak widths.

All samples were run with data-dependent acquisition. Eluted peptides were introduced into an Orbitrap Exploris 480 mass spectrometer (Thermo Scientific, San Jose, CA) equipped with a Nanospray Flex Ion source (Thermo Scientific, San Jose, CA) at 1.9 kV. When FAIMS was used, set voltages were −50V and −70V. For immunopeptidome analysis, a full-scan MS was acquired at a resolution of 60,000 from 350 to 1,800 m/z (Custom Normalized AGC target 100%, 50 ms max injection time). Each full scan was followed by a topN (cycle time 1.5 sec for each CV) of data-dependent MS2 scans at resolution 15,000, using an isolation width of 1.1 m/z, a collision energy of 34, a Custom Normalized ACG Target of 50%, and a max injection time of 120 ms max ion time. An isolation width of 1.1 m/z was used because HLA-II peptides tend to be longer (median 16 amino acids with a subset of peptides >40 amino acids), so the monoisotopic peak is not always the tallest peak in the isotope cluster and the mass spectrometer acquisition software places the tallest isotopic peak in the center of the isolation window in the absence of a specified offset. Dynamic exclusion was enabled with a repeat count of 1 and an exclusion duration of 10 s. Isotopes were excluded while dependent scans on a single charge state per precursor was disabled. Charge state screening for HLA-II data collection was enabled along with monoisotopic precursor selection (MIPS) using Peptide Mode to prevent triggering of MS/MS on precursor ions with charge state 2 to >5 (no FAIMS) or >6 (FAIMS), or unassigned.

### Interpretation of Immunopeptidomic LC-MS/MS Data

All peptide sequences were interpreted from MS/MS spectra using Spectrum Mill (v 7.1 pre-release) to search against a RefSeq-based sequence database mapped to the human reference genome (hg19) obtained via the UCSC Table Browser (genome.ucsc.edu/cgi-bin/hgTables), with the addition of 13 proteins encoded in the human mitochondrial genome, 602 common laboratory contaminant proteins, shared mutations from 26 tumor types in TCGA, and 553 human non-canonical small open reading frames for a total of 345,062 entries. For the DC feeding experiment, proteomes from Bifidobacterium longum, Ruminococcus gnavus, Clostridium clostridioforme, Veillonella parvula, Akkermansia muciniphila, and Bacteroides thetaiotaomicron were appended to the database above with the exclusion of the 26 shared TCGA mutations for a total of 329,287 entries.

Immunopeptidome data MS/MS spectra were excluded from searching if they did not have a precursor MH+ in the range of 600-4000, had a precursor charge >6, or had a minimum of <5 detected peaks. Merging of similar spectra with the same precursor m/z acquired in the same chromatographic peak was disabled. Prior to searches, all MS/MS spectra had to pass the spectral quality filter with a sequence tag length >1 (i.e., minimum of 3 masses separated by the in-chain masses of 2 amino acids) based on HLA v3 peak detection. MS/MS search parameters included: ESI-QEXACTIVE-HCD-HLA-v3 scoring parameters; no-enzyme specificity; fixed modification: carbamidomethylation of cysteine; variable modifications: cysteinylation of cysteine, oxidation of methionine, deamidation of asparagine, acetylation of protein N-termini, and pyroglutamic acid at peptide N-terminal glutamine; precursor mass shift range of −18 to 81 Da; precursor mass tolerance of ±10 ppm; product mass tolerance of ±10 ppm, and a minimum matched peak intensity of 30%. Peptide spectrum matches (PSMs) for individual spectra were automatically designated as confidently assigned using the Spectrum Mill auto-validation module to apply target-decoy based FDR estimation at the PSM level of <1.0% FDR.

Immunopeptidomics data was further cleaned by removing tryptic contaminants, peptides that did not map to human proteins, and non-specific binders that bound to negative control Strep-tactin or antibody IPs. DC feeding data was subjected to further quality filtering in order to decrease the rate of false positives. Data obtained from the DC feeding experiment were filtered to have a backbone cleavage score of >8 (HLA-II), a scored peak intensity >60, a score >7, and a delta parent mass ppm <|3|. Each peptide was assigned a tentative reference allele based on donor HLA-II genotype and the CAPTAN-core, with 9 out of 12 available alleles. In total, 295 peptides were deconvoluted with confidence >50% (62 filtered, 233 unfiltered) and 140 with confidence >75% (53 filtered, 87 unfiltered).

### Whole Proteome Data Generation of DC-Microbe Coculture and HLA IP Flow-Through

HLA-depleted DC-microbe coculture lysates and a small selection of monoallelic HLA IP depleted lysates (DPB*05:01 DPA1*02:02, DQB1*03:03 DQA1*03:01, DRB1*11:01, DRB1*11:01 PBS starvation, DRB1*11:01, Mock Luciferase) underwent denaturing lysis in SDS to prepare for S-Trap digestion. HLA depleted lysates were briefly thawed on ice for ˜15 min, and 10% SDS was added for a final concentration of 2.5% SDS to denature the lysate (˜1.5 mL final volume) that was prepared for S-Trap digestion.

Disulfide bonds were reduced in 5 mM DTT for 30 min at 25° C. (1000 rpm shaking), and cysteine residues alkylated in 10 mM IAA in the dark for 45 min at 25° C. (1000 rpm shaking). Lysates were transferred to a 15 mL conical tube for protein precipitation. 27% phosphoric acid was added at a 1:10 ratio of lysate volume to acidify, and proteins were precipitated with 6× sample volume of ice cold S-Trap buffer (90% methanol, 100 mM TEAB). The precipitate was transferred in successive loads of 3 mL to a Protifi S-Trap Midi and loaded with 1 min centrifugation at 4000×g, mixing the remaining precipitate thoroughly between transfers. The precipitated proteins were washed 4× with 3 mL S-Trap buffer at 4000×g for 1 min. To digest the deposited protein material, 350 uL digestion buffer (50 mM TEAB) containing both trypsin and endopeptidase C (LysC), each at 1:50 enzyme: substrate, was passed through each S-Trap column with 1 min centrifugation at 4000×g. The digestion buffer was then added back atop the S-Trap and the cartridges were left capped and shaking overnight at 25° C.

Peptide digests were eluted from the S-Trap, first with 500 uL 50 mM TEAB and next with 500 uL 0.1% FA, each for 30 sec at 1000×g. The final elution of 500 uL 50% ACN/0.1% FA was centrifuged for 1 min at 4000×g. Peptide concentration of the pooled elutions was estimated with a BCA assay and a 10 ug peptide aliquot and a 50 ug aliquot were frozen and dried using vacuum concentration.

Samples were reconstituted in 4.5 mM ammonium formate (pH 10) in 2% (vol/vol) acetonitrile and separated into four fractions using basic reversed phase fractionation on a 3 punchout C-18 Stage-tip. Fractions were eluted at 5%, 12.5%, 15%, and 50% ACN/4.5 mM ammonium formate (pH 10) and dried.

### Whole Proteome Sequencing by LC-MS/MS

Proteome samples were reconstituted in 3% ACN/5% and subjected to LC-MS/MS using data-dependent acquisition. Eluted peptides were introduced into an Orbitrap Exploris 480 mass spectrometer (Thermo Scientific, San Jose, CA) equipped with a Nanospray Flex Ion source (Thermo Scientific, San Jose, CA) at 1.8 kV. A full-scan MS was acquired at a resolution of 60,000 from 350 to 1,800 m/z (Custom Normalized AGC target 300%, 25 ms max injection time). Each full scan was followed by data-dependent MS2 scans using a cycle time of 2 s at resolution 15,000, using an isolation width of 0.7 m/z, a collision energy of 30, a Custom Normalized ACG Target of 30%, and a max injection time of 50 ms. Dynamic exclusion was enabled with a repeat count of 1 and an exclusion duration of 20 s. Charge state screening for whole proteome data collection was enabled along with monoisotopic precursor selection (MIPS) using Peptide Mode to prevent triggering of MS/MS on precursor ions with charge state 1, >6, or unassigned.

### Interpretation of Whole Proteome LC-MS/MS Data

For whole proteome data MS/MS spectra were excluded from searching if they did not have a precursor MH+ in the range of 600-6000, had a precursor charge >5, had a minimum of <5 detected peaks, or failed the spectral quality filter with a sequence tag length >0 (i.e., minimum of 2 masses separated by the in-chain masses of 1 amino acid) based on ESI-QEXACTIVE-HCD-v4-30-20 peak detection. Similar spectra with the same precursor m/z acquired in the same chromatographic peak were merged. MS/MS search was performed using Spectrum Mill (v 7.1 pre-release) and data was searched against a RefSeq-based sequence database mapped to the human reference genome (hg19) obtained via the UCSC Table Browser (genome.ucsc.edu/cgi-bin/hgTables), with the addition of 13 proteins encoded in the human mitochondrial genome, 602 common laboratory contaminant proteins, shared mutations from 26 tumor types in TCGA, and 553 human non-canonical small open reading frames for a total of 345,062 entries. Search parameters included: ESI-QEXACTIVE-HCD-v4-30-20 scoring parameters; Trypsin allow P specificity with a maximum of 4 missed cleavages; fixed modification: carbamidomethylation of cysteine and seleno-cysteine; variable modifications: oxidation of methionine, deamidation of asparagine, acetylation of protein N-termini, pyroglutamic acid at peptide N-terminal glutamine, and pyro-carbamidomethylation at peptide N-terminal cysteine; precursor mass shift range of −18 to 64 Da; precursor mass tolerance of ±20 ppm; product mass tolerance of ±20 ppm, and a minimum matched peak intensity of 30%. Peptide spectrum matches (PSMs) for individual spectra were automatically designated as confidently assigned using the Spectrum Mill auto-validation module to apply target-decoy based FDR estimation at the PSM level of <1.0% FDR. Protein level data was summarized by top uses shared (SGT) peptide grouping and contaminants (non-human proteins from species not found in the DC feeding experiment) were removed from the data.

Postfiltering, intensity-based absolute quantification (iBAQ) was performed on the whole proteome LC-MS/MS as described in (Schwanhäusser et al. 2011). Briefly, iBAQ values were calculated as follows: log 10 (totalIntensity/numObservableTrypticPeptides), the total precursor ion intensity for each protein was calculated in Spectrum Mill as the sum of the precursor ion chromatographic peak areas (in MS1 spectra) for each precursor ion with a peptide spectrum match (MS/MS spectrum) to the protein, and the numObservableTrypticPeptides for each protein was calculated using the Spectrum Mill Protein Database utility as the number of tryptic peptides with length 8-40 amino acids, with no missed cleavages allowed. Both log 10 transformed iBAQ values were median normalized.

### Analysis of Previously Published Immunopeptidome Data

Publicly available immunopeptidome datasets (MSV000083991, PXD004746, PXD004894, PXD005704, PXD012308, PXD010450) were downloaded and processed with the same methods as above.

### Estimating HLA-II Conservation Scores and Structural Modeling

HLA-II protein variant sequences were obtained from IMGT. HLA-II proteins which do not contain the entire sequence of the putative transmembrane region due to truncation were excluded. HLA-II proteins which have only partial sequences available were also excluded. HLA-II proteins with deletion at both termini were included if the peptide binding domain and the transmembrane domain sequences were intact and known. Total number of protein variants used for calculating conservation scores were 113 for HLA-DPA1, 355 for HLA-DPB1, 147 for HLA-DQA1, 319 for HLA-DQB1, and 353 for HLA-DRB. Multiple sequence alignment was generated using MUSCLE method using SnapGene and used to calculate conservation scores using ConSurf server (Glaser et al. 2003; Landau et al. 2005).

### Peptide Stimulation

To quantify cytokine responses in HLA-DRB1*03:01 positive donor PBMCs to microbial peptide epitopes, cytokine assays were performed. PBMCs were cultured at 37° C., 5% CO2 in RPMI 1640 supplemented with 10% fetal bovine serum (FBS) and 1% penicillin-streptomycin at a concentration of 5×10⁶ cells/mL for 18 h with peptide or control at a final concentration of 10 μg/mL peptide. For DMSO, the equivalent volume was used. Cytometric bead arrays (CBAs, BD Biosciences) for IL-2, IFNγ, IL-10 and IL-17A were used to analyze cytokine concentrations after in vitro stimulation. CBAs were performed on culture supernatants using manufacturer recommended protocols and analyzed on a CytoFLEX S flow cytometer. Interpolation of cytokine concentration (pg/mL) was based on reference standards and calculated with GraphPad Prism 9.3.1. Results were reported as mean Z-scores relative to negative controls (DMSO, CLIP [PVSKMRMATPLLMQA (SEQ ID NO: 91)], IGRP [IQHLQKDYRAYYTFL (SEQ ID NO: 92)]).

### DC7-HLA-DRB1*03:01 Tetramer Staining

PBMCs from HLA-DRB1*03:01 positive donors were thawed and rested overnight in RPMI 1640 10% FBS, 1% penicillin-streptomycin at 37° C., 5% CO2. A negative T cell magnetic enrichment (EasySep, Stemcell) was performed the following day. Enriched T cells were then treated with Dasatinib (Sigma) at 50 nM and 25 μg/mL Fc-block (BD biosciences) for 10 min at 37° C., 5% CO2 in RPMI 1640 10% FBS, 1% penicillin-streptomycin. 5 nM of APC and PE labeled DC7-HLA-DRB1*03:01 tetramers were then added for 30 min. Cells were washed once in EasySep buffer (PBS, 2% FBS, 1 mM EDTA) before a combined APC and PE magnetic enrichment (EasySep, Stemcell). Following enrichment, tetramer labeled T cells were resuspended in 1:1000 Fixable Viability Stain 510 (FVS510, BD Biosciences) in PBS for 10 min at RT, before surface staining for CD45, CD3, CD4, CD8, CD14 and CD19 at 1:200 in FACS buffer (PBS, 2% FBS) for 20 min at 4° C. Stained cells were then analyzed on a CytoFLEX LX flow cytometer.

Construct Design of Peptide-MHC—II-Fc (pMHCII-Fc)

pMHCII-Fc constructs were designed based on (Serra et al. 2019) with some modifications. Briefly, extracellular domains of HLA-II were fused to CH₂ and CH3 domain of the Fc region of human IgG followed by FLAG-tag for the alpha chain, and twin strep tags and 6× His tag for the beta chain. 20-mer peptides and a linker (GGGGTSGGSGGS (SEQ ID NO: 93)) were fused on the beta chain after the signal sequence (MVCLKFPGGSCMAALTVTLMVLSSPLALA (SEQ ID NO: 94)) followed by Glycine. Cysteine-trap was removed.

pMHCII-Fc Tetramer Generation

Alpha and beta chains were transiently co-transfected in ExpiCHO cells (Thermo). pMHCII-Fc was purified from the supernatant after a seven day growth period using nickel affinity chromatography (QIAGEN). Purified protein was biotinylated on the Avi-tag with BirA. Biotinylated pMHCII-Fc was run on superdex 200 Increase 10/300 GL (Cytiva), and fractions with no high molecular weight aggregates were chosen to multimerize. The moles of biotinylated pMHCII-Fc were calculated and assembled into tetramers as described in (Willis et al. 2021). Fluorescently-labeled streptavidin was added in five subsequent steps and allowed to assemble overnight at 4° C. in amber glass vials.

### Construct Design for TCR Reactivity Screen

For TCR, the cytoplasmic region of human CD3zeta was fused at the c-terminus of the transmembrane domain of TCR alpha and beta chains. Beta chain followed by P2A ribosomal skip sequence and alpha chain were cloned into pLX307. Sequences for human CD3 epsilon, zeta, delta, and gamma were obtained from (Dong et al. 2019), the four subunits were separated by P2A sequences and cloned into pLX307. HLA-II sequences were obtained as described above, and the cytoplasmic region of human CD3 zeta was fused at the c-terminus of the transmembrane domain of HLA-II alpha and beta chains. Peptides were fused to the N-termini of the beta chains followed by a linker (GGGGTSGGSGGS (SEQ ID NO: 93)). Alpha and beta chains were cloned separately into pLX307. All the constructs were synthesized and cloned by GenScript.

### Lentivirus Production

HEK293 cells were transiently transfected with 1 ug of lentiviral construct, 1 ug of PAX2 vector, and 0.1 ug of VSVg vector using Lipofectamine 2000 (Thermo) according to the manufacturer's instruction. 1 ml of viral supernatant were collected after 24 hours, and 1 ml of fresh medium was added. 2 ml of viral supernatant were collected 48-hour post-transfection. Cell debris were removed by centrifugation for 10 min at 500 g, and the combined viral supernatant was concentrated using Lenti-X concentrator (Takara) according to the manufacturer's instruction. Concentrated viruses were aliquoted and stored at −80° C.

### TCR Reactivity Screen

TCR and human CD3 were co-expressed in Expi293F cells as described above. ExpiFectamine transfection enhancers were added at 20 hours post-transfection as per the manufacturer's instructions. At 48 hours post-transfection, TCR-expressing Expi293F cells and peptide-MHC-II-expressing stable BW5147.3 cells were plated in 96-well plates with the ratios of 1:1 or 2:1. Cell densities ranged from 10,000 to 120,000 per well in 96-well plates. Cells were co-cultured in DMEM supplemented with 10% FBS for an additional 24 hours in a 37° C. incubator with 5% CO2. Surface HLA-II, TCR, and 4-1bb expression were then analyzed by flow-cytometry using fluorescence-conjugated specific antibodies for HLA-DQ (clone HLADQ1, Biolegend), TCR (IP26, Thermo Fisher Scientific), and mouse CD137 (clone 17B5, Biolegend) as described above.

### Quantification and Statistical Analysis

### Collecting and Processing External Datasets

IEDB (mono-allelic). The full MHC ligand table was retrieved from www.iedb.org/database_export_v3.php on Jun. 6, 2021. Ligands mapping to the human proteome and restricted to a single HLA-II were selected, yielding data for 46 DR, 54 DQ and 31 DP alleles. This resulted in 145,605 ligands, spanning 46,500 non-overlapping regions in 6,997 human proteins.

Reprocessed published data (mono-allelic). The mono-allelic data from (Abelin et al. 2019) was reprocessed using Spectrum Mill MS Proteomics (Software version 07.08.214) and deposited under MassIVE accession MSV000083991. Applicants retrieved 196,709 peptides, binding to 5 DP, 3 DQ, and 35 DR alleles. The sequences spanned 70,604 non-overlapping regions in 10,529 human proteins after de-nesting described below.

HLA ligand atlas (multi-allelic). The data described in (Marcu et al. 2021) was downloaded from hla-ligand-atlas.org/on Jun. 23, 2021 resulting in 333,194 peptides, de-convoluted to 18 DP, 39 DQ, and 24 DR alleles using NetMHCIIpan 4.0 as described below. The sequences spanned 125,905 non-overlapping regions in 6,607 human proteins after de-nesting described below.

Reprocessed published data (multi-allelic). Multi-allelic ligands were collected from (Abelin et al. 2019), MassIVE MSV000083991, (Racle et al. 2019), and Proteome data exchange PXD012308, resulting in 74,582 raw peptides, de-convoluted to 4 DP, 2 DQ, 9 DR alleles using NetMHCIIpan 4.0 as described below. The sequences spanned 18,429 non-overlapping ligand regions in 5,531 human proteins after de-nesting described below.

Mapping of peptides to ligand regions (de-nesting). The allele-specific ligand peptides were mapped to protein sequences in the UniProt reference, obtaining a single binary track per each allele and protein-marking each amino acid as present or absent in at least one binder. The obtained tracks were used to extract non-overlapping ligand regions, contiguous stretches of amino acids present in ligands.

Inference of allele-specific primary and secondary motifs. Ligands from mono-allelic data sets (this work, IEDB and (Abelin et al. 2019)) were merged and de-nested into non-overlapping ligand regions in human proteins as described above. Applicants selected 87 alleles with at least 250 unique ligands, with lengths between 7-50 amino acids, which is equal to the training set used for classification (see below). The merged ligand regions were processed with GibbsCluster 2.0 using the following parameters: 1-3 motif clusters, number of seeds 5, trash cluster, no preference for hydrophobic amino acids at P1 and expected motif of length 9 amino acids. The majority of runs produced two visually distinct motifs clusters (see Suppl. FIG. 2), termed primary and secondary motifs, with the smaller (secondary) motif cluster covering on average 35.7% of sequences. The motifs' probability matrices were clustered using Euclidean distance and shown on a dendrogram using complete linkage (R method hclust).

Inference of allele-specific consensus motifs. Ligands from mono-allelic data sets (this work, IEDB and ref. Abelin) were de-nested into non-overlapping ligand regions in human proteins as described above. Alleles from different datasets were not combined in this analysis and alleles with at least 30 unique regions were selected (140 out of 150). The list of non-overlapping epitopes was processed with GibbsCluster 2.0 using the following parameters: 1-3 motif clusters, number of seeds 5, trash cluster, no preference for hydrophobic amino acids at P1 and expected motif of length 9 amino acids.

This resulted in six candidate 9-mer motifs per allele; the consensus motif explaining at least 30 sequences was selected by maximum Kullback-Leibler divergence from the prior amino acid distribution as computed by GibbsCluster 2.0. The motifs were further aligned at P1 by selecting the position with lowest entropy within the first two positions, resulting in motifs of length 8 or 9. The majority of alleles had motifs of length 9 (10⁹ out of 140). The motifs' probability matrices were clustered using Euclidean distance and shown on a dendrogram using complete linkage (R method hclust).

Motif uncertainty analysis. Motifs resulting from GibbsCluster 2.0 are summarized in position frequency matrices P of size 9×20, whose columns sum to 1. The information content at each position Pᵢ, i=1 . . . 9, in a motif was estimated using the Shannon entropy (H), estimating the number of bits required to specify a distribution over amino acids j=A, C, D, . . . . Y:

$${H}_{i} = {\sum }_{j} {P}_{ij} {log}_{2} ( {P}_{ij} ) .$$

Larger values of Hᵢ reflect higher uncertainty at position i.

Contribution of alpha and beta chains to motif variation. DP and DQ isotypes can assume different pairings between alpha and beta chains. The contribution of different variants of the alpha chains (DQA and DPA) versus the beta chains (DQB and DPB) were assessed by statistical modeling of motif probability matrices. The 35 DP motifs consist of 3 different alpha chains and 15 different beta chains (3 and 12 appearing more than once, respectively). Similarly, for 33 DQ motifs there are 11 alpha chains and 14 different beta chains (10 and 7 appearing more than once). The related variant numbers were used as discrete covariates and motif probability matrices as dependent variables.

The probability vectors of size 20 at each position P₁-P₉ in a motif were modeled by two Dirichlet regression models, one for each chain:
Pᵢ˜Dirichlet (α),
or
Pᵢ˜Dirichlet (β),
   where α={01:03, 02:01, 02:02} for DP or α={01:01, 01:02, . . . , 06:01} for DQ, and β={01:01, 02:01, . . . , 17:01} for DP and β={02:01, 02:02, . . . , 06:04} for DQ. The model parameters are estimated by the Newton-Raphson algorithm (DirichletReg v0.7). The explained variance (R²) between the predicted mean and observed probability values is used to quantify individual model fits. The two models are compared based on the likelihood ratio test, estimating the contribution of each chain and associated significance values.

Mono-allelic binding core classifier training. The ligand set compiled from IEDB monoallelic MHC-II ligands, reprocessed Immunity (Abelin et al., 2019), and this work was split into five approximately equal parts as described above. This resulted in sets of eluted ligands for 150 different DR, DP, and DQ alleles.

Applicants selected 87 alleles with at least 50 unique ligands per training split, with lengths between 7-50 amino acids and maximum overlap of 5 amino acids. When multiple ligands overlap at a ligand region, one is randomly picked to approximate the observed length distribution. This resulted in data sets with a minimum of 378 ligands (DRB1*14:54) to a maximum of 7846 unique ligands (DRB1*11:01). To construct the negative (decoy) sets, for each ligand, Applicants sample five length-matched peptides which are not detected as binders in any of the 150 initial alleles. The list of ligands and decoys selected for each allele are provided in Table 1. The peptides are alloted into five approximately equal data splits, such that peptides from proteins in the same UniRef50 clusters cannot be present in two different splits to prevent test set leakage. In summary, 1,427,430 peptides containing 237,905 positives are used with 87 alleles having at least 50 unique ligands in each data split.

One binding core model is inferred for each of the 87 HLA-II alleles with at least 50 unique ligands in each of the five data splits. A binding core model, named CAPTAn-core, is a binary classification neural network, composed of two consecutive convolutional layers with width of 10 or 15 amino acids, followed by pooling, reduction and dropout layers (Goodfellow, Bengio, and Courville 2016). The final output is a probability that the input amino acid sequence contains a peptide ligand for the given HLA-II alleles. Depending on hyperparameters, the models contain between 56,449-97,345 parameters, which are tuned to minimize the binary entropy loss using the Adam optimization method (Kingma and Ba 2014). Out of five data splits, three are used for parameter optimization, one for hyperparameter tuning and early stopping and one for reporting final classification performance. The hyperparameters of layers are determined using grid search and the validation data split. Five models are tuned for each allele, each with one data split as an independent held-out data set on which the final performance on human proteins is reported. For non-human organisms, the predictions of five models are averaged. Further architectural and training details are provided in Supplementary Note 1.1.

Isotype-specific context model training. The isotype-specific (allele non-specific) classifiers were trained using the mono-allelic ligands for 150 alleles described above. In addition, Applicants included data for multi-allelic samples collected from (Abelin et al. 2019; Racle et al. 2019), and HLA Ligand Atlas (Marcu et al. 2021) as described above. Multi-allelic ligands were mapped to the most likely isotype (HLA-DP,-DR, or -DQ) using de-convolution based on NetMHCIIpan 4.0, restricting to corresponding HLA-II typed alleles (where available) and selecting the allele (and isotype) with highest rank-score. All peptides were then mapped to the proteome to obtain non-overlapping ligand regions as described above. In summary, the DR ligands spanned 11.9%, DP ligands 8.9%, and DQ ligands 5.9% of amino acids in 15,174 human proteins, for a combined 18.2% coverage.

The ligand regions merged for each of the three isotypes (HLA-DP,-DQ,-DR) define binary variables for each amino acid in a protein sequence. A recurrent neural network is trained to predict presence of ligand regions based on the amino acid sequence of a protein. It consists of a single convolutional layer (20 amino acid width), followed by two blocks of bi-directional, long-short term memory (LSTM) layers which scan the protein from both ends (Goodfellow, Bengio, and Courville 2016). The final probability of each amino acid belonging to a ligand region is predicted after multiple fully-connected layers and non-linear rectified linear units. The models contain between 127,363 parameters, which are tuned to minimize the binary entropy loss using the Adam optimization method (Kingma and Ba 2014). To maximize CPU utilization and minimize padding, proteins are clustered in 112 batches with approximately equal length. A gradient update is calculated once for each randomly-sampled half-batch to increase sequence diversity during training.

The data splits and their usage are aligned with the binding core models as described above; five models are tuned for each isotype, each with one data split as an independent held-out data set on which the final performance on human proteins is reported. For non-human organisms, the predictions of five models are averaged. Further architectural and training details are provided in Supplementary Note 1.2.

Combining the predictions of binding core and context models. The core and context models are combined in a final prediction using a weighted sum between core and context model. For each protein, core and context models are first run independently. For a given allele, CAPTAn-core predicts a probability for each amino acid being part of an allele-specific ligand by providing a consecutive 20 amino acid stretch. This probability is combined with the predicted probability of the corresponding context model. The context model predictions are aggregated in a 25 amino acid interval surrounding the prediction in question to more robustly account for the relevant context information. The relative weights given to the CAPTAn-context and CAPTAn-core models are selected from the interval [0-1] using grid search. This process is detailed further in Supplementary Note 1.3.

### Structural Feature Predictions and Statistical Analysis

Secreted proteins. Interproscan (v5.32-71.0, (M. Blum et al. 2021)) was run for all human proteins in UniProtKB to obtain predictions of signal peptides at the N-terminal end for each protein. The database used was SignalP_EUK (Nielsen 2017). Proteins with a predicted signal peptide were deemed secreted. This resulted in two features: protein-level “secreted” feature and amino acid “signal peptide”.

Transmembrane proteins. The prediction of transmembrane proteins was obtained with TMHMM (v2.0c, (Krogh et al. 2001)). Amino acids in each protein are classified as inside the cell (i.e., cytoplasmic side), outside the cell (o) or part of the transmembrane helix (h). The protein was deemed transmembrane if at least one helix was predicted. Regions within predicted transmembrane proteins were classified as “In membrane” or “Out of membrane” accordingly. Proteins without a single transmembrane helix were not counted, as recommended by the authors.

Domain predictions. The command hmmscan, part of HMMER3 (v 3.1, (Johnson, Eddy, and Portugaly 2010)), was run for all human proteins in UniProtKB. Domains were reported with an e-value threshold of 0.0001. An amino acid region was classified as in domain if it overlapped with at least one predicted PFAM domain. Three possible ways to compute overlap: hmm (the start of the alignment of domain with respect to the profile), ali (the start of the alignment of this domain with respect to the sequence) and env (the envelope defines a subsequence for which there is substantial probability mass supporting a homologous domain).

Solvent accessibility and disorder. Netsurfp (v2.0, (Klausen et al. 2019)) was run for all human proteins in UniProtKB to obtain a quantification of relative solvent accessibility (RSA) and disorder (both in [0, 1]). Amino acids with larger values are deemed more accessible to solvent and present in regions of higher disorder. HHblits was used as alignment-based protein sequence search against the UniClust30 database (v2017-04).

Statistical analysis of structural features. All amino acids within human proteins in UniProtKB were annotated with binary features or fractional features in [0, 1] (for disorder and RSA). The enrichment of each structural feature within allele-specific ligand sequences was computed using the training set for allele-specific classifiers, described above, where ligands represent 1 in 6 sequences (16.7%). The enrichment was quantified using Mann-Whitney rank-sum test comparing the presence of a feature in ligands versus decoys. The enrichment/depletion is visualized as the difference probability of feature in ligands versus the probability in all sequences. The predictive value of all collected features was assessed by providing the binary and fractional features as an additional input to context models, resulting in 10 additional variables for each amino acid (2 for secreted proteins, 3 for transmembrane features, 3 for domain features, 2 for RSA and disorder). The contribution of LSTM layers was assessed by comparing to the ablated models, termed CNN, with LSTM layers removed.

Allele-specific epitope predictors and settings. Maria (v. 1.0, (Chen et al. 2019)) was run with default parameters for 14 DR alleles overlapping with the combined mono-allelic training set. Each amino acid in the proteome was scored with Maria predicted binding probability (in [0, 1]) based on a 20-mer peptide starting at that amino acid. MixMHCIIpred (v1.2, (Racle et al. 2019)) was run with default parameters for 4 Dp, 3 DQ and 21 DR alleles overlapping with the combined mono-allelic training set. Each amino acid in the proteome was scored with raw MixMHCIIpred predicted binding probability (in [0, 1]) based on 20-mer peptide starting at that amino acid. NetMHCIIpan (v. 4.0, (Reynisson, Alvarez, et al. 2020)) was run with default parameters for all 150 alleles in the combined mono-allelic training set. Each amino acid in the proteome was scored with raw NetMHCIIpan score (in [0,1]) based on a 15-mer peptide starting at that amino acid. Raw score and percentile ranks were used where explicitly mentioned.

Evaluating predictions. The peptide mapping strategy described above selects at one protein for each peptide. Each protein in the set is thus associated with one or more microbial peptides. Using this set of proteins, Applicants apply the CAPTAn, NetMHCIIpan, MixMHCIIpred and Maria based on HLA-II alleles they support. The proteins were scanned in consecutive 20 aa stretches, each of which was scored by each method. After obtaining vectors of predicted scores for each amino acid in a protein, local maxima separated by at least 20 aa were selected as predicted ligand regions and ranked by descending predicted scores. Predictions overlapping with observed peptides in at least 7 amino acids (the minimum length of observed HLA-II ligands, having at least 1,500 representatives) were treated as correct.

TABLE 1
HLA-II alleles used in this study and its frequencies by population

| DPB1 | DPA1 | EUR | AFA | API | HIS |
|---|---|---|---|---|---|
| DPB1*01:01 | DPA1*02:01 | 4.30% | | 0.00% | |
| DPB1*02:01 | DPA1*02:02 | 0.00% | | 3.50% | |
| DPB1*02:02 | DPA1*02:02 | 0.00% | | 2.90% | |
| DPB1*03:01 | DPA1*02:02 | 0.00% | | 1.20% | |
| DPB1*05:01 | DPA1*02:02 | 1.50% | | 38.40% | |
| DPB1*05:01 | DPA1*02:01 | 0.00% | | 5.20% | |
| DPB1*09:01 | DPA1*02:01 | 0.70% | | 11.00% | |
| DPB1*10:01 | DPA1*02:01 | 1.60% | | 0.00% | |
| DPB1*11:01 | DPA1*02:01 | 2.30% | | 0.00% | |
| DPB1*13:01 | DPA1*02:01 | 1.40% | | 1.20% | |
| DPB1*14:01 | DPA1*02:01 | 1.20% | | 0.00% | |
| DPB1*14:01 | DPA1*02:02 | 0.00% | | 1.20% | |
| DPB1*17:01 | DPA1*02:01 | 1.50% | | 0.00% | |

| DQB1 | DQA1 | EUR | AFA | API | HIS |
|---|---|---|---|---|---|
| DQB1*02:01 | DQA1*02:01 | 0.00% | | 5.70% | |
| DQB1*02:01 | DQA1*05:01 | 13.20% | | 6.40% | |
| DQB1*02:02 | DQA1*02:01 | 11.80% | | 0.00% | |
| DQB1*03:01 | DQA1*03:03 | 6.50% | | 0.00% | |
| DQB1*03:01 | DQA1*05:01 | 0.00% | | 11.60% | |
| DQB1*03:01 | DQA1*05:05 | 11.60% | | 0.00% | |
| DQB1*03:01 | DQA1*06:01 | 0.10% | | 12.10% | |
| DQB1*03:02 | DQA1*03:01 | 9.60% | | 7.00% | |
| DQB1*03:03 | DQA1*02:01 | 3.70% | | 0.00% | |
| DQB1*03:03 | DQA1*03:01 | 0.00% | | 14.60% | |
| DQB1*04:01 | DQA1*03:01 | 0.00% | | 4.00% | |
| DQB1*04:02 | DQA1*04:01 | 2.30% | | 0.00% | |
| DQB1*05:01 | DQA1*01:01 | 10.90% | | 3.40% | |
| DQB1*05:02 | DQA1*01:01 | 0.00% | | 3.20% | |
| DQB1*05:02 | DQA1*01:02 | 1.20% | | 7.80% | |
| DQB1*05:03 | DQA1*01:04 | 2.00% | | 0.00% | |
| DQB1*06:01 | DQA1*01:02 | 0.00% | | 5.30% | |
| DQB1*06:01 | DQA1*01:03 | 0.70% | | 5.70% | |
| DQB1*06:03 | DQA1*01:03 | 5.70% | | 0.00% | |

| DRB1 | EUR | AFA | API | HIS |
|---|---|---|---|---|
| DRB1*04:06 | 0.00% | 0.10% | 2.10% | 0.20% |
| DRB1*04:11 | 0.00% | 0.10% | 0.00% | 2.40% |
| DRB1*11:01 | 5.70% | 8.70% | 5.10% | 4.20% |
| DRB1*13:04 | 0.00% | 1.30% | 0.00% | 0.60% |
| DRB1*14:02 | 0.00% | 0.10% | 0.00% | 2.40% |
| DRB1*14:04 | 0.00% | 0.00% | 2.00% | 0.00% |
| DRB1*14:05 | 0.00% | 0.00% | 1.80% | 0.00% |
| DRB1*14:06 | 0.00% | 0.00% | 0.30% | 2.60% |
| DRB1*16:02 | 0.10% | 1.50% | 2.00% | 2.10% |

Source: doi: 10.1186/s13073-020-00767-w

TABLE 2
Predicted microbial ligands and gene annotations, sorted by CAPTAn confidence
score and integrated with metagenomics and metatranscriptomics
data from the Integrative human microbiome project (HMP2).

| Pep- | | | | | | |
|---|---|---|---|---|---|---|
| tide | Organism | Protein | Predicted | | Observed | 9-mer |
| DC1 | Veillonella | WP_ | NAAVVIKDDKTPRGTRIFGP | | DENAAVVIKDDKTPRGTR | VIKDDKTPR |
| | parvula | 004695117.1 | (SEQ ID NO: 95) | | (SEQ ID NO: 96) | (SEQ ID NO: 97) |
| DC2 | Veillonella | WP_ | APFYRIVVADARAPREGRPV | | YRIVVADARAPREGRPV | VVADARAPR |
| | parvula | 004695565.1 | (SEQ ID NO: 98) | | (SEQ ID NO: 99) | (SEQ ID NO: 100) |
| DC3 | Ruminococcus | GNFAONKL_ | FTNKKLIVDIKEVKRPDKDA | | TNKKLIVDIKEVKRPDK | LIVDIKEVK |
| | gnavus | 01245 | (SEQ ID NO: 101) | | (SEQ ID NO: 102) | (SEQ ID NO: 103) |
| DC4 | Bifidobacterium | OHDAKFNF_ | YKPVLRLDADGKEVRTDENN | | TDENNFVVDLHLERIDHPQ | LDADGKEVR |
| | longum | 00523 | (SEQ ID NO: 104) | | (SEQ ID NO: 105) | (SEQ ID NO: 106) |
| DC5 | Bifidobacterium | OHDAKFNF_ | FVDGRVSIEVDPRLAHDTEN | | GRVSIEVDPRLAHDTEN | IEVDPRLAH |
| | longum | 01795 | (SEQ ID NO: 107) | | (SEQ ID NO: 108) | (SEQ ID NO: 109) |
| DC6 | Ruminococcus | GNFAONKL_ | TKGEIYVDGHEVRKEPEKVR | | RPTKGEIYVDGHEVRKEPEK | IYVDGHEVR |
| | gnavus | 01375 | (SEQ ID NO: 110) | | (SEQ ID NO: 111) | (SEQ ID NO: 112) |
| DC7 | Veillonella | WP_15669751 | FEAKVLEIDEKKNRLVLSRR | | EAKVLEIDEKKNRL | LEIDEKKNR |
| | parvula | 9.1 | (SEQ ID NO: 113) | | (SEQ ID NO: 114) | (SEQ ID NO: 115) |
| DC8 | Veillonella | WP_00469511 | GDVVKAVIVRSKKGIRRQDG | | | IVRSKKGIR |
| | parvula | 7.1 | (SEQ ID NO: 116) | | | (SEQ ID NO: 117) |
| | | | CAPTAn | NetMHCIIpan | MixMHCpred2 | Maria |
| Peptide | Allele | Score | Rank | Rank | Rank | Rank |
| DC1 | DRB1*03:01 | 0.8942 | 1 | 90 | 102 | 1259 |
| DC2 | DRB1*03:01 | 0.8919 | 3 | | 92 | 46 |
| DC3 | DRB1*03:01 | 0.8648 | 6 | 5 | 129 | 107 |
| DC4 | DRB1*03:01 | 0.8645 | 7 | 35 | 327 | 1768 |
| DC5 | DRB1*03:01 | 0.829 | 23 | 3 | 107 | 2309 |
| DC6 | DRB1*03:01 | 0.8218 | 33 | 6 | 193 | 350 |
| DC7 | DRB1*03:01 | 0.817 | 40 | 96 | 653 | 683 |
| DC8 | DRB1*03:01 | 0.8761 | 4 | 4668 | 1453 | |

| | | HMP2 | HMP2 |
|---|---|---|---|
| | Function of encoding proteins | Encoding | Metatranscriptomics |
| Peptide | (NCBI Blast search) | Genes | Prevalence |
| DC1 | 50S ribosomal protein L14 | 3 | 62 |
| DC2 | 30S ribosomal protein S16 | 1 | 58 |
| DC3 | 30S ribosomal protein S3 | 3 | 63 |
| DC4 | ribose 5-phosphate isomerase A | 3 | 114 |
| DC5 | transaldolase | 1 | 50 |
| DC6 | ABC transporter ATP-binding protein | 1 | 9 |
| DC7 | bifunctional 4-hydroxy-3-methylbut- | 4 | 138 |
| | 2-enyldiphosphate reductase/30S | | |
| | ribosomal protein S1 | | |
| DC8 | 50S ribosomal protein L14 | 2 | 54 |

### REFERENCES FOR EXAMPLE 1

Abelin, Jennifer G., Dewi Harjanto, Matthew Malloy, Prerna Suri, Tyler Colson, Scott P. Goulding, Amanda L. Creech, et al. 2019. “Defining HLA-II Ligand Processing and Binding Rules with Mass Spectrometry Enhances Cancer Epitope Prediction.” Immunity 51 (4): 766-79.e17.
Ahmed, Rizwan, Zahra Omidian, Adebola Giwa, Benjamin Cornwell, Neha Majety, David R. Bell, Sangyun Lee, et al. 2019. “A Public BCR Present in a Unique Dual-Receptor-Expressing Lymphocyte from Type 1 Diabetes Patients Encodes a Potent T Cell Autoantigen.” Cell 177 (6): 1583-99.e16.
Alfei, Francesca, Ping-Chih Ho, and Wan-Lin Lo. 2021. “DCision-Making in Tumors Governs T Cell Anti-Tumor Immunity.” Oncogene 40 (34): 5253-61.
Alvarez, Bruno, Birkir Reynisson, Carolina Barra, Søren Buus, Nicola Ternette, Tim Connelley, Massimo Andreatta, and Morten Nielsen. 2019. “NNAlign MA; MHC Peptidome Deconvolution for Accurate MHC Binding Motif Characterization and Improved T-Cell Epitope Predictions.” Molecular & Cellular Proteomics: MCP 18 (12): 2459-77.
Andreatta, Massimo, Bruno Alvarez, and Morten Nielsen. 2017. “GibbsCluster: Unsupervised Clustering and Alignment of Peptide Sequences.” Nucleic Acids Research 45 (W1): W458-63.
Andreatta, Massimo, Annalisa Nicastri, Xu Peng, Gemma Hancock, Lucy Dorrell, Nicola Ternette, and Morten Nielsen. 2019. “MS-Rescue: A Computational Pipeline to Increase the Quality and Yield of Immunopeptidomics Experiments.” Proteomics 19 (4): e1800357.
Baek, Minkyung, Frank DiMaio, Ivan Anishchenko, Justas Dauparas, Sergey Ovchinnikov, Gyu Rie Lee, Jue Wang, et al. 2021. “Accurate Prediction of Protein Structures and Interactions Using a Three-Track Neural Network.” Science 373 (6557): 871-76.
Balen, Peter van, Michel G. D. Kester, Wendy de Klerk, Pietro Crivello, Esteban Arrieta-Bolaños, Arnoud H. de Ru, Inge Jedema, et al. 2020. “Immunopeptidome Analysis of HLA-DPB1 Allelic Variants Reveals New Functional Hierarchies.” Journal of Immunology 204 (12): 3273-82.
Barra, Carolina, Bruno Alvarez, Sinu Paul, Alessandro Sette, Bjoern Peters, Massimo Andreatta, Søren Buus, and Morten Nielsen. 2018. “Footprints of Antigen Processing Boost MHC Class II Natural Ligand Predictions.” Genome Medicine 10 (1): 84.
Bartolo, Laurent, Sumbul Afroz, Yi-Gen Pan, Ruozhang Xu, Lea Williams, Chin-Fang Lin, Elliot S. Friedman, Phyllis A. Gimotty, Gary D. Wu, and Laura F. Su. 2021. “SARS-COV-2-Specific T Cells in Unexposed Adults Display Broad Trafficking Potential and Cross-React with Commensal Antigens.” bioRxiv: The Preprint Server for Biology, November. https://doi.org/10.1101/2021.11.29.470421.
Bepler, Tristan, and Bonnie Berger. 2021. “Learning the Protein Language: Evolution, Structure, and Function.” Cell Systems 12 (6): 654-69.e3.
Blum, Janice S., Pamela A. Wearsch, and Peter Cresswell. 2013. “Pathways of Antigen Processing.” Annual Review of Immunology 31 (January): 443-73.
Blum, Matthias, Hsin-Yu Chang, Sara Chuguransky, Tiago Grego, Swaathi Kandasaamy, Alex Mitchell, Gift Nuka, et al. 2021. “The InterPro Protein Families and Domains Database: 20 Years on.” Nucleic Acids Research 49 (D1): D344-54.
Borst, Jannie, Tomasz Ahrends, Nikolina Bąbała, Cornelis J. M. Melief, and Wolfgang Kastenmüller. 2018. “CD4+ T Cell Help in Cancer Immunology and Immunotherapy.” Nature Reviews. Immunology 18 (10): 635-47.
Bowes, J. H., and R. H. Kenten. 1948. “The Amino-Acid Composition and Titration Curve of Collagen.” Biochemical Journal 43 (3): 358-65.
Brown, Eric M., Xiaobo Ke, Daniel Hitchcock, Sarah Jeanfavre, Julian Avila-Pacheco, Toru Nakata, Timothy D. Arthur, et al. 2019. “Bacteroides-Derived Sphingolipids Are Critical for Maintaining Intestinal Homeostasis and Symbiosis.” Cell Host & Microbe 25 (5): 668-80.e7.
Carmona, Santiago J., Paula A. Sartor, María S. Leguizamón, Oscar E. Campetella, and Fernán Agüero. 2012. “Diagnostic Peptide Discovery: Prioritization of Pathogen Diagnostic Markers Using Multiple Features.” PloS One 7 (12): e50748.
Chemin, Karine, Sabrina Pollastro, Eddie James, Changrong Ge, Inka Albrecht, Jessica Herrath, Christina Gerstner, et al. 2016. “A Novel HLA-DRB1*10:01-Restricted T Cell Epitope From Citrullinated Type II Collagen Relevant to Rheumatoid Arthritis.” Arthritis & Rheumatology (Hoboken, N.J.) 68 (5): 1124-35.
Chen, Binbin, Michael S. Khodadoust, Niclas Olsson, Lisa E. Wagar, Ethan Fast, Chih Long Liu, Yagmur Muftuoglu, et al. 2019. “Predicting HLA Class II Antigen Presentation through Integrated Deep Learning.” Nature Biotechnology 37 (11): 1332-43.
Chong, Chloe, Fabio Marino, Huisong Pak, Julien Racle, Roy T. Daniel, Markus Müller, David Gfeller, George Coukos, and Michal Bassani-Sternberg. 2018. “High-Throughput and Sensitive Immunopeptidomics Platform Reveals Profound Interferony-Mediated Remodeling of the Human Leukocyte Antigen (HLA) Ligandome.” Molecular & Cellular Proteomics: MCP 17 (3): 533-48.
Dendrou, Calliope A., Jan Petersen, Jamie Rossjohn, and Lars Fugger. 2018. “HLA Variation and Disease.” Nature Reviews. Immunology 18 (5): 325-39.
Depommier, Clara, Amandine Everard, Céline Druart, Hubert Plovier, Matthias Van Hul, Sara Vieira-Silva, Gwen Falony, et al. 2019. “Supplementation with Akkermansia Muciniphila in Overweight and Obese Human Volunteers: A Proof-of-Concept Exploratory Study.” Nature Medicine, July. https://doi.org/10.1038/s41591-019-0495-2.
Dessen, A., C. M. Lawrence, S. Cupo, D. M. Zaller, and D. C. Wiley. 1997. “X-Ray Crystal Structure of HLA-DR4 (DRA*0101, DRB1*0401) Complexed with a Peptide from Human Collagen II.” Immunity 7 (4): 473-81.
Di Sante, Gabriele, Barbara Tolusso, Anna Laura Fedele, Elisa Gremese, Stefano Alivernini, Chiara Nicolò, Francesco Ria, and Gianfranco Ferraccioli. 2015. “Collagen Specific T-Cell Repertoire and HLA-DR Alleles: Biomarkers of Active Refractory Rheumatoid Arthritis.” EBioMedicine 2 (12): 2037-45.
Dong, De, Lvqin Zheng, Jianquan Lin, Bailing Zhang, Yuwei Zhu, Ningning Li, Shuangyu Xie, Yuhang Wang, Ning Gao, and Zhiwei Huang. 2019. “Structural Basis of Assembly of the Human T Cell Receptor-CD3 Complex.” Nature 573 (7775): 546-52.
Franzosa, Eric A., Alexandra Sirota-Madi, Julian Avila-Pacheco, Nadine Fornelos, Henry J. Haiser, Stefan Reinker, Tommi Vatanen, et al. 2019. “Gut Microbiome Structure and Metabolic Activity in Inflammatory Bowel Disease.” Nature Microbiology 4 (2): 293-305.
Gao, Shengyu, Ting-Wei Hsu, and Ming O. Li. 2021. “Immunity beyond Cancer Cells: Perspective from Tumor Tissue.” Trends in Cancer Research 7 (11): 1010-19.
Garde, Christian, Sri H. Ramarathinam, Emma C. Jappe, Morten Nielsen, Jens V. Kringelum, Thomas Trolle, and Anthony W. Purcell. 2019. “Improved Peptide-MHC Class II Interaction Prediction through Integration of Eluted Ligand and Peptide Affinity Data.” Immunogenetics 71 (7): 445-54.
Germain, R. N., and D. H. Margulies. 1993. “The Biochemistry and Cell Biology of Antigen Processing and Presentation.” Annual Review of Immunology 11:403-50.
Ghosh, P., M. Amaya, E. Mellins, and D. C. Wiley. 1995. “The Structure of an Intermediate in Class II MHC Maturation: CLIP Bound to HLA-DR3.” Nature 378 (6556): 457-62.
Glanville, Jacob, Huang Huang, Allison Nau, Olivia Hatton, Lisa E. Wagar, Florian Rubelt, Xuhuai Ji, et al. 2017. “Identifying Specificity Groups in the T Cell Receptor Repertoire.” Nature 547 (7661): 94-98.
Glaser, Fabian, Tal Pupko, Inbal Paz, Rachel E. Bell, Dalit Bechor-Shental, Eric Martz, and Nir Ben-Tal. 2003. “ConSurf: Identification of Functional Regions in Proteins by Surface-Mapping of Phylogenetic Information.” Bioinformatics 19 (1): 163-64.
Goodfellow, Ian, Yoshua Bengio, and Aaron Courville. 2016. Deep Learning. MIT Press.
Graham, Daniel B., Chengwei Luo, Daniel J. O'Connell, Ariel Lefkovith, Eric M. Brown, Moran Yassour, Mukund Varma, et al. 2018. “Antigen Discovery and Specification of Immunodominance Hierarchies for MHCII-Restricted Epitopes.” Nature Medicine 24 (11): 1762-72.
Grifoni, Alba, John Sidney, Randi Vita, Bjoern Peters, Shane Crotty, Daniela Weiskopf, and Alessandro Sette. 2021. “SARS-COV-2 Human T Cell Epitopes: Adaptive Immune Response against COVID-19.” Cell Host & Microbe 29 (7): 1076-92.
Heitmann, Jonas S., Tatjana Bilich, Claudia Tandler, Annika Nelde, Yacine Maringer, Maddalena Marconato, Julia Reusch, et al. 2022. “A COVID-19 Peptide Vaccine for the Induction of SARS-COV-2 T Cell Immunity.” Nature 601 (7894): 617-22.
Henrick, Bethany M., Lucie Rodriguez, Tadepally Lakshmikanth, Christian Pou, Ewa Henckel, Aron Arzoomand, Axel Olin, et al. 2021. “Bifidobacteria-Mediated Immune System Imprinting Early in Life.” Cell 184 (15): 3884-98.e11.
Honda, Kenya, and Dan R. Littman. 2016. “The Microbiota in Adaptive Immune Homeostasis and Disease.” Nature 535 (7610): 75-84.
Hong, Andrew L., Jennifer L. Guerriero, Mihir B. Doshi, Bryan D. Kynnap, Won Jun Kim, Anna C. Schinzel, Rebecca Modiste, et al. 2019. “MCL1 and DEDD Promote Urothelial Carcinoma Progression.” Molecular Cancer Research: MCR 17 (6): 1294-1304.
Hsing, Lianne C., and Alexander Y. Rudensky. 2005. “The Lysosomal Cysteine Proteases in MHC Class II Antigen Presentation.” Immunological Reviews 207 (October): 229-41.
Jensen, Kamilla Kjaergaard, Massimo Andreatta, Paolo Marcatili, Søren Buus, Jason A. Greenbaum, Zhen Yan, Alessandro Sette, Bjoern Peters, and Morten Nielsen. 2018. “Improved Methods for Predicting Peptide Binding Affinity to MHC Class II Molecules.” Immunology 154 (3): 394-406.
Johnson, L. Steven, Sean R. Eddy, and Elon Portugaly. 2010. “Hidden Markov Model Speed Heuristic and Iterative HMM Search Procedure.” BMC Bioinformatics 11 (August): 431.
Jones, E. Yvonne, Lars Fugger, Jack L. Strominger, and Christian Siebold. 2006. “MHC Class II Proteins and Disease: A Structural Perspective.” Nature Reviews. Immunology 6 (4): 271-82.
Jurewicz, Mollie M., and Lawrence J. Stern. 2019. “Class II MHC Antigen Processing in Immune Tolerance and Inflammation.” Immunogenetics 71 (3): 171-87.
Keeton, Roanne, Marius B. Tincho, Amkele Ngomti, Richard Baguma, Ntombi Benede, Akiko Suzuki, Khadija Khan, et al. 2022. “T Cell Responses to SARS-COV-2 Spike Cross-Recognize Omicron.” Nature, January. https://doi.org/10.1038/s41586-022-04460-3.
Kingma, Diederik P., and Jimmy Ba. 2014. “Adam: A Method for Stochastic Optimization.” arXiv [cs.LG]. arXiv. http://arxiv.org/abs/1412.6980.
Kjellén, Peter, Ulrica Brunsberg, Johan Broddefalk, Bjarke Hansen, Mikael Vestberg, Ianeric Ivarsson, Åke Engström, et al. 1998. “The Structural Basis of MHC Control of Collagen-Induced Arthritis; Binding of the Immunodominant Type II Collagen 256-270 Glycopeptide to H-2Aq and H-2Ap Molecules.” European Journal of Immunology 28 (2): 755-66.
Klaeger, Susan, Annie Apffel, Karl R. Clauser, Siranush Sarkizova, Giacomo Oliveira, Suzanna Rachimi, Phuong M. Le, et al. 2021. “Optimized Liquid and Gas Phase Fractionation Increases HLA-Peptidome Coverage for Primary Cell and Tissue Samples.” Molecular & Cellular Proteomics: MCP 20 (August): 100133.
Klausen, Michael Schantz, Martin Closter Jespersen, Henrik Nielsen, Kamilla Kjaergaard Jensen, Vanessa Isabell Jurtz, Casper Kaae Sønderby, Morten Otto Alexander Sommer, et al. 2019. “NetSurfP-2.0: Improved Prediction of Protein Structural Features by Integrated Deep Learning.” Proteins 87 (6): 520-27.
Krogh, A., B. Larsson, G. von Heijne, and E. L. Sonnhammer. 2001. “Predicting Transmembrane Protein Topology with a Hidden Markov Model: Application to Complete Genomes.” Journal of Molecular Biology 305 (3): 567-80.
Landau, Meytal, Itay Mayrose, Yossi Rosenberg, Fabian Glaser, Eric Martz, Tal Pupko, and Nir Ben-Tal. 2005. “ConSurf 2005: The Projection of Evolutionary Conservation Scores of Residues on Protein Structures.” Nucleic Acids Research 33 (Web Server issue): W299-302.
Lipsitch, Marc, Yonatan H. Grad, Alessandro Sette, and Shane Crotty. 2020. “Cross-Reactive Memory T Cells and Herd Immunity to SARS-COV-2.” Nature Reviews. Immunology 20 (11): 709-13.
Lloyd-Price, Jason, Cesar Arze, Ashwin N. Ananthakrishnan, Melanie Schirmer, Julian Avila-Pacheco, Tiffany W. Poon, Elizabeth Andrews, et al. 2019. “Multi-Omics of the Gut Microbial Ecosystem in Inflammatory Bowel Diseases.” Nature 569 (7758): 655-62.
Marcu, Ana, Leon Bichmann, Leon Kuchenbecker, Daniel Johannes Kowalewski, Lena Katharina Freudenmann, Linus Backert, Lena Mühlenbruch, et al. 2021. “HLA Ligand Atlas: A Benign Reference of HLA-Presented Peptides to Improve T-Cell-Based Cancer Immunotherapy.” Journal for Immunotherapy of Cancer 9 (4). https://doi.org/10.1136/jitc-2020-002071.
May, Damon H., Benjamin E. R. Rubin, Sudeb C. Dalai, Krishna Patel, Shahin Shafiani, Rebecca Elyanow, Matthew T. Noakes, Thomas M. Snyder, and Harlan S. Robins. 2021. “Immunosequencing and Epitope Mapping Reveal Substantial Preservation of the T Cell Immune Response to Omicron Generated by SARS-COV-2 Vaccines.” bioRxiv. https://doi.org/10.1101/2021.12.20.21267877.
Mistry, Jaina, Sara Chuguransky, Lowri Williams, Matloob Qureshi, Gustavo A. Salazar, Erik L. L. Sonnhammer, Silvio C. E. Tosatto, et al. 2021. “Pfam: The Protein Families Database in 2021.” Nucleic Acids Research 49 (D1): D412-19.
Naranbhai, Vivek, Anusha Nathan, Clarety Kaseke, Cristhian Berrios, Ashok Khatri, Shawn Choi, Matthew A. Getz, et al. 2022. “T Cell Reactivity to the SARS-COV-2 Omicron Variant Is Preserved in Most but Not All Prior Infected and Vaccinated Individuals.” medRxiv: The Preprint Server for Health Sciences, January. https://doi.org/10.1101/2022.01.04.21268586.
Neefjes, Jacques, Marlieke L. M. Jongsma, Petra Paul, and Oddmund Bakke. 2011. “Towards a Systems Understanding of MHC Class I and MHC Class II Antigen Presentation.” Nature Reviews. Immunology 11 (12): 823-36.
Nielsen, Henrik. 2017. “Predicting Secretory Proteins with SignalP.” Methods in Molecular Biology 1611:59-73.
Oh, David Y., and Lawrence Fong. 2021. “Cytotoxic CD4+ T Cells in Cancer: Expanding the Immune Effector Toolbox.” Immunity 54 (12): 2701-11.
Peters, Bjoern, Morten Nielsen, and Alessandro Sette. 2020. “T Cell Epitope Predictions.” Annual Review of Immunology 38 (April): 123-45.
Plichta, Damian R., Daniel B. Graham, Sathish Subramanian, and Ramnik J. Xavier. 2019. “Therapeutic Opportunities in Inflammatory Bowel Disease: Mechanistic Dissection of Host-Microbiome Relationships.” Cell 178 (5): 1041-56.
Plichta, Damian R., Juhi Somani, Matthieu Pichaud, Zachary S. Wallace, Ana D. Fernandes, Cory A. Perugino, Harri Lähdesmäki, et al. 2021. “Congruent Microbiome Signatures in Fibrosis-Prone Autoimmune Diseases: IgG4-Related Disease and Systemic Sclerosis.” Genome Medicine 13 (1): 35.
Pos, Wouter, Dhruv K. Sethi, Melissa J. Call, Monika-Sarah E. D. Schulze, Anne-Kathrin Anders, Jason Pyrdol, and Kai W. Wucherpfennig. 2012. “Crystal Structure of the HLA-DM-HLA-DR1 Complex Defines Mechanisms for Rapid Peptide Selection.” Cell 151 (7): 1557-68.
Racle, Julien, Justine Michaux, Georg Alexander Rockinger, Marion Arnaud, Sara Bobisse, Chloe Chong, Philippe Guillaume, et al. 2019. “Robust Prediction of HLA Class II Epitopes by Deep Motif Deconvolution of Immunopeptidomes.” Nature Biotechnology 37 (11): 1283-86.
Radwan, Jacek, Wiesław Babik, Jim Kaufman, Tobias L. Lenz, and Jamie Winternitz. 2020. “Advances in the Evolutionary Understanding of MHC Polymorphism.” Trends in Genetics: TIG 36 (4): 298-311.
Rappazzo, C. Garrett, Brooke D. Huisman, and Michael E. Birnbaum. 2020. “Repertoire-Scale Determination of Class II MHC Peptide Binding via Yeast Display Improves Antigen Prediction.” Nature Communications 11 (1): 4414.
Renz, Harald, and Chrysanthi Skevaki. 2021. “Early Life Microbial Exposures and Allergy Risks: Opportunities for Prevention.” Nature Reviews. Immunology 21 (3): 177-91.
Reyes-Vargas, Eduardo, Adam P. Barker, Zemin Zhou, Xiao He, and Peter E. Jensen. 2020. “HLA-DM Catalytically Enhances Peptide Dissociation by Sensing Peptide-MHC Class II Interactions throughout the Peptide-Binding Cleft.” The Journal of Biological Chemistry 295 (10): 2959-73.
Reynisson, Birkir, Bruno Alvarez, Sinu Paul, Bjoern Peters, and Morten Nielsen. 2020. “NetMHCpan-4.1 and NetMHCIIpan-4.0: Improved Predictions of MHC Antigen Presentation by Concurrent Motif Deconvolution and Integration of MS MHC Eluted Ligand Data.” Nucleic Acids Research 48 (W1): W449-54.
Reynisson, Birkir, Carolina Barra, Saghar Kaabinejadian, William H. Hildebrand, Bjoern Peters, and Morten Nielsen. 2020. “Improved Prediction of MHC II Antigen Presentation through Integration and Motif Deconvolution of Mass Spectrometry MHC Eluted Ligand Data.” Journal of Proteome Research 19 (6): 2304-15.
Reynolds, Lisa A., and B. Brett Finlay. 2017. “Early Life Factors That Affect Allergy Development.” Nature Reviews. Immunology 17 (8): 518-28.
Ricci, Alejandro D., Mauricio Brunner, Diego Ramoa, Santiago J. Carmona, Morten Nielsen, and Fernán Agüero. 2021. “APRANK: Computational Prioritization of Antigenic Proteins and Peptides From Complete Pathogen Proteomes.” Frontiers in Immunology 12 (July): 702552.
Robinson, James, Dominic J. Barker, Xenia Georgiou, Michael A. Cooper, Paul Flicek, and Steven G. E. Marsh. 2020. “IPD-IMGT/HLA Database.” Nucleic Acids Research 48 (D1): D948-55.
Schulze, Monika-Sarah E. D., Anne-Kathrin Anders, Dhruv K. Sethi, and Melissa J. Call. 2013. “Disruption of Hydrogen Bonds between Major Histocompatibility Complex Class II and the Peptide N-Terminus Is Not Sufficient to Form a Human Leukocyte Antigen-DM Receptive State of Major Histocompatibility Complex Class II.” PloS One 8 (7): e69228.
Schwanhäusser, Björn, Dorothea Busse, Na Li, Gunnar Dittmar, Johannes Schuchhardt, Jana Wolf, Wei Chen, and Matthias Selbach. 2011. “Global Quantification of Mammalian Gene Expression Control.” Nature 473 (7347): 337-42.
Serra, Pau, Nahir Garabatos, Santiswarup Singha, César Fandos, Josep Garnica, Patricia Solé, Daniel Parras, et al. 2019. “Increased Yields and Biological Potency of Knob-into-Hole-Based Soluble MHC Class II Molecules.” Nature Communications 10 (1): 4917.
Sette, Alessandro, and Shane Crotty. 2021. “Adaptive Immunity to SARS-COV-2 and COVID-19.” Cell 184 (4): 861-80.
Shrock, Ellen, Eric Fujimura, Tomasz Kula, Richard T. Timms, I-Hsiu Lee, Yumei Leng, Matthew L. Robinson, et al. 2020. “Viral Epitope Profiling of COVID-19 Patients Reveals Cross-Reactivity and Correlates of Severity.” Science 370 (6520). https://doi.org/10.1126/science.abd4250.
Stern, L. J., J. H. Brown, T. S. Jardetzky, J. C. Gorga, R. G. Urban, J. L. Strominger, and D. C. Wiley. 1994. “Crystal Structure of the Human Class II MHC Protein HLA-DR1 Complexed with an Influenza Virus Peptide.” Nature 368 (6468): 215-21.
Tunyasuvunakool, Kathryn, Jonas Adler, Zachary Wu, Tim Green, Michal Zielinski, Augustin Žídek, Alex Bridgland, et al. 2021. “Highly Accurate Protein Structure Prediction for the Human Proteome.” Nature 596 (7873): 590-96.
Unanue, Emil R., Vito Turk, and Jacques Neefjes. 2016. “Variations in MHC Class II Antigen Processing and Presentation in Health and Disease.” Annual Review of Immunology 34 (May): 265-97.
Vita, Randi, Swapnil Mahajan, James A. Overton, Sandeep Kumar Dhanda, Sheridan Martini, Jason R. Cantrell, Daniel K. Wheeler, Alessandro Sette, and Bjoern Peters. 2019. “The Immune Epitope Database (IEDB): 2018 Update.” Nucleic Acids Research 47 (D1): D339-43.
Vizcaíno, Juan Antonio, Peter Kubiniok, Kevin A. Kovalchik, Qing Ma, Jérôme D. Duquette, Ian Mongrain, Eric W. Deutsch, et al. 2020. “The Human Immunopeptidome Project: A Roadmap to Predict and Treat Immune Diseases.” Molecular & Cellular Proteomics: MCP 19 (1): 31-49.
Vyas, Jatin M., Annemarthe G. Van der Veen, and Hidde L. Ploegh. 2008. “The Known Unknowns of Antigen Processing and Presentation.” Nature Reviews. Immunology 8 (8): 607-18.
Willis, Richard A., Vasanthi Ramachandiran, John C. Shires, Ge Bai, Kelly Jeter, Donielle L. Bell, Lixia Han, et al. 2021. “Production of Class II MHC Proteins in Lentiviral Vector-Transduced HEK-293T Cells for Tetramer Staining Reagents.” Current Protocols 1 (2): e36.
Yin, Liusong, Peter Trenh, Abigail Guce, Marek Wieczorek, Sascha Lange, Jana Sticht, Wei Jiang, et al. 2014. “Susceptibility to HLA-DM Protein Is Determined by a Dynamic Conformation of Major Histocompatibility Complex Class II Molecule Bound with Peptide.” The Journal of Biological Chemistry 289 (34): 23449-64.
Zheng, Ming Z. M., and Linda M. Wakim. 2021. “Tissue Resident Memory T Cells in the Respiratory Tract.” Mucosal Immunology, October.

### Supplementary Notes: HLA-II Immunopeptidome Profiling and Deep Learning Reveal Features of Antigenicity to Inform Antigen Discovery

Applicants hereby outline the details of implemented model formulations and design choices for CAPTAn, consisting of submodules CAPTAn-core and CAPTAn-context.

### 1. Binding Core Models (CAPTAn-Core)

The first component of the modeling framework, CAPTAn-core, predicts peptide ligands for short peptides of length L € [7, 50].

### 1.1 Model Formulation

A classifier is trained for each of the 87 alleles in the study as follows. The input peptide sequences Sₚₑₚ with length up to L are represented as binary arrays Sₚₑₚ ∈{0, 1} 4×20 and associated with a binary variable y∈{0, 1} distinguishing binders and non-binders. The allele-specific classification task infers the conditional probability p(y=1|Sₚₑₚ)=f_{core} (Sₚₑₚ), where probability estimator f is implemented by a neural network.

In the following, Applicants assume operations that transform the sequence matrix. Upper-case bold characters represent matrices, lower-case bold characters are vectors and lower-case italic characters are scalars. Applicants use named functions for common operations in deep learning literature (Goodfellow et al., 2016), which are also defined in more detail below.

The input amino acid sequence is scanned with two layers of convolutional windows using a window size l, which approximates the lengths of ligands obtained in peptidomics assays.

$$A = reLU ( convolution ( {S}_{pep} , l ) ) ∈ {{R}_{+}}^{L \times 64} , l ∈ \{ 10 , 15 \} \qquad ( 1 )$$

$$B = reLU ( convolution ( A , l ) ) ∈ {R}^{L \times 64}$$

The activations at layer B are pooled within windows of size l/2, which assumes that the candidate binding core motifs are spaced by at least l/2 amino acids. Two additional hidden layers are added, followed by a dropout operation to control for overfitting.

$$C = pool ( B ) ∈ R \hat{} ( L \times 64 {d}_{C} ) , k = l / 2 \qquad ( 2 )$$

$$D = reLU ( linear ( C ) ) ∈ {R}_{+} \hat{} ( L \times {d}_{D} )$$

$$E = reLU ( linear ( D ) ) ∈ {R}_{+} \hat{} ( ( L \times {d}_{D} ) / 2 )$$

$$F = dropout ( E , {p}_{d} ) ∈ {R}_{+} \hat{} ( ( L \times {d}_{D} ) / 2 ) , {p}_{d} ∈ \{ 0 , 0.1 , 0.3 \}$$

The final probability estimate for the sequence Sₚₑₚ consists of finding activations with maximum values across the whole sequence length, conveying that binding core motifs or other important patterns can appear anywhere in the sequence. The activations in layer F are reduced (aggregated) across the sequence length by either maximum or sum across each latent dimension. This results in a vector of activations g that is now independent of sequence length. The final mapping produces a binding probability estimate z.

$$g = reduce ( F , op ) ∈ {R}_{+} \hat{} ( {d}_{D} / 2 ) , op ∈ \{ max , sum \} \qquad ( 3 )$$

$$z = {f}_{core} ( {S}_{pep} ) = sigmoid ( linear ( g ) ) ∈ ( 0 , 1 )$$

### 1.2 Parameter Optimization

The model training uses ligands for each allele separated into five approximately equal parts as described above. Three parts are used for parameter optimization and one part is used as stopping criterion (early stopping). The remaining part is used to evaluate the expected classification performance on unseen data.

Each of the 87 allele-specific models is trained five times, where each part of the training set is used as a test set once. The classification loss function is optimized with the Adam algorithm (Kingma and Ba, 2014) for at most 40 epochs, with early stopping if validation set loss stops decreasing after 5 consecutive epochs.

The loss at for single peptide sequence Sₚₑₚ {0, 1}^{L×20}, binder label y∈{0, 1} and core model prediction z=f_{core}(Sₚₑₚ)∈(0, 1) is computed as binary entropy

$${L}_{core} ( y , z ) = - ( y log ( z ) + ( 1 - y ) log ( 1 - z ) ) \qquad ( 4 )$$

and the total loss is averaged across all peptides a mini batches of size 128.

### 1.3 Hyperparameter Tuning

The following model hyperparameters are subject to a grid search:
1. convolution window size l∈{10, 15},
2. usage of the pooling operation yielding C (yes or no),
3. dimension of hidden layers, d_{D}∈{32, 128},
4. dropout probability p_{d} ∈{0, 0.1, 0.3},
5. reduction operation (sum or max).
   The grid search thus consists of 48 parameter configurations. The best performing parameter set is selected based on the lowest average validation loss in across five training data splits described in Section 1.2. The number of trainable model parameter ranges from 56,449 (for l=10, d_{D}=32)−97,345 (l=15, d_{D}=128).

### 2. Isotype-Specific Protein Context Model (CAPTAn-Context)

In the following, Applicants describe the context model use to predict general epitope regions for DP, DQ, DR isotypes. To this end, Applicants map all epitopes for alleles in the DP, DQ, DR isotype to their corresponding proteins of origin, obtaining a single binary sequence per each isotype and protein.

### 2.1 Model Formulation

The sequence is processed with 128 convolutional layers to capture informative sequence motifs. This is followed by two consecutive applications of forward and backward long short term memory (LSTM) units (Section 4) that map each position in a sequence to an embedding vector influenced by all the preceding and following positions. Thus each amino acid in a protein affects the predictions at all other amino acids, allowing to capture long-range dependencies.

The input consist of a whole protein sequence Sₚᵣₒ∈R_{L×20} and the target variables Y∈{0, 1}^{L}.

$$A = reLU ( convolution ( {S}_{pro} , l = 20 ) ) ∈ {R}^{L \times 128} \qquad ( 5 )$$

$$B = LSTM ( A ) ∈ {R}^{L \times 32}$$

$$C = bLSTM ( A ) ∈ {R}^{L \times 32}$$

$$D = concatenate ( B , C ) ∈ {R}^{L \times 64}$$

$$E = LSTM ( D ) ∈ {R}^{L \times 32}$$

$$F = bLSTM ( D ) ∈ {R}^{L \times 32}$$

$$G = concatenate ( E , F ) ∈ {R}^{L \times 64}$$

The final decision is obtained by combining dropout and reLU-linear layers and mapping the obtained outputs to probabilities at each position in the sequence.

$$H = reLU ( linear ( dropout ( G , p = 0.1 ) ) ) ∈ {R}^{L \times 48} \qquad ( 6 )$$

$$I = reLU ( linear ( dropout ( H , p = 0.07 ) ) ) ∈ {R}^{L \times 40}$$

$$J = reLU ( linear ( dropout ( I , p = 0.04 ) ) ) ∈ {R}^{L \times 33}$$

$$K = reLU ( lineardropout ( J , p = 0.03 ) ∈ {R}^{L \times 27}$$

$$z = {f}_{context} ( {S}_{pro} ) = sigmoid ( linear ( K ) ) ∈ {( 0 , 1 )}^{L}$$

### 2.2 Optimization and Training

The number of trainable parameters resulting from the model definition in Section 2.1 equals 127,363. Each of the 3 isotype-specific models is trained five times, where each part of the training set is used as a test set once. The training set splits are consistent with core model optimization in Section 1.2.

Protein sequences are split into 112 mini batches, which was the maximal number to ensure that each batch contained at least one protein with an epitope. To minimize the amount of padding due to different protein lengths, K-means clustering with K=112 is used to create mini batches of comparable logarithms of sequence length. Mini batches are further split in half prior to each gradient evaluation, so that gradients are computed on different sets of sequences in each iteration.

The classification loss function is optimized with the Adam algorithm (Kingma and Ba, 2014) for at most 100 epochs, with early stopping if validation set loss stops decreasing after 5 consecutive epochs.

The loss for a single sequence Sₚᵣₒ ∈{0, 1}^{L×20}, epitope regions y∈{0, 1}^{L}, and model prediction z=f_{context} (Sₚᵣₒ)∈(0, 1)^{L} is defined as the weighted binary entropy across all amino acids in a protein:

$${L}_{context} ( y , z ) = - {\sum }_{t = 1}^{L} {w}_{t} ( {y}_{t} log ( {z}_{t} ) + ( 1 - {y}_{t} ) log ( 1 - {z}_{t} ) ) \qquad ( 7 )$$

and the total loss is averaged across all sequences in a mini batch. Each position in a sequence is weighted using the following scheme:

$${w}_{t} = \{ \begin{matrix}\frac{( 1 - p ) L}{max ( 1 , {\sum }_{j = 1}^{L} 1 - {y}_{j} )} & if {y}_{t} = 0 , \\ 1 , & otherwise\end{matrix} . \qquad ( 8 )$$

Here, p is prior epitope probability for each of the three isotypes. From the point of view of the predictive distribution optimization, this ensures the same perceived prior probability of epitope regardless of sequence length. The negative examples in shorter sequences are penalized to prevent model exploiting the fact that epitopes appear more dense in shorter sequences. On the other hand, the equal weighing of positive examples ensures their ap proximately equal treatment across sequences of varying lengths.

### 3. Ensemble Model (CAPTAn)

The core and context models are combined to yield a final prediction using the following scheme. In it, Applicants assume that binding cores can be predicted within 20 amino acids of the starting positions, and that context information is aggregated across multiple neighboring positions using the maximum or sum.

For each protein and allele, the predictions for core and context models (for the relevant isotype) are computed as described above. The final prediction q (t) at each position is obtained as follows:

$$z = {f}_{context} ( S )$$

$$q ( t ) = {f}_{ensemble} = w reduce ( z ( t - a : t + b ) , op ) + ( 1 - w ) {f}_{core} ( S ( t : t + l ) ,$$

where l=20, while a (left interval), b (right interval) w (context model weight) and op (aggregating operation sum or max) are tunable parameters. The four parameters are optimized for each allele independently using grid search, where a∈{5, 10}, b∈{5, 10, 20}, w∈{0.05, 0.1, 0.15, . . . , 0.95} op∈{sum, max} and selected based on accuracy in top 100 prioritized epitopes using 30% of the training data.

### 4. Definitions

The following functions on input scalars x, vectors x∈R^{d} or matrices X∈R^{L×d} constitute the described models. In this application, L represents the sequence (peptide or protein) length, while d is the feature space dimension (amino acids).

The additional symbols listed as function arguments represent set hyperparameters, while other symbols in function definitions represent trainable parameters.

Sigmoid. Map x to the interval (0, 1).

$$sigmoid ( x ) = \frac{1}{1 + exp ( - x )}$$

Concatenate. Create a new vector by stacking two or more input vectors. concatenate (x, z)=(x₁, x₂, . . . x_{d1}, z₁, z₂, . . . , z_{d2})
Rectified linear units (reLU). Set negative x to zero.

$$reLU ( x ) = \{ \begin{matrix}x , & if x \geq 0 \\ 0 , & otherwise\end{matrix}$$

Linear units. Linear transform of an input vector with trainable parameters W and b.

$$linear ( x ) = Wx + b$$

Convolution (1D). Aggregate consecutive sequence elements in a window of size l, with trainable parameters W and b. The input X is assumed to represent a sequence in a matrix format where the rows represent sequence length L and columns sequence features.

$$convolution ( X , l ) = y , y ( t ) = {\sum }_{i = t}^{t + l} WX ( i ) + b$$

Dropout. Set elements of x to zero with probability p. Each function call assumes a randomly generated number r∈(0, 1).

$$dropout ( x , p ) = y , y ( t ) = \{ \begin{matrix}x ( t ) , & if r < 1 - p \\ 0 , & otherwise\end{matrix}$$

Pool. Select maximum element of x∈R^{d} in a pooling window of size l. Zero-padding is assumed for entries of y where t+1>d.

$$y ( t ) = \{ \begin{matrix}max ( x ( t : t + l ) ) , & if t + l \leq d \\ 0 , & otherwise\end{matrix} .$$

Reduce. Map x to a scalar using maximum or sum over its elements.
reduce (x, max)=max (x)

$$reduce ( x , sum ) = {\sum }_{t = 1}^{d} x ( t )$$

Long-short term memory unit (LSTM). A recurrent mapping where the elements on the input X are assumed to represent a sequence in a matrix format where the rows represent sequence length L and columns sequence features. The computation of output matrix H proceeds iteratively for each element in the sequence t=1 . . . d. The computation uses multiple internal states: the forget gate F(t), input gate G(t), internal state S(t), and the output gate Q(t). The value at H(t) is thus influenced by all previous inputs X(1: t−1) as well as current input X(t). All state variables include corresponding trainable parameters W and b.

LSTM(X)=H,

$$F ( t ) = sigmoid ( {b}_{f} + {U}_{f} X ( t ) + {W}_{f} H ( t - 1 ) )$$

$$G ( t ) = sigmoid ( {b}_{g} + {U}_{g} X ( t ) + {W}_{g} H ( t - 1 ) )$$

$$S ( t ) = F ( t ) S ( t - 1 ) + G ( t ) sigmoid ( {b}_{s} + {U}_{s} X + {W}_{s} H ( t - 1 ) )$$

$$Q ( t ) = sigmoid ( {b}_{q} + {U}_{q} X ( t ) + {W}_{q} H ( t - 1 ) )$$

H(t)=tan h(S(t))Q(t)

An analogous version is the backward computation (bLS™), where the computation proceeds in the reverse order, t=d, d−1, . . . , 1.

### REFERENCES FOR SUPPLEMENTARY NOTES

Ian Goodfellow, Yoshua Bengio, and Aaron Courville. Deep learning. MIT press, 2016.
Diederik P Kingma and Jimmy Ba. Adam: A method for stochastic optimization. ar Xiv preprint ar Xiv:1412.6980, 2014.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A computer-implemented method to generate one or more immunogenic peptides comprising one or more peptide binding motifs for use in immunological compositions, comprising:
a) receiving, by an acquisition engine communicatively coupled to a user device, one or more amino acid sequences;
b) transferring, by the acquisition engine, the one or more amino acid sequences to a deployed machine learning network communicatively coupled to the acquisition engine;
c) processing the one or more amino acid sequences with the deployed machine learning network, the deployed machine learning network generated and deployed from a training machine learning network; and
d) generating one or more immunogenic peptides comprising one or more peptide binding motifs.

2. The method of claim 1, further comprising:
e) receiving, by an acquisition engine communicatively coupled to a user device, a second one or more amino acid sequences;
f) transferring, by the acquisition engine, the second one or more amino acid sequences to a second deployed machine learning network communicatively coupled to the acquisition engine;
g) processing the second one or more amino acid sequences with the second deployed machine learning network, the second deployed machine learning network generated and deployed from a second training machine learning network;
h) generating one or more ligand regions of the second one or more amino acid sequences;
optionally further comprising:
i) transferring, by the acquisition engine, one or more immunogenic peptides comprising one or more peptide binding motifs and the one or more ligand regions to an ensemble network communicatively coupled to the acquisition engine;
j) processing the one or more peptide binding motifs and the one or more ligand regions with the ensemble network, optionally wherein the ensemble network comprises grid search;
k) generating a refined set of one or more immunogenic peptides comprising one or more peptide binding motifs; and
optionally further comprising preparing one or more immunogenic peptides for an immunological composition, optionally wherein the immunological composition is a protective vaccine or tolerizing vaccine composition comprising one or more antigenic epitopes and/or
optionally further comprising detecting whether one or more of the antigenic epitopes is present in a sample from a subject suffering from an infection, autoimmune disease, allergy, or cancer.

3.-4. (canceled)

5. The method of claim 2, wherein the deployed machine learning network and/or second deployed machine learning networks receive one or more features of the one or more amino acid sequences, optionally wherein the one or more features comprise binary, fractional, or both features, optionally wherein the fractional features comprise secreted protein features, transmembrane features, domain features, region features, relative solvent accessibility features, and disorder features.

6.-7. (canceled)

8. The method of claim 2, wherein the generated immunogenic peptides comprising one or more peptide binding motifs further comprise individual probability or confidence scores, optionally wherein the generated one or more ligand regions further comprise individual probability or confidence scores at each position in the ligand region;
wherein the immunogenic peptide comprising one or more peptide binding motifs is specific for one or more HLA II alleles selected from the group consisting of those in Table 1;
or any combination thereof.

9.-10. (canceled)

11. The method of claim 2, wherein the second one or more amino acid sequences comprise signaling regions, optionally wherein the signaling regions comprise adjacent and/or distant signaling regions, and optionally wherein the adjacent signaling regions comprise exopeptidase trimming sites and/or proline-rich cleavage motifs.

12.-13. (canceled)

14. The method of claim 2, wherein the second one or more amino acid sequences comprises a full length amino acid sequence of a protein, or wherein the second one or more amino acid sequences comprises the one or more amino acid sequences, wherein the one or more amino acid sequences further comprises one or more additional sequences up to the full length amino acid sequence of a source protein and optionally wherein the second one or more amino acid sequences are expanded sequences of the one or more amino acids.

15.-16. (canceled)

17. The method of claim 2, wherein the one or more amino acid sequences are of 7 to 100, 7 to 75, 7 to 50, or 7 to 25 amino acids in length;
wherein the one or more amino acid sequences have a maximum overlap of 10, 9, 8, 7, 6, or 5 amino acids;
wherein the one or more peptide binding motifs is between 5 to 100, 5 to 75, 5 to 50, 5 to 25 amino acids in length;
wherein the immunogenic peptide comprising one or more peptide binding motifs is 12 to 100, 12 to 75, 12 to 50, 12 to 25, or around 20 amino acids in length;
wherein the ligand region further comprises an additional 25 amino acids on either or both sides of the generated ligand region; and
or any combination thereof.

18.-22. (canceled)

23. The method of claim 2, wherein the deployed machine learning network and second deployed machine learning network independently comprises linear classifiers, logistic classifiers, Bayesian networks, random forest, neural networks, matrix factorization, hidden Markov model, support vector machine, K-means clustering, or K-nearest neighbor.

24. The method of claim 1, wherein the deployed machine learning network comprises a neural network, optionally wherein the neural network comprises a convolutional neural network, and optionally wherein the deployed machine learning network comprises embedding.

25.-26. (canceled)

27. The method of claim 2, wherein the second deployed machine learning network comprises a neural network, optionally wherein the neural network comprises a convolutional neural network or a recurrent neural network, and optionally wherein the deployed machine learning network comprises embedding.

28.-30. (canceled)

31. The method of claim 2, wherein the deployed machine learning network and second deployed machine learning network independently comprises unsupervised learning, supervised learning, semi-supervised learning, reinforcement learning, transfer learning, incremental learning, curriculum learning, or learning to learn,
optionally wherein the machine learning network in (b) is trained on HLA-II allele-specific peptidomics data;
optionally wherein the second machine learning network is trained on full length amino acid sequences of the source proteins mapped back from HLA-II allele-specific peptidomics data, thereby generating the one or more regions of full-length proteins affecting the one or more peptide binding motifs; and
optionally wherein training the first deployed machine learning network comprises decoys at a 4:1, 5:1, or 6:1 ratio to immunogenic peptides.

32.-35. (canceled)

36. The method of claim 2, wherein the second deployed machine learning network comprises bi-directional long-short term memory (LSTM), optionally wherein the bi-directional LSTM is performed more than once.

37. (canceled)

38. The method of claim 2, wherein the deployed machine learning network further comprising pooling, reduction, dropout steps, or any combination thereof and/or wherein parameters of the deployed machine learning network and/or the second deployed machine learning network is tuned to minimize binary entropy loss.

39. (canceled)

40. The method of claim 1, wherein the immunogenic peptide comprising one or more peptide binding motifs are specific to HLA-II alleles specific to a subject;
wherein the one or more amino acid sequences comprise full-length protein sequences;
wherein the one or more amino acid sequences are obtained by analyzing one or more genomic DNA sequences, optionally wherein the one or more genomic DNA sequences is a full genome sequence;
or any combination thereof.

41.-43. (canceled)

44. The method of claim 40, wherein the one or more genome sequences is derived from a target pathogen, a commensal microorganism, or a diseased cell, optionally wherein the pathogen is selected from the group consisting of a bacterium, a virus, a protozoon, and an allergen, and optionally wherein the diseased cell is a cancer cell.

45.-46. (canceled)

47. The method of claim 1, wherein the one or more amino acid sequences are derived from neoantigens.

48.-49. (canceled)

50. A system to generate one or more immunogenic peptides comprising one or more peptide binding motifs for use in immunological compositions, comprising:
a storage device; and
a processor communicatively coupled to the storage device, wherein the processor executes application code instructions that are stored in the storage device to cause the system to:
a) receive, by an acquisition engine communicatively coupled to a user device, one or more amino acid sequences;
b) transfer the one or more amino acid sequences with an acquisition engine communicatively coupled to a deployed machine learning network;
c) process the one or more amino acid sequences with a deployed machine learning network, the deployed machine learning network generated and deployed from a training machine learning network; and
d) generate the one or more immunogenic peptides comprising one or more immunogenic peptide binding motifs.

51. The system of claim 50, wherein processing of step c) further comprises:
e) receive, by an acquisition engine communicatively coupled to a user device, a second one or more amino acid sequences;
f) transfer the second one or more amino acid sequences with the acquisition engine communicatively coupled to a second deployed machine learning network;
g) process the second one or more amino acid sequences with the second deployed machine learning network, the second deployed machine learning network generated and deployed from a second training machine learning network; and
h) generate one or more ligand regions of the second one or more amino acid sequences;
optionally further comprising:
i) transferring, by the acquisition engine, one or more immunogenic peptides comprising one or more peptide binding motifs and the one or more ligand regions to an ensemble network communicatively coupled to the acquisition engine;
j) processing the one or more peptide binding motifs and the one or more ligand regions with the ensemble network, optionally wherein the ensemble network comprises grid search;
k) generating a refined set of one or more immunogenic peptides comprising one or more peptide binding motifs; and
optionally further comprising preparing one or more immunogenic peptides for an immunological composition, optionally wherein the immunological composition is a protective vaccine or tolerizing vaccine composition comprising one or more antigenic epitopes and/or
optionally further comprising detecting whether one or more of the antigenic epitopes is present in a sample from a subject suffering from an infection, autoimmune disease, allergy, or cancer.

52.-53. (canceled)

54. The system of claim 51, wherein the deployed machine learning network and/or second deployed machine learning networks receive one or more features of the one or more amino acid sequences, optionally wherein the one or more features comprise binary, fractional, or both features, optionally wherein the fractional features comprise secreted protein features, transmembrane features, domain features, region features, relative solvent accessibility features, and disorder features.

55.-56. (canceled)

57. The system of claim 51, wherein the generated immunogenic peptide comprising one or more peptide binding motifs further comprises individual probability or confidence scores, optionally wherein the generated one or more ligand regions further comprise individual probability or confidence scores at each position in the ligand region;
wherein the immunogenic peptide comprising one or more peptide binding motifs is specific for one or more HLA II alleles selected from the group consisting of those in Table 1;
or any combination thereof.

58.-59. (canceled)

60. The system of claim 51, wherein the second one or more amino acid sequences comprise signaling regions, optionally wherein the signaling regions comprise adjacent and/or distant signaling regions, and optionally wherein the adjacent signaling regions comprise exopeptidase trimming sites and/or proline-rich cleavage motifs.

61.-62. (canceled)

63. The system of claim 51, wherein the second one or more amino acid sequences comprises the amino acid sequence of a protein, or wherein the second one or more amino acid sequences comprises the one or more amino acid sequences, wherein the one or more amino acid sequences further comprises one or more additional sequences up to the full length amino acid sequence of a source protein and optionally wherein the second one or more amino acid sequences are expanded sequences of the one or more amino acids.

64.-65. (canceled)

66. The system of claim 51, wherein the one or more amino acid sequences are of 7 to 100, 7 to 75, 7 to 50, or 7 to 25 amino acids in length;
wherein the one or more amino acid sequences have a maximum overlap of 10, 9, 8, 7, 6, or 5 amino acids;
wherein the one or more peptide binding motifs is between 5 to 100, 5 to 75, 5 to 50, 5 to 25 amino acids in length;
wherein the immunogenic peptide comprising one or more peptide binding motifs is 12 to 100, 12 to 75, 12 to 50, 12 to 25, or around 20 amino acids in length;
wherein the ligand region further comprises an additional 25 amino acids on either or both sides of the generated ligand region; and
or any combination thereof.

67.-71. (canceled)

72. The system of claim 51, wherein the deployed machine learning network and second deployed machine learning network independently comprises linear classifiers, logistic classifiers, Bayesian networks, random forest, neural networks, matrix factorization, hidden Markov model, support vector machine, K-means clustering, or K-nearest neighbor.

73. The system of claim 50, wherein the deployed machine learning network comprises a neural network, optionally wherein the neural network comprises a convolutional neural network, and optionally wherein the deployed machine learning network comprises embedding.

74.-75. (canceled)

76. The system of claim 72, wherein the second deployed machine learning network comprises a neural network, optionally wherein the neural network comprises a convolutional neural network or a recurrent neural network, and optionally wherein the deployed machine learning network comprises embedding.

77.-79. (canceled)

80. The system of claim 51, wherein the deployed machine learning network and second deployed machine learning network independently comprises unsupervised learning, supervised learning, semi-supervised learning, reinforcement learning, transfer learning, incremental learning, curriculum learning, and learning to learn,
optionally wherein the machine learning network in (b) is trained on HLA-II allele-specific peptidomics data;
optionally wherein the second machine learning network is trained on full length amino acid sequences of the source proteins mapped back from HLA-II allele-specific peptidomics data, thereby generating the one or more regions of full-length proteins affecting the one or more peptide binding motifs; and
optionally wherein training the deployed machine learning network comprises decoys at a 4:1, 5:1, or 6:1 ratio to immunogenic peptides.

81.-84. (canceled)

85. The system of claim 51, wherein the second deployed machine learning network comprises bi-directional long-short term memory (LSTM), optionally wherein the bi-directional LSTM is performed more than once.

86. (canceled)

87. The system of claim 51, wherein the deployed machine learning network further comprising pooling, reduction, dropout steps, or any combination thereof and/or wherein parameters of the deployed machine learning network and/or the second deployed machine learning network is tuned to minimize binary entropy loss.

88. (canceled)

89. The system of claim 50, wherein the immunogenic peptide comprising peptide binding motifs are specific to HLA-II alleles specific to a subject;
wherein the one or more amino acid sequences comprise full-length protein sequences;
wherein the one or more amino acid sequences are obtained by analyzing one or more genomic DNA sequences, optionally wherein the one or more genomic DNA sequences is a full genome sequence;
or any combination thereof.

90.-92. (canceled)

93. The system of claim 89, wherein the one or more genome sequences is derived from a target pathogen, a commensal microorganism, or a diseased cell, optionally wherein the pathogen is selected from the group consisting of a bacterium, a virus, a protozoon, and an allergen, and optionally wherein the diseased cell is a cancer cell.

94.-95. (canceled)

96. The system of claim 50, wherein the one or more amino acid sequences are derived from neoantigens.

97.-98. (canceled)

99. A computer program product, comprising:
a non-transitory computer-readable storage device having computer-executable program instructions embodied thereon that when executed by a computer cause the computer to generate one or more immunogenic peptides comprising one or more peptide binding motifs for use in immunological compositions, the computer-executable program instructions comprising:
a) computer-executable program instructions to receive, by an acquisition engine communicatively coupled to a user device, one or more amino acid sequences;
b) computer-executable program instructions to transfer the one or more amino acid sequences with the acquisition engine communicatively coupled to a deployed machine learning network;
c) computer-executable program instructions to process the one or more amino acid sequences with the deployed machine learning network, the deployed machine learning network generated and deployed from a training machine learning network and communicatively coupled to the acquisition engine; and
d) computer-executable program instructions to generate one or more immunogenic peptides comprising one or more immunogenic peptide binding motifs.

100. The product of claim 99, wherein processing of step c) further comprises:
e) computer-executable program instructions to receive, by an acquisition engine communicatively coupled to a user device, a second one or more amino acid sequences;
f) transfer the second one or more amino acid sequences with the acquisition engine communicatively coupled to a second deployed machine learning network;
g) process the second one or more amino acid sequences with the second deployed machine learning network, the second deployed machine learning network generated and deployed from a second training machine learning network;
h) generate one or more ligand regions of the second one or more amino acid sequences;
optionally further comprising:
i) transfer the one or more immunogenic peptide comprising one or more peptide binding motifs and the one or more ligand regions with the acquisition engine communicatively coupled to an ensemble network;
j) process the one or more peptide binding motifs and the one or more ligand regions with the ensemble network, optionally wherein the ensemble network comprises grid search;
k) generate a refined set of the one or more immunogenic peptide comprising one or more peptide binding motifs; and
optionally further comprising prepare one or more immunogenic peptide comprising one or more peptides for an immunological composition, optionally wherein the immunological composition is a protective vaccine or tolerizing vaccine composition comprising one or more antigenic epitopes, optionally wherein the immunological composition is a protective vaccine or tolerizing vaccine composition comprising one or more antigenic epitopes and/or
optionally further comprising detect whether one or more of the antigenic epitopes is present in a sample from a subject suffering from an infection, autoimmune disease, allergy, or cancer.

101.-102. (canceled)

103. The product of claim 100, wherein the deployed machine learning network and/or second deployed machine learning networks receive one or more features of the one or more amino acid sequences, optionally wherein the one or more features comprise binary, fractional, or both features, optionally wherein the fractional features comprise secreted protein features, transmembrane features, domain features, region features, relative solvent accessibility features, and disorder features.

104.-105. (canceled)

106. The product of claim 100, wherein the generated one or more immunogenic peptide binding motifs further comprise individual probability or confidence scores, optionally wherein the generated one or more ligand regions further comprise individual probability or confidence scores at each position in the ligand region;
wherein the one or more immunogenic peptide binding motifs is specific for one or more HLA II alleles selected from the group consisting of those in Table 1;
or any combination thereof.

107.-108. (canceled)

109. The product of claim 100, wherein the second one or more amino acid sequences comprise signaling regions, optionally wherein the signaling regions comprise adjacent and/or distant signaling regions, and optionally wherein the adjacent signaling regions comprise exopeptidase trimming sites and/or proline-rich cleavage motifs.

110.-111. (canceled)

112. The product of claim 100, wherein the second one or more amino acid sequences comprises the full length amino acid sequence of a protein, or wherein the second one or more amino acid sequences comprises the one or more amino acid sequences, wherein the one or more amino acid sequences further comprises one or more additional sequences up to the full length amino acid sequence of a source protein and optionally wherein the second one or more amino acid sequences are expanded sequences of the one or more amino acids.

113.-114. (canceled)

115. The product of claim 100, wherein the one or more amino acid sequences are of 7 to 100, 7 to 75, 7 to 50, or 7 to 25 amino acids in length;
wherein the one or more amino acid sequences have a maximum overlap of 10, 9, 8, 7, 6, or 5 amino acids;
wherein the one or more peptide binding motifs is between 5 to 100, 5 to 75, 5 to 50, 5 to 25 amino acids in length;
wherein the immunogenic peptide comprising one or more peptide binding motifs is 12 to 100, 12 to 75, 12 to 50, 12 to 25, or around 20 amino acids in length;
wherein the ligand region further comprises an additional 25 amino acids on either or both sides of the generated ligand region; and
or any combination thereof.

116.-120. (canceled)

121. The product of claim 100, wherein the deployed machine learning network and second deployed machine learning network independently comprises linear classifiers, logistic classifiers, Bayesian networks, random forest, neural networks, matrix factorization, hidden Markov model, support vector machine, K-means clustering, or K-nearest neighbor.

122. The product of claim 99, wherein the deployed machine learning network comprises a neural network, optionally wherein the neural network comprises a convolutional neural network, and optionally wherein the deployed machine learning network comprises embedding.

123.-124. (canceled)

125. The product of claim 121, wherein the second deployed machine learning network comprises a neural network, optionally wherein the neural network comprises a convolutional neural network or a recurrent neural network, and optionally wherein the deployed machine learning network comprises embedding.

126.-128. (canceled)

129. The product of claim 100, wherein the deployed machine learning network and second deployed machine learning network independently comprises unsupervised learning, supervised learning, semi-supervised learning, reinforcement learning, transfer learning, incremental learning, curriculum learning, and learning to learn,
optionally wherein the machine learning network in (b) is trained on HLA-II allele-specific peptidomics data;
optionally wherein the second machine learning network is trained on full length amino acid sequences of the source proteins mapped back from HLA-II allele-specific peptidomics data, thereby generating the one or more regions of full-length proteins affecting the one or more peptide binding motifs; and
optionally wherein training the deployed machine learning network comprises decoys at a 4:1, 5:1, or 6:1 ratio to immunogenic peptides.

130.-133. (canceled)

134. The product of claim 100, wherein the second deployed machine learning network comprises bi-directional long-short term memory (LSTM), optionally wherein the bi-directional LSTM is performed more than once.

135. (canceled)

136. The product of claim 100, wherein the deployed machine learning network further comprising pooling, reduction, dropout steps, or any combination thereof and/or wherein parameters of the deployed machine learning network and/or the second deployed machine learning network is tuned to minimize binary entropy loss.

137. (canceled)

138. The product of claim 99, wherein the immunogenic peptide comprising peptide binding motifs are specific to HLA-II alleles specific to a subject;
wherein the one or more amino acid sequences comprise full-length protein sequences;
wherein the one or more amino acid sequences are obtained by analyzing one or more genomic DNA sequences, optionally wherein the one or more genomic DNA sequences is a full genome sequence;
or any combination thereof.

139.-141. (canceled)

142. The product of claim 138, wherein the one or more genome sequences is derived from a target pathogen, a commensal microorganism, or a diseased cell, optionally wherein the pathogen is selected from the group consisting of a bacterium, a virus, a protozoon, and an allergen, and optionally wherein the diseased cell is a cancer cell.

143.-144. (canceled)

145. The product of claim 99, wherein the one or more amino acid sequences are derived from neoantigens.

146.-147. (canceled)
